SEQUENCE LISTING (226 sequences)

SEQ ID NO: 1 - DNA, 135 bp, synthetic construct
  source 1..135: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 2 - protein, 45 aa, synthetic construct
TTTPAPRPPTPAPTIASQPLSLRPEACRPAAGGAVHTRGLDFACD

SEQ ID NO: 3 - DNA, 63 bp, synthetic construct
  source 1..63: mol_type = other DNA
atctacatctgggcgcccttggccgggacttgtggggtccttctcctgtcactggttatcacc

SEQ ID NO: 4 - protein, 21 aa, synthetic construct
IYIWAPLAGTCGVLLLSLVIT

SEQ ID NO: 5 - DNA, 111 bp, synthetic construct
  source 1..111: mol_type = other DNA
cggagggaccagaggctgccccccgatgcccacaagccccctgggggaggcagtttccggacccccatccaagaggagcaggccgacgcccactccaccctggccaagatc

SEQ ID NO: 6 - protein, 37 aa, synthetic construct
RRDQRLPPDAHKPPGGGSFRTPIQEEQADAHSTLAKI

SEQ ID NO: 7 - DNA, 336 bp, synthetic construct
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 8 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - DNA, 63 bp, synthetic construct
  source 1..63: mol_type = other DNA
ggctctggcgagggaaggggttccctgcttacttgcggcgacgtcgaagagaatcccggtccg

SEQ ID NO: 10 - protein, 21 aa, synthetic construct
GSGEGRGSLLTCGDVEENPGP

SEQ ID NO: 11 - DNA, 342 bp, synthetic construct
  source 1..342: mol_type = other DNA
aactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtcc

SEQ ID NO: 12 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 13 - DNA, 207 bp, synthetic construct
  source 1..207: mol_type = other DNA
actaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgc

SEQ ID NO: 14 - protein, 66 aa, synthetic construct
TTTPAPRPPTPAPTIASQPLSLRPEACRPAAGGAVHTRGLDFACDIYIWAPLAGTCGVLLLSLVIT

SEQ ID NO: 15 - DNA, 30 bp, synthetic construct
  source 1..30: mol_type = other DNA
ggcggtggtggctctggtggtggcggcagc

SEQ ID NO: 16 - protein, 10 aa, synthetic construct
GGGGSGGGGS

SEQ ID NO: 17 - DNA, 30 bp, synthetic construct
  source 1..30: mol_type = other DNA
ggccaggccggctccggaggaggaggatcc

SEQ ID NO: 18 - protein, 10 aa, synthetic construct
GQAGSGGGGS

SEQ ID NO: 19 - DNA, 12 bp, synthetic construct
  source 1..12: mol_type = other DNA
ggccaggccggc

SEQ ID NO: 20 - protein, 4 aa, synthetic construct
GQAG

SEQ ID NO: 21 - DNA, 33 bp, synthetic construct
  source 1..33: mol_type = other DNA
ggcggcggcggtagcggtggtggcggctccgga

SEQ ID NO: 22 - protein, 11 aa, synthetic construct
GGGGSGGGGSG

SEQ ID NO: 23 - DNA, 735 bp, synthetic construct
  source 1..735: mol_type = other DNA
caggtcaccttgaaggagtctggtcctgtgctggtgaaacccacagagaccctcacgctgacctgcaccgtctctgggttctcactcagtaatgctagaatgggtgtgacctggatccgtcagcccccagggaaggccctggagtggcttgcacacattttttcgaatgacgaaaaatcctacagtacatctctgaagagcaggctcaccatctccaaggacacttccaaaacccaggtggtccttaccatgaccaacatggaccctgtggacacagccacatattactgtgcacggatacgagattactatgacattagtagttattatgactactggggccagggaaccctggtcagcgtctcctcaggcggtggtggctctggtggtggcggcagcggcggtggtggctctgacatccagatgacccagtctccatctgccatgtctgcatctgtaggagacagagtcaccatcacttgtcgggcgagtcaggacattagcaattatttagcctggtttcagcagaaaccagggaaagtccctaagcgcctgatctatgctgcatccagtttgcaaagtggggtcccatcaaggttcagcggcagtggatcggggacagagttcactctcacaatcagcagcctgctgcctgaagattttgcaacttattactgtctacagcttaatagtttcccgttcacttttggcggagggaccaaggtggagatcaac

SEQ ID NO: 24 - DNA, 750 bp, synthetic construct
  source 1..750: mol_type = other DNA
gaagtgcagcttgtccagagcggagccgaagtgaagaagcctggcgagagcctgaagatcagctgcaagggctcaggttacggattcacaagttattggataggttgggtgcgccagatgcctggtaagggactggaatggatgggtatcattcatcccgatgatagcgacaccaaatacagcccaagctttcagggccaggtcacaatcagcgctgacaagagcatcagcaccgcctaccttcagtggtcgtctctgaaggccagcgacaccgcaatgtactactgtgcctctagctatttgcgtggcttgtggggaggctattttgactattggggccaaggtacactggtcaccgtcagcagcggcggtggtggctctggtggtggcggcagcggcggtggtggctctgagctccagagcgtgctgacccagcctcctagcgcaagcggcacccctggacagcgtgtgacaattagctgtagcggatcaagctcaaacattggctcaaattatgtgaattggtatcagcaattgcccggtacagcacccaaactgctcatttatggagattatcaacgacctagcggagtgcctgatcgttttagcggtagcaaaagcggcaccagcgcatcactggcaatttcaggcctgcgtagcgaagatgaggcggattattactgtgctacccgcgacgattcgttatctgggtctgtcgtttttggcaccggtacaaaactgaccgtgctg

SEQ ID NO: 25 - protein, 245 aa, synthetic construct
QVTLKESGPVLVKPTETLTLTCTVSGFSLSNARMGVTWIRQPPGKALEWLAHIFSNDEKSYSTSLKSRLTISKDTSKTQVVLTMTNMDPVDTATYYCARIRDYYDISSYYDYWGQGTLVSVSSGGGGSGGGGSGGGGSDIQMTQSPSAMSASVGDRVTITCRASQDISNYLAWFQQKPGKVPKRLIYAASSLQSGVPSRFSGSGSGTEFTLTISSLLPEDFATYYCLQLNSFPFTFGGGTKVEIN

SEQ ID NO: 26 - protein, 250 aa, synthetic construct
EVQLVQSGAEVKKPGESLKISCKGSGYGFTSYWIGWVRQMPGKGLEWMGIIHPDDSDTKYSPSFQGQVTISADKSISTAYLQWSSLKASDTAMYYCASSYLRGLWGGYFDYWGQGTLVTVSSGGGGSGGGGSGGGGSELQSVLTQPPSASGTPGQRVTISCSGSSSNIGSNYVNWYQQLPGTAPKLLIYGDYQRPSGVPDRFSGSKSGTSASLAISGLRSEDEADYYCATRDDSLSGSVVFGTGTKLTVL

SEQ ID NO: 27 - DNA, 612 bp, synthetic construct
  source 1..612: mol_type = other DNA
atggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgc

SEQ ID NO: 28 - protein, 204 aa, synthetic construct
MALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 29 - protein, 8 aa, synthetic construct
DYKDDDDK

SEQ ID NO: 30 - DNA, 732 bp, synthetic construct
  source 1..732: mol_type = other DNA
caggtgcagctggtgcagtctggggctgaggtgaagaagcctggggcctcagtgaaggtttcctgcaaggcatctggatacaccttcaccgattactatatgcactgggtgcgacaggcccctggacaagggcttgagtggatgggaataatcaaccctaatagtggtggaacaaattacgcacagaagttccagggcagagtcaccatgaccagggacacgtccacgagcacagtctacatggagctgagcagcctgagatctgaggacacggccgtgtattactgtgctagagattttagtgtgtatgctgctgcagatggcatggatgtctggggccaagggacattggtcaccgtctcttcaggtggaggcggttcaggcggaggtggctctggcggtggcggatcggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgacgcatccaatttggccaccggggtcccatcaaggttcagtggcagtggatctgggacagatttcactctcaccatcagcagtctgcaacctgaagattttgcaacttactactgtcaacagagtgatagttcacctctcactttcggccaagggaccaaggtggagatcaaa

SEQ ID NO: 31 - DNA, 723 bp, synthetic construct
  source 1..723: mol_type = other DNA
caggtgcagctggtgcagtctggggctgaggtgaagaagcctggggcctcagtgaaggtttcctgcaaggcatctggatacaccttcaccagctacgacattaattgggtgcgacaggcccctggacaagggcttgagtggatgggagtgatcaaccctagtggtggaggtacaagctacgcacagaagttccagggcagagtcaccatgaccagggacacgtccacgagcacagtctacatggagctgagcagcctgagatctgaggacacggccgtgtattactgtgctagatattctggctatgattgggtgtttgactactggggccaagggaccctggtcaccgtctcctcaggtggaggcggttcaggcggaggtggctctggcggtggcggatcggacatccagatgacccagtctccatcctccctgtctgcatctgtaggagacagagtcaccatcacttgccgggcaagtcagggcattggcagctggttagcttggtatcagcagaaaccagggaaagcccctaagctcctgatctatgctgcatccacattgcaaagtggggtcccatcaaggttcagtggcagtggatctgggacagatttcactctcaccatcagcagtctgcaacctgaagattttgcaacttactactgtcaacagagttacagtaccccttggactttcggccctgggaccaaagtggatatcaaa

SEQ ID NO: 32 - DNA, 732 bp, synthetic construct
  source 1..732: mol_type = other DNA
caggtgcagctggtgcagtctggggctgaggtgaagaagcctggggcctcagtgaaggtttcctgcaaggcatctggatacaccttcaccagctacgacatcaattgggtgcgacaggcccctggacaagggcttgagtggatgggagtcatcagccctagtggtggtggaacaagctacgcacagaagttccagggcagagtcaccatgaccagggacacgtccacgagcacagtctacatggagctgagcagcctgagatctgaggacacggccgtgtattactgtgctagatactacggcggcaaccccgaagggtggtacttcgatctctggggccgtggcaccctggtcactgtctcctcaggtggaggcggttcaggcggaggtggctctggcggtggcggatcggacatccagatgacccagtctccatcctccctgtctgcatctgtaggagacagagtcaccatcacttgccgggcaagtcagagcattggaaattggttagcctggtatcagcagaaaccagggaaagcccctaagctcctgatctatgacgcatccgatttggaaaccggggtcccatcaaggttcagtggcagtggatctgggacagatttcactctcaccatcagcagtctgcaacctgaagattttgcaacttactactgtcaacagagttacactaccccttggacgttcggccaagggaccaaggtggaaatcaaa

SEQ ID NO: 33 - DNA, 54 bp, synthetic construct
  source 1..54: mol_type = other DNA
ggcagcaccagcggcagcggcaagcccggctccggagagggcagcaccaagggc

SEQ ID NO: 34 - DNA, 732 bp, synthetic construct
  source 1..732: mol_type = other DNA
gacatccagatgacccagtctccatcctccctgtctgcatctgtaggagacagagtcaccatcacttgccgggcaagtcagggcattggcagctggttagcttggtatcagcagaaaccagggaaagcccctaagctcctgatctatgctgcatccacattgcaaagtggggtcccatcaaggttcagtggcagtggatctgggacagatttcactctcaccatcagcagtctgcaacctgaagattttgcaacttactactgtcaacagagttacagtaccccttggactttcggccctgggaccaaagtggatatcaaaggcagcaccagcggcagcggcaagcccggctccggagagggcagcaccaagggccaggtgcagctggtgcagtctggggctgaggtgaagaagcctggggcctcagtgaaggtttcctgcaaggcatctggatacaccttcaccagctacgacattaattgggtgcgacaggcccctggacaagggcttgagtggatgggagtgatcaaccctagtggtggaggtacaagctacgcacagaagttccagggcagagtcaccatgaccagggacacgtccacgagcacagtctacatggagctgagcagcctgagatctgaggacacggccgtgtattactgtgctagatattctggctatgattgggtgtttgactactggggccaagggaccctggtcaccgtctcctca

SEQ ID NO: 35 - DNA, 741 bp, synthetic construct
  source 1..741: mol_type = other DNA
gacatccagatgacccagtctccatcctccctgtctgcatctgtaggagacagagtcaccatcacttgccgggcaagtcagagcattggaaattggttagcctggtatcagcagaaaccagggaaagcccctaagctcctgatctatgacgcatccgatttggaaaccggggtcccatcaaggttcagtggcagtggatctgggacagatttcactctcaccatcagcagtctgcaacctgaagattttgcaacttactactgtcaacagagttacactaccccttggacgttcggccaagggaccaaggtggaaatcaaaggcagcaccagcggcagcggcaagcccggctccggagagggcagcaccaagggccaggtgcagctggtgcagtctggggctgaggtgaagaagcctggggcctcagtgaaggtttcctgcaaggcatctggatacaccttcaccagctacgacatcaattgggtgcgacaggcccctggacaagggcttgagtggatgggagtcatcagccctagtggtggtggaacaagctacgcacagaagttccagggcagagtcaccatgaccagggacacgtccacgagcacagtctacatggagctgagcagcctgagatctgaggacacggccgtgtattactgtgctagatactacggcggcaaccccgaagggtggtacttcgatctctggggccgtggcaccctggtcactgtctcctca

SEQ ID NO: 36 - DNA, 741 bp, synthetic construct
  source 1..741: mol_type = other DNA
gacatagttatgacccaatccccagatagtttggcggtttctctgggcgagagggcaacgattaattgtcgcgcatcaaagagcgtttcaacgagcggatattcttttatgcattggtaccagcaaaaacccggacaaccgccgaagctgctgatctacttggcttcaaatcttgagtctggggtgccggaccgattttctggtagtggaagcggaactgactttacgctcacgatcagttcactgcaggctgaggatgtagcggtctattattgccagcacagtagagaagtcccctggaccttcggtcaaggcacgaaagtagaaattaaaggcagcaccagcggcagcggcaagcccggctccggagagggcagcaccaagggccaggtccagttggtgcaaagcggggcggaggtgaaaaaacccggcgcttccgtgaaggtgtcctgtaaggcgtccggttatacgttcacgaactacgggatgaattgggttcgccaagcgccggggcagggactgaaatggatggggtggataaatacctacaccggcgaacctacatacgccgacgcttttaaagggcgagtcactatgacgcgcgataccagcatatccaccgcatacatggagctgtcccgactccggtcagacgacacggctgtctactattgtgctcgggactatggcgattatggcatggactactggggtcagggtacgactgtaacagttagtagt

SEQ ID NO: 37 - DNA, 732 bp, synthetic construct
  source 1..732: mol_type = other DNA
caggtccagttggtgcaaagcggggcggaggtgaaaaaacccggcgcttccgtgaaggtgtcctgtaaggcgtccggttatacgttcacgaactacgggatgaattgggttcgccaagcgccggggcagggactgaaatggatggggtggataaatacctacaccggcgaacctacatacgccgacgcttttaaagggcgagtcactatgacgcgcgataccagcatatccaccgcatacatggagctgtcccgactccggtcagacgacacggctgtctactattgtgctcgggactatggcgattatggcatggactactggggtcagggtacgactgtaacagttagtagtggtggaggcggttcaggcggaggtggctctggcggtggcggatcggacatagttatgacccaatccccagatagtttggcggtttctctgggcgagagggcaacgattaattgtcgcgcatcaaagagcgtttcaacgagcggatattcttttatgcattggtaccagcaaaaacccggacaaccgccgaagctgctgatctacttggcttcaaatcttgagtctggggtgccggaccgattttctggtagtggaagcggaactgactttacgctcacgatcagttcactgcaggctgaggatgtagcggtctattattgccagcacagtagagaagtcccctggaccttcggtcaaggcacgaaagtagaaattaaa

SEQ ID NO: 38 - DNA, 2181 bp, synthetic construct
  source 1..2181: mol_type = other DNA
atggccttaccagtgaccgccttgctcctgccgctggccttgctgctccacgccgccaggccggactacaaagacgatgacgataaaggcggtggtggctctggtggtggcggatcccaggtgcagctggtgcagtctggggctgaggtgaagaagcctggggcctcagtgaaggtttcctgcaaggcatctggatacaccttcaccgattactatatgcactgggtgcgacaggcccctggacaagggcttgagtggatgggaataatcaaccctaatagtggtggaacaaattacgcacagaagttccagggcagagtcaccatgaccagggacacgtccacgagcacagtctacatggagctgagcagcctgagatctgaggacacggccgtgtattactgtgctagagattttagtgtgtatgctgctgcagatggcatggatgtctggggccaagggacattggtcaccgtctcttcaggtggaggcggttcaggcggaggtggctctggcggtggcggatcggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgacgcatccaatttggccaccggggtcccatcaaggttcagtggcagtggatctgggacagatttcactctcaccatcagcagtctgcaacctgaagattttgcaacttactactgtcaacagagtgatagttcacctctcactttcggccaagggaccaaggtggagatcaaaaccacgacgccagcgccgcggccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaa

SEQ ID NO: 39 - DNA, 2172 bp, synthetic construct
  source 1..2172: mol_type = other DNA
atggccttaccagtgaccgccttgctcctgccgctggccttgctgctccacgccgccaggccggactacaaagacgatgacgataaaggcggtggtggctctggtggtggcggatcccaggtgcagctggtgcagtctggggctgaggtgaagaagcctggggcctcagtgaaggtttcctgcaaggcatctggatacaccttcaccagctacgacattaattgggtgcgacaggcccctggacaagggcttgagtggatgggagtgatcaaccctagtggtggaggtacaagctacgcacagaagttccagggcagagtcaccatgaccagggacacgtccacgagcacagtctacatggagctgagcagcctgagatctgaggacacggccgtgtattactgtgctagatattctggctatgattgggtgtttgactactggggccaagggaccctggtcaccgtctcctcaggtggaggcggttcaggcggaggtggctctggcggtggcggatcggacatccagatgacccagtctccatcctccctgtctgcatctgtaggagacagagtcaccatcacttgccgggcaagtcagggcattggcagctggttagcttggtatcagcagaaaccagggaaagcccctaagctcctgatctatgctgcatccacattgcaaagtggggtcccatcaaggttcagtggcagtggatctgggacagatttcactctcaccatcagcagtctgcaacctgaagattttgcaacttactactgtcaacagagttacagtaccccttggactttcggccctgggaccaaagtggatatcaaaaccacgacgccagcgccgcggccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaa

SEQ ID NO: 40 - DNA, 2181 bp, synthetic construct
  source 1..2181: mol_type = other DNA
atggccttaccagtgaccgccttgctcctgccgctggccttgctgctccacgccgccaggccggactacaaagacgatgacgataaaggcggtggtggctctggtggtggcggatcccaggtgcagctggtgcagtctggggctgaggtgaagaagcctggggcctcagtgaaggtttcctgcaaggcatctggatacaccttcaccagctacgacatcaattgggtgcgacaggcccctggacaagggcttgagtggatgggagtcatcagccctagtggtggtggaacaagctacgcacagaagttccagggcagagtcaccatgaccagggacacgtccacgagcacagtctacatggagctgagcagcctgagatctgaggacacggccgtgtattactgtgctagatactacggcggcaaccccgaagggtggtacttcgatctctggggccgtggcaccctggtcactgtctcctcaggtggaggcggttcaggcggaggtggctctggcggtggcggatcggacatccagatgacccagtctccatcctccctgtctgcatctgtaggagacagagtcaccatcacttgccgggcaagtcagagcattggaaattggttagcctggtatcagcagaaaccagggaaagcccctaagctcctgatctatgacgcatccgatttggaaaccggggtcccatcaaggttcagtggcagtggatctgggacagatttcactctcaccatcagcagtctgcaacctgaagattttgcaacttactactgtcaacagagttacactaccccttggacgttcggccaagggaccaaggtggaaatcaaaaccacgacgccagcgccgcggccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaa

SEQ ID NO: 41 - DNA, 2133 bp, synthetic construct
  source 1..2133: mol_type = other DNA
atggccttaccagtgaccgccttgctcctgccgctggccttgctgctccacgccgccaggccggacatccagatgacccagtctccatcctccctgtctgcatctgtaggagacagagtcaccatcacttgccgggcaagtcagggcattggcagctggttagcttggtatcagcagaaaccagggaaagcccctaagctcctgatctatgctgcatccacattgcaaagtggggtcccatcaaggttcagtggcagtggatctgggacagatttcactctcaccatcagcagtctgcaacctgaagattttgcaacttactactgtcaacagagttacagtaccccttggactttcggccctgggaccaaagtggatatcaaaggcagcaccagcggcagcggcaagcccggctccggagagggcagcaccaagggccaggtgcagctggtgcagtctggggctgaggtgaagaagcctggggcctcagtgaaggtttcctgcaaggcatctggatacaccttcaccagctacgacattaattgggtgcgacaggcccctggacaagggcttgagtggatgggagtgatcaaccctagtggtggaggtacaagctacgcacagaagttccagggcagagtcaccatgaccagggacacgtccacgagcacagtctacatggagctgagcagcctgagatctgaggacacggccgtgtattactgtgctagatattctggctatgattgggtgtttgactactggggccaagggaccctggtcaccgtctcctcagcggccgcaaccacgacgccagcgccgcggccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgc

SEQ ID NO: 42 - DNA, 2142 bp, synthetic construct
  source 1..2142: mol_type = other DNA
atggccttaccagtgaccgccttgctcctgccgctggccttgctgctccacgccgccaggccggacatccagatgacccagtctccatcctccctgtctgcatctgtaggagacagagtcaccatcacttgccgggcaagtcagagcattggaaattggttagcctggtatcagcagaaaccagggaaagcccctaagctcctgatctatgacgcatccgatttggaaaccggggtcccatcaaggttcagtggcagtggatctgggacagatttcactctcaccatcagcagtctgcaacctgaagattttgcaacttactactgtcaacagagttacactaccccttggacgttcggccaagggaccaaggtggaaatcaaaggcagcaccagcggcagcggcaagcccggctccggagagggcagcaccaagggccaggtgcagctggtgcagtctggggctgaggtgaagaagcctggggcctcagtgaaggtttcctgcaaggcatctggatacaccttcaccagctacgacatcaattgggtgcgacaggcccctggacaagggcttgagtggatgggagtcatcagccctagtggtggtggaacaagctacgcacagaagttccagggcagagtcaccatgaccagggacacgtccacgagcacagtctacatggagctgagcagcctgagatctgaggacacggccgtgtattactgtgctagatactacggcggcaaccccgaagggtggtacttcgatctctggggccgtggcaccctggtcactgtctcctcagcggccgcaaccacgacgccagcgccgcggccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgc

SEQ ID NO: 43 - DNA, 2142 bp, synthetic construct
  source 1..2142: mol_type = other DNA
atggccttaccagtgaccgccttgctcctgccgctggccttgctgctccacgccgccaggccggacatagttatgacccaatccccagatagtttggcggtttctctgggcgagagggcaacgattaattgtcgcgcatcaaagagcgtttcaacgagcggatattcttttatgcattggtaccagcaaaaacccggacaaccgccgaagctgctgatctacttggcttcaaatcttgagtctggggtgccggaccgattttctggtagtggaagcggaactgactttacgctcacgatcagttcactgcaggctgaggatgtagcggtctattattgccagcacagtagagaagtcccctggaccttcggtcaaggcacgaaagtagaaattaaaggcagcaccagcggcagcggcaagcccggctccggagagggcagcaccaagggccaggtccagttggtgcaaagcggggcggaggtgaaaaaacccggcgcttccgtgaaggtgtcctgtaaggcgtccggttatacgttcacgaactacgggatgaattgggttcgccaagcgccggggcagggactgaaatggatggggtggataaatacctacaccggcgaacctacatacgccgacgcttttaaagggcgagtcactatgacgcgcgataccagcatatccaccgcatacatggagctgtcccgactccggtcagacgacacggctgtctactattgtgctcgggactatggcgattatggcatggactactggggtcagggtacgactgtaacagttagtagtgcggccgcaaccacgacgccagcgccgcggccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgc

SEQ ID NO: 44 - DNA, 2115 bp, synthetic construct
  source 1..2115: mol_type = other DNA
atggccttaccagtgaccgccttgctcctgccgctggccttgctgctccacgccgccaggccgcaggtgcagctggtgcagtctggggctgaggtgaagaagcctggggcctcagtgaaggtttcctgcaaggcatctggatacaccttcaccagctacgacattaattgggtgcgacaggcccctggacaagggcttgagtggatgggagtgatcaaccctagtggtggaggtacaagctacgcacagaagttccagggcagagtcaccatgaccagggacacgtccacgagcacagtctacatggagctgagcagcctgagatctgaggacacggccgtgtattactgtgctagatattctggctatgattgggtgtttgactactggggccaagggaccctggtcaccgtctcctcaggtggaggcggttcaggcggaggtggctctggcggtggcggatcggacatccagatgacccagtctccatcctccctgtctgcatctgtaggagacagagtcaccatcacttgccgggcaagtcagggcattggcagctggttagcttggtatcagcagaaaccagggaaagcccctaagctcctgatctatgctgcatccacattgcaaagtggggtcccatcaaggttcagtggcagtggatctgggacagatttcactctcaccatcagcagtctgcaacctgaagattttgcaacttactactgtcaacagagttacagtaccccttggactttcggccctgggaccaaagtggatatcaaaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgc

SEQ ID NO: 45 - DNA, 2124 bp, synthetic construct
  source 1..2124: mol_type = other DNA
atggccttaccagtgaccgccttgctcctgccgctggccttgctgctccacgccgccaggccgcaggtgcagctggtgcagtctggggctgaggtgaagaagcctggggcctcagtgaaggtttcctgcaaggcatctggatacaccttcaccagctacgacatcaattgggtgcgacaggcccctggacaagggcttgagtggatgggagtcatcagccctagtggtggtggaacaagctacgcacagaagttccagggcagagtcaccatgaccagggacacgtccacgagcacagtctacatggagctgagcagcctgagatctgaggacacggccgtgtattactgtgctagatactacggcggcaaccccgaagggtggtacttcgatctctggggccgtggcaccctggtcactgtctcctcaggtggaggcggttcaggcggaggtggctctggcggtggcggatcggacatccagatgacccagtctccatcctccctgtctgcatctgtaggagacagagtcaccatcacttgccgggcaagtcagagcattggaaattggttagcctggtatcagcagaaaccagggaaagcccctaagctcctgatctatgacgcatccgatttggaaaccggggtcccatcaaggttcagtggcagtggatctgggacagatttcactctcaccatcagcagtctgcaacctgaagattttgcaacttactactgtcaacagagttacactaccccttggacgttcggccaagggaccaaggtggaaatcaaaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgc

SEQ ID NO: 46 - DNA, 2124 bp, synthetic construct
  source 1..2124: mol_type = other DNA
atggccttaccagtgaccgccttgctcctgccgctggccttgctgctccacgccgccaggccgcaggtccagttggtgcaaagcggggcggaggtgaaaaaacccggcgcttccgtgaaggtgtcctgtaaggcgtccggttatacgttcacgaactacgggatgaattgggttcgccaagcgccggggcagggactgaaatggatggggtggataaatacctacaccggcgaacctacatacgccgacgcttttaaagggcgagtcactatgacgcgcgataccagcatatccaccgcatacatggagctgtcccgactccggtcagacgacacggctgtctactattgtgctcgggactatggcgattatggcatggactactggggtcagggtacgactgtaacagttagtagtggtggaggcggttcaggcggaggtggctctggcggtggcggatcggacatagttatgacccaatccccagatagtttggcggtttctctgggcgagagggcaacgattaattgtcgcgcatcaaagagcgtttcaacgagcggatattcttttatgcattggtaccagcaaaaacccggacaaccgccgaagctgctgatctacttggcttcaaatcttgagtctggggtgccggaccgattttctggtagtggaagcggaactgactttacgctcacgatcagttcactgcaggctgaggatgtagcggtctattattgccagcacagtagagaagtcccctggaccttcggtcaaggcacgaaagtagaaattaaaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgc

SEQ ID NO: 47 - protein, 244 aa, synthetic construct
QVQLVQSGAEVKKPGASVKVSCKASGYTFTDYYMHWVRQAPGQGLEWMGIINPNSGGTNYAQKFQGRVTMTRDTSTSTVYMELSSLRSEDTAVYYCARDFSVYAAADGMDVWGQGTLVTVSSGGGGSGGGGSGGGGSDIQMTQSPSSLSASVGDRVTITCRASQSISSYLNWYQQKPGKAPKLLIYDASNLATGVPSRFSGSGSGTDFTLTISSLQPEDFATYYCQQSDSSPLTFGQGTKVEIK

SEQ ID NO: 48 - protein, 241 aa, synthetic construct
QVQLVQSGAEVKKPGASVKVSCKASGYTFTSYDINWVRQAPGQGLEWMGVINPSGGGTSYAQKFQGRVTMTRDTSTSTVYMELSSLRSEDTAVYYCARYSGYDWVFDYWGQGTLVTVSSGGGGSGGGGSGGGGSDIQMTQSPSSLSASVGDRVTITCRASQGIGSWLAWYQQKPGKAPKLLIYAASTLQSGVPSRFSGSGSGTDFTLTISSLQPEDFATYYCQQSYSTPWTFGPGTKVDIK

SEQ ID NO: 49 - protein, 244 aa, synthetic construct
QVQLVQSGAEVKKPGASVKVSCKASGYTFTSYDINWVRQAPGQGLEWMGVISPSGGGTSYAQKFQGRVTMTRDTSTSTVYMELSSLRSEDTAVYYCARYYGGNPEGWYFDLWGRGTLVTVSSGGGGSGGGGSGGGGSDIQMTQSPSSLSASVGDRVTITCRASQSIGNWLAWYQQKPGKAPKLLIYDASDLETGVPSRFSGSGSGTDFTLTISSLQPEDFATYYCQQSYTTPWTFGQGTKVEIK

SEQ ID NO: 50 - protein, 18 aa, synthetic construct
GSTSGSGKPGSGEGSTKG

SEQ ID NO: 51 - protein, 244 aa, synthetic construct
DIQMTQSPSSLSASVGDRVTITCRASQGIGSWLAWYQQKPGKAPKLLIYAASTLQSGVPSRFSGSGSGTDFTLTISSLQPEDFATYYCQQSYSTPWTFGPGTKVDIKGSTSGSGKPGSGEGSTKGQVQLVQSGAEVKKPGASVKVSCKASGYTFTSYDINWVRQAPGQGLEWMGVINPSGGGTSYAQKFQGRVTMTRDTSTSTVYMELSSLRSEDTAVYYCARYSGYDWVFDYWGQGTLVTVSS

SEQ ID NO: 52 - protein, 247 aa, synthetic construct
DIQMTQSPSSLSASVGDRVTITCRASQSIGNWLAWYQQKPGKAPKLLIYDASDLETGVPSRFSGSGSGTDFTLTISSLQPEDFATYYCQQSYTTPWTFGQGTKVEIKGSTSGSGKPGSGEGSTKGQVQLVQSGAEVKKPGASVKVSCKASGYTFTSYDINWVRQAPGQGLEWMGVISPSGGGTSYAQKFQGRVTMTRDTSTSTVYMELSSLRSEDTAVYYCARYYGGNPEGWYFDLWGRGTLVTVSS

SEQ ID NO: 53 - protein, 247 aa, synthetic construct
DIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIKGSTSGSGKPGSGEGSTKGQVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSS

SEQ ID NO: 54 - protein, 244 aa, synthetic construct
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSSGGGGSGGGGSGGGGSDIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIK

SEQ ID NO: 55 - protein, 726 aa, synthetic construct
MALPVTALLLPLALLLHAARPDYKDDDDKGGGGSGGGGSQVQLVQSGAEVKKPGASVKVSCKASGYTFTDYYMHWVRQAPGQGLEWMGIINPNSGGTNYAQKFQGRVTMTRDTSTSTVYMELSSLRSEDTAVYYCARDFSVYAAADGMDVWGQGTLVTVSSGGGGSGGGGSGGGGSDIQMTQSPSSLSASVGDRVTITCRASQSISSYLNWYQQKPGKAPKLLIYDASNLATGVPSRFSGSGSGTDFTLTISSLQPEDFATYYCQQSDSSPLTFGQGTKVEIKTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 56 - protein, 723 aa, synthetic construct
MALPVTALLLPLALLLHAARPDYKDDDDKGGGGSGGGGSQVQLVQSGAEVKKPGASVKVSCKASGYTFTSYDINWVRQAPGQGLEWMGVINPSGGGTSYAQKFQGRVTMTRDTSTSTVYMELSSLRSEDTAVYYCARYSGYDWVFDYWGQGTLVTVSSGGGGSGGGGSGGGGSDIQMTQSPSSLSASVGDRVTITCRASQGIGSWLAWYQQKPGKAPKLLIYAASTLQSGVPSRFSGSGSGTDFTLTISSLQPEDFATYYCQQSYSTPWTFGPGTKVDIKTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 57 - protein, 726 aa, synthetic construct
MALPVTALLLPLALLLHAARPDYKDDDDKGGGGSGGGGSQVQLVQSGAEVKKPGASVKVSCKASGYTFTSYDINWVRQAPGQGLEWMGVISPSGGGTSYAQKFQGRVTMTRDTSTSTVYMELSSLRSEDTAVYYCARYYGGNPEGWYFDLWGRGTLVTVSSGGGGSGGGGSGGGGSDIQMTQSPSSLSASVGDRVTITCRASQSIGNWLAWYQQKPGKAPKLLIYDASDLETGVPSRFSGSGSGTDFTLTISSLQPEDFATYYCQQSYTTPWTFGQGTKVEIKTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 58 - protein, 711 aa, synthetic construct
MALPVTALLLPLALLLHAARPDIQMTQSPSSLSASVGDRVTITCRASQGIGSWLAWYQQKPGKAPKLLIYAASTLQSGVPSRFSGSGSGTDFTLTISSLQPEDFATYYCQQSYSTPWTFGPGTKVDIKGSTSGSGKPGSGEGSTKGQVQLVQSGAEVKKPGASVKVSCKASGYTFTSYDINWVRQAPGQGLEWMGVINPSGGGTSYAQKFQGRVTMTRDTSTSTVYMELSSLRSEDTAVYYCARYSGYDWVFDYWGQGTLVTVSSAAATTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 59 - protein, 714 aa, synthetic construct
MALPVTALLLPLALLLHAARPDIQMTQSPSSLSASVGDRVTITCRASQSIGNWLAWYQQKPGKAPKLLIYDASDLETGVPSRFSGSGSGTDFTLTISSLQPEDFATYYCQQSYTTPWTFGQGTKVEIKGSTSGSGKPGSGEGSTKGQVQLVQSGAEVKKPGASVKVSCKASGYTFTSYDINWVRQAPGQGLEWMGVISPSGGGTSYAQKFQGRVTMTRDTSTSTVYMELSSLRSEDTAVYYCARYYGGNPEGWYFDLWGRGTLVTVSSAAATTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 60 - protein, 714 aa, synthetic construct
MALPVTALLLPLALLLHAARPDIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIKGSTSGSGKPGSGEGSTKGQVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSSAAATTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 61 - protein, 705 aa, synthetic construct
MALPVTALLLPLALLLHAARPQVQLVQSGAEVKKPGASVKVSCKASGYTFTSYDINWVRQAPGQGLEWMGVINPSGGGTSYAQKFQGRVTMTRDTSTSTVYMELSSLRSEDTAVYYCARYSGYDWVFDYWGQGTLVTVSSGGGGSGGGGSGGGGSDIQMTQSPSSLSASVGDRVTITCRASQGIGSWLAWYQQKPGKAPKLLIYAASTLQSGVPSRFSGSGSGTDFTLTISSLQPEDFATYYCQQSYSTPWTFGPGTKVDIKTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 62 - protein, 708 aa, synthetic construct
MALPVTALLLPLALLLHAARPQVQLVQSGAEVKKPGASVKVSCKASGYTFTSYDINWVRQAPGQGLEWMGVISPSGGGTSYAQKFQGRVTMTRDTSTSTVYMELSSLRSEDTAVYYCARYYGGNPEGWYFDLWGRGTLVTVSSGGGGSGGGGSGGGGSDIQMTQSPSSLSASVGDRVTITCRASQSIGNWLAWYQQKPGKAPKLLIYDASDLETGVPSRFSGSGSGTDFTLTISSLQPEDFATYYCQQSYTTPWTFGQGTKVEIKTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 63 - protein, 708 aa, synthetic construct
MALPVTALLLPLALLLHAARPQVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSSGGGGSGGGGSGGGGSDIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIKTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 64 - protein, 501 aa, synthetic construct
MALPVTALLLPLALLLHAARPDYKDDDDKGGGGSGGGGSQVQLVQSGAEVKKPGASVKVSCKASGYTFTDYYMHWVRQAPGQGLEWMGIINPNSGGTNYAQKFQGRVTMTRDTSTSTVYMELSSLRSEDTAVYYCARDFSVYAAADGMDVWGQGTLVTVSSGGGGSGGGGSGGGGSDIQMTQSPSSLSASVGDRVTITCRASQSISSYLNWYQQKPGKAPKLLIYDASNLATGVPSRFSGSGSGTDFTLTISSLQPEDFATYYCQQSDSSPLTFGQGTKVEIKTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 65 - protein, 498 aa, synthetic construct
MALPVTALLLPLALLLHAARPDYKDDDDKGGGGSGGGGSQVQLVQSGAEVKKPGASVKVSCKASGYTFTSYDINWVRQAPGQGLEWMGVINPSGGGTSYAQKFQGRVTMTRDTSTSTVYMELSSLRSEDTAVYYCARYSGYDWVFDYWGQGTLVTVSSGGGGSGGGGSGGGGSDIQMTQSPSSLSASVGDRVTITCRASQGIGSWLAWYQQKPGKAPKLLIYAASTLQSGVPSRFSGSGSGTDFTLTISSLQPEDFATYYCQQSYSTPWTFGPGTKVDIKTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 66 - protein, 501 aa, synthetic construct
MALPVTALLLPLALLLHAARPDYKDDDDKGGGGSGGGGSQVQLVQSGAEVKKPGASVKVSCKASGYTFTSYDINWVRQAPGQGLEWMGVISPSGGGTSYAQKFQGRVTMTRDTSTSTVYMELSSLRSEDTAVYYCARYYGGNPEGWYFDLWGRGTLVTVSSGGGGSGGGGSGGGGSDIQMTQSPSSLSASVGDRVTITCRASQSIGNWLAWYQQKPGKAPKLLIYDASDLETGVPSRFSGSGSGTDFTLTISSLQPEDFATYYCQQSYTTPWTFGQGTKVEIKTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - protein, 486 aa, synthetic construct
MALPVTALLLPLALLLHAARPDIQMTQSPSSLSASVGDRVTITCRASQGIGSWLAWYQQKPGKAPKLLIYAASTLQSGVPSRFSGSGSGTDFTLTISSLQPEDFATYYCQQSYSTPWTFGPGTKVDIKGSTSGSGKPGSGEGSTKGQVQLVQSGAEVKKPGASVKVSCKASGYTFTSYDINWVRQAPGQGLEWMGVINPSGGGTSYAQKFQGRVTMTRDTSTSTVYMELSSLRSEDTAVYYCARYSGYDWVFDYWGQGTLVTVSSAAATTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - protein, 489 aa, synthetic construct
MALPVTALLLPLALLLHAARPDIQMTQSPSSLSASVGDRVTITCRASQSIGNWLAWYQQKPGKAPKLLIYDASDLETGVPSRFSGSGSGTDFTLTISSLQPEDFATYYCQQSYTTPWTFGQGTKVEIKGSTSGSGKPGSGEGSTKGQVQLVQSGAEVKKPGASVKVSCKASGYTFTSYDINWVRQAPGQGLEWMGVISPSGGGTSYAQKFQGRVTMTRDTSTSTVYMELSSLRSEDTAVYYCARYYGGNPEGWYFDLWGRGTLVTVSSAAATTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 69 - protein, 489 aa, synthetic construct
MALPVTALLLPLALLLHAARPDIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIKGSTSGSGKPGSGEGSTKGQVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSSAAATTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 70 - protein, 480 aa, synthetic construct
MALPVTALLLPLALLLHAARPQVQLVQSGAEVKKPGASVKVSCKASGYTFTSYDINWVRQAPGQGLEWMGVINPSGGGTSYAQKFQGRVTMTRDTSTSTVYMELSSLRSEDTAVYYCARYSGYDWVFDYWGQGTLVTVSSGGGGSGGGGSGGGGSDIQMTQSPSSLSASVGDRVTITCRASQGIGSWLAWYQQKPGKAPKLLIYAASTLQSGVPSRFSGSGSGTDFTLTISSLQPEDFATYYCQQSYSTPWTFGPGTKVDIKTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 71 - protein, 483 aa, synthetic construct
MALPVTALLLPLALLLHAARPQVQLVQSGAEVKKPGASVKVSCKASGYTFTSYDINWVRQAPGQGLEWMGVISPSGGGTSYAQKFQGRVTMTRDTSTSTVYMELSSLRSEDTAVYYCARYYGGNPEGWYFDLWGRGTLVTVSSGGGGSGGGGSGGGGSDIQMTQSPSSLSASVGDRVTITCRASQSIGNWLAWYQQKPGKAPKLLIYDASDLETGVPSRFSGSGSGTDFTLTISSLQPEDFATYYCQQSYTTPWTFGQGTKVEIKTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 72 - protein, 483 aa, synthetic construct
MALPVTALLLPLALLLHAARPQVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSSGGGGSGGGGSGGGGSDIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIKTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 73 - protein, 26 aa, synthetic construct
QVQLVQSGAEVKKPGASVKVSCKASG

SEQ ID NO: 74 - protein, 26 aa, synthetic construct
QVQLVQSGAEVKKPGSSVKVSCKASG

SEQ ID NO: 75 - protein, 26 aa, synthetic construct
EVQLLESGGGLVQPGGSLRLSCAASG

SEQ ID NO: 76 - protein, 26 aa, synthetic construct
EVQLVESGGGLVKPGGSLRLSCAASG

SEQ ID NO: 77 - protein, 13 aa, synthetic construct
WVRQAPGQGLEWM

SEQ ID NO: 78 - protein, 13 aa, synthetic construct
WVRQAPGQGLEWV

SEQ ID NO: 79 - protein, 13 aa, synthetic construct
WVRQAPGKGLEWV

SEQ ID NO: 80 - protein, 13 aa, synthetic construct
WVRQAPGQGLEWI

SEQ ID NO: 81 - protein, 34 aa, synthetic construct
QKFQGRVTMTRDTSTSTVYMELSSLRSEDTAVYY

SEQ ID NO: 82 - protein, 34 aa, synthetic construct
QRFQGRVTMTRDTSTSTVYMELSSLRSEDTAVYY

SEQ ID NO: 83 - protein, 34 aa, synthetic construct
HKFQGRVTMTRDTSTSTVYMELSSLRSEDTAVYY

SEQ ID NO: 84 - protein, 34 aa, synthetic construct
QKFQGRVTITADESTSTAYMELSSLRSEDTAVYY

SEQ ID NO: 85 - protein, 34 aa, synthetic construct
LKFQGRVTMTRDTSTSTVYMELSSLRSEDTAVYY

SEQ ID NO: 86 - protein, 34 aa, synthetic construct
QNFQGRVTMTRDTSTSTVYMELSSLRSEDTAVYY

SEQ ID NO: 87 - protein, 34 aa, synthetic construct
QMFQGRVTMTRDTSTSTVYMELSSLRSEDTAVYY

SEQ ID NO: 88 - protein, 34 aa, synthetic construct
QEFQGRVTMTRDTSTSTVYMELSSLRSEDTAVYY

SEQ ID NO: 89 - protein, 34 aa, synthetic construct
EEFQGRVTITADESTSTAYMELSSLRSEDTAVYY

SEQ ID NO: 90 - protein, 34 aa, synthetic construct
QTFQGRVTMTRDTSTSTVYMELSSLRSEDTAVYY

SEQ ID NO: 91 - protein, 34 aa, synthetic construct
QSFQGRVTMTRDTSTSTVYMELSSLRSEDTAVYY

SEQ ID NO: 92 - protein, 34 aa, synthetic construct
DSVKGRFTISRDNSKNTLYLQMNSLRAEDTAVYY

SEQ ID NO: 93 - protein, 34 aa, synthetic construct
QNLQGRVTMTRDTSTSTVYMELSSLRSEDTAVYY

SEQ ID NO: 94 - protein, 34 aa, synthetic construct
PKFQGRVTITADESTSTAYMELSSLRSEDTAVYY

SEQ ID NO: 95 - protein, 34 aa, synthetic construct
DSVKGRFTISRDDSKNTLYLQMNSLKTEDTAVYY

SEQ ID NO: 96 - protein, 34 aa, synthetic construct
DKFQGRVTMTRDTSTSTVYMELSSLRSEDTAVYY

SEQ ID NO: 97 - protein, 10 aa, synthetic construct
GQGTLVTVSS

SEQ ID NO: 98 - protein, 10 aa, synthetic construct
GRGTLVTVSS

SEQ ID NO: 99 - protein, 10 aa, synthetic construct
GQGTTVTVSS

SEQ ID NO: 100 - protein, 10 aa, synthetic construct
GQGTMVTVSS

SEQ ID NO: 101 - protein, 10 aa, synthetic construct
GKGTTVTVSS

SEQ ID NO: 102 - protein, 9 aa, synthetic construct
YTFTDYYMH

SEQ ID NO: 103 - protein, 9 aa, synthetic construct
YTFTSYDIN

SEQ ID NO: 104 - protein, 13 aa, synthetic construct
GIINPNSGGTNYA

SEQ ID NO: 105 - protein, 13 aa, synthetic construct
GVINPSGGGTSYA

SEQ ID NO: 106 - protein, 13 aa, synthetic construct
GVISPSGGGTSYA

SEQ ID NO: 107 - protein, 17 aa, synthetic construct
CARDFSVYAAADGMDVW

SEQ ID NO: 108 - protein, 14 aa, synthetic construct
CARYSGYDWVFDYW

SEQ ID NO: 109 - protein, 17 aa, synthetic construct
CARYYGGNPEGWYFDLW

SEQ ID NO: 110 - protein, 5 aa, synthetic construct
NYGMN

SEQ ID NO: 111 - protein, 17 aa, synthetic construct
WINTYTGEPTYADAFKG

SEQ ID NO: 112 - protein, 7 aa, synthetic construct
GDYGMDY

SEQ ID NO: 113 - protein, 23 aa, synthetic construct
DIQMTQSPSSLSASVGDRVTITC

SEQ ID NO: 114 - protein, 23 aa, synthetic construct
DIVMTQSPLSLPVTPGEPASISC

SEQ ID NO: 115 - protein, 23 aa, synthetic construct
EIVMTQSPATLSVSPGERATLSC

SEQ ID NO: 116 - protein, 23 aa, synthetic construct
DIVMTQSPDSLAVSLGERATINC

SEQ ID NO: 117 - protein, 15 aa, synthetic construct
WYQQKPGKAPKLLIY

SEQ ID NO: 118 - protein, 15 aa, synthetic construct
WYLQKPGQSPQLLIY

SEQ ID NO: 119 - protein, 15 aa, synthetic construct
WYQQKPGQAPRLLIY

SEQ ID NO: 120 - protein, 15 aa, synthetic construct
WYQQKPGQPPKLLIY

SEQ ID NO: 121 - protein, 31 aa, synthetic construct
GVPSRFSGSGSGTDFTLTISSLQPEDFATYY

SEQ ID NO: 122 - protein, 31 aa, synthetic construct
GVPDRFSGSGSGTDFTLKISRVEAEDVGVYY

SEQ ID NO: 123 - protein, 31 aa, synthetic construct
GIPARFSGSGSGTEFTLTISSLQSEDFAVYY

SEQ ID NO: 124 - protein, 31 aa, synthetic construct
GVPDRFSGSGSGTDFTLTISSLQAEDVAVYY

SEQ ID NO: 125 - protein, 9 aa, synthetic construct
GQGTRLEIK

SEQ ID NO: 126 - protein, 9 aa, synthetic construct
GQGTKLEIK

SEQ ID NO: 127 - protein, 9 aa, synthetic construct
GQGTKVEIK

SEQ ID NO: 128 - protein, 9 aa, synthetic construct
GGGTKVEIK

SEQ ID NO: 129 - protein, 9 aa, synthetic construct
GPGTKVDIK

SEQ ID NO: 130 - protein, 9 aa, synthetic construct
GGGTKLEIK

SEQ ID NO: 131 - protein, 11 aa, synthetic construct
RASQSISSYLN

SEQ ID NO: 132 - protein, 11 aa, synthetic construct
RASQGIGSWLA

SEQ ID NO: 133 - protein, 11 aa, synthetic construct
RASQSIGNWLA

SEQ ID NO: 134 - protein, 7 aa, synthetic construct
DASNLAT

SEQ ID NO: 135 - protein, 7 aa, synthetic construct
AASTLQS

SEQ ID NO: 136 - protein, 7 aa, synthetic construct
DASDLET

SEQ ID NO: 137 - protein, 11 aa, synthetic construct [sequence omitted]

SEQ ID NO: 138 - protein, 11 aa, synthetic construct [sequence omitted]

SEQ ID NO: 139 - protein, 11 aa, synthetic construct [sequence omitted]

SEQ ID NO: 140 - protein, 15 aa, synthetic construct
RASKSVSTSGYSFMH

SEQ ID NO: 141 - protein, 7 aa, synthetic construct
LASNLES

SEQ ID NO: 142 - protein, 9 aa, synthetic construct
QHSREVPWT

SEQ ID NO: 143 - DNA, 366 bp, synthetic construct
  source 1..366: mol_type = other DNA
caggtgcagctggtgcagtctggggctgaggtgaagaagcctggggcctcagtgaaggtttcctgcaaggcatctggatacaccttcaccgattactatatgcactgggtgcgacaggcccctggacaagggcttgagtggatgggaataatcaaccctaatagtggtggaacaaattacgcacagaagttccagggcagagtcaccatgaccagggacacgtccacgagcacagtctacatggagctgagcagcctgagatctgaggacacggccgtgtattactgtgctagagattttagtgtgtatgctgctgcagatggcatggatgtctggggccaagggacattggtcaccgtctcttca

SEQ ID NO: 144 - DNA, 357 bp, synthetic construct
  source 1..357: mol_type = other DNA
caggtgcagctggtgcagtctggggctgaggtgaagaagcctggggcctcagtgaaggtttcctgcaaggcatctggatacaccttcaccagctacgacattaattgggtgcgacaggcccctggacaagggcttgagtggatgggagtgatcaaccctagtggtggaggtacaagctacgcacagaagttccagggcagagtcaccatgaccagggacacgtccacgagcacagtctacatggagctgagcagcctgagatctgaggacacggccgtgtattactgtgctagatattctggctatgattgggtgtttgactactggggccaagggaccctggtcaccgtctcctca

SEQ ID NO: 145 - DNA, 366 bp, synthetic construct
  source 1..366: mol_type = other DNA
caggtgcagctggtgcagtctggggctgaggtgaagaagcctggggcctcagtgaaggtttcctgcaaggcatctggatacaccttcaccagctacgacatcaattgggtgcgacaggcccctggacaagggcttgagtggatgggagtcatcagccctagtggtggtggaacaagctacgcacagaagttccagggcagagtcaccatgaccagggacacgtccacgagcacagtctacatggagctgagcagcctgagatctgaggacacggccgtgtattactgtgctagatactacggcggcaaccccgaagggtggtacttcgatctctggggccgtggcaccctggtcactgtctcctca

SEQ ID NO: 146 - DNA, 321 bp, synthetic construct
  source 1..321: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgacgcatccaatttggccaccggggtcccatcaaggttcagtggcagtggatctgggacagatttcactctcaccatcagcagtctgcaacctgaagattttgcaacttactactgtcaacagagtgatagttcacctctcactttcggccaagggaccaaggtggagatcaaa

SEQ ID NO: 147 - DNA, 321 bp, synthetic construct
  source 1..321: mol_type = other DNA
gacatccagatgacccagtctccatcctccctgtctgcatctgtaggagacagagtcaccatcacttgccgggcaagtcagggcattggcagctggttagcttggtatcagcagaaaccagggaaagcccctaagctcctgatctatgctgcatccacattgcaaagtggggtcccatcaaggttcagtggcagtggatctgggacagatttcactctcaccatcagcagtctgcaacctgaagattttgcaacttactactgtcaacagagttacagtaccccttggactttcggccctgggaccaaagtggatatcaaa

SEQ ID NO: 148 - DNA, 321 bp, synthetic construct
  source 1..321: mol_type = other DNA
gacatccagatgacccagtctccatcctccctgtctgcatctgtaggagacagagtcaccatcacttgccgggcaagtcagagcattggaaattggttagcctggtatcagcagaaaccagggaaagcccctaagctcctgatctatgacgcatccgatttggaaaccggggtcccatcaaggttcagtggcagtggatctgggacagatttcactctcaccatcagcagtctgcaacctgaagattttgcaacttactactgtcaacagagttacactaccccttggacgttcggccaagggaccaaggtggaaatcaaa

SEQ ID NO: 149 - DNA, 354 bp, synthetic construct
  source 1..354: mol_type = other DNA
caggtccagttggtgcaaagcggggcggaggtgaaaaaacccggcgcttccgtgaaggtgtcctgtaaggcgtccggttatacgttcacgaactacgggatgaattgggttcgccaagcgccggggcagggactgaaatggatggggtggataaatacctacaccggcgaacctacatacgccgacgcttttaaagggcgagtcactatgacgcgcgataccagcatatccaccgcatacatggagctgtcccgactccggtcagacgacacggctgtctactattgtgctcgggactatggcgattatggcatggactactggggtcagggtacgactgtaacagttagtagt

SEQ ID NO: 150 - DNA, 333 bp, synthetic construct
  source 1..333: mol_type = other DNA
gacatagttatgacccaatccccagatagtttggcggtttctctgggcgagagggcaacgattaattgtcgcgcatcaaagagcgtttcaacgagcggatattcttttatgcattggtaccagcaaaaacccggacaaccgccgaagctgctgatctacttggcttcaaatcttgagtctggggtgccggaccgattttctggtagtggaagcggaactgactttacgctcacgatcagttcactgcaggctgaggatgtagcggtctattattgccagcacagtagagaagtcccctggaccttcggtcaaggcacgaaagtagaaattaaa

SEQ ID NO: 151 - protein, 122 aa, synthetic construct
QVQLVQSGAEVKKPGASVKVSCKASGYTFTDYYMHWVRQAPGQGLEWMGIINPNSGGTNYAQKFQGRVTMTRDTSTSTVYMELSSLRSEDTAVYYCARDFSVYAAADGMDVWGQGTLVTVSS

SEQ ID NO: 152 - protein, 119 aa, synthetic construct
QVQLVQSGAEVKKPGASVKVSCKASGYTFTSYDINWVRQAPGQGLEWMGVINPSGGGTSYAQKFQGRVTMTRDTSTSTVYMELSSLRSEDTAVYYCARYSGYDWVFDYWGQGTLVTVSS

SEQ ID NO: 153 - protein, 122 aa, synthetic construct
QVQLVQSGAEVKKPGASVKVSCKASGYTFTSYDINWVRQAPGQGLEWMGVISPSGGGTSYAQKFQGRVTMTRDTSTSTVYMELSSLRSEDTAVYYCARYYGGNPEGWYFDLWGRGTLVTVSS

SEQ ID NO: 154 - protein, 107 aa, synthetic construct
DIQMTQSPSSLSASVGDRVTITCRASQSISSYLNWYQQKPGKAPKLLIYDASNLATGVPSRFSGSGSGTDFTLTISSLQPEDFATYYCQQSDSSPLTFGQGTKVEIK

SEQ ID NO: 155 - protein, 107 aa, synthetic construct
DIQMTQSPSSLSASVGDRVTITCRASQGIGSWLAWYQQKPGKAPKLLIYAASTLQSGVPSRFSGSGSGTDFTLTISSLQPEDFATYYCQQSYSTPWTFGPGTKVDIK

SEQ ID NO: 156 - protein, 107 aa, synthetic construct
DIQMTQSPSSLSASVGDRVTITCRASQSIGNWLAWYQQKPGKAPKLLIYDASDLETGVPSRFSGSGSGTDFTLTISSLQPEDFATYYCQQSYTTPWTFGQGTKVEIK

SEQ ID NO: 157 - protein, 118 aa, synthetic construct
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSS

SEQ ID NO: 158 - protein, 111 aa, synthetic construct
DIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIK

SEQ ID NO: 159 - DNA, 20 bp, synthetic construct
  source 1..20: mol_type = other DNA
ccgatcgtgaagcgcctgct

SEQ ID NO: 160 - DNA, 20 bp, synthetic construct
  source 1..20: mol_type = other DNA
cgagaaggcggtcaagagcc

SEQ ID NO: 161 - DNA, 20 bp, synthetic construct
  source 1..20: mol_type = other DNA
cttggtgttgacgttctgcg

SEQ ID NO: 162 - DNA, 20 bp, synthetic construct
  source 1..20: mol_type = other DNA
ctctgctgtttcaccatcca

SEQ ID NO: 163 - DNA, 20 bp, synthetic construct
  source 1..20: mol_type = other DNA
cccggcttgggcggtgctcc

SEQ ID NO: 164 - DNA, 20 bp, synthetic construct
  source 1..20: mol_type = other DNA
cgactaccagttgtccaagc

SEQ ID NO: 165 - DNA, 20 bp, synthetic construct
  source 1..20: mol_type = other DNA
gactgagttattggcgtggc

SEQ ID NO: 166 - DNA, 20 bp, synthetic construct
  source 1..20: mol_type = other DNA
gaatgttccattgataagcc

SEQ ID NO: 167 - DNA, 20 bp, synthetic construct
  source 1..20: mol_type = other DNA
gagaggctgaggtttgcccc

SEQ ID NO: 168 - DNA, 20 bp, synthetic construct
  source 1..20: mol_type = other DNA
cctcaccatgcctagccttt

SEQ ID NO: 169 - DNA, 20 bp, synthetic construct
  source 1..20: mol_type = other DNA
cgatctggggtgggagttcg

SEQ ID NO: 170 - DNA, 20 bp, synthetic construct
  source 1..20: mol_type = other DNA
gtttgaccgctccacgagcc

SEQ ID NO: 171 - DNA, 20 bp, synthetic construct
  source 1..20: mol_type = other DNA
cccctaccatgactttattc

SEQ ID NO: 172 - DNA, 20 bp, synthetic construct
  source 1..20: mol_type = other DNA
attgcactcatcagagctac

SEQ ID NO: 173 - DNA, 20 bp, synthetic construct
  source 1..20: mol_type = other DNA
agtcatggtaggggagcttg

SEQ ID NO: 174 - DNA, 20 bp, synthetic construct
  source 1..20: mol_type = other DNA
tgctggcgatacgcgtccac

SEQ ID NO: 175 - DNA, 20 bp, synthetic construct
  source 1..20: mol_type = other DNA
gtgagcaatcccccgggcga

SEQ ID NO: 176 - DNA, 20 bp, synthetic construct
  source 1..20: mol_type = other DNA
aacgtgcggtgggatcgtgc

SEQ ID NO: 177 - DNA, 23 bp, synthetic construct
  source 1..23: mol_type = other DNA
tcaccaagcccgcgaccaatggg

SEQ ID NO: 178 - DNA, 23 bp, synthetic construct
  source 1..23: mol_type = other DNA
gctttggtcccattggtcgcggg

SEQ ID NO: 179 - DNA, 23 bp, synthetic construct
  source 1..23: mol_type = other DNA
accctcctccggcatcgccgcgg

SEQ ID NO: 180 - DNA, 20 bp, synthetic construct
  source 1..20: mol_type = other DNA
gctttggtcccattggtcgc

SEQ ID NO: 181 - DNA, 20 bp, synthetic construct
  source 1..20: mol_type = other DNA
ctcaccagattcccgaaggt

SEQ ID NO: 182 - DNA, 20 bp, synthetic construct
  source 1..20: mol_type = other DNA
ccgccttgtcatcaaccgtc

SEQ ID NO: 183 - DNA, 20 bp, synthetic construct
  source 1..20: mol_type = other DNA
tctgcgttcaggggtaagcg

SEQ ID NO: 184 - DNA, 20 bp, synthetic construct
  source 1..20: mol_type = other DNA
gcgcttacccctgaacgcag

SEQ ID NO: 185 - DNA, 20 bp, synthetic construct
  source 1..20: mol_type = other DNA
cgcagaggaccatgtccccg

SEQ ID NO: 186 - DNA, 20 bp, synthetic construct
  source 1..20: mol_type = other DNA
agattcccgaaggtaggaga

SEQ ID NO: 187 - DNA, 20 bp, synthetic construct
  source 1..20: mol_type = other DNA
ctggtattggggttccatta

SEQ ID NO: 188 - DNA, 20 bp, synthetic construct
  source 1..20: mol_type = other DNA
tactcaatgcgtacattggt

SEQ ID NO: 189 - DNA, 20 bp, synthetic construct
  source 1..20: mol_type = other DNA
atactcaatgcgtacattgg

SEQ ID NO: 190 - DNA, 20 bp, synthetic construct
  source 1..20: mol_type = other DNA
tccggaaaggccaggatgcg

SEQ ID NO: 191 - DNA, 20 bp, synthetic construct
  source 1..20: mol_type = other DNA
cttgtccaggccacgcatcc

SEQ ID NO: 192 - DNA, 23 bp, synthetic construct
  source 1..23: mol_type = other DNA
taatctggtggacctcatgaagg

SEQ ID NO: 193 - DNA, 23 bp, synthetic construct
  source 1..23: mol_type = other DNA
tcggttggcaaacgtccgaaagg

SEQ ID NO: 194 - DNA, 23 bp, synthetic construct
  source 1..23: mol_type = other DNA
agcaagctgccgcagatcgcagg

SEQ ID NO: 195 - DNA, 20 bp, synthetic construct
  source 1..20: mol_type = other DNA
agcaagctgccgcagatcgc

SEQ ID NO: 196 - DNA, 20 bp, synthetic construct
  source 1..20: mol_type = other DNA
ggagctgatggtaaatctgc

SEQ ID NO: 197 - DNA, 20 bp, synthetic construct
  source 1..20: mol_type = other DNA
ttgaaggtttaattccgcat

SEQ ID NO: 198 - DNA, 20 bp, synthetic construct
  source 1..20: mol_type = other DNA
aacaactatcgttaccacag

SEQ ID NO: 199 - DNA, 20 bp, synthetic construct
  source 1..20: mol_type = other DNA
cgaggagcccatgatgggca

SEQ ID NO: 200 - DNA, 20 bp, synthetic construct
  source 1..20: mol_type = other DNA
gggcaatgtgctggtgtgct

SEQ ID NO: 201 - DNA, 20 bp, synthetic construct
  source 1..20: mol_type = other DNA
cagtgacaccacaaagtagt

SEQ ID NO: 202 - DNA, 23 bp, synthetic construct
  source 1..23: mol_type = other DNA
tcaccaagcccgcgaccaatggg

SEQ ID NO: 203 - DNA, 23 bp, synthetic construct
  source 1..23: mol_type = other DNA
tcaccaagcccgcgaccaatggg

SEQ ID NO: 204 - protein, 6 aa, synthetic construct
QGIGSW

SEQ ID NO: 205 - protein, 8 aa, synthetic construct
GYTFTSYD

SEQ ID NO: 206 - protein, 8 aa, synthetic construct
ISPSGGGT

SEQ ID NO: 207 - protein, 15 aa, synthetic construct
ARYYGGNPEGWYFDL

SEQ ID NO: 208 - protein, 15 aa, synthetic construct
GGGGSGGGGSGGGGS

SEQ ID NO: 209 - protein, 23 aa, synthetic construct
VINPSGGGTSYAQKFQGQSIGNW

SEQ ID NO: 210 - protein, 12 aa, synthetic construct
YSGYDWVFDYDA

SEQ ID NO: 211 - protein, 18 aa, synthetic construct
QQSYSTPWTQQSYTTPWT

SEQ ID NO: 212 - DNA, 23 bp, synthetic construct
  source 1..23: mol_type = other DNA
agtggttcgggcgctggttaccc

SEQ ID NO: 213 - protein, 183 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 214 - protein, 462 aa, synthetic construct
QVQLVQSGAEVKKPGASVKVSCKASGYTFTDYYMHWVRQAPGQGLEWMGIINPNSGGTNYAQKFQGRVTMTRDTSTSTVYMELSSLRSEDTAVYYCARDFSVYAAADGMDVWGQGTLVTVSSGGGGSGGGGSGGGGSDIQMTQSPSSLSASVGDRVTITCRASQSISSYLNWYQQKPGKAPKLLIYDASNLATGVPSRFSGSGSGTDFTLTISSLQPEDFATYYCQQSDSSPLTFGQGTKVEIKTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 215 - protein, 459 aa, synthetic construct
QVQLVQSGAEVKKPGASVKVSCKASGYTFTSYDINWVRQAPGQGLEWMGVINPSGGGTSYAQKFQGRVTMTRDTSTSTVYMELSSLRSEDTAVYYCARYSGYDWVFDYWGQGTLVTVSSGGGGSGGGGSGGGGSDIQMTQSPSSLSASVGDRVTITCRASQGIGSWLAWYQQKPGKAPKLLIYAASTLQSGVPSRFSGSGSGTDFTLTISSLQPEDFATYYCQQSYSTPWTFGPGTKVDIKTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 216 - protein, 462 aa, synthetic construct
QVQLVQSGAEVKKPGASVKVSCKASGYTFTSYDINWVRQAPGQGLEWMGVISPSGGGTSYAQKFQGRVTMTRDTSTSTVYMELSSLRSEDTAVYYCARYYGGNPEGWYFDLWGRGTLVTVSSGGGGSGGGGSGGGGSDIQMTQSPSSLSASVGDRVTITCRASQSIGNWLAWYQQKPGKAPKLLIYDASDLETGVPSRFSGSGSGTDFTLTISSLQPEDFATYYCQQSYTTPWTFGQGTKVEIKTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 217 - protein, 465 aa, synthetic construct
DIQMTQSPSSLSASVGDRVTITCRASQGIGSWLAWYQQKPGKAPKLLIYAASTLQSGVPSRFSGSGSGTDFTLTISSLQPEDFATYYCQQSYSTPWTFGPGTKVDIKGSTSGSGKPGSGEGSTKGQVQLVQSGAEVKKPGASVKVSCKASGYTFTSYDINWVRQAPGQGLEWMGVINPSGGGTSYAQKFQGRVTMTRDTSTSTVYMELSSLRSEDTAVYYCARYSGYDWVFDYWGQGTLVTVSSAAATTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 218 - protein, 468 aa, synthetic construct
DIQMTQSPSSLSASVGDRVTITCRASQSIGNWLAWYQQKPGKAPKLLIYDASDLETGVPSRFSGSGSGTDFTLTISSLQPEDFATYYCQQSYTTPWTFGQGTKVEIKGSTSGSGKPGSGEGSTKGQVQLVQSGAEVKKPGASVKVSCKASGYTFTSYDINWVRQAPGQGLEWMGVISPSGGGTSYAQKFQGRVTMTRDTSTSTVYMELSSLRSEDTAVYYCARYYGGNPEGWYFDLWGRGTLVTVSSAAATTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 219 - protein, 468 aa, synthetic construct
DIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIKGSTSGSGKPGSGEGSTKGQVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSSAAATTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 220 - protein, 459 aa, synthetic construct; identical to SEQ ID NO: 215

SEQ ID NO: 221 - protein, 462 aa, synthetic construct; identical to SEQ ID NO: 216

SEQ ID NO: 222 - protein, 462 aa, synthetic construct
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSSGGGGSGGGGSGGGGSDIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIKTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 223 - skipped

SEQ ID NO: 224 - protein, 9 aa, synthetic construct
QQSYSTPWT

SEQ ID NO: 225 - protein, 8 aa, synthetic construct
INPSGGGT

SEQ ID NO: 226 - protein, 12 aa, synthetic construct
ARYSGYDWVFDY

(19) United States
(12) Patent Application Publication
(10) Pub. No.: US 2023/0390392 A1
(43) Pub. Date: Dec. 7, 2023

(54) MULTIPLEX GENE EDITED CELLS FOR CD70-DIRECTED CANCER IMMUNOTHERAPY

(71) Applicants: Nkarta, Inc., South San Francisco, CA (US); CRISPR THERAPEUTICS AG, Zug (CH)

(72) Inventors: James Barnaby Trager, Albany, CA (US); Ivan Chan, Millbrae, CA (US); Chao Guo, San Francisco, CA (US); Luxuan Guo Buren, San Francisco, CA (US); Alexandra Leida Liana Lazetic, San Jose, CA (US); Mary-Lee Dequéant, South Boston, MA (US); Hanspeter Waldner, South Boston, MA (US); Changan Guo, South Boston, MA (US); Chandirasegaran Massilamany, South Boston, MA (US); Ming-Hong Xie, Foster City, CA (US); Elizabeth N. Koch, South Boston, MA (US); Jacob Usadi, South Boston, MA (US); Parin Sripakdeevong, South Boston, MA (US)

(21) Appl. No.: 18/179,201

(22) Filed: Mar. 6, 2023

Related U.S. Application Data
(60) Provisional application No. 63/268,967, filed on Mar. 7, 2022.

(51) Int. Cl. A61K 39/00; C12N 15/11; C12N 9/22; C12N 15/90; C12N 5/0783; C07K 16/28; C07K 14/725; A61P 35/00

(52) U.S. Cl. CPC A61K 39/4631; C12N 15/11; C12N 9/22; C12N 15/907; C12N 5/0646; C07K 16/2875; C07K 14/7051; A61K 39/4613; A61K 39/464416; A61P 35/00; C12N 2310/20; C12N 2800/80; A61K 2239/13; C07K 2319/03; C07K 2319/02

(57) ABSTRACT

Several embodiments of the methods and compositions disclosed herein relate to immune cells that are engineered to express chimeric antigen receptors (CAR) and/or genetically modified to reduce potential side effects of cellular immunotherapy. Several embodiments relate to genetic modifications to the immune cells, such as Natural Killer (NK) cells, to reduce, substantially, reduce, or eliminate expression of a combination of genes and their corresponding proteins. In several embodiments, one edit is to reduce expression of a marker by the immune cells that would otherwise cause them to be self-targeted by the CAR and at least two additional gene edits to enhance the cytotoxicity and/or persistence of the resulting cells. In several embodiments, the CAR targets CD70, and in some embodiments is used for renal cell carcinoma immunotherapy.

### CROSS REFERENCE TO RELATED APPLICATIONS

This application claims the benefit of priority to U.S. Provisional Patent Application No. 63/268,967, filed Mar. 7, 2022, the entire contents of each of which is incorporated by reference herein.

### FIELD

Several embodiments disclosed herein relate to methods and compositions comprising genetically engineered cells for cancer immunotherapy, in particular cells engineered to have reduced expression of certain markers that are also present on target cells. In several embodiments, the present disclosure relates to cells engineered to express chimeric antigen receptors and have reduced expression of one or more markers that enhance the efficacy, persistence, and/or reduce potential side effects when the cells are used in cancer immunotherapy

### BACKGROUND

As further knowledge is gained about various cancers and what characteristics a cancerous cell has that can be used to specifically distinguish that cell from a healthy cell, therapeutics are under development that leverage the distinct features of a cancerous cell. Immunotherapies that employ engineered immune cells are one approach to treating cancers.

### INCORPORATION BY REFERENCE OF MATERIAL IN SEQUENCE LISTING FILE

This application incorporates by reference the Sequence Listing contained in the following XML file being submitted concurrently herewith: File name: NKT.086A_ST26.xml; created on Mar. 5, 2023 and is 249,856 bytes in size.

### SUMMARY

Immunotherapy presents a new technological advancement in the treatment of disease, wherein immune cells are engineered to express certain targeting and/or effector molecules that specifically identify and react to diseased or damaged cells. This represents a promising advance due, at least in part, to the potential for specifically targeting diseased or damaged cells, as opposed to more traditional approaches, such as chemotherapy, where all cells are impacted, and the desired outcome is that sufficient healthy cells survive to allow the patient to live. One immunotherapy approach is the recombinant expression of chimeric receptors in immune cells to achieve the targeted recognition and destruction of aberrant cells of interest.

Accordingly, provided for herein is a population of genetically engineered natural killer (NK) cells for cancer immunotherapy, comprising a plurality of NK cells engineered to express a chimeric antigen receptor (CAR) comprising a tumor binding domain, a transmembrane domain, and a cytotoxic signaling complex, wherein the tumor binding domain targets CD70 and comprises an scFv comprising an amino acid sequence having at least about 85%, 90%, 95%, 97% (or more) sequence identity to one or more of SEQ ID NOs: 52, 47, 48, 49, 50, 51, 53 or 54, wherein the NK cells comprise a genomic disruption within a CD70 protein gene target sequence that comprises any one of SEQ ID NO: 180 or 177-179, wherein the NK cells also comprise a genomic disruption within of a cytokine-inducible SH2-containing protein gene target sequence that comprises any one of SEQ ID NO: 191 or 186-190, and wherein the NK cells comprise at least one additional genomic disruption within a gene target sequence, and wherein the genetically engineered NK cells comprising said genomic disruptions exhibit one or more of enhanced expansion capability, enhanced cytotoxicity against target cells, and enhanced persistence, as compared to NK cells not comprising said genomic disruptions. In several embodiments, the NK cells have been expanded in culture.

In several embodiments, the NK cells also comprise a genomic disruption within a target sequence of a Casitas B-lineage lymphoma-b (Cbl-b) protein-encoding gene target sequence that comprises any one of SEQ ID NO: 195, 192, 193, or 194. In several embodiments, the genomic disruption within the target sequence of the CD70 protein-encoding gene, the target sequence of the CIS protein-encoding gene, and/or the target sequence of the Cbl-b protein encoding gene comprises an endonuclease-mediated indel. In several embodiments, the plurality of NK cells comprise a genomic disruption within a plurality of protein encoding gene target sequences that comprises at least three of SEQ ID NO: 177-195. In several embodiments, the genomic disruptions within a protein encoding gene target sequence comprise an endonuclease-mediated indel.

Also provided is a population of genetically engineered NK cells for cancer immunotherapy, comprising a plurality of NK cells that have been expanded in culture, wherein the plurality of NK cells are engineered to express a CAR comprising a tumor binding domain, a transmembrane domain, and a cytotoxic signaling complex, wherein the tumor binding domain targets CD70, wherein the NK cells comprise a genomic disruption within a CD70 protein gene target sequence that comprises any one of SEQ ID NO: 177-180, wherein said genomic disruption comprises and endonuclease-mediated indel, wherein the NK cells comprise a genomic disruption within of a cytokine-inducible SH2-containing protein gene target sequence that comprises any one of SEQ ID NO: 186-191, and wherein the NK cells comprise at least one additional genomic disruption within a gene target sequence, and wherein the genetically engineered NK cells comprising said genomic disruptions exhibit one or more of enhanced expansion capability, enhanced cytotoxicity against target cells, and enhanced persistence, as compared to NK cells not comprising said genomic disruptions.

Further, in several embodiments, there is provided a population of genetically engineered NK cells for cancer immunotherapy, comprising a plurality of NK cells that have been expanded in culture, wherein the plurality of NK cells are engineered to express a CAR comprising a tumor binding domain, a transmembrane domain, and a cytotoxic signaling complex, wherein the tumor binding domain targets CD70, wherein the NK cells are genetically edited to express reduced levels of CD70 as compared to a non-edited NK cell that has been expanded in culture, and wherein the reduced CD70 expression was engineered through introducing a genomic disruption in an endogenous CD70 gene, wherein the NK cells are genetically edited to express reduced levels of a cytokine-inducible SH2-containing (CIS) protein encoded by a CISH gene as compared to a non-edited NK cell, wherein the reduced CIS expression was engineered through introducing a genomic disruption in a CISH gene, and wherein the genetically engineered NK cells exhibit one or more of enhanced expansion capability, enhanced cytotoxicity against target cells, and enhanced persistence, as compared to NK cells expressing native levels of CIS, and wherein the NK cells are genetically edited to introduce a genomic disruption in at two or more additional genes to reduce expression of a protein encoded by said two or more additional genes as compared to a NK cell not edited at said genes.

Also provided is a population of genetically engineered NK cells for cancer immunotherapy, comprising a plurality of NK cells engineered to express a CAR comprising a tumor binding domain, a transmembrane domain, and a cytotoxic signaling complex, wherein the tumor binding domain targets CD70 and comprises an scFv comprising an amino acid sequence having at least about 85%, 90%, 95%, 97% (or more) sequence identity to one or more of SEQ ID NOs: 47-49 or 51-54, wherein the plurality of NK cells comprise a genomic disruption within a gene target sequence that comprises at least three of SEQ ID NO: 177-195, optionally wherein said genomic disruption comprises an endonuclease-mediated indel.

In several embodiments, there is provided a method for treating cancer in a subject comprising, administering to the subject a population of genetically engineered immune cells, comprising a plurality of NK cells that have been expanded in culture and engineered to express a CAR comprising a tumor binding domain, a transmembrane domain, and a cytotoxic signaling complex, wherein the tumor binding domain targets CD70 and comprises an scFv comprising an amino acid sequence having at least about 85%, 90%, 95%, 97% (or more) sequence identity to one or more of SEQ ID NOs: 47-49 or 51-54, wherein the NK cells comprise a genomic disruption within a CD70 protein gene target sequence that comprises any one of SEQ ID NO: 177-180, optionally wherein said genomic disruption comprises and endonuclease-mediated indel, wherein the NK cells comprise a genomic disruption within of a cytokine-inducible SH2-containing protein gene target sequence that comprises any one of SEQ ID NO: 186-191, and wherein the NK cells comprise at least one additional genomic disruption within a gene target sequence, and wherein the genetically engineered NK cells comprising said genomic disruptions exhibit one or more of enhanced expansion capability, enhanced cytotoxicity against target cells, and enhanced persistence, as compared to NK cells not comprising said genomic disruptions.

In several embodiments, the tumor binding domain comprises a heavy chain variable region and a light chain variable region, wherein the heavy chain variable region comprises a CDR-H1, CDR-H2, and CDR-H3, and the light chain variable region comprises a CDR-L1, CDR-L2, and CDR-L3, and wherein the CDR-H1 comprises a sequence having at least about 85%, 90%, 95%, 97% (or more) sequence identity to one or more sequences selected from SEQ ID NOs: 205, 102, 103, and 110, the CDR-H2 comprises a sequence having at least about 85%, 90%, 95%, 97% (or more) sequence identity to one or more sequences selected from SEQ ID NOs: 206, 104, 105, 106, and 111, the CDR-H3 comprises a sequence having at least about 85%, 90%, 95%, 97% (or more) sequence identity to one or more sequences selected from SEQ ID NOs: 207, 107, 108, 109, and 112, the CDR-L1 comprises a sequence having at least about 85%, 90%, 95%, 97% (or more) sequence identity to one or more sequences selected from SEQ ID NOs: 209, 131, 132, 133, and 140, the CDR-L2 comprises a sequence having at least about 85%, 90%, 95%, 97% (or more) sequence identity to one or more sequences selected from SEQ ID NOs: 210, 134, 135, 136, and 141, and the CDR-L3 comprises a sequence having at least about 85%, 90%, 95%, 97% (or more) sequence identity to one or more sequences selected from SEQ ID NOs: 211, 137, 138, 139, and 142.

In several embodiments, the tumor binding domain comprises a VH, wherein the VH comprises an amino acid sequence having at least about 85%, 90%, 95%, 97% (or more) sequence identity to one or more of the amino acid sequences of SEQ ID NOs: 153, 151, 152 and 157. In several embodiments, the tumor binding domain comprises a VH, wherein the VH is encoded by a polynucleotide comprising a sequence having at least about 85%, 90%, 95%, 97% (or more) sequence identity to one or more of the polynucleotides of SEQ ID NOs: 145, 143, 144, 146 and 149. In several embodiments, the tumor binding domain comprises a VL, wherein the VL comprises an amino acid sequence having at least about 85%, 90%, 95%, 97% (or more) sequence identity to one or more of the amino acid sequences of SEQ ID NOs: 156, 154, 155 and 158. In several embodiments, the tumor binding domain comprises a VL, wherein the VL is encoded by a polynucleotide comprising a sequence having at least about 85%, 90%, 95%, 97% (or more) sequence identity to one or more of the polynucleotides of SEQ ID NOs: 148, 146, 147 and 150.

In several embodiments, the tumor binding domain comprises a VL and a VH, wherein the VL comprises an amino acid sequence having at least about 85%, 90%, 95%, 97% (or more) sequence identity to the amino acid sequence of SEQ ID NO: 156, wherein the VH comprises an amino acid sequence having at least about 85%, 90%, 95%, 97% (or more) sequence identity to the amino acid sequence of SEQ ID NO: 153. In several embodiments, the tumor binding domain comprises a VL and a VH, wherein the VL comprises an amino acid sequence having at least about 85%, 90%, 95%, 97% (or more) sequence identity to the amino acid sequence of SEQ ID NO: 155, wherein the VH comprises an amino acid sequence having at least about 85%, 90%, 95%, 97% (or more) sequence identity to the amino acid sequence of SEQ ID NO: 152. In several embodiments, the tumor binding domain comprises a VL and a VH, wherein the VL comprises an amino acid sequence having at least about 85%, 90%, 95%, 97% (or more) sequence identity to the amino acid sequence of SEQ ID NO: 157, wherein the VH comprises an amino acid sequence having at least about 85%, 90%, 95%, 97% (or more) sequence identity to the amino acid sequence of SEQ ID NO: 158.

In several embodiments, the tumor binding domain comprises an scFv, wherein the scFv comprises an amino acid sequence having at least about 85%, 90%, 95%, 97% (or more) sequence identity to one or more of SEQ ID NOs: 52, 47-49, 51 and 53-54. In several embodiments, the tumor binding domain comprises an scFv, wherein the scFv comprises a VH and a VL linked by a linker comprising the sequence of SEQ ID NO: 50. In several embodiments, the tumor binding domain comprises an scFv comprising the amino acid sequence of any one of SEQ ID NOS: 52, 51, and 53. In several embodiments, the tumor binding domain comprises a single chain variable fragment (scFv), wherein the scFv is encoded by a polynucleotide comprising a sequence having at least 95% sequence identity to one or more of the polynucleotides of SEQ ID NOs: 35, 30-32, 34, 36 and 37.

Also provided is a population of genetically engineered natural killer (NK) cells, comprising a plurality of NK cells engineered to express a chimeric antigen receptor (CAR) comprising a tumor binding domain, a transmembrane domain, and a cytotoxic signaling complex, wherein the tumor binding domain targets CD70, wherein the NK cells comprise a genomic disruption within a CD70 protein gene target sequence that comprises SEQ ID NO: 180, wherein the NK cells also comprise a genomic disruption within of a cytokine-inducible SH2-containing protein gene target sequence that comprises SEQ ID NO: 191, and wherein the NK cells also comprise a genomic disruption within the CBLB protein gene target sequence that comprises SEQ ID NO:195.

In several embodiments, the tumor binding domain comprises an scFv, wherein the scFv comprises a heavy chain variable region (VH) that comprises a CDR-H1, a CDR-H2, and a CDR-H3 comprising the sequences of SEQ ID NOS: 205, 206 respectively, a light chain variable region (VL) comprising a CDR-L1, a CDR-L2, and a CDR-L3 comprising the sequences of SEQ ID NOS: 209, 210, and 211, respectively; and a linker between the VH and VL comprising the sequence of SEQ ID NO:50.

In several embodiments, the tumor binding domain comprises an scFv comprising the amino acid sequence of any one of SEQ ID NOS: 52, 51, and 53. In several embodiments, the tumor binding domain comprises a single chain variable fragment (scFv), wherein the scFv is encoded by a polynucleotide comprising a sequence having at least 95% sequence identity to one or more of the polynucleotides of SEQ ID NOs: 35, 30-32, 34, 36 and 37.

In several embodiments, the tumor binding domain comprises a heavy chain variable region (VH), wherein the VH is encoded by a polynucleotide comprising a sequence having at least about 85%, 90%, 95%, 97% (or more) sequence identity to one or more of the polynucleotides of SEQ ID NOs: 143-146 and 149. In several embodiments, the tumor binding domain comprises a light chain variable region (VL), wherein the VL is encoded by a polynucleotide comprising a sequence having at least about 85%, 90%, 95%, 97% (or more) sequence identity to one or more of the polynucleotides of SEQ ID NOs: 146-148 and 150.

In several embodiments, the tumor binding domain comprises a single chain variable fragment (scFv), wherein the scFv is encoded by a polynucleotide comprising a sequence having at least about 85%, 90%, 95%, 97% (or more) sequence identity to one or more of the polynucleotides of SEQ ID NOs: 30-32 and 34-37.

In several embodiments, the cytotoxic signaling complex comprises an OX40 subdomain and a CD3zeta subdomain. In several embodiments, the OX40 subdomain comprises the amino acid sequence of SEQ ID NO:6. In several embodiments, the OX40 subdomain is encoded by a sequence having at least about 85%, 90%, 95%, 97% (or more) sequence identity to SEQ ID NO: 5. In several embodiments, the CD3zeta subdomain comprises the amino acid sequence of SEQ ID NO:8. In several embodiments, the CD3zeta subdomain is encoded by a sequence having at least about 85%, 90%, 95%, 97% (or more) sequence identity to SEQ ID NO: 7.

In several embodiments, the NK cells are engineered to express membrane bound IL-15 (mbIL15). In several embodiments, the mbIL15 is bicistronically encoded on a polynucleotide encoding the CAR. In several embodiments, the mbIL15 comprises the amino acid sequence of SEQ ID NO:213. In some embodiments, the mbIL15 is encoded by a sequence having at least about 85%, 90%, 95%, 97% (or more) sequence identity to SEQ ID NO: 27. In several embodiments, the polynucleotide encoding the CAR and the mbIL15 comprises a sequence having at least about 85%, 90%, 95%, 97% (or more) sequence identity to one or more of the polynucleotides of SEQ ID NOs: 38-46.

In several embodiments, the CAR comprises an amino acid sequence having at least about 85%, 90%, 95%, 97% (or more) sequence identity to one or more of the amino acid sequences of SEQ ID NOs: 214-222.

In several embodiments, the engineered NK cells are edited at CD70, CISH, and CBLB. In several embodiments, the engineered NK cells comprise a genomic disruption within a CD70 protein gene target sequence that comprises SEQ ID NO:180, a genomic disruption within a CIS protein gene target sequence that comprises SEQ ID NO:191, and a genomic disruption within a CBLB protein gene target sequence that comprises SEQ ID NO:195.

In several embodiments, the engineered NK cells are edited at CD70, CISH, CBLB, and an additional target gene. In several embodiments, the expression of CD70 is substantially reduced as compared to an NK cell not edited with respect to CD70, the expression of CIS is substantially reduced as compared to an NK cell not edited with respect to CISH, and the expression of CBLB is substantially reduced as compared to an NK cell not edited with respect to CBLB. In several embodiments, the NK cells do not express a detectable level of CD70, CIS, or CBLB protein.

In several embodiments, the gene editing introduce the genomic disruption is made using a CRISPR-Cas system. In several embodiments, the CRISPR-Cas system comprises a Cas selected from Cas9, Csn2, Cas4, Cpf1, C2c1, C2c3, Cas13a, Cas13b, Cas13c, CasX, CasY, and combinations thereof. In several embodiments, the Cas is Cas9.

In several embodiments, the CD70 that is targeted by the tumor binding domain is expressed by a solid tumor.

Also provided herein is a population of genetically engineered natural killer (NK) cells for cancer immunotherapy, comprising a plurality of NK cells that have been expanded in culture, wherein the NK cells are engineered to express a chimeric antigen receptor (CAR) comprising a tumor binding domain, a transmembrane domain, and a cytotoxic signaling complex, wherein the tumor binding domain targets CD70, wherein the NK cells comprise a genomic disruption within a CD70 protein gene target sequence that comprises any one of SEQ ID NO: 180 or 177-180 wherein said genomic disruption comprises and endonuclease-mediated indel, wherein the NK cells also comprise a genomic disruption within of a cytokine-inducible SH2-containing protein gene target sequence that comprises any one of SEQ ID NO: 186-191, and wherein the NK cells comprise at least one additional genomic disruption within a gene target sequence, and wherein the genetically engineered NK cells comprising said genomic disruptions exhibit one or more of enhanced expansion capability, enhanced cytotoxicity against target cells, and enhanced persistence, as compared to NK cells not comprising said genomic disruptions.

Also provided herein is a population of genetically engineered natural killer (NK) cells for cancer immunotherapy, comprising a plurality of NK cells that have been expanded in culture, wherein the NK cells are engineered to express a chimeric antigen receptor (CAR) comprising a tumor binding domain, a transmembrane domain, and a cytotoxic signaling complex, wherein the tumor binding domain targets CD70, wherein the NK cells are genetically edited to express reduced levels of CD70 as compared to a non-edited NK cell that has been expanded in culture, and wherein the reduced CD70 expression was engineered through introducing a genomic disruption in an endogenous CD70 gene, wherein the NK cells are also genetically edited to express reduced levels of a cytokine-inducible SH2-containing (CIS) protein encoded by a CISH gene as compared to a non-edited NK cell, wherein the reduced CIS expression was engineered through introducing a genomic disruption in a CISH gene, wherein the genetically engineered NK cells comprising said genomic disruptions exhibit one or more of enhanced expansion capability, enhanced cytotoxicity against target cells, and enhanced persistence, as compared to NK cells expressing native levels of CIS and wherein the NK cells are genetically edited to introduce a genomic disruption in at two or more additional genes to reduce expression of a protein encoded by said two or more additional genes as compared to a NK cell not edited at said genes.

In several embodiments, the cells and methods provided for herein are used for the treatment of renal cell carcinoma, or a metastasis from renal cell carcinoma. Additionally provided herein are uses of the genetically engineered NK cells according to embodiments disclosed herein in the treatment of a cancer. In several embodiments, the cancer is a CD70-expressing cancer. In some embodiments, the cancer comprises a solid tumor. Also provided herein are methods of treating a cancer in a subject by administering an immune cell as described herein. In some embodiments, the administration treats, inhibits, or prevents progression of the cancer. Further provided are uses of the genetically engineered NK cells according to embodiments disclosed herein in the manufacture of a medicament for the treatment of cancer.

Provided for herein is an anti-CD70 CAR, wherein the CAR comprises an anti-CD70 binding domain, an OX40 domain, and a CD3zeta domain, wherein the anti-CD70 CAR comprises an amino acid sequence having at least 95% sequence identity to one or more of the amino acid sequences of SEQ ID NOs: 218, 214-217, or 219-222, or a portion thereof capable of generating cytotoxic signals upon binding to CD70 on a target cell.

An anti-CD70 chimeric antigen receptor (CAR), wherein the CAR comprises an anti-CD70 binding domain, an OX40 domain, and a CD3zeta domain, wherein the anti-CD70 CAR comprises an amino acid sequence having at least about 85%, 90%, 95%, 97% (or more) sequence identity to one or more of the amino acid sequences of SEQ ID NOs: 64-72, or a portion thereof capable of generating cytotoxic signals upon binding to CD70 on a target cell. In several embodiments, the anti-CD70 binding domain comprises an scFv having at least about 85%, 90%, 95%, 97% (or more) sequence identity to any sequence selected from SEQ ID NOs: 52, 47-49, 51, and 53-54.

Also provided for herein is a cell comprising such an anti-CD70 CAR. In several embodiments, the cells is an immune cell. In several embodiments, the cell is an NK cell. In several embodiments, the cell comprises at least three genomic disruptions within at least three gene target sequences selected from SEQ ID NOs: 159-201. In several embodiments, the cell comprises genomic disruptions within the protein encoding gene target sequences of SEQ ID NOs: 180, 191, and 195. Also provided herein are methods of treating cancer in a subject by administering such as CAR or such a cell. Uses of such cells or such CARs for the treatment of a cancer or for the manufacture of a medicament for the treatment of cancer are also provided.

Still additional embodiments, provide for a method for generating a population of genetically engineered immune cells, comprising introducing an endonuclease and at least one unique gRNA into the immune cells to induce a genomic disruption within at least one gene target sequence, introducing an endonuclease and at least one additional unique gRNA into the immune cells to induce an additional genomic disruption within an additional gene target sequence, and transducing the immune cells with a viral vector encoding a CD70-targeting CAR. In several embodiments, the endonuclease and gRNA are induced by electroporating the cells. In several embodiments, the cells comprise NK cells. In several embodiments, no more than three unique gRNAs are introduced at a time. In several embodiments, no more than two unique gRNAs are introduced at a time. In several embodiments, the cells are expanded in culture for a period of time prior to the first introduction.

Also provided for is a method for generating a population of genetically engineered immune cells, comprising expanding the immune cells in culture, introducing an endonuclease and no more than two unique gRNA into the immune cells to induce a genomic disruption within two distinct gene target sequences, culturing the cells for an additional period of time introducing an additional endonuclease and no more than two additional unique gRNA into the immune cells to induce additional genomic disruptions within no more than two additional gene target sequences, and transducing the immune cells with a viral vector encoding a CD70-targeting CAR. In several embodiments, the endonucleases and gRNA are induced by electroporating the cells. In several embodiments, the cells comprise NK cells. In several embodiments, only one additional type of gRNA is used in the second introduction. In several embodiments, the gRNAs target CD70, CISH, or CBLB genes.

In several embodiments, there is a provided a pharmaceutical composition that comprises a population of engineered NK cells that comprise a genomic disruption within a gene target sequence that comprises at least three of SEQ ID NO: 159-203, wherein said genomic disruption optionally comprises an endonuclease-mediated indel.

In several embodiments, there is provided a pharmaceutical composition that comprises a population of engineered natural killer cells that comprise a genomic disruption within a gene target sequence that comprises at least three of SEQ ID NO: 177-195, wherein said genomic disruption optionally comprises an endonuclease-mediated indel.

In several embodiments, there is provided a pharmaceutical composition that comprises a population of engineered natural killer cells that comprise a genomic disruption within a gene target sequence that comprises at least two of SEQ ID NO: 177-195, wherein said genomic disruption optionally comprises an endonuclease-mediated indel, and wherein engineered NK cells express a CD70-targeting CAR comprising an scFv comprising an amino acid sequence having at least about 85%, 90%, 95%, 97% (or more) sequence identity to one or more of SEQ ID NOs: 52, 47-49, 51, and 53-54. In several embodiments, the engineered natural killer cells comprise genomic disruptions within target gene sequences of SEQ ID NOS: 180, 191, and 195. In several embodiments of the pharmaceutical compositions provided for, the genomic disruption comprises an endonuclease-mediated indel.

Some embodiments relate to a method comprising administering an immune cell as described herein to a subject in need. In some embodiments, the subject has cancer. In some embodiments, the administration treats, inhibits, or prevents progression of the cancer.

Several embodiments provide for uses of the genetically edited cells, anti-CD70 scFvs, anti-CD70 CARs, and/or the polynucleotides or amino acid sequences disclosed herein in the treatment or prevention of cancer.

### BRIEF DESCRIPTION OF THE DRAWINGS

FIG. 1 depicts non-limiting schematics of tumor-directed chimeric antigen receptors.

FIG. 2 depicts summary data of various characteristics of non-limiting embodiments of CD70-targeting CARs according to the present disclosure.

FIG. 3 depicts representative data related to persistence of CAR expression by gene edited NK cells.

FIG. 4 depicts representative data related to the percentage of gene edited NK cells present overtime in a culture.

FIG. 5 shows representative data related to the expansion capacity of gene edited NK cells.

FIG. 6 shows a schematic of a non-limiting embodiment of process flow for generation and analysis of gene edited NK cells.

FIGS. 7A-7B show representative flow cytometry data related gene editing to knock out CD70 expression in NK cells from two donors.

FIGS. 8A-8H show representative flow cytometry data related maintenance of reduced CD70 expression by NK cells 10 days after transduction with a non-limiting embodiment of CD70 CAR.

FIGS. 9A-9H shows representative flow cytometry data related maintenance of reduced CD70 expression by NK cells 14 days after transduction with a non-limiting embodiment of CD70 CAR.

FIGS. 10A-10B show representative Tracking of Indels by Decomposition (TIDE) indel analysis data related to the efficacy of gene editing to knock down CD70 expression after transduction with a non-limiting embodiment of CD70 CAR with 10A showing data from a first donor and 10B showing data from a second donor.

FIG. 10C shows data from two different donors related to the persistence of CD70/CISH KO NK cells expressing non-limiting embodiments of CD70 CARs in the absence of interleukin-2 (IL2).

FIGS. 11A-11D show representative in vitro cytotoxicity data (Bright-Glo™ Assay) against low CD70-expressing Panc05 tumor cells using the indicated non-limiting anti-CD70 CARs expressed by NK cells from a first and second donor and tested on Day 14 (11A and 11C, respectively) and Day 17 (11B and 11D, respectively) post-electroporation (EP).

FIGS. 12A-12B show representative in vitro cytotoxicity data (IncuCyte® Assay) against low CD70-expressing Panc05 tumor cells using the indicated non-limiting anti-CD70 CARs expressed by NK cells from a first donor (12A) and a second donor (12B).

FIGS. 13A-13B show representative in vitro cytotoxicity data (IncuCyte® Assay) against moderate CD70-expressing ACHN tumor cells using two tumor cell re-challenges and using the indicated non-limiting anti-CD70 CARs expressed by NK cells from a first donor (13A) and a second donor (13B).

FIGS. 14A-14C show representative in vitro cytotoxicity data (IncuCyte® Assay) against high CD70-expressing 786-O tumor cells using one tumor cell re-challenge and using the indicated non-limiting anti-CD70 CARs expressed by NK cells from a first donor (14A) and a second donor (14C). FIG. 14B shows data regarding cytotoxicity collected at the time of the vertical line in FIG. 14A.

FIGS. 15A-15D show representative expression data (measured as both percentage of CAR-positive cells and mean fluorescence intensity (e.g., density of expression)) at day 10 and day 14 post-electroporation (EP) for a first donor (15A-15B) and a second donor (15C-15D).

FIGS. 16A-16E show representative in vivo data that demonstrates that non-limiting embodiments of CD70-directed CARs as provided for herein show anti-tumor activity in a 786-O renal carcinoma xenograft animal model.

FIGS. 17A-17B show representative in vivo data demonstrating that non-limiting embodiments of CD70-directed CARs as provided for herein show anti-tumor activity in a 786-O renal carcinoma xenograft animal model (17A) and that CAR-positive cells exhibit persistent presence in the bloodstream for several weeks.

FIGS. 18A-18B show schematics for various non-limiting embodiments of gene editing protocols. FIG. 18A shows a Day 0 approach where the gene editing occurs on resting cells. FIG. 18B shows a Day 6 approach where the gene editing occurs on activated cells.

FIGS. 19A-19B show reduction in protein expression after gene editing was performed using the Day 0 approach of FIG. 18A. FIG. 19A shows reductions in CBLB protein. FIG. 19B shows reduction in CIS protein.

FIGS. 20A-20C show data related to the enrichment of CD70 CAR-positive gene edited cells in culture over time. FIG. 20A shows the percentage of CD70 CAR-positive gene edited NK cells at day 11 post-editing. FIG. 20B shows the percentage of CD70 CAR-positive gene edited NK cells at day 21 post-editing. FIG. 20C shows the percentage of CD70 CAR-positive gene edited NK cells at day 28 post-editing.

FIGS. 21A-21C show data related to the expansion of gene edited cells. FIG. 21A shows the fold expansion of the edited cells prior to transduction with a CD70 CAR. FIG. 21B shows the fold expansion of the gene edited cells after being transduced with a CD70 CAR. FIG. 21C shows the fold expansion of the CD70 CAR-expressing gene edited NK cells at Day 14 post-editing.

FIGS. 21D-E show representative in vitro cytotoxicity data (IncuCyte® Assay) against moderate CD70-expressing ACHN cells (21D) and high-expressing 786-O cells (21E) using the indicated non-limiting anti-CD70 CARs expressed by NK cells from a donor.

FIGS. 21F-G show representative in vitro cytotoxicity data (IncuCyte® Assay) against moderate CD70-expressing ACHN cells (21F) and high-expressing 786-O cells (21G) using one tumor cell re-challenge and using the indicated non-limiting anti-CD70 CARs expressed by NK cells from a donor.

FIGS. 22A-22F show cytotoxicity data. FIGS. 22A-22B show cytotoxicity data at day 14 post-electroporation (EP) with multiple challenges of ACHN tumor cells. FIG. 22A shows data in the absence of TGF beta. FIG. 22B shows data in the presence of TGF beta. FIGS. 22C-22D show cytotoxicity data at day 21 post-electroporation (EP) with multiple challenges of ACHN tumor cells. FIG. 22C shows data in the absence of TGF beta. FIG. 22D shows data in the presence of TGF beta. FIGS. 22E-22F show cytotoxicity data at day 28 post-electroporation (EP) with multiple challenges of ACHN tumor cells. FIG. 22E shows data in the absence of TGF beta. FIG. 22F shows data in the presence of TGF beta.

FIGS. 23A-23B show reduction in protein expression after gene editing was performed using the Day 6 approach of FIG. 18B. FIG. 23A shows reductions in CBLB protein. FIG. 23B shows reduction in CIS protein.

FIGS. 24A-24B show data related to the enrichment of CD70 CAR-positive gene edited cells in culture over time. FIG. 24A shows the percentage of CD70 CAR-positive gene edited NK cells at day 10 post-expansion. FIG. 24B shows the percentage of CD70 CAR-positive gene edited NK cells at day 15 post-expansion.

FIGS. 25A-25B show cytotoxicity data at day 14 post-expansion with multiple challenges of ACHN tumor cells. FIG. 25A shows data in the absence of TGF beta. FIG. 25B shows data in the presence of TGF beta.

FIGS. 26A-26B show long-term in vivo cytotoxicity data. FIG. 26A shows data indicated that multiplex gene-edited NK cells control tumor growth more effectively than controls over 45 days (in a 786-O model). FIG. 26B shows similar data with an A-498 xenograft model.

FIG. 27 shows a schematic outline an assessment of off-target gene editing.

FIG. 28 shows a schematic workflow of a non-limiting embodiment of off-target gene editing.

FIG. 29A shows data related to the predicted number of off target sites for the indicated guide RNAs (gRNAs) and the median next generation sequencing (NGS) read coverage across the sites.

FIG. 29B shows additional data related to the predicted number of off target sites for the indicated guide RNAs and the median NGS read coverage across the sites, and includes all of the data shown in FIG. 29A.

FIG. 30A shows data related to the calculated on-targeting editing (by TIDE and hybrid capture analyses) and data indicating the absence of off-target editing, based on the gRNAs and donors shown in FIG. 29A.

FIG. 30B shows data related to the calculated on-targeting editing (by TIDE and hybrid capture analyses) and data indicating the absence of off-target editing, based on the gRNAs and donors shown in FIG. 29B.

FIG. 31 shows a non-limiting schematic for the workflow for assessment of chromosomal translocation post-editing.

FIG. 32 shows data related to indel frequency after single and double edits using the CISH-15 gRNA in two donors.

FIG. 33A shows data related to indel frequency after single edits using the CISH-10 or CISH-15 gRNA in two donors.

FIG. 33B shows additional data compared to FIG. 33A, related to indel frequency after single edits using the indicated gRNAs in four donors.

FIG. 34 shows data related to CD70 indel frequency after single, dual, or triple edits using the CISH-10 or CISH-15 gRNA in two donors.

FIGS. 35A-35G show data related to CD70 expression in non-transduced NK cells from a first donor at day 13 after the indicated gene edits. FIG. 35A shows an isotype control. FIG. 35B shows an electroporation (EP) control. FIG. 35C shows editing of a CD70 gene. FIG. 35D shows editing of CD70 and CISH (using the CISH-15 gRNA). FIG. 35E shows editing of CD70 and CBLB. FIG. 35F shows editing of CD70, CBLB, and CISH (using the CISH-15 gRNA). FIG. 35G shows editing of CD70 and CISH (using the CISH-10 gRNA).

FIGS. 36A-36G show data related to CD70 expression in non-transduced NK cells from a second donor at day 13 after the indicated gene edits. FIG. 36A shows an isotype control. FIG. 36B shows an EP control. FIG. 36C shows editing of a CD70 gene. FIG. 36D shows editing of CD70 and CISH (using the CISH-15 gRNA). FIG. 36E shows editing of CD70 and CBLB. FIG. 36F shows editing of CD70, CBLB, and CISH (using the CISH-15 gRNA). FIG. 36G shows editing of CD70 and CISH (using the CISH-10 gRNA).

FIGS. 37A-37B show data related to CBLB, and optionally CISH (using the CISH-15 gRNA), editing. FIG. 37A shows data related to the indel frequency after single or dual edits in a first donor. FIG. 37B shows data related to the indel frequency after single or dual edits in a second donor.

FIG. 38 shows information related to a non-limiting experimental design to assess chromosomal translocation.

FIG. 39 shows data related to the indel frequency for certain non-limiting multiple gene edits.

FIGS. 40A-40B show non-limiting embodiments of gene editing approaches. FIG. 40A shows a single electroporation (EP) approach. FIG. 40B shows a dual EP approach.

FIGS. 41A-41C show non-limiting embodiments of gene editing approaches when 2 electroporations (EPs) are used. FIG. 41A shows a first configuration of edits. FIG. 41B shows a second configuration of edits. FIG. 41C shows a third configuration of edits.

FIG. 42 shows data related to chromosomal translocation rate with a single electroporation (EP) approach (three simultaneous edits).

FIG. 43 shows data related to chromosomal translocation rate with a first electroporation (EP) performed to accomplish dual edits to CD70 and CISH, using the indicated CISH gRNA.

FIG. 44 shows data related to chromosomal translocation rate with a first electroporation (EP) performed to accomplish dual edits to CD70 and CBLB and optional configurations for an EP1/EP2 approach.

FIG. 45A shows data related to the knockout efficiency of CBLB, CISH, and CD70 in CBLB/CISH/CD70 KO NK cells expressing the indicated non-limiting anti-CD70 CARs, compared to CBLB/CISH/CD70 KO NK cells not expressing a CAR (Triple KO) or NK cells mock-electroporated and not expressing a CAR (EP only).

FIG. 45B shows data related to the persistence of CBLB/CISH/CD70 KO NK cells expressing the indicated non-limiting anti-CD70 CARs in the absence of interleukin-2 (IL2).

FIG. 45C shows data related to the expression of molecules associated with activation in CBLB/CISH/CD70 KO NK cells expressing the indicated non-limiting anti-CD70 CARs cultured with target cells at an E:T ratio of 1:2 or 1:4.

FIG. 46A shows tumor volume (TV) change from baseline (top panel) and tumor volume (TV) (bottom panel) in a 786-O murine tumor model treated with CISH/CBLB/CD70 NK cells expressing the indicated non-limiting anti-CD70 CARs, CISH/CBLB/CD70 KO NK cells not expressing a CAR (Triple KO), or vehicle.

FIG. 46B shows the persistence of NK cells expressing the indicated non-limiting anti-CD70 CARs in the same mice shown in FIG. 46A.

### DETAILED DESCRIPTION

Some embodiments of the methods and compositions provided herein relate to engineered immune cells and combinations of the same for use in immunotherapy. In several embodiments, the engineered cells are engineered in multiple ways, for example, to express a cytotoxicity-inducing receptor complex. As used herein, the term “cytotoxic receptor complexes” shall be given its ordinary meaning and shall also refer to (unless otherwise indicated), Chimeric Antigen Receptors (CARs). In several embodiments, the cells are further engineered to achieve a modification of the reactivity of the cells against non-tumor tissue and/or other therapeutic cells. In several embodiments, natural killer (NK) cells are also engineered to express a cytotoxicity-inducing receptor complex (e.g., a chimeric antigen receptor or chimeric receptor), such as for example targeting CD70 expressing tumor cells. In several embodiments, the NK cells are genetically edited to reduce and/or eliminate certain markers/proteins that would otherwise inhibit or limit the therapeutic efficacy of the CAR-expressing NK cells. In several embodiments, certain markers/proteins have expression that is upregulated or otherwise induced by one or more processes undertaken to engineer and/or expand the NK cells. For example, in several embodiments, the process of expanding NK cells in culture results in substantially increased CD70 expression by the NK cells. In those embodiments wherein a CD70 CAR is engineered to be expressed by expanded NK cells, the CAR would actually target, not only a CD70-expressing tumor, but other engineered and expanded NK cells as well. Thus, for example, in several embodiments, therapeutic NK cells are engineered to express a CAR that targets CD70 and are likewise genetically edited to knock out CD70 expression on the NK cells themselves, which, if present, would cause the CAR-expressing NK cells to target the tumor and the therapeutic NK cells as well. This would otherwise create a self-limiting therapeutic effect, which could allow for tumor expansion and progression of the cancer.

CRISPR-Cas, a RNA-guided endonuclease-based genome editing technology has been extensively used for precise gene editing. With a short guide RNA (gRNA), an endonuclease (one example of which is Cas9, though many others exist) can be guided to the target site for precise gene editing. The gRNA determines the efficacy and specificity of gene editing by endonuclease. gRNAs are custom-designed to target specific loci in the genome and to recruit the endonuclease to that site. The recruited endonuclease induces specific double-strand breaks inside double-strand DNA that trigger DNA repair pathways. For example, non-homologous end joining pathways can be exploited to introduce a frameshift mutation(s) for gene knock out. Homologous directed repair pathways can be exploited for gene substitution or gene knock-in using supplied template DNA. CRISPR/Cas genome editing has been widely researched in many systems, including bacteria, plants, and mammals and is widely regarded as having therapeutic potential. However, one of the major challenges associated with gRNAs are the potential off-target effects. For example, if there are more than three nucleotide mismatches between the gRNA and the target sequence the gRNA can target (and thus recruit the endonuclease) to a site in the genome that was not intended to be targeted. Off-target effects can include small insertions or deletions at genomic sites with homology to a gRNA, and more rarely, large scale events such as chromosomal translocations, inversions, or deletions. Such off-target effects raise potential safety issues, particularly in the context of therapies (e.g., cell therapies) intended for treatment of humans. Thus, the identification of suitable gRNAs for a given desired edit to the genome are important to minimize off-target effects, while still maintaining high on-target editing efficiency. According to embodiments provided for herein and to overcome these challenges, the gRNAs provided herein have been demonstrated to show high on-target editing efficiency and low off-target effects.

The term “anticancer effect” refers to a biological effect which can be manifested by various means, including but not limited to, a decrease in tumor volume, a decrease in the number of cancer cells, a decrease in the number of metastases, an increase in life expectancy, decrease in cancer cell proliferation, decrease in cancer cell survival, and/or amelioration of various physiological symptoms associated with the cancerous condition.

### Cell Types

Some embodiments of the methods and compositions provided herein relate to a cell such as an immune cell. For example, an immune cell, such as an NK cell or a T cell, may be engineered to include a chimeric receptor such as a CD70-directed chimeric receptor, or engineered to include a nucleic acid encoding said chimeric receptor as described herein. Still additional embodiments relate to the further genetic manipulation of the cells (e.g., donor NK cells) to reduce, disrupt, minimize and/or eliminate the expression of one or more markers/proteins by the NK cells, resulting in an enhancement of the efficacy and/or persistence of the engineered NK cells.

Traditional anti-cancer therapies relied on a surgical approach, radiation therapy, chemotherapy, or combinations of these methods. As research led to a greater understanding of some of the mechanisms of certain cancers, this knowledge was leveraged to develop targeted cancer therapies. Targeted therapy is a cancer treatment that employs certain drugs that target specific genes or proteins found in cancer cells or cells supporting cancer growth, (like blood vessel cells) to reduce or arrest cancer cell growth. More recently, genetic engineering has enabled approaches to be developed that harness certain aspects of the immune system to fight cancers. In some cases, a patient's own immune cells are modified to specifically eradicate that patient's type of cancer. Various types of immune cells can be used, such as T cells, Natural Killer (NK cells), or combinations thereof, as described in more detail below.

To facilitate cancer immunotherapies, there are provided for herein polynucleotides, polypeptides, and vectors that encode chimeric antigen receptors (CAR) that comprise a target binding moiety (e.g., an extracellular binder of a ligand, or a tumor marker-directed chimeric receptor, expressed by a cancer cell) and a cytotoxic signaling complex. For example, some embodiments include a polynucleotide, polypeptide, or vector that encodes, for example a chimeric antigen receptor directed against a tumor marker, for example, CD70, to facilitate targeting of an immune cell to a cancer and exerting cytotoxic effects on the cancer cell. Also provided are engineered immune cells (e.g., NK cells and/or T cells) expressing such CARs. Methods of treating cancer and other uses of such cells for cancer immunotherapy are also provided for herein.

Engineered Cells for Immunotherapy

In several embodiments, cells of the immune system are engineered to have enhanced cytotoxic effects against target cells, such as tumor cells. For example, a cell of the immune system may be engineered to include a tumor-directed chimeric receptor and/or a tumor-directed CAR as described herein. In several embodiments, white blood cells or leukocytes, are used, since their native function is to defend the body against growth of abnormal cells and infectious disease. There are a variety of types of white bloods cells that serve specific roles in the human immune system, and are therefore a preferred starting point for the engineering of cells disclosed herein. White blood cells include granulocytes and agranulocytes (presence or absence of granules in the cytoplasm, respectively). Granulocytes include basophils, eosinophils, neutrophils, and mast cells. Agranulocytes include lymphocytes and monocytes. Cells such as those that follow or are otherwise described herein may be engineered to include a chimeric antigen receptor, such as a CD70-directed CAR, or a nucleic acid encoding the CAR. In several embodiments, the cells are optionally engineered to co-express a membrane-bound interleukin 15 (mbIL15) domain. As discussed in more detail below, in several embodiments, the therapeutic cells, are further genetically modified enhance the cytotoxicity and/or persistence of the cells. In several embodiments, the genetic modification enhances the ability of the cell to resist signals emanating from the tumor microenvironment that would otherwise cause a reduced efficacy or shortened lifespan of the therapeutic cells.

Monocytes for Immunotherapy

Monocytes are a subtype of leukocyte. Monocytes can differentiate into macrophages and myeloid lineage dendritic cells. Monocytes are associated with the adaptive immune system and serve the main functions of phagocytosis, antigen presentation, and cytokine production. Phagocytosis is the process of uptake cellular material, or entire cells, followed by digestion and destruction of the engulfed cellular material. In several embodiments, monocytes are used in connection with one or more additional engineered cells as disclosed herein. Some embodiments of the methods and compositions described herein relate to a monocyte that includes a tumor-directed CAR, or a nucleic acid encoding the tumor-directed CAR. Several embodiments of the methods and compositions disclosed herein relate to monocytes engineered to express a CAR that targets a tumor marker, for example, CD70, and optionally include a membrane-bound interleukin 15 (mbIL15) domain.

Lymphocytes for Immunotherapy

Lymphocytes, the other primary sub-type of leukocyte include T cells (cell-mediated, cytotoxic adaptive immunity), natural killer cells (cell-mediated, cytotoxic innate immunity), and B cells (humoral, antibody-driven adaptive immunity). While B cells are engineered according to several embodiments, disclosed herein, several embodiments also relate to engineered T cells or engineered NK cells (mixtures of T cells and NK cells are used in some embodiments, either from the same donor, or different donors). Several embodiments of the methods and compositions disclosed herein relate to lymphocytes engineered to express a CAR that targets a tumor marker, for example, CD70, and optionally include a membrane-bound interleukin 15 (mbIL15) domain.

T Cells for Immunotherapy

T cells are distinguishable from other lymphocytes sub-types (e.g., B cells or NK cells) based on the presence of a T-cell receptor on the cell surface. T cells can be divided into various different subtypes, including effector T cells, helper T cells, cytotoxic T cells, memory T cells, regulatory T cells, natural killer T cell, mucosal associated invariant T cells and gamma delta T cells. In some embodiments, a specific subtype of T cell is engineered. In some embodiments, a mixed pool of T cell subtypes is engineered. In some embodiments, there is no specific selection of a type of T cells to be engineered to express the cytotoxic receptor complexes disclosed herein. In several embodiments, specific techniques, such as use of cytokine stimulation are used to enhance expansion/collection of T cells with a specific marker profile. For example, in several embodiments, activation of certain human T cells, e.g. CD4+ T cells, CD8+ T cells is achieved through use of CD3 and/or CD28 as stimulatory molecules. In several embodiments, there is provided a method of treating or preventing cancer or an infectious disease, comprising administering a therapeutically effective amount of T cells expressing the cytotoxic receptor complex and/or a homing moiety as described herein. In several embodiments, the engineered T cells are autologous cells, while in some embodiments, the T cells are allogeneic cells. Several embodiments of the methods and compositions disclosed herein relate to T cells engineered to express a CAR that targets a tumor marker, for example, CD70, and optionally include a membrane-bound interleukin 15 (mbIL15) domain.

NK Cells for Immunotherapy

In several embodiments, there is provided a method of treating or preventing cancer or an infectious disease, comprising administering a therapeutically effective amount of natural killer (NK) cells expressing the cytotoxic receptor complex and/or a homing moiety as described herein. In several embodiments, the engineered NK cells are autologous cells, while in some embodiments, the NK cells are allogeneic cells. In several embodiments, NK cells are preferred because the natural cytotoxic potential of NK cells is relatively high. In several embodiments, it is unexpectedly beneficial that the engineered cells disclosed herein can further upregulate the cytotoxic activity of NK cells, leading to an even more effective activity against target cells (e.g., tumor or other diseased cells). Some embodiments of the methods and compositions described herein relate to NK cells engineered to express a CAR that targets a tumor marker, for example, CD70, and optionally include a membrane-bound interleukin 15 (mbIL15) domain. In several embodiments, the NK cells are engineered to express a CAR that binds to CD70. In some embodiments, the NK cells are engineered to express a membrane-bound interleukin 15 (mbIL15) domain. In some embodiments, the NK cells engineered to express the CAR are engineered to also express (e.g., bicistronically express) a membrane-bound interleukin 15 (mbIL15) domain. Thus, in some embodiments, the NK cells are engineered to bicistronically express the CAR and mbIL15.

In several embodiments, primary NK cells are used. In several embodiments, the primary NK cells are isolated from peripheral blood mononuclear cells (PBMCs).

In several embodiments, immortalized NK cells are used and are subject to gene editing and/or engineering, as disclosed herein. In some embodiments, the NK cells are derived from cell line NK-92. NK-92 cells are derived from NK cells, but lack major inhibitory receptors displayed by normal NK cells, while retaining the majority of activating receptors. Some embodiments of NK-92 cells described herein related to NK-92 cell engineered to silence certain additional inhibitory receptors, for example, SMAD3, allowing for upregulation of interferon-γ (IFNγ), granzyme B, and/or perforin production. Additional information relating to the NK-92 cell line is disclosed in WO 1998/49268 and U.S. Patent Application Publication No. 2002/0068044 and incorporated in their entireties herein by reference. NK-92 cells are used, in several embodiments, in combination with one or more of the other cell types disclosed herein. For example, in one embodiment, NK-92 cells are used in combination with NK cells as disclosed herein. In an additional embodiment, NK-92 cells are used in combination with T cells as disclosed herein.

Hematopoietic Stem Cells for Cancer Immunotherapy

In some embodiments, hematopoietic stem cells (HSCs) are used in the methods of immunotherapy disclosed herein. In several embodiments, the cells are engineered to express a homing moiety and/or a cytotoxic receptor complex. HSCs are used, in several embodiments, to leverage their ability to engraft for long-term blood cell production, which could result in a sustained source of targeted anti-cancer effector cells, for example to combat cancer remissions. In several embodiments, this ongoing production helps to offset anergy or exhaustion of other cell types, for example due to the tumor microenvironment. In several embodiments allogeneic HSCs are used, while in some embodiments, autologous HSCs are used. In several embodiments, HSCs are used in combination with one or more additional engineered cell type disclosed herein. Some embodiments of the methods and compositions described herein relate to a stem cell, such as a hematopoietic stem cell engineered to express a CAR that targets a tumor marker, for example, CD70, and optionally include a membrane-bound interleukin 15 (mbIL15) domain.

Induced Pluripotent Stem Cells

In some embodiments, immune cells are derived (differentiated) from pluripotent stem cells (PSCs). In some embodiments, immune cells (e.g., NK cells) derived from induced pluripotent stem cells (iPSCs) are used in the method of immunotherapy disclosed herein. iPSCs are used, in several embodiments, to leverage their ability to differentiate and derive into non-pluripotent cells, including, but not limited to, CD34 cells, hemogenic endothelium cells, HSCs (hematopoietic stem and progenitor cells), hematopoietic multipotent progenitor cells, T cell progenitors, NK cell progenitors, T cells, NKT cells, NK cells, and B cells comprising one or several genetic modifications at selected sites through differentiating iPSCs or less differentiated cells comprising the same genetic modifications at the same selected sites. In several embodiments, the iPSCs are used to generate iPSC-derived NK or T cells. In several embodiments, the cells are engineered to express a homing moiety and/or a cytotoxic receptor complex. In several embodiments, iPSCs are used in combination with one or more additional engineered cell type disclosed herein. Some embodiments of the methods and compositions described herein relate to a stem cell, such as an induced pluripotent stem cell engineered to express a CAR that targets a tumor marker, for example, CD70, and optionally a membrane-bound interleukin 15 (mbIL15) co-stimulatory domain.

Genetic Editing of Immune Cells

As discussed above, a variety of cell types can be utilized in cellular immunotherapy. Further, as elaborated on in more detail below, and shown in the Examples, genetic modifications can be made to these cells in order to enhance one or more aspects of their efficacy (e.g., cytotoxicity) and/or persistence (e.g., active life span).

In several embodiments, genetic manipulation of NK cells is employed to further enhance the efficacy and/or persistence of the NK cells. For example, in several embodiments, expression of various markers/proteins is reduced, substantially reduced, or knocked out (eliminated) through gene editing techniques. Depending on the embodiment, this may include gene editing to reduce expression of one or more of CD70 protein encoded by a CD70 gene, a cytokine-inducible SH2-containing (CIS) protein encoded by a CISH gene, and/or a Cbl proto-oncogene B (CBLB) protein encoded by a CBLB gene. In several embodiments, reduced expression is accomplished through targeted introduction of DNA breakage, and subsequent DNA repair mechanism. In several embodiments, double strand breaks of DNA are repaired by non-homologous end joining (NHEJ), wherein enzymes are used to directly join the DNA ends to one another to repair the break. In several embodiments, however, double strand breaks are repaired by homology directed repair (HDR), which is advantageously more accurate, thereby allowing sequence specific breaks and repair. HDR uses a homologous sequence as a template for regeneration of missing DNA sequences at the break point, such as a vector with the desired genetic elements (e.g., an insertion element to disrupt the coding sequence of the target protein, such as CD70, CBLB, and/or CISH) within a sequence that is homologous to the flanking sequences of a double strand break. This will result in the desired change (e.g., insertion) being inserted at the site of the DSB.

In several embodiments, gene editing is accomplished by one or more of a variety of engineered nucleases. In several embodiments, restriction enzymes are used, particularly when double strand breaks are desired at multiple regions. In several embodiments, a bioengineered nuclease is used. Depending on the embodiment, one or more of a Zinc Finger Nuclease (ZFN), transcription-activator like effector nuclease (TALEN), meganuclease and/or clustered regularly interspaced short palindromic repeats (CRISPR/Cas9) system are used to specifically edit the genes encoding one or more target proteins, such as CD70, CBLB, and/or CISH. In some embodiments, a CRISPR/Cas9 system is used to genetically edit a target gene, such as CD70. In some embodiments, a CRISPR/Cas9 system is used to genetically edit a target gene, such as CISH. In some embodiments, a CRISPR/Cas9 system is used to genetically edit a target gene, such as CBLB.

Meganucleases are characterized by their capacity to recognize and cut large DNA sequences (from 14 to 40 base pairs). In several embodiments, a meganuclease from the LAGLIDADG family is used, and is subjected to mutagenesis and screening to generate a meganuclease variant that recognizes a unique sequence(s), such as a specific site in a gene encoding a target protein of interest. In several embodiments, two or more meganucleases, or functions fragments thereof, are fused to create a hybrid enzymes that recognize a desired target sequence within the gene encoding a target protein of interest, such as CD70, CBLB, and/or CISH.

In contrast to meganucleases, ZFNs and TALEN function based on a non-specific DNA cutting catalytic domain which is linked to specific DNA sequence recognizing peptides such as zinc fingers or transcription activator-like effectors (TALEs). Advantageously, the ZFNs and TALENs thus allow sequence-independent cleavage of DNA, with a high degree of sequence-specificity in target recognition. Zinc finger motifs naturally function in transcription factors to recognize specific DNA sequences for transcription. The C-terminal part of each finger is responsible for the specific recognition of the DNA sequence. While the sequences recognized by ZFNs are relatively short, (e.g., ˜3 base pairs), in several embodiments, combinations of 2, 3, 4, 5, 6, 7, 8, 9, 10 or more zinc fingers whose recognition sites have been characterized are used, thereby allowing targeting of specific sequences, such as a portion of the gene encoding a target protein normally expressed by NK cells, such as CD70, CBLB, and/or CISH. The combined ZFNs are then fused with the catalytic domain(s) of an endonuclease, such as Fokl (optionally a Fokl heterodimer), in order to induce a targeted DNA break. Additional information on uses of ZFNs to edit a target gene of interest, such as CD70 or CISH can be found in U.S. Pat. No. 9,597,357, which is incorporated by reference herein.

Transcription activator-like effector nucleases (TALENs) are specific DNA-binding proteins that feature an array of 33 or 34-amino acid repeats. Like ZFNs, TALENs are a fusion of a DNA cutting domain of a nuclease to TALE domains, which allow for sequence-independent introduction of double stranded DNA breaks with highly precise target site recognition. TALENs can create double strand breaks at the target site that can be repaired by error-prone non-homologous end-joining (NHEJ), resulting in gene disruptions through the introduction of small insertions or deletions. Advantageously, TALENs are used in several embodiments, at least in part due to their higher specificity in DNA binding, reduced off-target effects, and ease in construction of the DNA-binding domain.

CRISPRs (Clustered Regularly Interspaced Short Palindromic Repeats) are genetic elements that bacteria use as protection against viruses. The repeats are short sequences that originate from viral genomes and have been incorporated into the bacterial genome. Cas (CRISPR associated proteins) process these sequences and cut matching viral DNA sequences. By introducing plasmids containing Cas genes and specifically constructed CRISPRs into eukaryotic cells, the eukaryotic genome can be cut at any desired position. Additional information on CRISPR can be found in US Patent Publication No. 2014/0068797, which is incorporated by reference herein. In several embodiments, native CD70 expression by NK cells is disrupted or substantially eliminated by targeting the CD70 encoding gene with a CRISPR/Cas system. In several embodiments, one or more additional target proteins, normally expressed by an NK cells is disrupted or substantially eliminated by targeting the corresponding encoding gene with a CRISPR/Cas system. Depending on the embodiment, one or more of a cytokine-inducible SH2-containing protein encoded by a CISH gene, a Cbl proto-oncogene B protein encoded by a CBLB gene, and/or a CD70 gene is targeted with a CRISPR/Cas system. Depending on the embodiment, a Class 1 or Class 2 Cas is used. In several embodiments, a Class 1 Cas is used, and the Cas type is selected from the following types: I, IA, IB, IC, ID, IE, IF, IU, III, IIIA, IIIB, IIIC, IIID, IV IVA, IVB, and combinations thereof. In several embodiments, the Cas is selected from the group consisting of Cas3, Cas8a, Cas5, Cas8b, Cas8c, Cas10d, Cse1, Cse2, Csy1, Csy2, Csy3, GSUO0054, Cas10, Csm2, Cmr5, Cas10, Csx11, Csx10, Csf1, and combinations thereof. In several embodiments, a Class 2 Cas is used, and the Cas type is selected from the following types: II, IIA, IIB, IIC, V, VI, and combinations thereof. In several embodiments, the Cas is selected from the group consisting of Cas9, Csn2, Cas4, Cpf1, C2c1, C2c3, Cas13a (previously known as C2c2), Cas13b, Cas13c, CasX, CasY and combinations thereof. In several embodiments, the Cas is Cas9. In some embodiments, class 2 CasX is used, wherein CasX is capable of forming a complex with a guide nucleic acid and wherein the complex can bind to a target DNA, and wherein the target DNA comprises a non-target strand and a target strand. In some embodiments, class 2 CasY is used, wherein CasY is capable of binding and modifying a target nucleic acid and/or a polypeptide associated with target nucleic acid.

As discussed herein, in several embodiments NK cells are used for immunotherapy. In several embodiments provided for herein, gene editing of an NK cells imparts to the cell various beneficial characteristics such as, for example, enhanced proliferation, enhanced cytotoxicity, and/or enhanced persistence. In several embodiments provided for herein, gene editing of the NK cell can advantageously impart to the edited NK cell the ability to resist and/or overcome various inhibitory signals that are generated in the tumor microenvironment. It is known that tumors generate a variety of signaling molecules that are intended to reduce the anti-tumor effects of immune cells. As discussed in more detail below, in several embodiments, gene editing of the NK cell limits this tumor microenvironment suppressive effect on the NK cells, T cells, combinations of NK and T cells, or any edited/engineered immune cell provided for herein.

As discussed below, in several embodiments, gene editing is employed to reduce or knockout expression of target proteins, for example by disrupting the underlying gene encoding the protein. In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, the gene is completely knocked out, such that expression of the target protein is undetectable. In several embodiments, gene editing is used to “knock in” or otherwise enhance expression of a target protein. In several embodiments, expression of a target protein can be enhanced by about 30%, about 40%, about 50%, about 60%, about 70%, about 75%, about 80%, about 85%, about 90%, about 95%, about 97%, about 98%, about 99%, or more (including any amount between those listed).

In accordance with additional embodiments, other modulators of one or more aspects of NK cell (or T cell) function are modulated through gene editing. A variety of cytokines impart either negative (as with TGF-beta above) or positive signals to immune cells. By way of non-limiting example, IL15 is a positive regulator of NK cells, which as disclosed herein, can enhance one or more of NK cell homing, NK cell migration, NK cell expansion/proliferation, NK cell cytotoxicity, and/or NK cell persistence. To keep NK cells in check under normal physiological circumstances, a cytokine-inducible SH2-containing protein (CIS, encoded by the CISH gene) acts as a critical negative regulator of IL-15 signaling in NK cells. As discussed herein, IL15 biology impacts multiple aspects of NK cell functionality, including, but not limited to, proliferation/expansion, activation, cytotoxicity, persistence, homing, migration, among others. Thus, according to several embodiments, editing CISH enhances the functionality of NK cells across multiple functionalities, leading to a more effective and long-lasting NK cell therapeutic. In several embodiments, inhibitors of CIS are used in conjunction with engineered NK cell administration. In several embodiments, the CIS expression is knocked down or knocked out through gene editing of the CISH gene, for example, by use of CRISPR-Cas editing. Small interfering RNA, antisense RNA, TALENs or zinc fingers are used in other embodiments. In some embodiments CIS expression in T cells is knocked down through gene editing.

In several embodiments, CISH gene editing endows an NK cell with enhanced ability to home to a target site. In several embodiments, CISH gene editing endows an NK cell with enhanced ability to migrate, e.g., within a tissue in response to, for example chemoattractants or away from repellants. In several embodiments, CISH gene editing endows an NK cell with enhanced ability to be activated, and thus exert, for example, anti-tumor effects. In several embodiments, CISH gene editing endows an NK cell with enhanced proliferative ability, which in several embodiments, allows for generation of robust NK cell numbers from a donor blood sample. In addition, in such embodiments, NK cells edited for CISH and engineered to express a CAR are more readily, robustly, and consistently expanded in culture. In several embodiments, CISH gene editing endows an NK cell with enhanced cytotoxicity. In several embodiments, the editing of CISH synergistically enhances the cytotoxic effects of engineered NK cells and/or engineered T cells that express a CAR.

In several embodiments, CISH gene editing activates or inhibits a wide variety of pathways. The CIS protein is a negative regulator of IL15 signaling by way of, for example, inhibiting JAK-STAT signaling pathways. These pathways would typically lead to transcription of IL15-responsive genes (including CISH). In several embodiments, knockdown of CISH disinhibits JAK-STAT (e.g., JAK1-STAT5) signaling and there is enhanced transcription of IL15-responsive genes. In several embodiments, knockout of CISH yields enhanced signaling through mammalian target of rapamycin (mTOR), with corresponding increases in expression of genes related to cell metabolism and respiration. In several embodiments, knockout of CISH yields IL15 induced increased expression of IL-2Rα (CD25), but not IL-15Rα or IL-2/15Rβ, enhanced NK cell membrane binding of IL15 and/or IL2, increased phosphorylation of STAT-3 and/or STAT-5, and elevated expression of the antiapoptotic proteins, such as Bcl-2. In several embodiments, CISH knockout results in IL15-induced upregulation of selected genes related to mitochondrial functions (e.g., electron transport chain and cellular respiration) and cell cycle. Thus, in several embodiments, knockout of CISH by gene editing enhances the NK cell cytotoxicity and/or persistence, at least in part via metabolic reprogramming. In several embodiments, negative regulators of cellular metabolism, such as TXNIP, are downregulated in response to CISH knockout. In several embodiments, promotors for cell survival and proliferation including BIRC5 (Survivin), TOP2A, CKS2, and RACGAP1 are upregulated after CISH knockout, whereas antiproliferative or proapoptotic proteins such as TGFB1, ATM, and PTCH1 are downregulated. In several embodiments, CISH knockout alters the state (e.g., activates or inactivates) signaling via or through one or more of CXCL-10, IL2, TNF, IFNg, IL13, IL4, Jnk, PRF1, STAT5, PRKCQ, IL2 receptor Beta, SOCS2, MYD88, STAT3, STAT1, TBX21, LCK, JAK3, IL& receptor, ABL1, IL9, STAT5A, STAT5B, Tcf7, PRDM1, and/or EOMES.

In several embodiments, editing CBLB enhances the functionality of NK cells across multiple functionalities, leading to a more effective and long-lasting NK cell therapeutic. CBLB is an E3 ubiquitin ligase and a negative regulator of NK cell activation. CBLB reduces NK cell degranulation and cytotoxicity. Editing CBLB impacts multiple aspects of NK cell functionality, including, but not limited to, proliferation, cytotoxicity, increased IFNγ production among others. In several embodiments, inhibitors of CBLB are used in conjunction with engineered NK cell administration. In several embodiments, the CBLB expression is knocked down or knocked out through gene editing of the CBLB gene, for example, by use of CRISPR-Cas editing. Small interfering RNA, antisense RNA, TALENs or zinc fingers are used in other embodiments. In some embodiments CBLB expression in T cells is knocked down through gene editing.

In several embodiments, CBLB gene editing enhances NK cell resistance to TAM receptor (Tyro-3, Axl and Mer) mediated inhibition. In several embodiments, CBLB gene editing endows an NK cell with enhanced ability to be activated, and thus exert, for example, anti-tumor effects. In several embodiments, CBLB gene editing endows an NK cell with enhanced proliferative ability, which in several embodiments, allows for generation of robust NK cell numbers from a donor blood sample. In addition, in such embodiments, NK cells edited for CBLB and engineered to express a CAR are more readily, robustly, and consistently expanded in culture. In several embodiments, CBLB gene editing endows an NK cell with enhanced cytotoxicity. In several embodiments, the editing of CBLB synergistically enhances the cytotoxic effects of engineered NK cells and/or engineered T cells that express a CAR.

In several embodiments, a gene that is disrupted, knocked out, or otherwise altered to reduce expression of the encoded protein is CD70. In several embodiments, CD70 expression is disrupted (e.g., knocked out) in NK cells because NK cells naturally express relatively high levels of CD70, and if expression were maintained at native levels, an anti-CD70 CAR expressing NK cell would target not only a CD70-expressing tumor cell, but also other NK cells (whether native NK cells or those expressing the CD70 CAR). Thus, in several embodiments, gene editing is used to knockout CD70 expression by NK cells, such that engineered NK cells expressing an anti-CD70 CAR are not targeting the therapeutic NK cells as well as a CD70-expressing tumor. In several embodiments, inhibitors of CD70 are used in conjunction with engineered NK cell administration. In several embodiments, the CD70 expression is knocked down or knocked out through gene editing of the CD70 gene, for example, by use of CRISPR-Cas editing. Small interfering RNA, antisense RNA, TALENs or zinc fingers are used in other embodiments. In some embodiments CD70 expression in T cells is knocked down through gene editing.

In several embodiments, gene editing of the immune cells can also provide unexpected enhancement in the expansion, persistence and/or cytotoxicity of the edited immune cell. As disclosed herein, engineered cells (e.g., those expressing a CAR) may also be edited, the combination of which provides for a robust cell for immunotherapy. In several embodiments, the edits allow for unexpectedly improved NK cell expansion, persistence and/or cytotoxicity. In several embodiments, knockout of CISH expression in NK cells removes a potent negative regulator of IL15-mediated signaling in NK cells, disinhibits the NK cells and allows for one or more of enhanced NK cell homing, NK cell migration, activation of NK cells, expansion, cytotoxicity and/or persistence. In several embodiments, knockout of CBLB expression in NK cells increases the resistance of NK cells to TAM-mediated inhibition. In additional embodiments, CD70 is knocked out in NK cells such that engineered NK cells expressing an anti-CD70 CAR are not targeting the therapeutic NK cells as well as a CD70-expressing tumor. Additionally, in several embodiments, the editing can enhance NK and/or T cell function in the otherwise suppressive tumor microenvironment. In several embodiments, CISH gene editing results in enhanced NK cell expansion, persistence and/or cytotoxicity without requiring Notch ligand being provided exogenously.

### Extracellular Domains (Tumor Binder)

Some embodiments of the compositions and methods described herein relate to a chimeric antigen receptor that includes an extracellular domain that comprises a tumor-binding domain (also referred to as an antigen-binding protein or antigen-binding domain) as described herein. The tumor binding domain, depending on the embodiment, targets, for example CD70.

In some embodiments, the antigen-binding domain is derived from or comprises wild-type or non-wild-type sequence of an antibody, an antibody fragment, an scFv, a Fv, a Fab, a (Fab′)2, a single domain antibody (SDAB), a vH or vL domain, a camelid VHH domain, or a non-immunoglobulin scaffold such as a DARPIN, an affibody, an affilin, an adnectin, an affitin, a repebody, a fynomer, an alphabody, an avimer, an atrimer, a centyrin, a pronectin, an anticalin, a kunitz domain, an Armadillo repeat protein, an autoantigen, a receptor or a ligand. In some embodiments, the tumor-binding domain contains more than one antigen binding domain.

Antigen-Binding Proteins

There are provided, in several embodiments, antigen-binding proteins. As used herein, the term “antigen-binding protein” shall be given its ordinary meaning, and shall also refer to a protein comprising an antigen-binding fragment that binds to an antigen and, optionally, a scaffold or framework portion that allows the antigen-binding fragment to adopt a conformation that promotes binding of the antigen-binding protein to the antigen. In some embodiments, the antigen is a cancer antigen (e.g., CD70) or a fragment thereof. In some embodiments, the antigen-binding fragment comprises at least one CDR from an antibody that binds to the antigen. In some embodiments, the antigen-binding fragment comprises all three CDRs from the heavy chain of an antibody that binds to the antigen or from the light chain of an antibody that binds to the antigen. In still some embodiments, the antigen-binding fragment comprises all six CDRs from an antibody that binds to the antigen (three from the heavy chain and three from the light chain). In several embodiments, the antigen-binding fragment comprises one, two, three, four, five, or six CDRs from an antibody that binds to the antigen, and in several embodiments, the CDRs can be any combination of heavy and/or light chain CDRs. The antigen-binding fragment in some embodiments is an antibody fragment.

Non-limiting examples of antigen-binding proteins include antibodies, antibody fragments (e.g., an antigen-binding fragment of an antibody), antibody derivatives, and antibody analogs. Further specific examples include, but are not limited to, a single-chain variable fragment (scFv), a nanobody (e.g. VH domain of camelid heavy chain antibodies; VHH fragment,), a Fab fragment, a Fab′ fragment, a F(ab′)2 fragment, a Fvfragment, a Fd fragment, and a complementarity determining region (CDR) fragment. These molecules can be derived from any mammalian source, such as human, mouse, rat, rabbit, or pig, dog, or camelid. Antibody fragments may compete for binding of a target antigen with an intact (e.g., native) antibody and the fragments may be produced by the modification of intact antibodies (e.g. enzymatic or chemical cleavage) orsynthesized de novo using recombinant DNA technologies or peptide synthesis. The antigen-binding protein can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen-binding protein as well as wholly synthetic scaffolds comprising, for example, a biocompatible polymer. In addition, peptide antibody mimetics (“PAMs”) can be used, as well as scaffolds based on antibody mimetics utilizing fibronectin components as a scaffold.

In some embodiments, the antigen-binding protein comprises one or more antibody fragments incorporated into a single polypeptide chain or into multiple polypeptide chains. For instance, antigen-binding proteins can include, but are not limited to, a diabody; an intrabody; a domain antibody (single VL or VH domain or two or more VH domains joined by a peptide linker); a maxibody (2 scFvs fused to Fc region); a triabody; a tetrabody; a minibody (scFv fused to CH3 domain); a peptibody (one or more peptides attached to an Fc region); a linear antibody (a pair of tandem Fd segments (VH-CH1-VH-CH1) which, together with complementary light chain polypeptides, form a pair of antigen binding regions); a small modular immunopharmaceutical; and immunoglobulin fusion proteins (e.g. IgG-scFv, IgG-Fab, 2scFv-IgG, 4scFv-IgG, VH-IgG, IgG-VH, and Fab-scFv-Fc).

In some embodiments, the antigen-binding protein has the structure of an immunoglobulin. As used herein, the term “immunoglobulin” shall be given its ordinary meaning, and shall also refer to a tetrameric molecule, with each tetramer comprising two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function.

Within light and heavy chains, the variable (V) and constant regions (C) are joined by a “J” region of about 12 or more amino acids, with the heavy chain also including a “D” region of about 10 more amino acids. The variable regions of each light/heavy chain pair form the antibody binding site such that an intact immunoglobulin has two binding sites.

Immunoglobulin chains exhibit the same general structure of relatively conserved framework regions (FR) joined by three hypervariable regions, also called complementarity determining regions or CDRs. From N-terminus to C-terminus, both light and heavy chains comprise the domains FR1, CDR1, FR2, CDR2, FR3, CDR3 and FR4.

Human light chains are classified as kappa and lambda light chains. An antibody “light chain”, refers to the smaller of the two types of polypeptide chains present in antibody molecules in their naturally occurring conformations. Kappa (K) and lambda (A) light chains refer to the two major antibody light chain isotypes. A light chain may include a polypeptide comprising, from amino terminus to carboxyl terminus, a single immunoglobulin light chain variable region (VL) and a single immunoglobulin light chain constant domain (CL).

Heavy chains are classified as mu (p), delta (A), gamma (γ), alpha (a), and epsilon (s), and define the antibody's isotype as IgM, IgD, IgG, IgA, and IgE, respectively. An antibody “heavy chain” refers to the larger of the two types of polypeptide chains present in antibody molecules in their naturally occurring conformations, and which normally determines the class to which the antibody belongs. A heavy chain may include a polypeptide comprising, from amino terminus to carboxyl terminus, a single immunoglobulin heavy chain variable region (VH), an immunoglobulin heavy chain constant domain 1 (CH1), an immunoglobulin hinge region, an immunoglobulin heavy chain constant domain 2 (CH2), an immunoglobulin heavy chain constant domain 3 (CH3), and optionally an immunoglobulin heavy chain constant domain 4 (CH4).

The IgG-class is further divided into subclasses, namely, IgG1, IgG2, IgG3, and IgG4. The IgA-class is further divided into subclasses, namely IgA1 and IgA2. The IgM has subclasses including, but not limited to, IgM1 and IgM2. The heavy chains in IgG, IgA, and IgD antibodies have three domains (CH1, CH2, and CH3), whereas the heavy chains in IgM and IgE antibodies have four domains (CH1, CH2, CH3, and CH4). The immunoglobulin heavy chain constant domains can be from any immunoglobulin isotype, including subtypes. The antibody chains are linked together via inter-polypeptide disulfide bonds between the CL domain and the CH1 domain (e.g., between the light and heavy chain) and between the hinge regions of the antibody heavy chains.

In some embodiments, the antigen-binding protein is an antibody. The term “antibody”, as used herein, refers to a protein, or polypeptide sequence derived from an immunoglobulin molecule which specifically binds with an antigen. Antibodies can be monoclonal, or polyclonal, multiple or single chain, or intact immunoglobulins, and may be derived from natural sources or from recombinant sources. Antibodies can be tetramers of immunoglobulin molecules. The antibody may be “humanized”, “chimeric” or non-human. An antibody may include an intact immunoglobulin of any isotype, and includes, for instance, chimeric, humanized, human, and bispecific antibodies. An intact antibody will generally comprise at least two full-length heavy chains and two full-length light chains. Antibody sequences can be derived solely from a single species, or can be “chimeric,” that is, different portions of the antibody can be derived from two different species as described further below. Unless otherwise indicated, the term “antibody” also includes antibodies comprising two substantially full-length heavy chains and two substantially full-length light chains provided the antibodies retain the same or similar binding and/or function as the antibody comprised of two full length light and heavy chains. For example, antibodies having 1, 2, 3, 4, or 5 amino acid residue substitutions, insertions or deletions at the N-terminus and/or C-terminus of the heavy and/or light chains are included in the definition provided that the antibodies retain the same or similar binding and/or function as the antibodies comprising two full length heavy chains and two full length light chains. Examples of antibodies include monoclonal antibodies, polyclonal antibodies, chimeric antibodies, humanized antibodies, human antibodies, bispecific antibodies, and synthetic antibodies. There is provided, in some embodiments, monoclonal and polyclonal antibodies. As used herein, the term “polyclonal antibody” shall be given its ordinary meaning, and shall also refer to a population of antibodies that are typically widely varied in composition and binding specificity. As used herein, the term “monoclonal antibody” (“mAb”) shall be given its ordinary meaning, and shall also refer to one or more of a population of antibodies having identical sequences. Monoclonal antibodies bind to the antigen at a particular epitope on the antigen.

In some embodiments, the antigen-binding protein is a fragment or antigen-binding fragment of an antibody. The term “antibody fragment” refers to at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′)2, Fv fragments, scFv antibody fragments, disulfide-linked Fvs (sdFv), a Fd fragment consisting of the VH and CHI domains, linear antibodies, single domain antibodies such as sdAb (either vL or vH), camelid vHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23: 1126-1136, 2005). Antigen binding fragments can also be grafted into scaffolds based on polypeptides such as a fibronectin type III (Fn3) (see U.S. Pat. No. 6,703,199, which describes fibronectin polypeptide mini bodies). An antibody fragment may include a Fab, Fab′, F(ab′)2, and/or Fv fragment that contains at least one CDR of an immunoglobulin that is sufficient to confer specific antigen binding to a cancer antigen (e.g., CD70). Antibody fragments may be produced by recombinant DNA techniques or by enzymatic or chemical cleavage of intact antibodies.

In some embodiments, Fab fragments are provided. A Fab fragment is a monovalent fragment having the VL, VH, CL and CH1 domains; a F(ab′)2 fragment is a bivalent fragment having two Fab fragments linked by a disulfide bridge at the hinge region; a Fd fragment has the VH and CH1 domains; an Fv fragment has the VL and VH domains of a single arm of an antibody; and a dAb fragment has a VH domain, a VL domain, or an antigen-binding fragment of a VH or VL domain. In some embodiments, these antibody fragments can be incorporated into single domain antibodies, single-chain antibodies, maxibodies, minibodies, intrabodies, diabodies, triabodies, tetrabodies, v-NAR and bis-scFv. In some embodiments, the antibodies comprise at least one CDR as described herein.

There is also provided for herein, in several embodiments, single-chain variable fragments. As used herein, the term “single-chain variable fragment” (“scFv”) shall be given its ordinary meaning, and shall also refer to a fusion protein in which a VL and a VH region are joined via a linker (e.g., a synthetic sequence of amino acid residues) to form a continuous protein chain wherein the linker is long enough to allow the protein chain to fold back on itself and form a monovalent antigen binding site). For the sake of clarity, unless otherwise indicated as such, a “single-chain variable fragment” is not an antibody or an antibody fragment as defined herein. Diabodies are bivalent antibodies comprising two polypeptide chains, wherein each polypeptide chain comprises VH and VL domains joined by a linker that is configured to reduce or not allow for pairing between two domains on the same chain, thus allowing each domain to pair with a complementary domain on another polypeptide chain. According to several embodiments, if the two polypeptide chains of a diabody are identical, then a diabody resulting from their pairing will have two identical antigen binding sites. Polypeptide chains having different sequences can be used to make a diabody with two different antigen binding sites. Similarly, tribodies and tetrabodies are antibodies comprising three and four polypeptide chains, respectively, and forming three and four antigen binding sites, respectively, which can be the same or different.

In several embodiments, the antigen-binding protein comprises one or more CDRs. As used herein, the term “CDR” shall be given its ordinary meaning, and shall also referto the complementarity determining region (also termed “minimal recognition units” or “hypervariable region”) within antibody variable sequences. The CDRs permit the antigen-binding protein to specifically bind to a particular antigen of interest. There are three heavy chain variable region CDRs (CDR-H1, CDR-H2 and CDR-H3) and three light chain variable region CDRs (CDR-L1, CDR-L2 and CDR-L3). The CDRs in each of the two chains typically are aligned by the framework regions to form a structure that binds specifically to a specific epitope or domain on the target protein. From N-terminus to C-terminus, naturally-occurring light and heavy chain variable regions both typically conform to the following order of these elements: FW1, CDR1, FW2, CDR2, FW3, CDR3, FW4. For heavy chain variable regions, the order is typically: FW-H1, CDR-H1, FW-H2, CDR-H2, FW-H3, CDR-H3, and FW-H4 from N-terminus to C-terminus. For light chain variable regions, the order is typically: FW-L1, CDR-L1, FW-L2, CDR-L2, FW-L3, CDR-L3, FW-L4 from N-terminus to C-terminus. A numbering system has been devised for assigning numbers to amino acids that occupy positions in each of these domains. This numbering system is defined in Kabat Sequences of Proteins of Immunological Interest (1987 and 1991, NIH, Bethesda, MD), orChothia & Lesk, 1987, J. Mol. Biol. 196:901-917; Chothia et al., 1989, Nature 342:878-883. Complementarity determining regions (CDRs) and framework regions (FR) of a given antibody may be identified using this system. Other numbering systems for the amino acids in immunoglobulin chains include IMGT® (the international ImMunoGeneTics information system; Lefranc et al, Dev. Comp. Immunol. 29:185-203; 2005) and AHo (Honegger and Pluckthun, J. Mol. Biol. 309(3):657-670; 2001). The binding domains disclosed herein may utilize CDRs defined according to any of these systems. For any given embodiment containing more than one CDR, the CDRs may be defined in accordance with any of Kabat, Chothia, extended, IMGT, Paratome, AbM, and/or conformational definitions, or a combination of any of the foregoing. Any of the CDRs, either separately orwithin the context of variable domains, can be interpreted by one of skill in the art under any of these numbering systems as appropriate. One or more CDRs may be incorporated into a molecule either covalently or noncovalently to make it an antigen-binding protein.

In some embodiments, the antigen-binding proteins provided herein comprise one or more CDR(s) as part of a larger polypeptide chain. In some embodiments, the antigen-binding proteins covalently link the one or more CDR(s) to another polypeptide chain. In some embodiments, the antigen-binding proteins incorporate the one or more CDR(s) noncovalently. In some embodiments, the antigen-binding proteins may comprise at least one of the CDRs described herein incorporated into a biocompatible framework structure. In some embodiments, the biocompatible framework structure comprises a polypeptide or portion thereof that is sufficient to form a conformationally stable structural support, or framework, or scaffold, which is able to display one or more sequences of amino acids that bind to an antigen (e.g., CDRs, a variable region, etc.) in a localized surface region. Such structures can be a naturally occurring polypeptide or polypeptide “fold” (a structural motif), or can have one or more modifications, such as additions, deletions and/or substitutions of amino acids, relative to a naturally occurring polypeptide or fold. Depending on the embodiment, the scaffolds can be derived from a polypeptide of a variety of different species (or of more than one species), such as a human, a non-human primate or other mammal, other vertebrate, invertebrate, plant, bacteria or virus.

The term “consensus sequence” as used herein with regard to sequences refers to the generalized sequence representing all of the different combinations of permissible amino acids at each location of a group of sequences. A consensus sequence may provide insight into the conserved regions of related sequences where the unit (e.g. amino acid or nucleotide) is the same in most or all of the sequences, and regions that exhibit divergence between sequences. In the case of antibodies, the consensus sequence of a CDR may indicate amino acids that are important or dispensable for antigen binding. It is envisioned that consensus sequences may be prepared with any of the sequences provided herein, and the resultant various sequences derived from the consensus sequence can be validated to have similar effects as the template sequences.

In some embodiments, the antibody or binding fragment thereof comprises a combination of a CDR-H1, CDR-H2, CDR-H3, CDR-L1, CDR-L2, and a CDR-L3 where one or more of these CDRs is defined by a consensus sequence. The consensus sequences provided herein have been derived from the alignments of CDRs provided for herein. However, it is envisioned that alternative alignments may be done (e.g. using global or local alignment, or with different algorithms, such as Hidden Markov Models, seeded guide trees, Needleman-Wunsch algorithm, or Smith-Waterman algorithm) and as such, alternative consensus sequences can be derived.

Depending on the embodiment, the biocompatible framework structures are based on protein scaffolds or skeletons other than immunoglobulin domains. In some such embodiments, those framework structures are based on fibronectin, ankyrin, lipocalin, neocarzinostain, cytochrome b, CP1 zinc finger, PST1, coiled coil, LACI-D1, Z domain and/or tendamistat domains.

As used herein, the term “chimeric antibody” shall be given its ordinary meaning, and shall also refer to an antibody that contains one or more regions from one antibody and one or more regions from one or more other antibodies. For example, the framework regions of antigen-binding proteins disclosed herein that target, for example, CD70, may be derived from one or more different antibodies, such as a human antibody, or from a humanized antibody. In one example of a chimeric antibody, a portion of the heavy and/or light chain is identical with, homologous to, or derived from an antibody from a particular species or belonging to a particular antibody class or subclass, while the remainder of the chain(s) is/are identical with, homologous to, or derived from an antibody or antibodies from another species or belonging to another antibody class or subclass. Also provided herein are fragments of such antibodies that exhibit the desired biological activity. In some embodiments, the CARs disclosed herein comprise an anti-CD70 binding domain.

In some embodiments, the anti-CD70 binding domain comprises a VH and VL coupled by a linker. In some embodiments, the anti-CD70 binding domain is an scFv. In several embodiments, the CARs disclosed herein comprise an scFv as the binder for the tumor antigen. In several embodiments, the scFv is encoded by a polynucleotide comprising a sequence that has at least about 85%, about 90%, about 95%, or more, sequence identity to one or more of SEQ ID NOs: 23-24, 30-32, and/or 34-37. In several embodiments, the scFv is encoded by a polynucleotide comprising the sequence set forth in SEQ ID NO:23. In several embodiments, the scFv is encoded by a polynucleotide comprising the sequence set forth in SEQ ID NO:24. In several embodiments, the scFv is encoded by a polynucleotide comprising the sequence set forth in SEQ ID NO:30. In several embodiments, the scFv is encoded by a polynucleotide comprising the sequence set forth in SEQ ID NO:31. In several embodiments, the scFv is encoded by a polynucleotide comprising the sequence set forth in SEQ ID NO:32. In several embodiments, the scFv is encoded by a polynucleotide comprising the sequence set forth in SEQ ID NO:34. In several embodiments, the scFv is encoded by a polynucleotide comprising the sequence set forth in SEQ ID NO:35. In several embodiments, the scFv is encoded by a polynucleotide comprising the sequence set forth in SEQ ID NO:36. In several embodiments, the scFv is encoded by a polynucleotide comprising the sequence set forth in SEQ ID NO:37. In several embodiments, the scFv comprises an amino acid sequence that has at least about 85%, about 90%, about 95%, or more, sequence identity to one or more of SEQ ID NOs: 25-26, 47-49, and/or 51-54. In some embodiments, the scFv comprises the amino acid sequence set forth in SEQ ID NO:25. In some embodiments, the scFv comprises the amino acid sequence set forth in SEQ ID NO:26. In some embodiments, the scFv comprises the amino acid sequence set forth in SEQ ID NO:47. In some embodiments, the scFv comprises the amino acid sequence set forth in SEQ ID NO:48. In some embodiments, the scFv comprises the amino acid sequence set forth in SEQ ID NO:49. In some embodiments, the scFv comprises the amino acid sequence set forth in SEQ ID NO:51. In some embodiments, the scFv comprises the amino acid sequence set forth in SEQ ID NO:52. In some embodiments, the scFv comprises the amino acid sequence set forth in SEQ ID NO:53. In some embodiments, the scFv comprises the amino acid sequence set forth in SEQ ID NO:54.

In several embodiments, the various domains/subdomains are separated by a linker such as, a GS3 linker (SEQ ID NO: 208) or a GS2 linker (SEQ ID NOs: 15 and 16, nucleotide and protein, respectively) is used (or a GSn linker). Other linkers used according to various embodiments disclosed herein include, but are not limited to those encoded by SEQ ID NOs: 17, 19, or 21. In several embodiments, other linkers comprise the peptide sequence of one of SEQ ID NOs: 18, 20, or 22. In some embodiments, the linker comprises the sequence of SEQ ID NO:50. This provides the potential to separate the various component parts of the receptor complex along the polynucleotide, which can enhance expression, stability, and/or functionality of the receptor complex.

### Cytotoxic Signaling Complex

Some embodiments of the compositions and methods described herein relate to a chimeric antigen receptor (e.g., a CAR directed to CD70) that includes a cytotoxic signaling complex. As disclosed herein, according to several embodiments, the provided cytotoxic receptor complexes comprise one or more transmembrane and/or intracellular domains that initiate cytotoxic signaling cascades upon the extracellular domain(s) binding to ligands on the surface of target cells.

In several embodiments, the cytotoxic signaling complex comprises at least one transmembrane domain, at least one co-stimulatory domain, and/or at least one signaling domain. In some embodiments, more than one component part makes up a given domain—e.g., a co-stimulatory domain may comprise two subdomains. Moreover, in some embodiments, a domain may serve multiple functions, for example, a transmembrane domain may also serve to provide signaling function.

### Transmembrane Domains

Some embodiments of the compositions and methods described herein relate to chimeric receptors (e.g., tumor antigen-directed CARs and/or ligand-directed chimeric receptors) that comprise a transmembrane domain. In several embodiments in which a transmembrane domain is employed, the portion of the transmembrane protein employed retains at least a portion of its normal transmembrane domain.

In several embodiments, however, the transmembrane domain comprises at least a portion of CD8, a transmembrane glycoprotein normally expressed on both T cells and NK cells. In several embodiments, the transmembrane domain comprises CD8a. In several embodiments, the transmembrane domain comprises a CD8a transmembrane domain. In several embodiments, the CD8a transmembrane domain has the nucleic acid sequence of SEQ ID NO: 3. In several embodiments, the CD8a transmembrane domain is truncated or modified and has at least 70%, at least 75%, at least 80%, at least 85%, at least 90%, at least 95% sequence identity with the CD8a having the sequence of SEQ ID NO: 3. In several embodiments, the CD8a transmembrane domain comprises the amino acid sequence of SEQ ID NO: 4. In several embodiments, the CD8a transmembrane domain is truncated or modified and has at least 70%, at least 75%, at least 80%, at least 85%, at least 90%, at least 95% sequence identity with the CD8a having the sequence of SEQ ID NO: 4.

In several embodiments, the transmembrane domain is coupled to a “hinge” domain. In several embodiments, the “hinge” domain of CD8a has the nucleic acid sequence of SEQ ID NO: 1. In several embodiments, the CD8a hinge is truncated or modified and has at least 70%, at least 75%, at least 80%, at least 85%, at least 90%, at least 95% sequence identity with the CD8a having the sequence of SEQ ID NO: 1. In several embodiments, the “hinge” of CD8a comprises the amino acid sequence of SEQ ID NO: 2. In several embodiments, the CD8a hinge can be truncated or modified, such that it has at least 70%, at least 75%, at least 80%, at least 85%, at least 90%, at least 95% sequence identity with the sequence of SEQ ID NO: 2.

In several embodiments, the CD8a hinge and CD8a transmembrane domain are used together (referred to herein as the CD8 hinge/transmembrane complex). In several embodiments, CD8 hinge/transmembrane complex is encoded by the nucleic acid sequence of SEQ ID NO: 13. In several embodiments, the CD8 hinge/transmembrane complex is truncated or modified and has at least 70%, at least 75%, at least 80%, at least 85%, at least 90%, at least 95% sequence identity with the CD8 hinge/transmembrane complex having the sequence of SEQ ID NO: 13. In several embodiments, the CD8 hinge/transmembrane complex comprises the amino acid sequence of SEQ ID NO: 14. In several embodiments, the CD8 hinge/transmembrane complex hinge is truncated or modified and has at least 70%, at least 75%, at least 80%, at least 85%, at least 90%, at least 95% sequence identity with the CD8 hinge/transmembrane complex having the sequence of SEQ ID NO: 14.

### Stimulatory Domains

Some embodiments of the compositions and methods described herein relate to chimeric antigen receptors that comprise a stimulatory domain. In addition to the various transmembrane domains and signaling domains (and the combination transmembrane/signaling domains), additional stimulating molecules can be provided, in several embodiments. These can be certain molecules that, for example, further enhance activity of the immune cells. Cytokines may be used in some embodiments. For example, certain interleukins, such as IL-2 and/or IL-15 as non-limiting examples, are used. In some embodiments, the immune cells for therapy are engineered to express such molecules as a secreted form. In additional embodiments, such stimulatory domains are engineered to be membrane bound, acting as autocrine stimulatory molecules (or even as paracrine stimulators to neighboring cells).

In several embodiments, the NK cells disclosed herein are engineered to express interleukin 15 (IL15, IL-15). In some embodiments, the IL15 is expressed from a separate cassette on the construct comprising any one of the CARs disclosed herein. In some embodiments, the IL15 is expressed in the same cassette as any one of the CARs disclosed herein, optionally separated by a cleavage site, for example, a proteolytic cleavage site or a T2A, P2A, E2A, or F2A self-cleaving peptide cleavage site. In some embodiments, the IL15 is a membrane-bound IL15 (mbIL15). In some embodiments, the mbIL15 comprises a native IL15 sequence, such as a human native IL15 sequence, and at least one transmembrane domain. In some embodiments, the native IL15 sequence is encoded by a sequence having at least 85%, at least 90%, at least 95% sequence identity to SEQ ID NO: 11. In some embodiments, the native IL15 sequence comprise a peptide sequence having at least 85%, at least 90%, at least 95% sequence identity to SEQ ID NO: 12. In some embodiments, the native IL15 sequence comprises the amino acid sequence of SEQ ID NO: 12. In some embodiments, the at least one transmembrane domain comprises a CD8 transmembrane domain. In some embodiments, the mbIL15 may comprise additional components, such as a leader sequence and/or a hinge sequence. In some embodiments, the leader sequence is a CD8 leader sequence. In some embodiments, the hinge sequence is a CD8 hinge sequence.

In some embodiments, the tumor antigen-directed CARs and/or tumor ligand-directed chimeric receptors are encoded by a polynucleotide that encodes for one or more cytosolic protease cleavage sites. Such sites are recognized and cleaved by a cytosolic protease, which can result in separation (and separate expression) of the various component parts of the receptor encoded by the polynucleotide. In some embodiments, the tumor antigen-directed CARs and/or tumor ligand-directed chimeric receptor are encoded by a polynucleotide that encodes for one or more self-cleaving peptides, for example a T2A cleavage site, a P2A cleavage site, an E2A cleavage site, and/or an F2A cleavage site. As a result, depending on the embodiment, the various constituent parts of an engineered cytotoxic receptor complex can be delivered to an NK cell or T cell in a single vector or by multiple vectors. Thus, as shown schematically, in the Figures, a construct can be encoded by a single polynucleotide, but also include a cleavage site, such that downstream elements of the constructs are expressed by the cells as a separate protein (as is the case in some embodiments with IL-15). In several embodiments, a T2A cleavage site is used. In several embodiments, a T2A cleavage site has the nucleic acid sequence of SEQ ID NO: 9. In several embodiments, T2A cleavage site can be truncated or modified, such that it has at least 70%, at least 75%, at least 80%, at least 85%, at least 90%, at least 95% sequence identity with the sequence of SEQ ID NO: 9. In several embodiments, the T2A cleavage site comprises the amino acid sequence of SEQ ID NO: 10. In several embodiments, the T2A cleavage site is truncated or modified and has at least 70%, at least 75%, at least 80%, at least 85%, at least 90%, at least 95% sequence identity with the T2A cleavage site having the sequence of SEQ ID NO: 10.

In several embodiments, NK cells are engineered to express membrane-bound interleukin 15 (mbIL15). In such embodiments, mbIL15 expression on the NK enhances the cytotoxic effects of the engineered NK cell by enhancing the proliferation and/or longevity of the NK cells. In several embodiments, the mbIL15 is encoded by the same polynucleotide as the CAR, though a separate vector may also be used. In several embodiments, mbIL15 has the nucleic acid sequence of SEQ ID NO: 27. In several embodiments, mbIL15 can be truncated or modified, such that it has at least 70%, at least 75%, at least 80%, at least 85%, at least 90%, at least 95% sequence identity with the sequence of SEQ ID NO: 27. In several embodiments, the mbIL15 comprises the amino acid sequence of SEQ ID NO: 28. In several embodiments, the mbIL15 is truncated or modified and has at least 70%, at least 75%, at least 80%, at least 85%, at least 90%, at least 95% sequence identity with the mbIL15 having the sequence of SEQ ID NO: 28. In several embodiments, the mbIL15 comprises the amino acid sequence of SEQ ID NO: 213. In several embodiments, the mbIL15 is truncated or modified and has at least 70%, at least 75%, at least 80%, at least 85%, at least 90%, at least 95% sequence identity with the mbIL15 having the sequence of SEQ ID NO: 213. Membrane-bound IL15 sequences are explored in PCT publications WO 2018/183385 and WO 2020/056045, each of which is hereby expressly incorporated by reference in its entirety and pertaining to membrane-bound IL15 sequences.

### Signaling Domains

Some embodiments of the compositions and methods described herein relate to a chimeric receptor (e.g., tumor antigen-directed CARs and/or tumor ligand-directed chimeric receptors) that includes a signaling domain. For example, immune cells engineered according to several embodiments disclosed herein may comprise at least one subunit of the CD3 T cell receptor complex (or a fragment thereof). In several embodiments, the signaling domain comprises the CD3zeta subunit. In several embodiments, the CD3zeta is encoded by the nucleic acid sequence of SEQ ID NO: 7. In several embodiments, the CD3zeta can be truncated or modified, such that it has at least 70%, at least 75%, at least 80%, at least 85%, at least 90%, at least 95% sequence identity with the CD3zeta having the sequence of SEQ ID NO: 7. In several embodiments, the CD3zeta domain comprises the amino acid sequence of SEQ ID NO: 8. In several embodiments, the CD3zeta domain is truncated or modified and has at least 70%, at least 75%, at least 80%, at least 85%, at least 90%, at least 95% sequence identity with the CD3zeta domain having the sequence of SEQ ID NO: 8.

In several embodiments, unexpectedly enhanced signaling is achieved through the use of multiple signaling domains whose activities act synergistically. For example, in several embodiments, the signaling domain further comprises an OX40 domain. In several embodiments, the OX40 domain is an intracellular signaling domain. In several embodiments, the OX40 intracellular signaling domain has the nucleic acid sequence of SEQ ID NO: 5. In several embodiments, the OX40 intracellular signaling domain can be truncated or modified, such that it has at least 70%, at least 75%, at least 80%, at least 85%, at least 90%, at least 95% sequence identity with the OX40 having the sequence of SEQ ID NO: 5. In several embodiments, the OX40 intracellular signaling domain comprises the amino acid sequence of SEQ ID NO: 6. In several embodiments, the OX40 intracellular signaling domain is truncated or modified and has at least 70%, at least 75%, at least 80%, at least 85%, at least 90%, at least 95% sequence identity with the OX40 intracellular signaling domain having the sequence of SEQ ID NO: 6. In several embodiments, OX40 is used as the sole transmembrane/signaling domain in the construct, however, in several embodiments, OX40 can be used with one or more other domains. For example, combinations of OX40 and CD3zeta are used in some embodiments. By way of further example, combinations of CD28, OX40, 4-1 BB, and/or CD3zeta are used in some embodiments.

### Chimeric Antigen Receptor Constructs

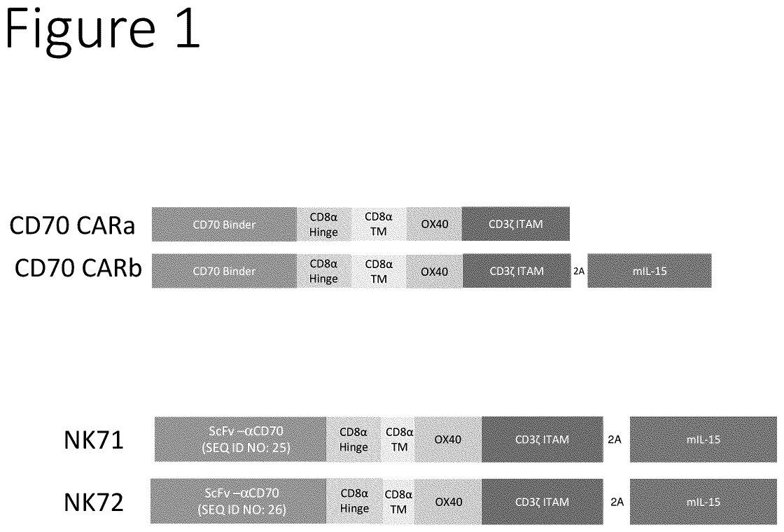

In several embodiments, there are provided for herein a variety of cytotoxic receptor complexes (also referred to as cytotoxic receptors) are provided for herein with the general structure of a chimeric antigen receptor. FIG. 1 shows a schematic of a CD70 directed CAR. CARa is a schematic of a non-limiting embodiment of a CAR. CARb shows a schematic of a polynucleotide encoding the CAR, as well as the optional T2A and mbIL15. FIG. 1 also depicts two non-limiting polynucleotide constructs NK71 and NK72, which target CD70 (including the optional T2A and mbIL15).

As shown in the figures, several embodiments of the polynucleotide encoding a CAR include an anti-tumor binder, a CD8a hinge domain, a CD8a transmembrane domain, an OX40 domain, a CD3ζ domain (such as a CD3ζ ITAM domain), a 2A cleavage site, and/or a membrane-bound IL-15 domain (though, as above, in several embodiments soluble IL-15 is used). In several embodiments, the binding and activation functions are engineered to be performed by separate domains. In several embodiments, the general structure of the chimeric antigen receptor construct includes a hinge and/or transmembrane domain. These may, in some embodiments, be fulfilled by a single domain, or a plurality of subdomains may be used, in several embodiments. The receptor complex further comprises a signaling domain, which transduces signals after binding of the homing moiety to the target cell, ultimately leading to the cytotoxic effects on the target cell. In several embodiments, the complex further comprises a co-stimulatory domain, which operates, synergistically, in several embodiments, to enhance the function of the signaling domain. Expression of these complexes in immune cells, such as NK cells and/or T cells, allows the targeting and destruction of particular target cells, such as cancerous cells that express a given tumor marker. Some such receptor complexes comprise an extracellular domain comprising an anti-CD70 moiety, or CD70-binding moiety, that binds CD70 on the surface of target cells and activates the engineered cell. The CD3zeta ITAM subdomain may act in concert as a signaling domain. The IL-15 domain, e.g., mbIL-15 domain, may act as a stimulatory domain. The IL-15 domain, e.g. mbIL-15 domain, may render immune cells (e.g., NK or T cells) expressing it particularly efficacious against target tumor cells. It shall be appreciated that the IL-15 domain, such as an mbIL-15 domain, can, in accordance with several embodiments, be encoded on a separate construct. Additionally, each of the components may be encoded in one or more separate constructs.

Disclosed herein in some embodiments are anti-CD70 binding domains. In some embodiments, the anti-CD70 binding domains are scFvs. These anti-CD70 binding domains are specific for and/or preferentially bind to CD70. The anti-CD70 binding domains disclosed herein may be incorporated into any one of the chimeric antigen receptor constructs disclosed herein. The anti-CD70 binding domains disclosed herein may furthermore be expressed by a cell, either separately or within an anti-CD70 CAR.

In some embodiments, the anti-CD70 binding domain comprises a polynucleotide sequence that is at least 70%, 75%, 80%, 85%, 90%, 91%, 92%, 93%, 94%, 95%, 96%, 97%, 98%, 99%, or more, or a range defined by any two of the aforementioned percentages, identical to, or derived from, the sequence of either SEQ ID NO: 23 and/or SEQ ID NO: 24.

In some embodiments, the anti-CD70 binding domain comprises a heavy chain variable region and a light chain variable region. In some embodiments, the heavy chain variable region comprises a CDR-H1, CDR-H2, and CDR-H3 and the light chain variable region comprises a CDR-L1, CDR-L2, and CDR-L3. In some embodiments, the CDR-H1 comprises a sequence having at least 75%, 80%, 85%, 90%, 95%, 99%, or 100% sequence identity to a sequence selected from SEQ ID NOs: 102-103 or 110; the CDR-H2 comprises a sequence having at least 75%, 80%, 85%, 90%, 95%, 99%, or 100% sequence identity to a sequence selected from SEQ ID NOs: 104-106 or 111; the CDR-H3 comprises a sequence having at least 75%, 80%, 85%, 90%, 95%, 99%, or 100% sequence identity to a sequence selected from SEQ ID NOs: 107-109 or 112; the CDR-L1 comprises a sequence having at least 75%, 80%, 85%, 90%, 95%, 99%, or 100% sequence identity to a sequence selected from SEQ ID NOs: 131-133 or 140; the CDR-L2 comprises a sequence having at least 75%, 80%, 85%, 90%, 95%, 99%, or 100% sequence identity to a sequence selected from SEQ ID NOs: 134-136 or 141; and the CDR-L3 comprises a sequence having at least 75%, 80%, 85%, 90%, 95%, 99%, or 100% sequence identity to a sequence selected from SEQ ID NOs: 137-139 or 142.

In some embodiments of the anti-CD70 binding domains, the heavy chain variable region comprises an amino acid sequence having at least 75%, 80%, 85%, 90%, 95%, 99%, or 100% sequence identity to any sequence selected from SEQ ID NOs: 151-153 and 157. In some embodiments, the light chain variable region comprises an amino acid sequence having at least 75%, 80%, 85%, 90%, 95%, 99%, or 100% sequence identity to any sequence selected from SEQ ID NOs: 154-156 and 158.

In some embodiments of the anti-CD70 binding domains: 1) the heavy chain variable region comprises the CDR-H1, CDR-H2, CDR-H3 within SEQ ID NO: 151 and the light chain variable region comprises the CDR-L1, CDR-L2, CDR-L3 within SEQ ID NO: 154; 2) the heavy chain variable region comprises the CDR-H1, CDR-H2, CDR-H3 within SEQ ID NO: 152 and the light chain variable region comprises the CDR-L1, CDR-L2, CDR-L3 within SEQ ID NO: 155; 3) the heavy chain variable region comprises the CDR-H1, CDR-H2, CDR-H3 within SEQ ID NO: 153 and the light chain variable region comprises the CDR-L1, CDR-L2, CDR-L3 within SEQ ID NO: 156; 4) the heavy chain variable region comprises the CDR-H1, CDR-H2, CDR-H3 within SEQ ID NO: 157 and the light chain variable region comprises the CDR-L1, CDR-L2, CDR-L3 within SEQ ID NO: 158. Other light chain variable regions, heavy chain variable regions, scFvs and CARs targeting CD70 can be found in US Patent Publication No: 2022/0002424, which is incorporated by reference herein in its entirety.

In some embodiments of the anti-CD70 binding domains: 1) the heavy chain variable region comprises SEQ ID NO: 151 and the light chain variable region comprises SEQ ID NO: 154; 2) the heavy chain variable region comprises SEQ ID NO: 152 and the light chain variable region comprises SEQ ID NO: 155; 3) the heavy chain variable region comprises SEQ ID NO: 153 and the light chain variable region comprises SEQ ID NO: 156; or 4) the heavy chain variable region comprises SEQ ID NO: 157 and the light chain variable region comprises SEQ ID NO: 158.

In some embodiments of the anti-CD70 binding domains, the heavy chain variable region and/or light chain variable region comprise a framework. In some embodiments, the heavy chain variable region comprises a FW-H1, FW-H2, FW-H3, and FW-H4. In some embodiments, the heavy chain variable region comprises the order of FW-H1, CDR-H1, FW-H2, CDR-H2, FW-H3, CDR-H3, and FW-H4 from N-terminus to C-terminus. In some embodiments, the light chain variable region comprises a FW-L1, FW-L2, FW-L3, and FW-L4. In some embodiments, the light chain variable region comprises the order of FW-11, CDR-L1, FW-L2, CDR-L2, FW-L3, CDR-L3, FW-L4 from N-terminus to C-terminus. In some embodiments, the FW-H1 comprises a sequence having at least 95%, 99%, or 100% sequence identity to a sequence selected from SEQ ID NOs: 73-76; the FW-H2 comprises a sequence having at least 95%, 99%, or 100% sequence identity to a sequence selected from SEQ ID NOs: 77-80; the FW-H3 comprises a sequence having at least 95%, 99%, or 100% sequence identity to a sequence selected from SEQ ID NOs: 81-96; the FW-H4 comprises a sequence having at least 95%, 99%, or 100% sequence identity to a sequence selected from SEQ ID NOs: 97-101; the FW-L1 comprises a sequence having at least 95%, 99%, or 100% sequence identity to a sequence selected from SEQ ID NOs: 113-116; the FW-L2 comprises a sequence having at least 95%, 99%, or 100% sequence identity to a sequence selected from SEQ ID NOs: 117-120; the FW-L3 comprises a sequence having at least 95%, 99%, or 100% sequence identity to a sequence selected from SEQ ID NOs: 121-124; the FW-L4 comprises a sequence having at least 95%, 99%, or 100% sequence identity to a sequence selected from SEQ ID NOs: 125-130.

In some embodiments of the anti-CD70 binding domains, the heavy chain variable domain is encoded by a nucleic acid sequence having at least 75%, 80%, 85%, 90%, 95%, 99%, or 100% sequence identity to any sequence selected from SEQ ID NOs: 143-145 and 149.

In some embodiments of the anti-CD70 binding domains, the light chain variable domain is encoded by a nucleic acid sequence having at least 75%, 80%, 85%, 90%, 95%, 99%, or 100% sequence identity to any sequence selected from SEQ ID NOs: 146-148 and 150.

In some embodiments, the anti-CD70 binding domain is an antibody, Fab′ fragment, F(ab′)₂ fragment, or scFv.

In several embodiments, the anti-CD70 binding domain is encoded by a polynucleotide sequence comprising a sequence that is at least 70%, 75%, 80%, 85%, 90%, 91%, 92%, 93%, 94%, 95%, 96%, 97%, 98%, 99%, or more, or a range defined by any two of the aforementioned percentages, identical to the sequence of one or more of SEQ ID NOs: 30-32 or 34-37. In several embodiments, the anti-CD70 binding domain comprises an amino acid sequence that has at least about 85%, about 90%, about 95%, or more, sequence identity to one or more of SEQ ID NOs: 47-49 or 51-54.

In some embodiments, the anti-CD70 binding domain comprises a heavy chain variable region (VH) comprising a CDR-H1, a CDR-H2, and a CDR-H3. In some embodiments, the CDR-H1 comprises the amino acid sequence set forth in SEQ ID NO: 102, 103, 110, or 205. In some embodiments, the CDR-H2 comprises the amino acid sequence set forth in SEQ ID NO: 104, 105, 106, 111, 206, or 225. In some embodiments, the CDR-H3 comprises the amino acid sequence set forth in SEQ ID NO: 107, 108, 109, 112, 207, or 226. In some embodiments, the VH comprises a CDR-H1, a CDR-H2, and a CDR-H3 comprising the amino acid sequences set forth in SEQ ID NOS: 205, 206, and 207, respectively. In some embodiments, the VH comprises a CDR-H1, a CDR-H2, and a CDR-H3 comprising the amino acid sequences set forth in SEQ ID NOS: 110, 111, and 112, respectively. In some embodiments, the VH comprises a CDR-H1, a CDR-H2, and a CDR-H3 comprising the amino acid sequences set forth in SEQ ID NOS: 205, 225, and 226, respectively. In some embodiments, the VH comprises the amino acid sequence set forth in SEQ ID NO: 151, 152, 153, or 157. In some embodiments, the VH comprises the amino acid sequence set forth in SEQ ID NO: 151. In some embodiments, the VH comprises the amino acid sequence set forth in SEQ ID NO: 152. In some embodiments, the VH comprises the amino acid sequence set forth in SEQ ID NO: 153. In some embodiments, the VH comprises the amino acid sequence set forth in SEQ ID NO: 157.

In some embodiments, the anti-CD70 binding domain comprises a light chain variable region (VL) comprising a CDR-L1, a CDR-L2, and a CDR-L3. In some embodiments, the CDR-L1 comprises the amino acid sequences set forth in SEQ ID NO: 131, 132, 133, 140, 204, or 209. In some embodiments, the CDR-L2 comprises the amino acid sequences set forth in SEQ ID NO: 134, 135, 136, 141, 210, or 223. In some embodiments, the CDR-L3 comprises the amino acid sequences set forth in SEQ ID NO: 137, 138, 139, 142, 211, or 224. In some embodiments, the VL comprises a CDR-L1, a CDR-L2, and a CDR-L3 comprising the amino acid sequences set forth in SEQ ID NOS: 209, 210, and 211, respectively. In some embodiments, the VL comprises a CDR-L1, a CDR-L2, and a CDR-L3 comprising the amino acid sequences set forth in SEQ ID NOS: 140, 141, and 142, respectively. In some embodiments, the VL comprises a CDR-L1, a CDR-L2, and a CDR-L3 comprising the amino acid sequences set forth in SEQ ID NOS: 204, 223, and 224, respectively. In some embodiments, the VL comprises the amino acid sequence set forth in SEQ ID NO:154, 155, 156, or 158. In some embodiments, the VL comprises the amino acid sequence set forth in SEQ ID NO:154. In some embodiments, the VL comprises the amino acid sequence set forth in SEQ ID NO:155. In some embodiments, the VL comprises the amino acid sequence set forth in SEQ ID NO:156. In some embodiments, the VL comprises the amino acid sequence set forth in SEQ ID NO:158.

In some embodiments, the VH comprises a CDR-H1, a CDR-H2, and a CDR-H3 comprising the amino acid sequences set forth in SEQ ID NOS: 205, 206, and 207, respectively; and the VL comprises a CDR-L1, a CDR-L2, and a CDR-L3 comprising the amino acid sequences set forth in SEQ ID NOS: 209, 210, and 211, respectively. In some embodiments, the VH comprises the amino acid sequence set forth in SEQ ID NO:153 and the VL comprises the amino acid sequence set forth in SEQ ID NO:156.

In some embodiments, the VH comprises a CDR-H1, a CDR-H2, and a CDR-H3 comprising the amino acid sequences set forth in SEQ ID NOS: 110, 111, and 112, respectively; and the VL comprises a CDR-L1, a CDR-L2, and a CDR-L3 comprising the amino acid sequences set forth in SEQ ID NOS: 140, 141, and 142, respectively. In some embodiments, the VH comprises the amino acid sequence set forth in SEQ ID NO:157 and the VL comprises the amino acid sequence set forth in SEQ ID NO:158.

In some embodiments, the VH comprises a CDR-H1, a CDR-H2, and a CDR-H3 comprising the amino acid sequences set forth in SEQ ID NOS: 205, 225, and 226, respectively; and the VL comprises a CDR-L1, a CDR-L2, and a CDR-L3 comprising the amino acid sequences set forth in SEQ ID NOS: 204, 223, and 224, respectively. In some embodiments, the VH comprises the amino acid sequence set forth in SEQ ID NO:152 and the VL comprises the amino acid sequence set forth in SEQ ID NO:155.

In some embodiments, the anti-CD70 binding domain comprises a VH and VL coupled by a linker. In some embodiments, the anti-CD70 binding domain is an scFv. In several embodiments, the CARs disclosed herein comprise an scFv as the binder for the tumor antigen. In several embodiments, the linker comprises the amino acid sequence of SEQ ID NO: 50 or 208.

In several embodiments, the scFv comprises an amino acid sequence that has at least about 85%, about 90%, about 95%, or more, sequence identity to one or more of SEQ ID NOs: 25-26, 47-49, and/or 51-54. In some embodiments, the scFv comprises the amino acid sequence set forth in SEQ ID NO:25. In some embodiments, the scFv comprises the amino acid sequence set forth in SEQ ID NO:26. In some embodiments, the scFv comprises the amino acid sequence set forth in SEQ ID NO:47. In some embodiments, the scFv comprises the amino acid sequence set forth in SEQ ID NO:48. In some embodiments, the scFv comprises the amino acid sequence set forth in SEQ ID NO:49. In some embodiments, the scFv comprises the amino acid sequence set forth in SEQ ID NO:51. In some embodiments, the scFv comprises the amino acid sequence set forth in SEQ ID NO:52. In some embodiments, the scFv comprises the amino acid sequence set forth in SEQ ID NO:53. In some embodiments, the scFv comprises the amino acid sequence set forth in SEQ ID NO:54.

Also disclosed herein are CARs. In some embodiments, the CARs are anti-CD70 CARs. In some embodiments, the CARs comprise one or more of the anti-CD70 binding domains disclosed herein.

In some embodiments, the CARs further comprise an OX40 subdomain and a CD3zeta subdomain. In several embodiments, the OX40 subdomain is encoded by a sequence having at least 95% sequence identity to SEQ ID NO: 5. In several embodiments, the OX40 subdomain comprises an amino acid sequence having at least 85%, at least 90%, at least 95%, at least 99%, or at least 100% sequence identity to SEQ ID NO: 6. In several embodiments, the OX40 subdomain comprises the amino acid sequence of SEQ ID NO: 6. In several embodiments, the CD3zeta subdomain is encoded by a sequence having at least 95% sequence identity to SEQ ID NO: 7. In several embodiments, the CD3zeta subdomain comprises an amino acid sequence having at least 85%, at least 90%, at least 95%, at least 99%, or at least 100% sequence identity to SEQ ID NO: 8. In several embodiments, the CD3zeta subdomain comprises the amino acid sequence of SEQ ID NO: 8. In several embodiments, the mbIL15 is encoded by a sequence having at least 95% sequence identity to SEQ ID NO: 27. In several embodiments, the mbIL15 comprises the amino acid sequence of SEQ ID NO: 213. In several embodiments, the one or more of SEQ ID NOS: 30-32 and/or 34-37, the polynucleotide encoding the OX40 subdomain, the polynucleotide encoding the CD3zeta subdomain, and the polynucleotide encoding mbIL15 are arranged in a 5′ to 3′ orientation within the polynucleotide.

In several embodiments, an anti-CD70 CAR is provided and is encoded by a polynucleotide having at least 70%, 75%, 80%, 85%, 90%, 91%, 92%, 93%, 94%, 95%, 96%, 97%, 98%, 99%, or more, or a range defined by any two of the aforementioned percentages, identical to the sequence of one or more of SEQ ID NOs: 40-46 or a portion thereof (e.g. a portion excluding the mbIL15 sequence and/or self-cleaving peptide sequence). In several embodiments, the CAR comprises an amino acid sequence having at least 70%, 75%, 80%, 85%, 90%, 91%, 92%, 93%, 94%, 95%, 96%, 97%, 98%, 99%, or more, or a range defined by any two of the aforementioned percentages, identical to the sequence of one or more of SEQ ID NOs: 55-63, or a portion thereof (e.g. a portion excluding the mbIL15 sequence and/or self-cleaving peptide sequence). In several embodiments, the CAR comprises an amino acid sequence having at least 70%, 75%, 80%, 85%, 90%, 91%, 92%, 93%, 94%, 95%, 96%, 97%, 98%, 99%, or more, or a range defined by any two of the aforementioned percentages, identical to the sequence of one or more of SEQ ID NOs: 64-72, or a portion thereof. In several embodiments, the CAR comprises an amino acid sequence having at least 70%, 75%, 80%, 85%, 90%, 91%, 92%, 93%, 94%, 95%, 96%, 97%, 98%, 99%, or more, or a range defined by any two of the aforementioned percentages, identical to the sequence of any one of SEQ ID NOs: 214-222. In several embodiments, the CAR comprises an amino acid sequence of any one of SEQ ID NOs: 214-222.

In several embodiments, there is provided a polynucleotide encoding an anti-CD70 binding domain/CD8a hinge/CD8a transmembrane domain/OX40/CD3zeta chimeric antigen receptor complex (see FIG. 1, CD70 CARa). The polynucleotide comprises or is composed of an anti-CD70 binding domain, a CD8alpha hinge, a CD8a transmembrane domain, an OX40 domain, and a CD3zeta domain as described herein. In several embodiments, the polynucleotide further encodes mbIL15 (see FIG. 1, CD70CARb). In several embodiments, this anti-CD70 binding domain comprises an scFv. In several embodiments, the anti-CD70 scFv is encoded by a nucleic acid molecule having a sequence according to any one of SEQ ID NOS: 30-32 or 34-37. In several embodiments, the anti-CD70 scFv is encoded by a nucleic acid sequence that shares at least about 80%, at least about 85%, at least about 90%, at least about 95%, at least about 96%, at least about 97%, at least about 98%, or at least about 99%, sequence identity, homology and/or functional equivalence with any one of SEQ ID NOS: 30-32 or 34-37. In several embodiments, the scFv comprises an amino acid having at least about 80%, at least about 85%, at least about 90%, at least about 95%, at least about 96%, at least about 97%, at least about 98%, or at least about 99%, sequence identity, homology and/or functional equivalence with any one of SEQ ID NOS: 47-49 or 51-54. In several embodiments, an anti-CD70 CAR is encoded by a nucleic acid sequence that shares at least about 80%, at least about 85%, at least about 90%, at least about 95%, at least about 96%, at least about 97%, at least about 98%, or at least about 99%, sequence identity, homology and/or functional equivalence with any one of SEQ ID NOS: 38-46, or a portion thereof (e.g. a portion excluding the mbIL15 sequence and/or self-cleaving peptide sequence). In several embodiments, the anti-CD70 CAR comprises an amino acid sequence having at least about 80%, at least about 85%, at least about 90%, at least about 95%, at least about 96%, at least about 97%, at least about 98%, or at least about 99%, sequence identity, homology and/or functional equivalence with any one of SEQ ID NOS: 55-63, or a portion thereof (e.g. a portion excluding the mbIL15 sequence and/or self-cleaving peptide sequence). In several embodiments, the anti-CD70 CAR comprises an amino acid sequence having at least about 80%, at least about 85%, at least about 90%, at least about 95%, at least about 96%, at least about 97%, at least about 98%, or at least about 99%, sequence identity, homology and/or functional equivalence with any one of SEQ ID NOS: 64-72, or a portion thereof. In several embodiments, the anti-CD70 CAR comprises an amino acid sequence having at least about 80%, at least about 85%, at least about 90%, at least about 95%, at least about 96%, at least about 97%, at least about 98%, or at least about 99%, sequence identity, homology and/or functional equivalence with any one of SEQ ID NOS: 214-222, or a portion thereof.

In several embodiments, there is provided a polynucleotide encoding an anti-CD70 scFv/CD8a hinge/CD8a transmembrane domain/OX40/CD3zeta chimeric antigen receptor complex (see FIG. 1, NK71). The polynucleotide comprises or is composed of an anti CD70 scFv encoded by a nucleic acid sequence that shares at least about 80%, at least about 85%, at least about 90%, at least about 95%, at least about 96%, at least about 97%, at least about 98%, or at least about 99%, sequence identity, homology and/or functional equivalence with SEQ ID NO: 23. In several embodiments, the polynucleotide further encodes mbIL15. In several embodiments, the polynucleotide encodes an scFv that shares at least about 80%, at least about 85%, at least about 90%, at least about 95%, at least about 96%, at least about 97%, at least about 98%, or at least about 99%, sequence identity, homology and/or functional equivalence with SEQ ID NO: 25.

In several embodiments, there is provided a polynucleotide encoding an anti CD70 scFv/CD8a hinge/CD8a transmembrane domain/OX40/CD3zeta chimeric antigen receptor complex (see FIG. 1, NK72). The polynucleotide comprises or is composed of an anti CD70 scFv encoded by a nucleic acid sequence that shares at least about 80%, at least about 85%, at least about 90%, at least about 95%, at least about 96%, at least about 97%, at least 98%, or at least about 99%, sequence identity, homology and/or functional equivalence with SEQ ID NO: 24. In several embodiments, the polynucleotide further encodes mbIL15. In several embodiments, the polynucleotide encodes an scFv that shares at least about 80%, at least about 85%, at least about 90%, at least about 95%, at least about 96%, at least about 97%, at least about 98%, or at least about 99%, sequence identity, homology and/or functional equivalence with SEQ ID NO: 26. However, in several embodiments, the anti-CD70 CARs disclosed herein do not comprise the scFv of SEQ ID NO: 25 or 26.

Also provided herein are natural killer (NK) cells expressing any of the anti-CD70 CARs described herein. In some embodiments, the CAR comprises the amino acid sequence of any one of SEQ ID NOS:214-222. In some embodiments, the CAR comprises the amino acid sequence of SEQ ID NO:214. In some embodiments, the CAR comprises the amino acid sequence of SEQ ID NO:215. In some embodiments, the CAR comprises the amino acid sequence of SEQ ID NO:216. In some embodiments, the CAR comprises the amino acid sequence of SEQ ID NO:217. In some embodiments, the CAR comprises the amino acid sequence of SEQ ID NO:218. In some embodiments, the CAR comprises the amino acid sequence of SEQ ID NO:219. In some embodiments, the CAR comprises the amino acid sequence of SEQ ID NO:220. In some embodiments, the CAR comprises the amino acid sequence of SEQ ID NO:221. In some embodiments, the CAR comprises the amino acid sequence of SEQ ID NO:222.

In several embodiments, there is provided a population of genetically engineered natural killer cells for cancer immunotherapy. In some embodiments, the population comprises a plurality of NK cells that have been expanded in culture. In some embodiments, at least a portion of the plurality of NK cells is engineered to express a chimeric antigen receptor comprising a tumor binding domain, a transmembrane domain, and a cytotoxic signaling complex. In some embodiments, the tumor binding domain targets CD70 and is encoded by a polynucleotide comprising a sequence having at least 85%, at least 90%, or at least 95% sequence identity to SEQ ID NO: 23 or 24. In some embodiments, the tumor binding domain targets CD70 and comprises an amino acid sequence having at least 85%, at least 90%, or at least 95% or greater sequence identity to SEQ ID NO: 25 or 26.

In some embodiments, the NK cells are genetically edited to express reduced levels of CD70 as compared to a non-edited NK cell that has been expanded in culture. In some embodiments, the reduced CD70 expression was engineered through editing of an endogenous CD70 gene. In some embodiments, the NK cells are further genetically edited to express reduced levels of a CIS protein encoded by a CISH gene as compared to a non-engineered NK cell. In some embodiments, the reduced CIS expression was engineered through editing of a CISH gene. In some embodiments, the genetically engineered NK cells exhibit one or more of enhanced expansion capability, enhanced cytotoxicity against target cells, and enhanced persistence, as compared to NK cells expressing native levels of CIS. In some embodiments, the NK cells are further genetically edited to express reduced levels of a CD70 protein. In some embodiments, the reduced CD70 expression was achieved through editing of a gene encoding said CD70. In some embodiments, the genetically engineered NK cells exhibit one or more of enhanced expansion capability, enhanced cytotoxicity against target cells and enhanced persistence, as compared to NK cells expressing native levels of CD70. In some embodiments, the NK cells are further genetically edited to express reduced levels of a CBLB protein. In some embodiments, the reduced CBLB expression was achieved through editing of a gene encoding said CBLB protein. In some embodiments, the genetically engineered NK cells exhibit one or more of enhanced expansion capability, enhanced cytotoxicity against target cells and enhanced persistence, as compared to NK cells expressing native levels of the CBLB protein.

In some embodiments, the tumor binding domain targets CD70 and is encoded by a polynucleotide comprising a sequence having at least 85%, at least 90%, or at least 95% sequence identity to SEQ ID NO: 30-32 or 34-37. In some embodiments, the tumor binding domain targets CD70 and comprises an amino acid sequence having at least 85%, at least 90%, or at least 95% or greater sequence identity to SEQ ID NO: 47-49 or 51-54. In some embodiments, the NK cells are genetically edited to express reduced levels of CD70 as compared to a non-edited NK cell that has been expanded in culture. In some embodiments, the reduced CD70 expression was engineered through editing of an endogenous CD70 gene. In some embodiments, the NK cells are further genetically edited to express reduced levels of a CIS protein encoded by a CISH gene as compared to a non-engineered NK cell. In some embodiments, the reduced CIS expression was engineered through editing of a CISH gene. In some embodiments, the genetically engineered NK cells exhibit one or more of enhanced expansion capability, enhanced cytotoxicity against target cells, and enhanced persistence, as compared to NK cells expressing native levels of CIS. In some embodiments, the NK cells are further genetically edited to express reduced levels of a CD70 protein. In some embodiments, the reduced CD70 expression was achieved through editing of a gene encoding said CD70. In some embodiments, the genetically engineered NK cells exhibit one or more of enhanced expansion capability, enhanced cytotoxicity against target cells and enhanced persistence, as compared to NK cells expressing native levels of CD70. In some embodiments, the NK cells are further genetically edited to express reduced levels of a CBLB protein. In some embodiments, the reduced CBLB expression was achieved through editing of a gene encoding said CBLB protein. In some embodiments, the genetically engineered NK cells exhibit one or more of enhanced expansion capability, enhanced cytotoxicity against target cells and enhanced persistence, as compared to NK cells expressing native levels of the CBLB protein.

Also disclosed herein are cells comprising any one of the anti-CD70 binding domains disclosed herein and/or any one of the CARs disclosed herein. In some embodiments, the cell is an immune cell. In some embodiments, the cell is an NK cell or a T cell. In some embodiments, the cell is genetically edited to express a reduced level of CISH, CBLB, CD70, or any combination thereof, as compared to a non-engineered cell. In some embodiments, the cell is genetically edited with one or more guide RNAs having at least 95% sequence identity to SEQ ID NOs: 159-201. In some embodiments, the cells comprise a genomic disruption within a target sequence of the CD70 gene, the target sequence selected from any one of SEQ ID NOS:177-180. In some embodiments, the cells comprise a genomic disruption within a target sequence of the CISH gene, the target sequence selected from any one of SEQ ID NOS:181-191. In some embodiments, the cells comprise a genomic disruption within a target sequence of the CBLB gene, the target sequence selected from any one of SEQ ID NOS:192-195. In some embodiments, the cells comprise a genomic disruption within a target sequence of the CD70 gene, the target sequence selected from any one of SEQ ID NOS:177-180; a genomic disruption within a target sequence of the CISH gene, the target sequence selected from any one of SEQ ID NOS:181-191; and a genomic disruption within a target sequence of the CBLB gene, the target sequence selected from any one of SEQ ID NOS:192-195. In some embodiments, the cells comprise a genomic disruption within the target sequence of SEQ ID NO:180; a genomic disruption within the target sequence SEQ ID NO:191; and a genomic disruption within the target sequence of SEQ ID NO:195.

Unless indicated otherwise to the contrary, the sequences provided for guide RNAs (gRNAs) that are recited using deoxyribonucleotides refer to the target DNA sequence (which is complementary to the corresponding non-target DNA sequence to which the gRNA binds) and shall be considered as also referencing those RNA guides used in practice (e.g., employing ribonucleotides, where the ribonucleotide uracil is used in lieu of deoxyribonucleotide thymine or vice-versa where thymine is used in lieu of uracil, wherein both are complementary base pairs to adenine when reciting either an RNA or DNA sequence). In other words, the sequences provided for particular gRNAs in Table 1 are identical to the gRNA sequences used in practice, except that the gRNA sequences include uracil in lieu of thymine. For example, a gRNA with the sequence TCACCAAGCCCGCGACCAATGGG (SEQ ID NO: 202) shall also refer to the following sequence UCACCAAGCCCGCGACCAAUGGG (SEQ ID NO: 203) or a gRNA with sequence UCACCAAGCCCGCGACCAAUGGG (SEQ ID NO: 203) shall also refer to the following sequence TCACCAAGCCCGCGACCAATGGG (SEQ ID NO: 202). Further, the non-target DNA sequence to which a particular gRNA sequence binds is complementary to the sequence of the particular gRNA. For example, a gRNA with the provided sequence of TCACCAAGCCCGCGACCAATGGG (SEQ ID NO: 202) binds to a non-target DNA sequence of AGTGGTTCGGGCGCTGGTTACCC (SEQ ID NO: 212). In this situation, the corresponding target DNA sequence, which is complementary to the non-target DNA sequence, is TCACCAAGCCCGCGACCAATGGG (SEQ ID NO: 202).

Table 1 provides a non-limiting list of gRNAs that are used to edit the indicated target genes. gRNAs that have at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to those gRNAs listed is also within the scope of the present disclosure.

TABLE 1
Example Guide RNA Sequences

| SEQ ID | | | |
|---|---|---|---|
| NO: | Target | Name | Sequence |
| 159 | SMAD3 | SMAD3 guide RNA 1 | CCGATCGTGAAGCGCCTGCT |
| 160 | SMAD3 | SMAD3 guide RNA 2 | CGAGAAGGCGGTCAAGAGCC |
| 161 | SMAD3 | SMAD3 guide RNA 3 | CTTGGTGTTGACGTTCTGCG |
| 162 | MAPKAPK3 | MAPKAPK guide RNA 1 | CTCTGCTGTTTCACCATCCA |
| 163 | MAPKAPK3 | MAPKAPK guide RNA 2 | CCCGGCTTGGGCGGTGCTCC |
| 164 | MAPKAPK3 | MAPKAPK guide RNA 3 | CGACTACCAGTTGTCCAAGC |
| 165 | CEACAM1 | CEACAM1 guide RNA 1 | GACTGAGTTATTGGCGTGGC |
| 166 | CEACAM1 | CEACAM1 guide RNA 2 | GAATGTTCCATTGATAAGCC |
| 167 | CEACAM1 | CEACAM1 guide RNA 3 | GAGAGGCTGAGGTTTGCCCC |
| 168 | DDIT4 | DDIT4 guide RNA 1 | CCTCACCATGCCTAGCCTTT |
| 169 | DDIT4 | DDIT4 guide RNA 2 | CGATCTGGGGTGGGAGTTCG |
| 170 | DDIT4 | DDIT4 guide RNA 3 | GTTTGACCGCTCCACGAGCC |
| 171 | TGFBR2 | TGFBR2-1 | CCCCTACCATGACTTTATTC |
| 172 | TGFBR2 | TGFBR2-2 | ATTGCACTCATCAGAGCTAC |
| 173 | TGFBR2 | TGFBR2-3 | AGTCATGGTAGGGGAGCTTG |
| 174 | TGFBR2 | TGFBR2-4 | TGCTGGCGATACGCGTCCAC |
| 175 | TGFBR2 | TGFBR2-5 | GTGAGCAATCCCCCGGGCGA |
| 176 | TGFBR2 | TGFBR2-6 | AACGTGCGGTGGGATCGTGC |
| 177 | CD70 | CD70-1 | TCACCAAGCCCGCGACCAATGGG |
| 178 | CD70 | CD70-2 | GCTTTGGTCCCATTGGTCGCGGG |
| 179 | CD70 | CD70 -3 | ACCCTCCTCCGGCATCGCCGCGG |
| 180 | CD70 | CD70-4 | GCTTTGGTCCCATTGGTCGC |
| 181 | CISH | CISH-1 | CTCACCAGATTCCCGAAGGT |
| 182 | CISH | CISH-2 | CCGCCTTGTCATCAACCGTC |
| 183 | CISH | CISH-3 | TCTGCGTTCAGGGGTAAGCG |
| 184 | CISH | CISH-4 | GCGCTTACCCCTGAACGCAG |
| 185 | CISH | CISH-5 | CGCAGAGGACCATGTCCCCG |
| 186 | CISH | CISH-9 | AGATTCCCGAAGGTAGGAGA |
| 187 | CISH | CISH-10 | CTGGTATTGGGGTTCCATTA |
| 188 | CISH | CISH-12 | TACTCAATGCGTACATTGGT |
| 189 | CISH | CISH-13 | ATACTCAATGCGTACATTGG |
| 190 | CISH | CISH-14 | TCCGGAAAGGCCAGGATGCG |
| 191 | CISH | CISH-15 | CTTGTCCAGGCCACGCATCC |
| 192 | E3 ubiquitin-protein | CBLB-1 | TAATCTGGTGGACCTCATGAAGG |
| | ligase CBL-B | | |
| 193 | E3 ubiquitin-protein | CBLB-2 | TCGGTTGGCAAACGTCCGAAAGG |
| | ligase CBL-B | | |
| 194 | E3 ubiquitin-protein | CBLB-3 | AGCAAGCTGCCGCAGATCGCAGG |
| | ligase CBL-B | | |
| 195 | E3 ubiquitin-protein | CBLB-4 | AGCAAGCTGCCGCAGATCGC |
| | ligase CBL-B | | |
| 196 | NKG2A | NKG2A-1 | GGAGCTGATGGTAAATCTGC |
| 197 | NKG2A | NKG2A-2 | TTGAAGGTTTAATTCCGCAT |
| 198 | NKG2A | NKG2A-3 | AACAACTATCGTTACCACAG |
| 199 | Adenosine Receptor 2 | A2AR-1 | CGAGGAGCCCATGATGGGCA |
| 200 | Adenosine Receptor 2 | A2AR-2 | GGGCAATGTGCTGGTGTGCT |
| 201 | Adenosine Receptor 2 | A2AR-3 | CAGTGACACCACAAAGTAGT |

### Treatment Methods, Administration, and Dosing

Some embodiments relate to a method of treating, ameliorating, inhibiting, or preventing cancer with a cell or immune cell comprising a chimeric antigen receptor and/or an activating chimeric receptor, as disclosed herein. In some embodiments, the method includes treating or preventing cancer. In some embodiments, the method includes administering a therapeutically effective amount of immune cells expressing a tumor-directed chimeric antigen receptor and/or tumor-directed chimeric receptor as described herein. Examples of types of cancer that may be treated as such are described herein.

Disclosed herein are methods of treating cancer in a subject. In some embodiments, the methods comprise administering to the subject any one of the anti-CD70 binding domains disclosed herein, any one of the CARs disclosed herein, or any one of the cells disclosed herein, or any combination thereof.

Also disclosed herein are uses of any one of the anti-CD70 binding domains disclosed herein, any one of the CARs disclosed herein, any one of the cells disclosed herein, or any combination thereof for the treatment of cancer. Also disclosed herein are uses of any one of the anti-CD70 binding domains disclosed herein, any one of the CARs disclosed herein, any one of the cells disclosed herein, or any combination thereof in the manufacture of a medicament for the treatment of cancer.

In certain embodiments, treatment of a subject with a genetically engineered cell(s) described herein achieves one, two, three, four, or more of the following effects, including, for example: (i) reduction or amelioration the severity of disease or symptom associated therewith; (ii) reduction in the duration of a symptom associated with a disease; (iii) protection against the progression of a disease or symptom associated therewith; (iv) regression of a disease or symptom associated therewith; (v) protection against the development or onset of a symptom associated with a disease; (vi) protection against the recurrence of a symptom associated with a disease; (vii) reduction in the hospitalization of a subject; (viii) reduction in the hospitalization length; (ix) an increase in the survival of a subject with a disease; (x) a reduction in the number of symptoms associated with a disease; (xi) an enhancement, improvement, supplementation, complementation, or augmentation of the prophylactic or therapeutic effect(s) of another therapy. Each of these comparisons are versus, for example, a different therapy for a disease, which includes a cell-based immunotherapy for a disease using cells that do not express the constructs disclosed herein

Administration can be by a variety of routes, including, without limitation, intravenous, intra-arterial, subcutaneous, intramuscular, intrahepatic, intraperitoneal and/or local delivery to an affected tissue. Doses of immune cells such as NK and/or T cells can be readily determined for a given subject based on their body mass, disease type and state, and desired aggressiveness of treatment, but range, depending on the embodiments, from about 10⁵ cells per kg to about 10¹² cells per kg (e.g., 10⁵-10⁷, 10-10¹⁰, 10¹⁰-10¹² and overlapping ranges therein). In one embodiment, a dose escalation regimen is used. In several embodiments, a range of immune cells such as NK and/or T cells is administered, for example between about 1×10⁶ cells/kg to about 1×10⁸ cells/kg. In several embodiments, a range of immune cells such as NK and/or T cells is administered, for example between about 300×10⁶ cells to about 10×10⁹ cells. In several embodiments, a range of immune cells such as NK cells is administered, for example between about 1×10⁶ cells/kg to about 1×10⁸ cells/kg. In several embodiments, a range of immune cells such as NK cells is administered, for example between about 300×10⁶ cells to about 10×10⁹ cells. In some embodiments, about 300×10⁶ NK cells are administered. In some embodiments, about 1×10⁹ NK cells are administered. In some embodiments, about 1.5×10⁹ NK cells are administered.

Depending on the embodiment, various types of cancer can be treated. In several embodiments, the cancer is a CD70-expressing cancer.

In several embodiments, hepatocellular carcinoma is treated. Additional embodiments provided for herein include treatment or prevention of the following non-limiting examples of cancers including, but not limited to, acute lymphoblastic leukemia (ALL), acute myeloid leukemia (AML), adrenocortical carcinoma, Kaposi sarcoma, lymphoma, gastrointestinal cancer, appendix cancer, central nervous system cancer, basal cell carcinoma, bile duct cancer, bladder cancer, bone cancer, brain tumors (including but not limited to astrocytomas, spinal cord tumors, brain stem glioma, glioblastoma, craniopharyngioma, ependymoblastoma, ependymoma, medulloblastoma, medulloepithelioma), breast cancer, bronchial tumors, Burkitt lymphoma, cervical cancer, colon cancer, chronic lymphocytic leukemia (CLL), chronic myelogenous leukemia (CML), chronic myeloproliferative disorders, ductal carcinoma, endometrial cancer, esophageal cancer, gastric cancer, Hodgkin lymphoma, non-Hodgkin lymphoma, hairy cell leukemia, renal cell cancer, leukemia, oral cancer, nasopharyngeal cancer, liver cancer, lung cancer (including but not limited to, non-small cell lung cancer, (NSCLC) and small cell lung cancer), pancreatic cancer, bowel cancer, lymphoma, melanoma, ocular cancer, ovarian cancer, pancreatic cancer, prostate cancer, pituitary cancer, uterine cancer, and vaginal cancer.

In some embodiments, also provided herein are nucleic acid and amino acid sequences that have sequence identity and/or homology of at least 80%, 85%, 90%, 95%, 96%, 97%, 98%, 99% (and ranges therein) as compared with the respective nucleic acid or amino acid sequences of SEQ ID NOS. 1-203 (or combinations of two or more of SEQ ID NOS: 1-203) and that also exhibit one or more of the functions as compared with the respective SEQ ID NOS. 1-203 (or combinations of two or more of SEQ ID NOS: 1-203) including but not limited to, (i) enhanced proliferation, (ii) enhanced activation, (iii) enhanced cytotoxic activity against cells presenting ligands to which NK cells harboring receptors encoded by the nucleic acid and amino acid sequences bind, (iv) enhanced homing to tumor or infected sites, (v) reduced off target cytotoxic effects, (vi) enhanced secretion of immunostimulatory cytokines and chemokines (including, but not limited to IFNg, TNFa, IL-22, CCL3, CCL4, and CCL5), (vii) enhanced ability to stimulate further innate and adaptive immune responses, and (viii) combinations thereof.

In some embodiments, also provided herein are nucleic acid and amino acid sequences that have sequence identity and/or homology of at least 80%, 85%, 90%, 95%, 96%, 97%, 98%, 99% (and ranges therein) as compared with the respective nucleic acid or amino acid sequences of SEQ ID NOS. 1-226 (or combinations of two or more of SEQ ID NOS: 1-226) and that also exhibit one or more of the functions as compared with the respective SEQ ID NOS. 1-226 (or combinations of two or more of SEQ ID NOS: 1-226) including but not limited to, (i) enhanced proliferation, (ii) enhanced activation, (iii) enhanced cytotoxic activity against cells presenting ligands to which NK cells harboring receptors encoded by the nucleic acid and amino acid sequences bind, (iv) enhanced homing to tumor or infected sites, (v) reduced off target cytotoxic effects, (vi) enhanced secretion of immunostimulatory cytokines and chemokines (including, but not limited to IFNg, TNFa, IL-22, CCL3, CCL4, and CCL5), (vii) enhanced ability to stimulate further innate and adaptive immune responses, and (viii) combinations thereof.

Additionally, in several embodiments, there are provided amino acid sequences that correspond to any of the nucleic acids disclosed herein, while accounting for degeneracy of the nucleic acid code. Furthermore, those sequences (whether nucleic acid or amino acid) that vary from those expressly disclosed herein, but have functional similarity or equivalency are also contemplated within the scope of the present disclosure. The foregoing includes mutants, truncations, substitutions, or other types of modifications.

In several embodiments, polynucleotides encoding the disclosed cytotoxic receptor complexes are mRNA. In some embodiments, the polynucleotide is DNA. In some embodiments, the polynucleotide is operably linked to at least one regulatory element for the expression of the cytotoxic receptor complex.

Additionally provided, according to several embodiments, is a vector comprising the polynucleotide encoding any of the polynucleotides provided for herein, wherein the polynucleotides are optionally operatively linked to at least one regulatory element for expression of a cytotoxic receptor complex. In several embodiments, the vector is a retrovirus.

Further provided herein are engineered immune cells (such as NK and/or T cells) comprising the polynucleotide, vector, or cytotoxic receptor complexes as disclosed herein. Further provided herein are compositions comprising a mixture of engineered immune cells (such as NK cells and/or engineered T cells), each population comprising the polynucleotide, vector, or cytotoxic receptor complexes as disclosed herein. Additionally, there are provided herein compositions comprising a mixture of engineered immune cells (such as NK cells and/or engineered T cells), each population comprising the polynucleotide, vector, or cytotoxic receptor complexes as disclosed herein and the T cell population having been genetically modified to reduce/eliminate gvHD and/or HvD. In some embodiments, the NK cells and the T cells are from the same donor. In some embodiments, the NK cells and the T cells are from different donors. In several embodiments, one or more genes are edited (e.g., knockout or knock in) in order to impart one or more enhanced functions or characteristics to the edited cells. For example, in several embodiments CIS protein is substantially reduced by editing the CISH, which leads to enhanced NK cell proliferation, cytotoxicity and/or persistence.

Doses of immune cells such as NK cells or T cells can be readily determined for a given subject based on their body mass, disease type and state, and desired aggressiveness of treatment, but range, depending on the embodiments, from about 10⁵ cells per kg to about 10¹² cells per kg (e.g., 10⁵-10⁷, 10⁷-10¹⁰, 10¹⁰-10¹² and overlapping ranges therein). In one embodiment, a dose escalation regimen is used. In several embodiments, a range of NK cells is administered, for example between about 1×10⁶ cells/kg to about 1×10⁸ cells/kg. Depending on the embodiment, various types of cancer or infection disease can be treated.

### Cancer Types

Some embodiments of the compositions and methods described herein relate to administering immune cells comprising a tumor-directed chimeric antigen receptor and/or tumor-directed chimeric receptor to a subject with cancer. Various embodiments provided for herein include treatment or prevention of the following non-limiting examples of cancers, including both solid and suspension tumors. Examples of cancer include, but are not limited to, acute lymphoblastic leukemia (ALL), acute myeloid leukemia (AML), adrenocortical carcinoma, Kaposi sarcoma, lymphoma, gastrointestinal cancer, appendix cancer, central nervous system cancer, basal cell carcinoma, bile duct cancer, bladder cancer, bone cancer, brain tumors (including but not limited to astrocytomas, spinal cord tumors, brain stem glioma, craniopharyngioma, ependymoblastoma, ependymoma, medulloblastoma, medulloepithelioma), breast cancer, bronchial tumors, Burkitt lymphoma, cervical cancer, colon cancer, chronic lymphocytic leukemia (CLL), chronic myelogenous leukemia (CML), chronic myeloproliferative disorders, ductal carcinoma, endometrial cancer, esophageal cancer, gastric cancer, Hodgkin lymphoma, non-Hodgkin lymphoma, hairy cell leukemia, renal cell cancer, leukemia, oral cancer, nasopharyngeal cancer, liver cancer, lung cancer (including but not limited to, non-small cell lung cancer, (NSCLC) and small cell lung cancer), pancreatic cancer, bowel cancer, lymphoma, melanoma, ocular cancer, ovarian cancer, pancreatic cancer, prostate cancer, pituitary cancer, uterine cancer, and vaginal cancer. In several embodiments, the cancer comprises a solid tumor. In some embodiments, the cancer is esophageal cancer. In some embodiments, the cancer is head and neck cancer. In some embodiments, the cancer is lung cancer. In some embodiments, the cancer is liver cancer. In some embodiments, the cancer is colorectal cancer. In some embodiments, the cancer is bladder cancer. In some embodiments, the cancer is cervical cancer. In some embodiments, the cancer is endometrial cancer. In some embodiments, the cancer is ovarian cancer. In some embodiments, the cancer is uterine cancer. In some embodiments, the cancer is melanoma.

### Cancer Targets

Some embodiments of the compositions and methods described herein relate to immune cells comprising a chimeric receptor that targets a cancer antigen. Non-limiting examples of target antigens include: CD70, CD5, CD19; CD123; CD22; CD30; CD171; CS1 (also referred to as CD2 subset 1, CRACC, SLAMF7, CD319, and 19A24); TNF receptor family member B cell maturation (BCMA); CD38; DLL3; G protein coupled receptor class C group 5, member D (GPRC5D); epidermal growth factor receptor (EGFR) CD138; prostate-specific membrane antigen (PSMA); Fms Like Tyrosine Kinase 3 (FLT3); KREMEN2 (Kringle Containing Transmembrane Protein 2), ALPPL2, Claudin 4, Claudin 6, C-type lectin-like molecule-1 (CLL-1 or CLECL1); CD33; epidermal growth factor receptor variant III (EGFRviii); ganglioside G2 (GD2); ganglioside GD3 (aNeu5Ac(2-8)aNeu5Ac(2-3)bDGalp(I-4)bDGlcp(I-I)Cer);); Tn antigen ((Tn Ag) or (GalNAca-Ser/Thr)); prostate-specific membrane antigen (PSMA); Receptor tyrosine kinase-like orphan receptor 1 (ROR1); Fms Like Tyrosine Kinase 3 (FLT3); Tumor-associated glycoprotein 72 (TAG72); CD38; CD44v6; a glycosylated CD43 epitope expressed on acute leukemia or lymphoma but not on hematopoietic progenitors, a glycosylated CD43 epitope expressed on non-hematopoietic cancers, Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-IIRa); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha (FRa or FR1); Folate receptor beta (FRb); Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); sialyl Lewis adhesion molecule (sLe); ganglioside GM3 (aNeu5Ac(2-3)bDClalp(I-4)bDGlcp(I-I)Cer); transglutaminase 5 (TGS5); high molecular weight-melanoma associated antigen (HMWMAA); o-acetyl-GD2 ganglioside (OAcGD2); tumor endothelial marker 1 (TEM1/CD248); tumor endothelial marker 7-related (TEM7R); claudin 6 (CLDN6); thyroid stimulating hormone receptor (TSHR); G protein coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-la); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; survivin; telomerase; prostate carcinoma tumor antigen-1 (PCT A-1 or Galectin 8), melanoma antigen recognized by T cells 1 (MelanA or MARTI); Rat sarcoma (Ras) mutant; human Telomerase; reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin BI; v-myc avian myelocytomatosis viral oncogene neuroblastoma derived homolog (MYCN); Ras Homolog Family Member C (RhoC); Tyrosinase-related protein 2 (TRP-2); Cytochrome P450 IB 1 (CYPIB 1); CCCTC-Binding Factor (Zinc Finger Protein)-Like (BORIS or Brother of the Regulator ofImprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Gly cation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLLI), MPL, Biotin, c-MYC epitope Tag, CD34, LAMP1 TROP2, GFRalpha4, CDH17, CDH6, NYBR1, CDH19, CD200R, Slea (CA19.9; Sialyl Lewis Antigen); Fucosyl-GMI, PTK7, gpNMB, CDH1-CD324, DLL3, CD276/B7H3, ILI IRa, IL13Ra2, CD179b-IGLII, TCRgamma-delta, NKG2D, CD32 (FCGR2A), Tn ag, Timl-/HVCR1, CSF2RA (GM-CSFR-alpha), TGFbetaR2, Lews Ag, TCR-betal chain, TCR-beta2 chain, TCR-gamma chain, TCR-delta chain, FITC, Leutenizing hormone receptor (LHR), Follicle stimulating hormone receptor (FSHR), Gonadotropin Hormone receptor (CGHR or GR), CCR4, GD3, SLAMF6, SLAMF4, HIV1 envelope glycoprotein, HTLVI-Tax, CMV pp65, EBV-EBNA3c, KSHV K8.1, KSHV-gH, influenza A hemagglutinin (HA), GAD, PDL1, Guanylyl cyclase C (GCC), auto antibody to desmoglein 3 (Dsg3), auto antibody to desmoglein 1 (Dsgl), HLA, HLA-A, HLA-A2, HLA-B, HLA-C, HLA-DP, HLA-DM, HLA-DOA, HLA-DOB, HLA-DQ, HLA-DR, HLA-G, IgE, CD99, Ras G12V, Tissue Factor 1 (TF1), AFP, GPRC5D, Claudinl 8.2 (CLD18A2 or CLDN18A.2)), P-glycoprotein, STEAP1, Livl, Nectin-4, Cripto, gpA33, BST1/CD157, low conductance chloride channel, and the antigen recognized by TNT antibody.

### NON-LIMITING EMBODIMENTS

Among the embodiments provided herein are:

1. A population of genetically engineered natural killer (NK) cells for cancer immunotherapy, comprising:
a plurality of NK cells engineered to express a chimeric antigen receptor (CAR) comprising a tumor binding domain, a transmembrane domain, and a cytotoxic signaling complex,
wherein the tumor binding domain targets CD70,
wherein the tumor binding domain comprises an scFv comprising an amino acid sequence having at least about 90% sequence identity to one or more of SEQ ID NOs: 47-49 or 51-54,
wherein the NK cells comprise a genomic disruption within a CD70 protein gene target sequence that comprises any one of SEQ ID NO: 177-180, optionally wherein said genomic disruption comprises and endonuclease-mediated indel,
wherein the NK cells comprise a genomic disruption within of a cytokine-inducible SH2-containing protein gene target sequence that comprises any one of SEQ ID NO: 186-191, and
wherein the NK cells comprise at least one additional genomic disruption within a gene target sequence, and
wherein the genetically engineered NK cells comprising said genomic disruptions exhibit one or more of enhanced expansion capability, enhanced cytotoxicity against target cells, and enhanced persistence, as compared to NK cells not comprising said genomic disruptions.

2. The plurality of NK cells of Embodiment 1, wherein the NK cells have been expanded in culture.

3. A population of genetically engineered natural killer (NK) cells for cancer immunotherapy, comprising:
a plurality of NK cells that have been expanded in culture,
wherein the plurality of NK cells are engineered to express a chimeric antigen receptor (CAR) comprising a tumor binding domain, a transmembrane domain, and a cytotoxic signaling complex,
wherein the tumor binding domain targets CD70,
wherein the NK cells comprise a genomic disruption within a CD70 protein gene target sequence that comprises any one of SEQ ID NO: 177-180, wherein said genomic disruption comprises and endonuclease-mediated indel,
wherein the NK cells comprise a genomic disruption within of a cytokine-inducible SH2-containing protein gene target sequence that comprises any one of SEQ ID NO: 186-191, and
wherein the NK cells comprise at least one additional genomic disruption within a gene target sequence, and
wherein the genetically engineered NK cells comprising said genomic disruptions exhibit one or more of enhanced expansion capability, enhanced cytotoxicity against target cells, and enhanced persistence, as compared to NK cells not comprising said genomic disruptions.

4. A population of genetically engineered natural killer (NK) cells for cancer immunotherapy, comprising:
a plurality of NK cells that have been expanded in culture,
wherein the plurality of NK cells are engineered to express a chimeric antigen receptor (CAR) comprising a tumor binding domain, a transmembrane domain, and a cytotoxic signaling complex,
wherein the tumor binding domain targets CD70,
wherein the NK cells are genetically edited to express reduced levels of CD70 as compared to a non-edited NK cell that has been expanded in culture, and wherein the reduced CD70 expression was engineered through introducing a genomic disruption in an endogenous CD70 gene,
wherein the NK cells are genetically edited to express reduced levels of a cytokine-inducible SH2-containing (CIS) protein encoded by a CISH gene as compared to a non-edited NK cell, wherein the reduced CIS expression was engineered through introducing a genomic disruption in a CISH gene, and
wherein the genetically engineered NK cells exhibit one or more of enhanced expansion capability, enhanced cytotoxicity against target cells, and enhanced persistence, as compared to NK cells expressing native levels of CIS, and
wherein the NK cells are genetically edited to introduce a genomic disruption in at two or more additional genes to reduce expression of a protein encoded by said two or more additional genes as compared to a NK cell not edited at said genes.

5. A population of genetically engineered natural killer (NK) cells for cancer immunotherapy, comprising:
a plurality of NK cells engineered to express a chimeric antigen receptor (CAR) comprising a tumor binding domain, a transmembrane domain, and a cytotoxic signaling complex,
wherein the tumor binding domain targets CD70,
wherein the tumor binding domain comprises an scFv comprising an amino acid sequence having at least about 90% sequence identity to one or more of SEQ ID NOs: 47-49 or 51-54,
wherein the plurality of NK cells comprise a genomic disruption within a gene target sequence that comprises at least three of SEQ ID NO: 177-195, optionally wherein said genomic disruption comprises an endonuclease-mediated indel.

6. The population of genetically engineered NK cells of any one of Embodiments 1 to 5, wherein the tumor binding domain comprises a heavy chain variable region and a light chain variable region, wherein the heavy chain variable region comprises a CDR-H1, CDR-H2, and CDR-H3, and the light chain variable region comprises a CDR-L1, CDR-L2, and CDR-L3, and wherein
the CDR-H1 comprises a sequence having at least 95% sequence identity to one or more sequences selected from SEQ ID NOs: 102, 103, and 110;
the CDR-H2 comprises a sequence having at least 95% sequence identity to one or more sequences selected from SEQ ID NOs: 104, 105, 106, and 111;
the CDR-H3 comprises a sequence having at least 95% sequence identity to one or more sequences selected from SEQ ID NOs: 107, 108, 109, and 112;
the CDR-L1 comprises a sequence having at least 95% sequence identity to one or more sequences selected from SEQ ID NOs: 131, 132, 133, and 140;
the CDR-L2 comprises a sequence having at least 95% sequence identity to one or more sequences selected from SEQ ID NOs: 134, 135, 136, and 141; and
the CDR-L3 comprises a sequence having at least 95% sequence identity to one or more sequences selected from SEQ ID NOs: 137, 138, 139, and 142.

7. The population of genetically engineered NK cells of any one of Embodiments 1 to 6, wherein the tumor binding domain comprises a VH, wherein the VH comprises an amino acid sequence having at least 95% sequence identity to one or more of the amino acid sequences of SEQ ID NOs: 151-153 and 157.

8. The population of genetically engineered NK cells of any one of Embodiments 1 to 7, wherein the tumor binding domain comprises a VL, wherein the VL comprises an amino acid sequence having at least 95% sequence identity to one or more of the amino acid sequences of SEQ ID NOs: 154-156 and 158.

9. The population of genetically engineered NK cells of any one of Embodiments 1 to 8, wherein the tumor binding domain comprises a VL and a VH, wherein the VL comprises an amino acid sequence having at least 95% sequence identity to the amino acid sequence of SEQ ID NO: 156, wherein the VH comprises an amino acid sequence having at least 95% sequence identity to the amino acid sequence of SEQ ID NO: 153.

10. The population of genetically engineered NK cells of any one of Embodiments 1 to 8, wherein the tumor binding domain comprises a VL and a VH, wherein the VL comprises an amino acid sequence having at least 95% sequence identity to the amino acid sequence of SEQ ID NO: 155, wherein the VH comprises an amino acid sequence having at least 95% sequence identity to the amino acid sequence of SEQ ID NO: 152.

11. The population of genetically engineered NK cells of any one of Embodiments 1 to 8, wherein the tumor binding domain comprises a VL and a VH, wherein the VL comprises an amino acid sequence having at least 95% sequence identity to the amino acid sequence of SEQ ID NO: 157, wherein the VH comprises an amino acid sequence having at least 95% sequence identity to the amino acid sequence of SEQ ID NO: 158.

12. The population of genetically engineered NK cells of any one of Embodiments 1 to 11, wherein the tumor binding domain comprises an scFv, wherein the scFv comprises an amino acid sequence having at least 95% sequence identity to one or more of SEQ ID NOs: 47-49 and 51-54.

13. The population of genetically engineered NK cells of any one of Embodiments 1 to 12, wherein the tumor binding domain comprises a heavy chain variable region (VH), wherein the VH is encoded by a polynucleotide comprising a sequence having at least 95% sequence identity to one or more of the polynucleotides of SEQ ID NOs: 143-146 and 149.

14. The population of genetically engineered NK cells of any one of Embodiments 1 to 13, wherein the tumor binding domain comprises a light chain variable region (VL), wherein the VL is encoded by a polynucleotide comprising a sequence having at least 95% sequence identity to one or more of the polynucleotides of SEQ ID NOs: 146-148 and 150.

15. The population of genetically engineered NK cells of any one of Embodiments 1 to 14, wherein the tumor binding domain comprises a single chain variable fragment (scFv), wherein the scFv is encoded by a polynucleotide comprising a sequence having at least 95% sequence identity to one or more of the polynucleotides of SEQ ID NOs: 30-32 and 34-37.

16. The population of genetically engineered NK cells of any one of Embodiments 1 to 15, wherein the cytotoxic signaling complex comprises an OX40 subdomain and a CD3zeta subdomain.

17. The population of genetically engineered NK cells according Embodiment 16, wherein the OX40 subdomain is encoded by a sequence having at least 95% sequence identity to SEQ ID NO: 5.

18. The population of genetically engineered NK cells according to any one of Embodiments 16 to 17, wherein the CD3zeta subdomain is encoded by a sequence having at least 95% sequence identity to SEQ ID NO: 7.

19. The population of genetically engineered NK cells of any one of Embodiments 1 to 18, wherein the NK cells are engineered to express membrane bound IL-15 (mbIL15).

20. The population of genetically engineered NK cells of Embodiment 19, wherein the mbIL15 is bicistronically encoded on a polynucleotide encoding the CAR.

21. The population of genetically engineered NK cells according to any one of Embodiments 19 or 20, wherein the mbIL15 is encoded by a sequence having at least 95% sequence identity to SEQ ID NO: 27.

22. The population of genetically engineered NK cells according to any one of Embodiments 20 to 21, wherein polynucleotide encoding the CAR and the mbIL15 comprises a sequence having at least 95% sequence identity to one or more of the polynucleotides of SEQ ID NOs: 38-46.

23. The population of genetically engineered NK cells according to any one of Embodiments 1 to 22, wherein the CAR comprises an amino acid sequence having at least 95% sequence identity to one or more of the amino acid sequences of SEQ ID NOs: 64-72.

24. The population of genetically engineered NK cells according to any one of Embodiments 1 to 23, wherein the engineered NK cells are edited at CD70, CISH, and CBLB.

25. The population of genetically engineered NK cells according to any one of Embodiments 1 to 24, wherein the engineered NK cells are edited at CD70, CISH, CBLB, and an additional target gene.

26. The population of genetically engineered NK cells Embodiment 25, wherein expression of CD70 is substantially reduced as compared to an NK cell not edited with respect to CD70, wherein expression of CIS is substantially reduced as compared to an NK cell not edited with respect to CISH, and wherein expression of CBLB is substantially reduced as compared to an NK cell not edited with respect to CBLB.

27. The population of genetically engineered NK cells of Embodiment 25 or 26, wherein the NK cells do not express a detectable level of CD70, CIS, or CBLB protein.

28. The population of genetically engineered NK cells according to any one of Embodiments 1 to 27, wherein the gene editing introducing the genomic disruption is made using a CRISPR-Cas system.

29. The population of genetically engineered NK cells of Embodiment 28 wherein the CRISPR-Cas system comprises a Cas selected from Cas9, Csn2, Cas4, Cpf1, C2c1, C2c3, Cas13a, Cas13b, Cas13c, CasX, CasY, and combinations thereof.

30. The population of genetically engineered NK cells of Embodiment 28 or 29, wherein the Cas is Cas9.

31. A method of treating cancer in a subject, comprising administering to the subject the population of genetically engineered NK cells according to any one of the preceding Embodiments.

32. The method of Embodiment 31, wherein the cancer is renal cell carcinoma, or a metastasis from renal cell carcinoma.

33. Use of the population of genetically engineered NK cells according to any one of Embodiments 1 to 30 in the treatment of a cancer.

34. Use of the genetically engineered NK cells according to any one of Embodiments 1 to 30 in the manufacture of a medicament for the treatment of cancer.

35. A method for treating cancer in a subject comprising,
administering to the subject a population of genetically engineered NK cells, comprising:
a plurality of NK cells that have been expanded in culture,
wherein the plurality of NK cells is engineered to express a chimeric antigen receptor (CAR) comprising a tumor binding domain, a transmembrane domain, and a cytotoxic signaling complex,
wherein the tumor binding domain targets CD70,
wherein the tumor binding domain comprises an scFv comprising an amino acid sequence having at least about 90% sequence identity to one or more of SEQ ID NOs: 47-49 or 51-54,
wherein the NK cells comprise a genomic disruption within a CD70 protein gene target sequence that comprises any one of SEQ ID NO: 177-180, optionally wherein said genomic disruption comprises and endonuclease-mediated indel,
wherein the NK cells comprise a genomic disruption within of a cytokine-inducible SH2-containing protein gene target sequence that comprises any one of SEQ ID NO: 186-191, and
wherein the NK cells comprise at least one additional genomic disruption within a gene target sequence, and
wherein the genetically engineered NK cells comprising said genomic disruptions exhibit one or more of enhanced expansion capability, enhanced cytotoxicity against target cells, and enhanced persistence, as compared to NK cells not comprising said genomic disruptions.

36. The method of Embodiment 35, wherein the NK cells are further genetically edited to express reduced levels of a CBLB protein encoded by a CBLB gene as compared to a non-edited NK cell.

37. The method of Embodiment 35 or 36, wherein the tumor binding domain comprises a heavy chain variable region and a light chain variable region, wherein the heavy chain variable region comprises a CDR-H1, CDR-H2, and CDR-H3, and the light chain variable region comprises a CDR-L1, CDR-L2, and CDR-L3, and wherein
the CDR-H1 comprises a sequence having at least 95% sequence identity to one or more sequences selected from SEQ ID NOs: 102, 103, and 110;
the CDR-H2 comprises a sequence having at least 95% sequence identity to one or more sequences selected from SEQ ID NOs: 104, 105, 106, and 111;
the CDR-H3 comprises a sequence having at least 95% sequence identity to one or more sequences selected from SEQ ID NOs: 107, 108, 109, and 112;
the CDR-L1 comprises a sequence having at least 95% sequence identity to one or more sequences selected from SEQ ID NOs: 131, 132, 133, and 140;
the CDR-L2 comprises a sequence having at least 95% sequence identity to one or more sequences selected from SEQ ID NOs: 134, 135, 136, and 141; and
the CDR-L3 comprises a sequence having at least 95% sequence identity to one or more sequences selected from SEQ ID NOs: 137, 138, 139, and 142.

38. The method of any one of Embodiments 35 to 37, wherein the tumor binding domain comprises a VH, wherein the VH comprises an amino acid sequence having at least 95% sequence identity to one or more of the amino acid sequences of SEQ ID NOs: 151-153 and 157, and wherein the tumor binding domain comprises a VL, wherein the VL comprises an amino acid sequence having at least 95% sequence identity to one or more of the amino acid sequences of SEQ ID NOs: 154-156 and 158.

39. The method of any one of Embodiments 35 to 38, wherein the tumor binding domain comprises a VL and a VH, wherein the VL comprises an amino acid sequence having at least 95% sequence identity to the amino acid sequence of SEQ ID NO: 156, wherein the VH comprises an amino acid sequence having at least 95% sequence identity to the amino acid sequence of SEQ ID NO: 153.

40. The method of any one of Embodiments 35 to 38, wherein the tumor binding domain comprises a VL and a VH, wherein the VL comprises an amino acid sequence having at least 95% sequence identity to the amino acid sequence of SEQ ID NO: 155, wherein the VH comprises an amino acid sequence having at least 95% sequence identity to the amino acid sequence of SEQ ID NO: 152.

41. The method of any one of Embodiments 35 to 38, wherein the tumor binding domain comprises a VL and a VH, wherein the VL comprises an amino acid sequence having at least 95% sequence identity to the amino acid sequence of SEQ ID NO: 157, wherein the VH comprises an amino acid sequence having at least 95% sequence identity to the amino acid sequence of SEQ ID NO: 158.

42. The method of any one of Embodiments 35 to 41, wherein the tumor binding domain comprises an scFv, wherein the scFv comprises an amino acid sequence having at least 95% sequence identity to one or more of SEQ ID NOs: 47-49 and 51-54.

43. The method of any one of Embodiments 35 to 42, wherein the cytotoxic signaling complex comprises an OX40 subdomain and a CD3zeta subdomain.

44. The method of Embodiment 43, wherein the OX40 subdomain is encoded by a sequence having at least 95% sequence identity to SEQ ID NO: 5, wherein the CD3zeta subdomain is encoded by a sequence having at least 95% sequence identity to SEQ ID NO: 7

45. The method of any one of Embodiments 35 to 44, wherein the NK cells are engineered to express membrane bound IL-15 (mbIL15).

46. The method of Embodiment 45, wherein the mbIL15 is bicistronically encoded on a polynucleotide encoding the CAR.

47. The method of Embodiment 46, wherein polynucleotide encoding the CAR and the mbIL15 comprises a sequence having at least 95% sequence identity to one or more of the polynucleotides of SEQ ID NOs: 38-46.

48. The method of any one of Embodiments 45 to 47, wherein the mbIL15 is encoded by a sequence having at least 95% sequence identity to SEQ ID NO: 27.

49. The method of any one of Embodiments 35 to 48, wherein the CAR comprises an amino acid sequence having at least 95% sequence identity to one or more of the amino acid sequences of SEQ ID NOs: 64 to 72.

50. The method according to any one of Embodiments 35 to 49, wherein the gene editing is made using a CRISPR-Cas system, and wherein the Cas comprises a Cas9 enzyme.

51. An anti-CD70 chimeric antigen receptor (CAR), wherein the CAR comprises an anti-CD70 binding domain, an OX40 domain, and a CD3zeta domain wherein the anti-CD70 CAR comprises an amino acid sequence having at least 95% sequence identity to one or more of the amino acid sequences of SEQ ID NOs: 64-72, or a portion thereof capable of generating cytotoxic signals upon binding to CD70 on a target cell.

52. An anti-CD70 chimeric antigen receptor (CAR), wherein the CAR comprises an anti-CD70 binding domain, an OX40 domain, and a CD3zeta domain wherein the anti-CD70 CAR comprises an amino acid sequence having at least 95% sequence identity to one or more of the amino acid sequences of SEQ ID NOs: 64-72, or a portion thereof capable of generating cytotoxic signals upon binding to CD70 on a target cell.

53. The anti-CD70 CAR of Embodiment 51 or 52, wherein the anti-CD70 binding domain comprises an scFv having at least 95%, 99%, or 100% sequence identity to any sequence selected from SEQ ID NOs: 47-49 and 51-54.

54. A cell comprising the anti-CD70 CAR of any one of Embodiments 51 to 53.

55. The cell of Embodiment 54, wherein the cell is an immune cell.

56. The cell of Embodiment 54 or 55, wherein the cell is an NK cell.

57. The cell of any one of Embodiments 54 to 56, wherein the cell comprises at least three genomic disruptions within at least three gene target sequences selected from SEQ ID NOs: 159-201.

58. A method of treating cancer in a subject comprising administering to the subject the CAR of any one of Embodiments 51 to 53, or the cell of any one of Embodiments 54 to 56.

59. Use of the anti-CD70 CAR of any one of Embodiments 51 to 53, or the cell of any one of Embodiments 54 to 56 for the treatment of a cancer.

60. Use of the anti-CD70 CAR of any one of Embodiments 51 to 53, or the cell of any one of Embodiments 54 to 56 in the manufacture of a medicament for the treatment of cancer.

61. A method for generating a population of genetically engineered immune cells, comprising: introducing an endonuclease and at least one unique gRNA into the immune cells to induce a genomic disruption within at least one gene target sequence, introducing an endonuclease and at least one additional unique gRNA into the immune cells to induce an additional genomic disruption within an additional gene target sequence, and transducing the immune cells with a viral vector encoding a CD70-targeting CAR.

62. The method of Embodiment 61, wherein the endonuclease and gRNA are induced by electroporating the cells.

63. The method of Embodiment 61 or 62, wherein the cells comprise NK cells.

64. The method according to any one of Embodiments 61 to 63, wherein no more than three unique gRNAs are introduced at a time.

65. The method according to any one of Embodiments 61 to 64, wherein no more than two unique gRNAs are introduced at a time.

66. The method according to any one of Embodiments 61 to 65, wherein cells are expanded in culture for a period of time prior to the first introduction.

67. A method for generating a population of genetically engineered immune cells, comprising: expanding the immune cells in culture,
introducing an endonuclease and no more than two unique gRNA into the immune cells to induce a genomic disruption within two distinct gene target sequences,
culturing the cells for an additional period of time
introducing an additional endonuclease and no more than two additional unique gRNA into the immune cells to induce additional genomic disruptions within no more than two additional gene target sequences, and
transducing the immune cells with a viral vector encoding a CD70-targeting CAR.

68. The method of Embodiment 67, wherein the endonucleases and gRNA are induced by electroporating the cells.

69. The method of Embodiment 67 or 68, wherein the cells comprise NK cells.

70. The method according to any one of Embodiments 61 to 69, wherein only one additional type of gRNA is used in the second introduction.

71. The method according to any one of Embodiments 61 to 70, wherein the gRNAs target CD70, CISH, or CBLB genes.

72. A pharmaceutical composition that comprises a population of engineered natural killer cells that comprise a genomic disruption within a gene target sequence that comprises at least three of SEQ ID NO: 159-203, wherein said genomic disruption optionally comprises an endonuclease-mediated indel.

73. A pharmaceutical composition that comprises a population of engineered natural killer cells that comprise a genomic disruption within a gene target sequence that comprises at least three of SEQ ID NO: 177-195, wherein said genomic disruption optionally comprises an endonuclease-mediated indel.

74. A pharmaceutical composition that comprises a population of engineered natural killer cells that comprise a genomic disruption within a gene target sequence that comprises at least two of SEQ ID NO: 177-195, wherein said genomic disruption optionally comprises an endonuclease-mediated indel, and wherein engineered NK cells express a CD70-targeting CAR comprising an scFv comprising an amino acid sequence having at least about 90% sequence identity to one or more of SEQ ID NOs: 47-49 and 51-54.

75. The pharmaceutical composition of any one of Embodiments 72-74, wherein said genomic disruption comprises an endonuclease-mediated indel.

### EXAMPLES

The following are non-limiting descriptions of experimental methods and materials that were used in examples disclosed below.

### Example 1—CD70 Binder Screening

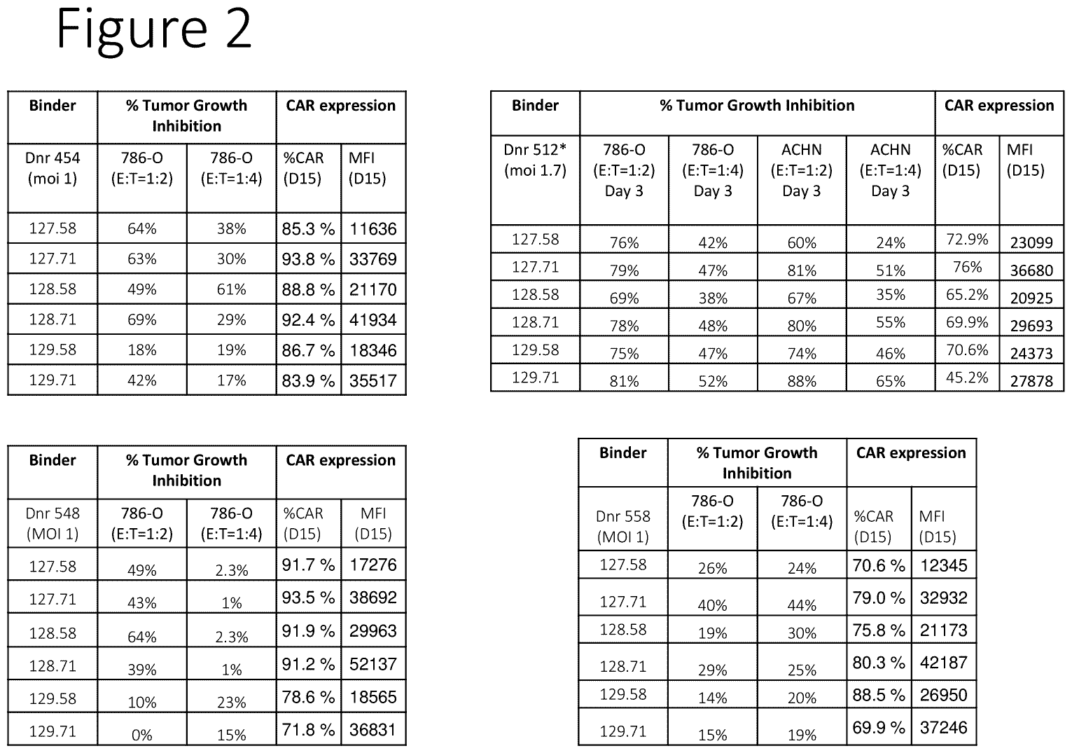

A screening of various CD70 binders was conducted to characterize selected features of the binders to determine if they met various thresholds to advance to further experimental protocols related to multiplex gene editing (Table E1). FIG. 2 summarizes the characterization of certain binders in terms of their ability to inhibit tumor growth in an in vitro assay and the durability or persistence of expression of the CARs incorporating the binders on Day 15 of a process in which NK cells are transduced with a retroviral vector encoding the CAR construct, which in this example, targets CD70. The “128 Series” construct employs an scFv with a vH-GS3 linker-vL format. The “127 Series” construct employs a vL-linker-vH format, with the linker being an alternative linker having at least 80% sequence identity to the linker of SEQ ID NO:50 (encoded by a polynucleotide having at least 80% sequence identity to SEQ ID NO: 33.). The “129 Series” has selected mutations in an mbIL15 that is encoded bicistronically on the polynucleotide encoding the CAR, but is expressed separately. The mutations comprise mutations in a hinge sequence that alter one or more cysteine residues to, for example, a serine or alanine residue. Various CD70 binders are indicated by an additional numeric identifier, in this experiment, either the 58 or 71 binder. Together the two numerals indicate the binder identity and the structure—in other words, NK128.58 employs scFv number 58 using the vH-GS3 linker-vL format. As discussed herein, various transmembrane and signaling domains can be used. These non-limiting embodiments of CAR constructs provided for herein employ a CD8 alpha hinge and transmembrane domain, an OX40 co-stimulatory domain and a CD3 zeta signaling domain.

TABLE E1
Non-Limiting CD70 Binders (SEQ ID NOS)

| Binder | CDR-H1 | CDR-H2 | CDR-H3 | VH | CDR-L1 | CDR-L2 | CDR-L3 | VL | Linker | scFv |
|---|---|---|---|---|---|---|---|---|---|---|
| 127.58 | 205 | 225 | 226 | 152 | 204 | 223 | 224 | 155 | 50 | 51 |
| 127.71 | 205 | 206 | 207 | 153 | 209 | 210 | 211 | 156 | 50 | 52 |
| 128.58 | 205 | 225 | 226 | 152 | 204 | 223 | 224 | 155 | 208 | 48 |
| 128.71 | 205 | 206 | 207 | 153 | 209 | 210 | 211 | 156 | 208 | 49 |
| 146 | 110 | 111 | 112 | 157 | 140 | 141 | 142 | 158 | 50 | 53 |
| 147 | 110 | 111 | 112 | 157 | 140 | 141 | 142 | 158 | 208 | 54 |

As shown in FIG. 2, which is replicated data from engineered NK cells from 4 donors, with the cells also being edited to reduce CD70 expression on the NK cells (e.g., to avoid fratricide) and also edited to knock out CISH expression. The primary trend of the cytotoxicity data is that the 127 and 128 series CARs exhibited relatively consistent tumor growth inhibition within a given donor's cells. As expected, the ability to inhibit tumor growth was greater at a 1:2 effector:target (E:T) ratio, as compared to a 1:4 E:T. The 129 series CARs appeared to be less robust in terms of inhibition of tumor growth. At 15 days post-electroporation (EP), each of the CARs was still expressed on most of the population of NK cells (as measured by % CAR (D15)). While there was some variability, within those CD70 CAR positive cells, the intensity (e.g., number of CD70 CARs expressed by a cell) was relatively high.

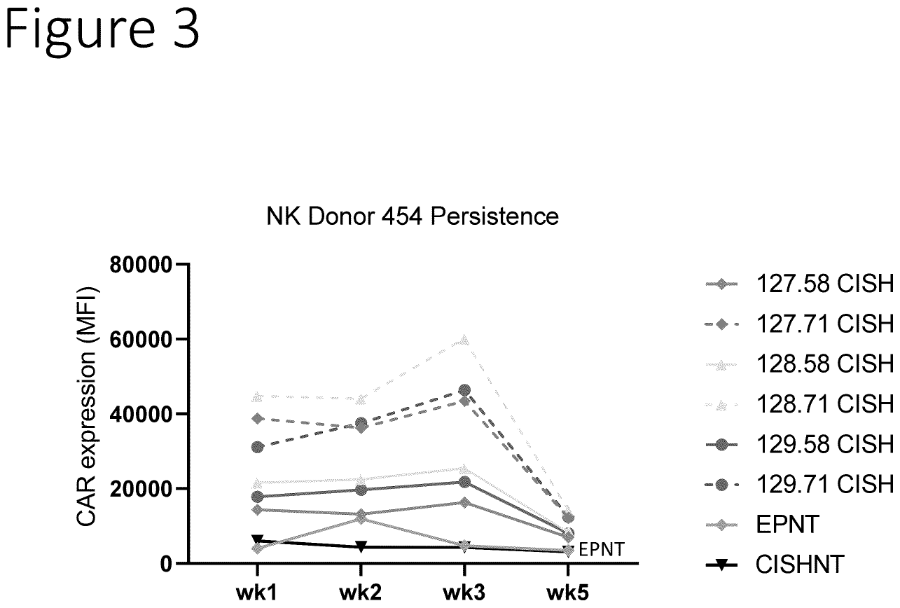

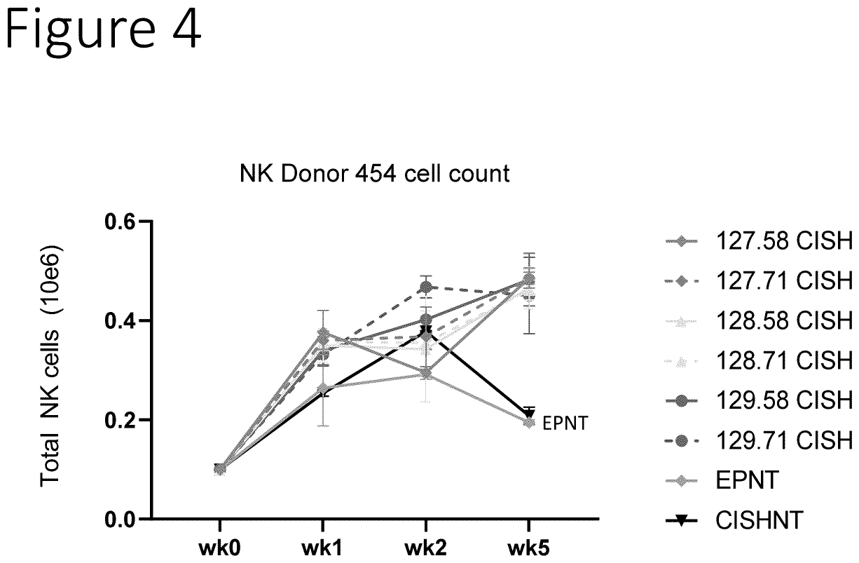

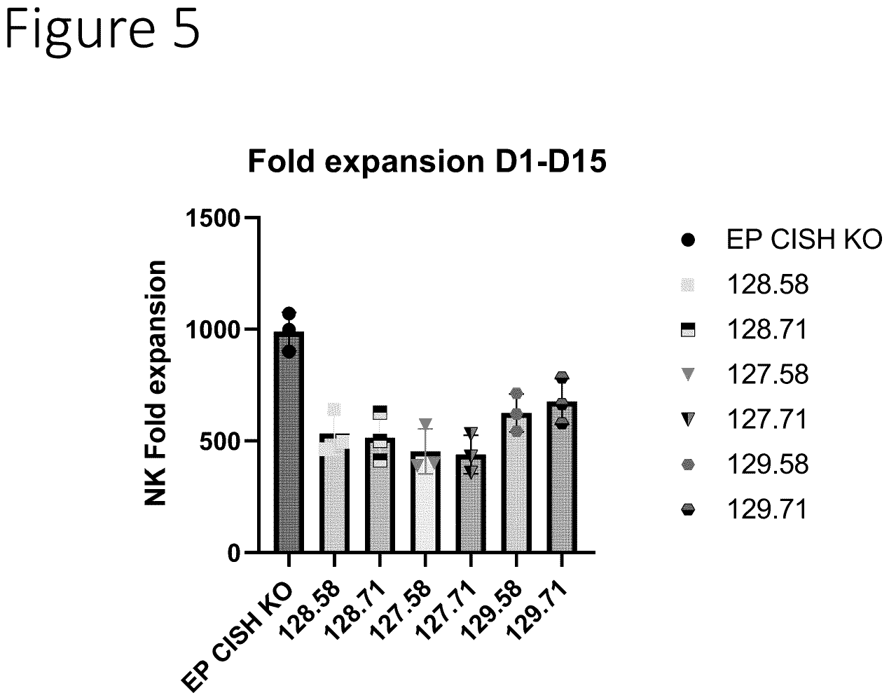

FIG. 3 shows representative data related to persistence of CAR expression over several weeks. While the constructs expressing the “71” CAR appear to have elevated persistence across at least the first three weeks, these data are important in that they demonstrate that each of the selected CAR constructs are well expressed by NK cells for several weeks. Similar trends were seen with corresponding data from two other donors (not shown). FIG. 4 shows data related to the overall NK cell count present in a culture at 5 weeks post-EP. These data show that, irrespective of the CAR construct expressed by the NK cells, there is little variability in the cell counts, meaning that no CAR induces particularly adverse effects on the NK cell survival. In a similar vein, FIG. 5 shows data that indicates that, irrespective of the CAR being expressed, there is limited variability in the ability of the NK cell population to expand during the first 15 days of culturing.

To further characterize the 58 and 71 binders, the binders were formatted as full IgG1 and assessed by flow cytometry for binding to human primary epithelial cells. The primary epithelial cell types included bronchial, kidney, pancreatic, stomach, liver, spleen, esophageal, colonic, small intestine, and alveolar cells. As a positive control, binding to CD70-expressing cell lines was also assessed. Neither of the binders were observed to bind to the tested human primarily epithelial cells, whereas they did bind to CD70-expressing cell lines (data not shown).

Taken together these data demonstrate that anti-CD70 CAR constructs can be stably expressed by gene edited NK cells, can control tumor growth, and have limited inhibitory effects on expansion and NK cell population numbers.

### Example 2—Further Characterization of Gene Edited NK Cells Expressing Selected CD70 CARs

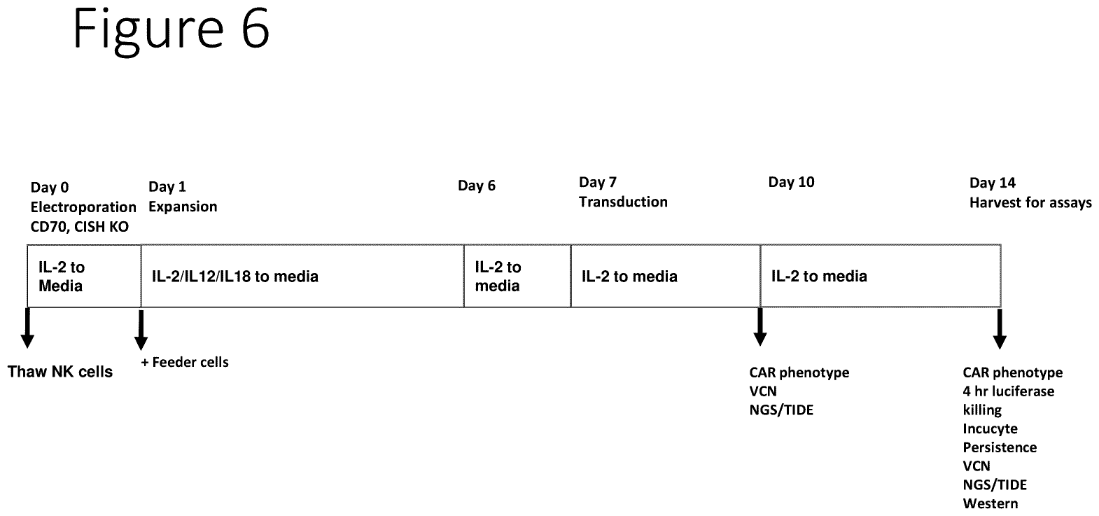

Additional in vitro experiments were performed to further assess NK cells that are dual gene edited and engineered to express selected CD70-targeting CARs. In this series of experiments, the constructs tested are the NK128.58, NK128.71, NK127.58, NK127.71, NK146 and NK147, the latter two employing the same scFv architecture as the NK127 and NK128 series, respectively. The NK cells are also edited at both CISH and CD70 to reduce CIS and CD70 protein expression, respectively. FIG. 6 shows a non-limiting schematic of the production and assessment of the gene edited and CAR-expressing NK cells.

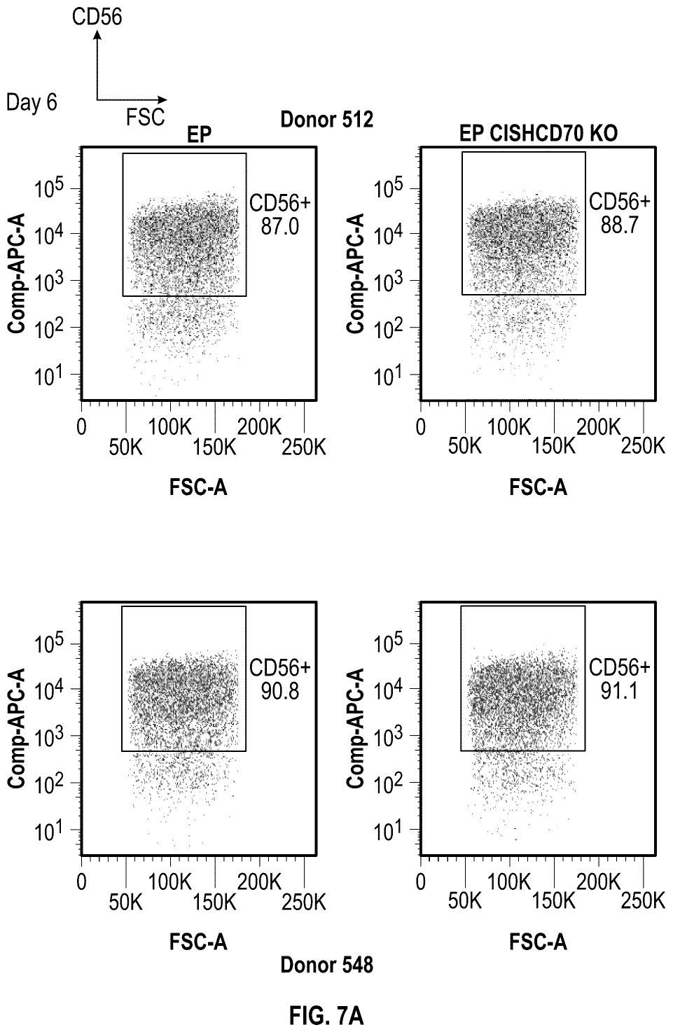

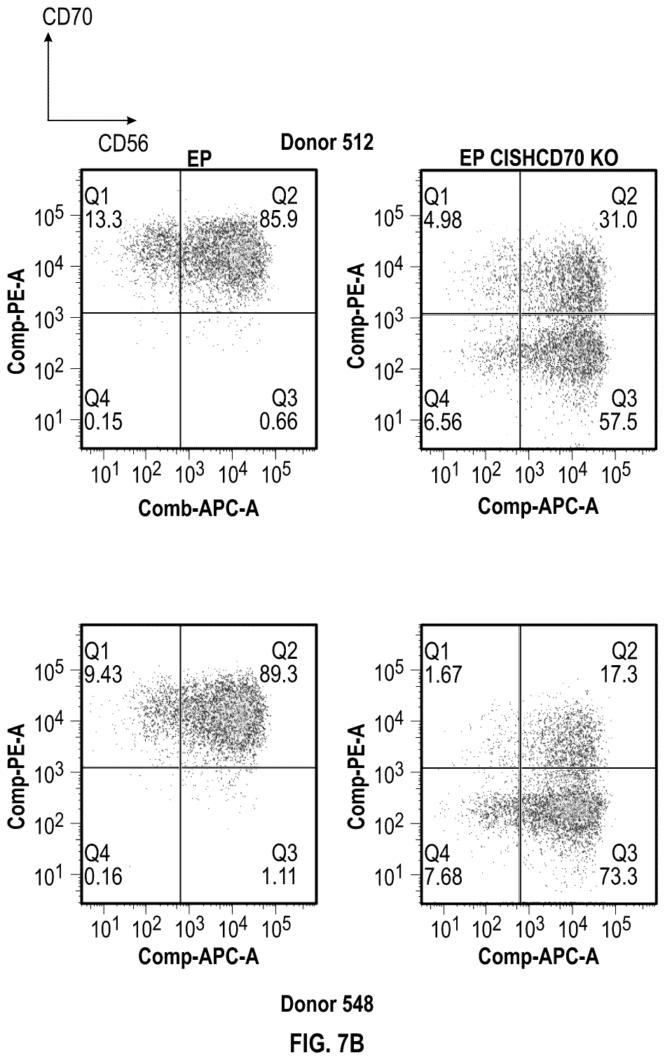

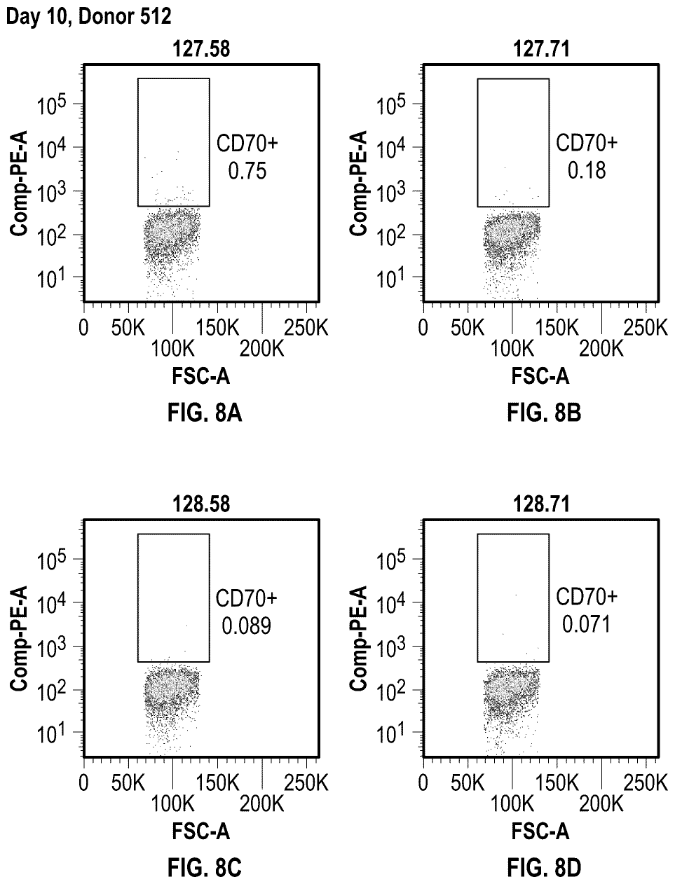

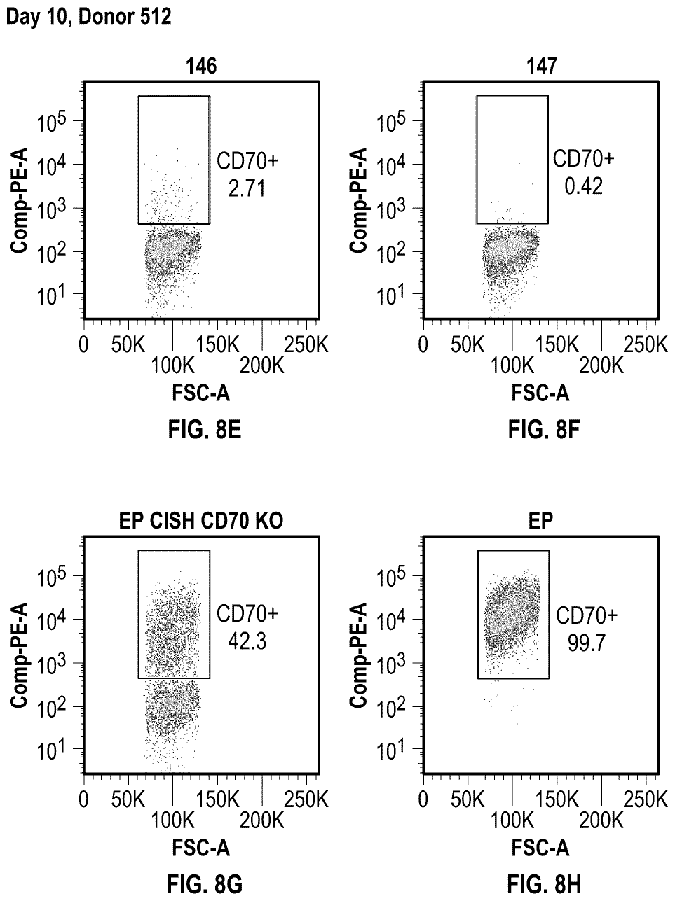

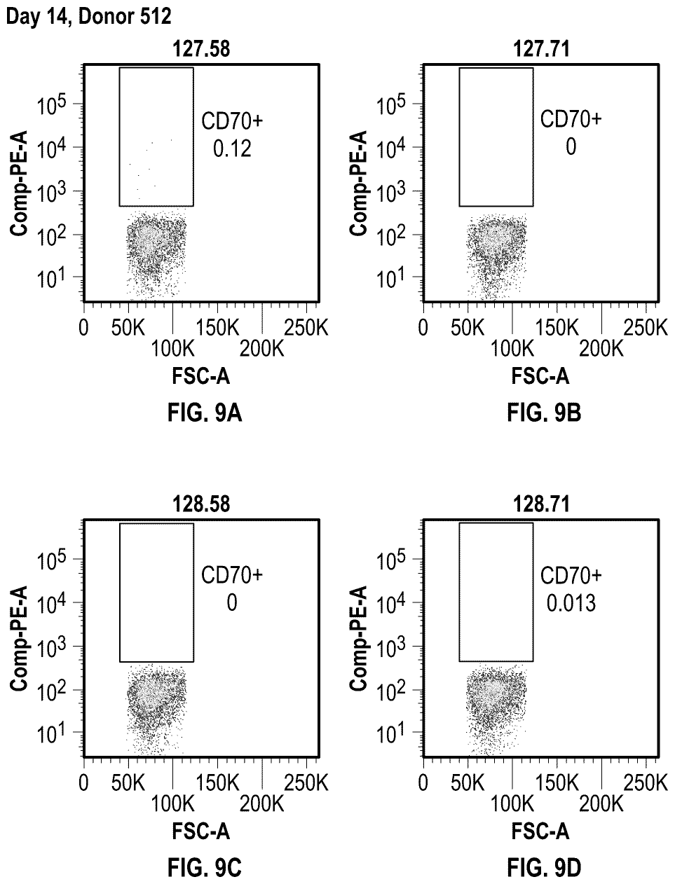

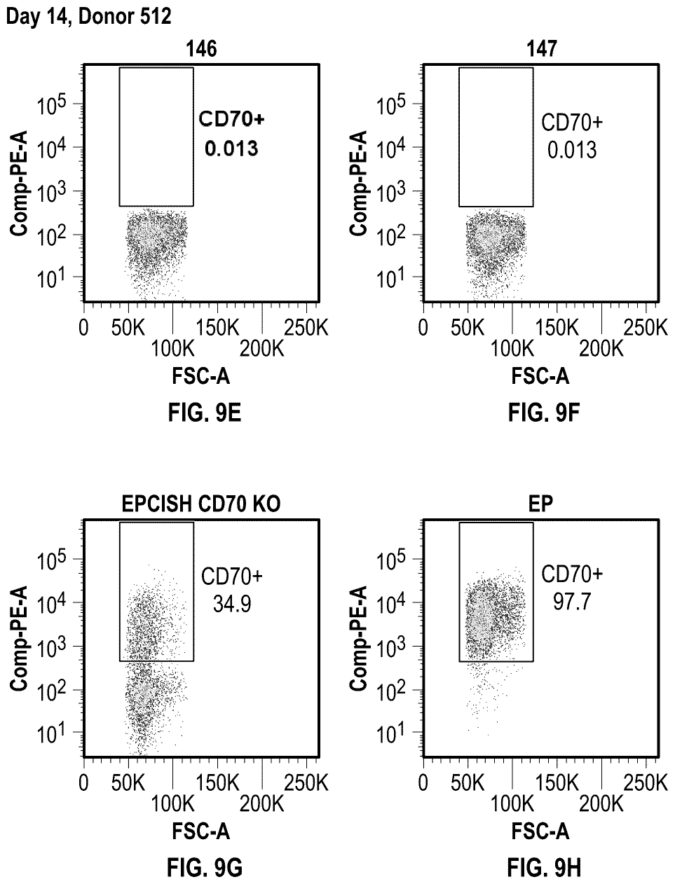

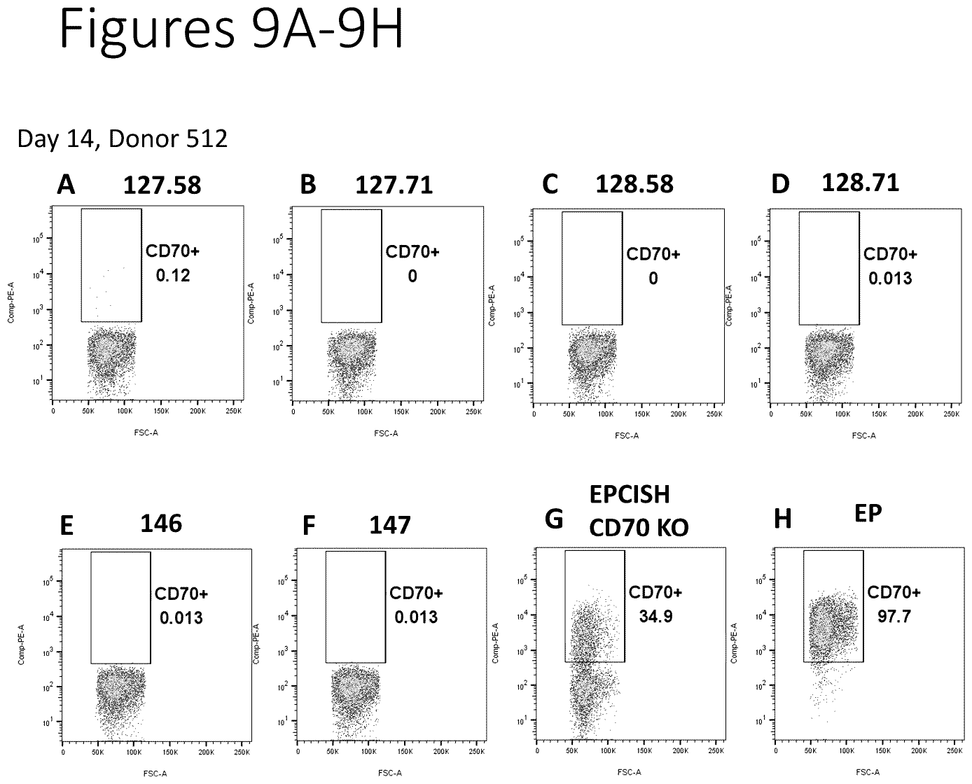

FIGS. 7A-7B show flow cytometry data related to CD70 expression by NK cells from two donors (donor 512, top row; donor 548, bottom row) at 6 days post-gene editing. FIG. 7A shows CD56 expression (representing prevalence of NK cells) data and confirms that the gene editing process (e.g., electroporation and introduction of CISH/CD70 gRNAs) does not cause reduction in NK cell numbers. FIG. 7B shows that, as compared to the EP control, the introduction of CISH/CD70 gRNAs and an endonuclease results in substantial reduction in CD70 expression (right column). FIGS. 8A-8H show data related to CD70 expression at Day 10, after the gene edited cells were transduced with a viral vector encoding the indicated anti-CD70 CAR construct. As shown in FIG. 8G, CD70/CISH editing reduces the degree of CD70 expression by the NK cells (as compared to the EP control in FIG. 8H). Each of FIGS. 8A-8F show minimal CD70 expression (reduced even as compared to the non-transduced but gene edited cells in FIG. 8G). This reduction indicates that each of the indicated CAR constructs is functionally effective, as the near-zero CD70 expression reflects fratricide on the remaining gene edited NK cells that still express some amount of CD70. At least with respect to cytotoxicity directed against residual CD70-expressing NK cells, each of the CD70 CAR constructs appear similar in their effectiveness. FIGS. 9A-9H show corresponding data collected at Day 14, with CD70 expressing cells again essentially eliminated in each group where a CD70 CAR was expressed. Similar Day 10 and 14 data was generated for the second donor (not shown).

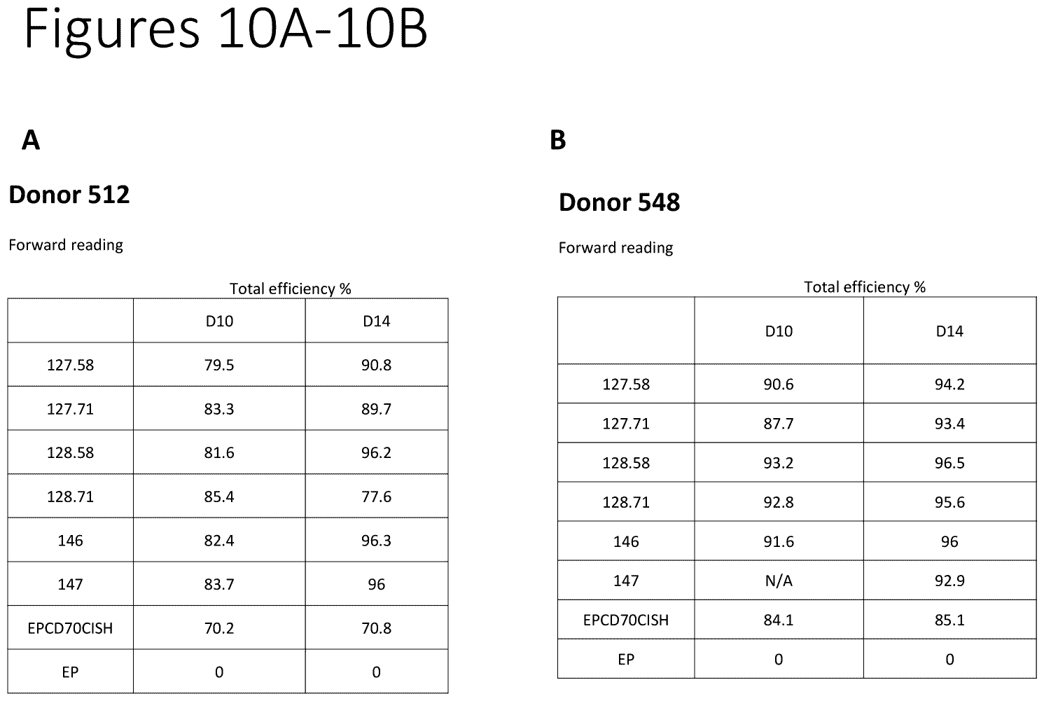

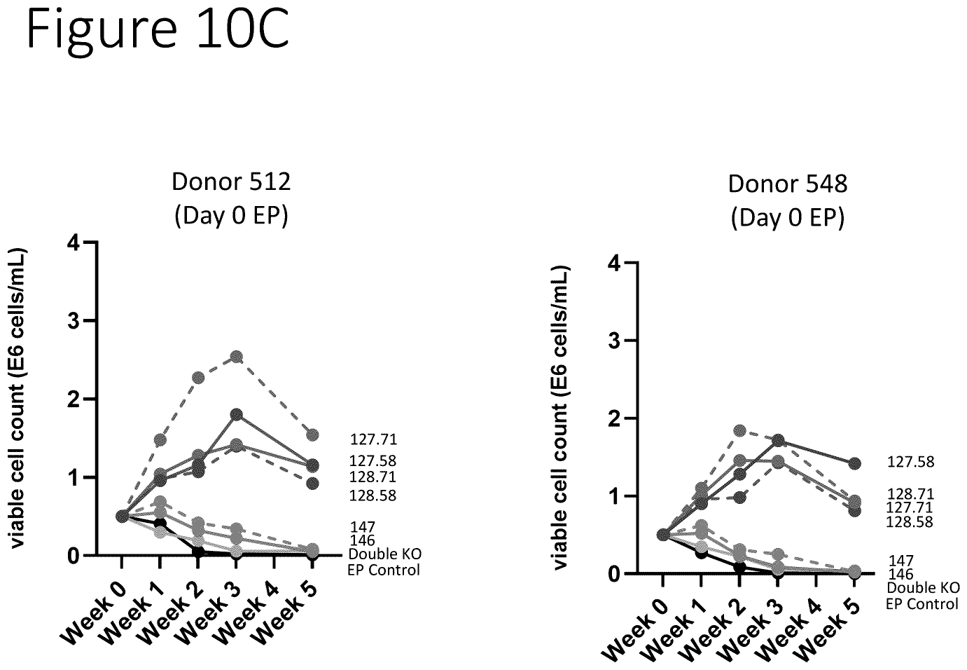

FIGS. 10A-10B show TIDE indel analysis for each of the two donors (10A/10B respectively). These data show that the gene editing efficiency is approximately 70-85% at Day 10, with a trend to efficiency of at least about 90% in the Day 14 analysis.

In a further experiment, NK cells from the same two donors were knocked out for CD70 and CISH via electroporation with CD70- and CISH-targeting gRNAs on Day 0 and engineered to express one of the exemplary CD70 CARs. NK cells knocked out for CD70 and CISH but not expressing a CAR (Double KO) or mock electroporated cells (EP only) served as controls. The persistence of the cells in the absence of IL-2 was assessed in culture over five weeks. Cells expressing the 127 and 128 series CARs exhibited increased persistence in the absence of IL-2 (FIG. 10C).

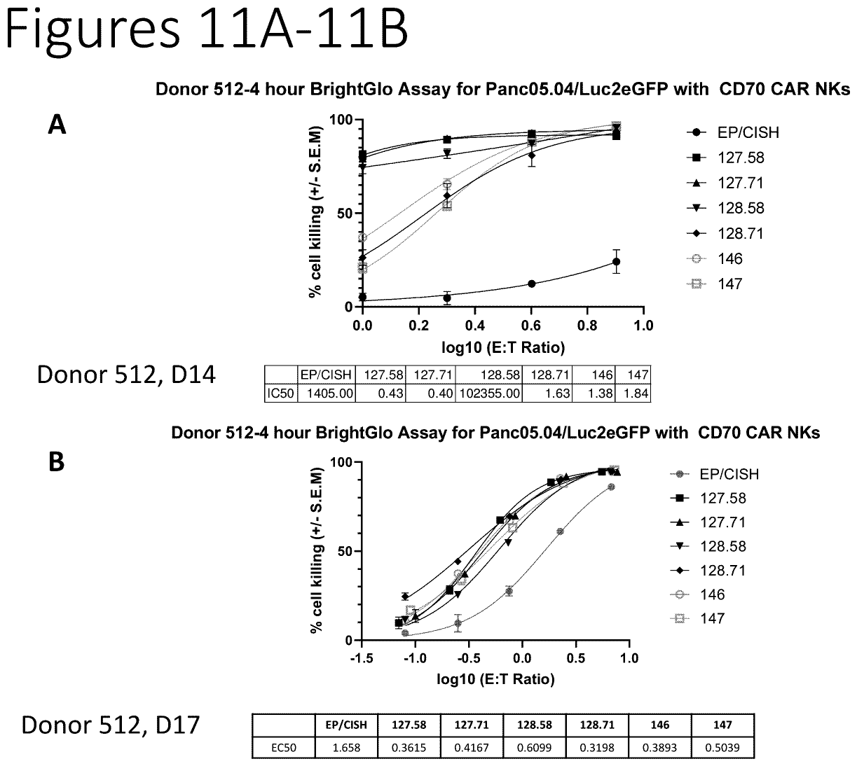

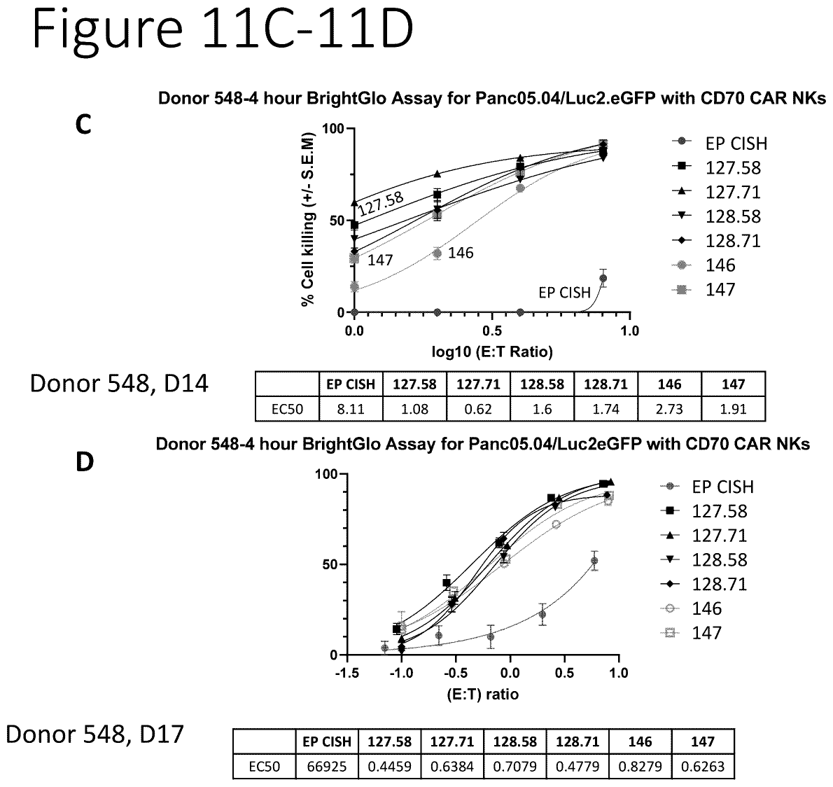

Turning to assessment of the cytotoxicity of the gene-edited and CD70 CAR-expressing NK cells, Bright-Glo™ analysis was conducted to determine the IC₅₀ of the various CAR constructs against Panc05 cells, which express low levels of CD70. FIGS. 11A and 11C show data collected from a first and second donor, respectively, on Day 14 post-gene editing. At this time-point, each of the CAR-expressing cell populations exhibited enhanced cytotoxicity over the CISH-edited NK cells not expressing a CAR (EP CISH). The 127.58, 127.71 and 128.58 CAR-expressing NK cells appeared somewhat more potent as compared with certain other CAR-expressing NK cells, such as NK128.71. When assessing the cytotoxicity profiles in the same donors at Day 17, the various CD70 CAR-expressing cells performed somewhat more consistently (FIGS. 11B and 11D). Importantly, each of the groups tested effectively eliminated tumor cells, despite the target cells expressing low levels of CD70.

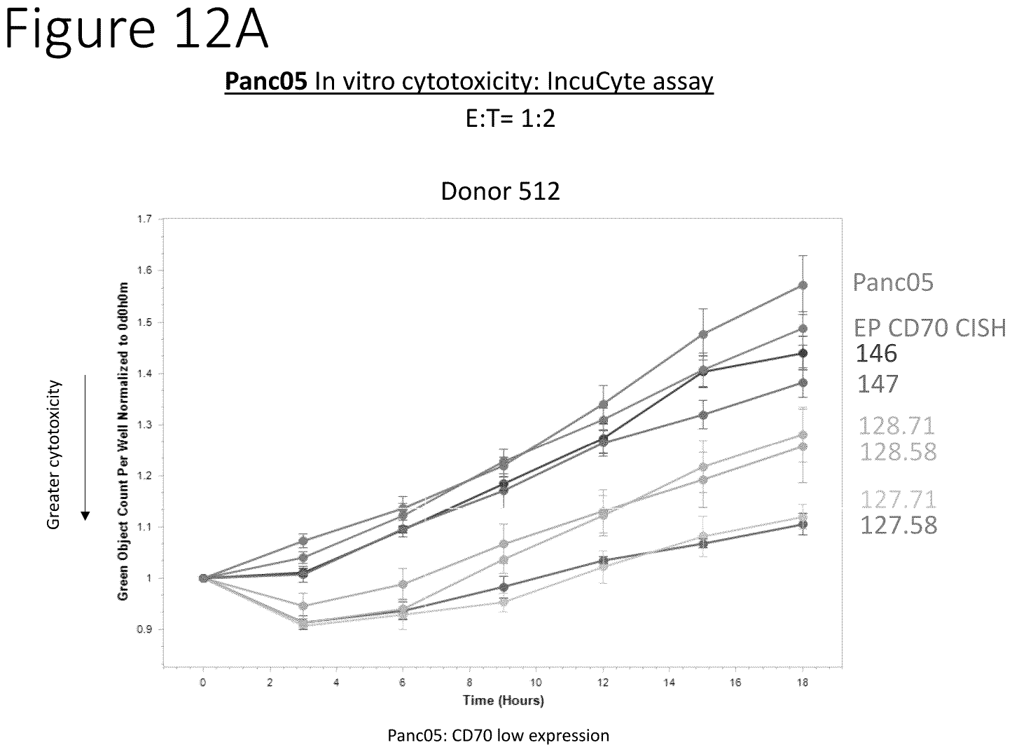

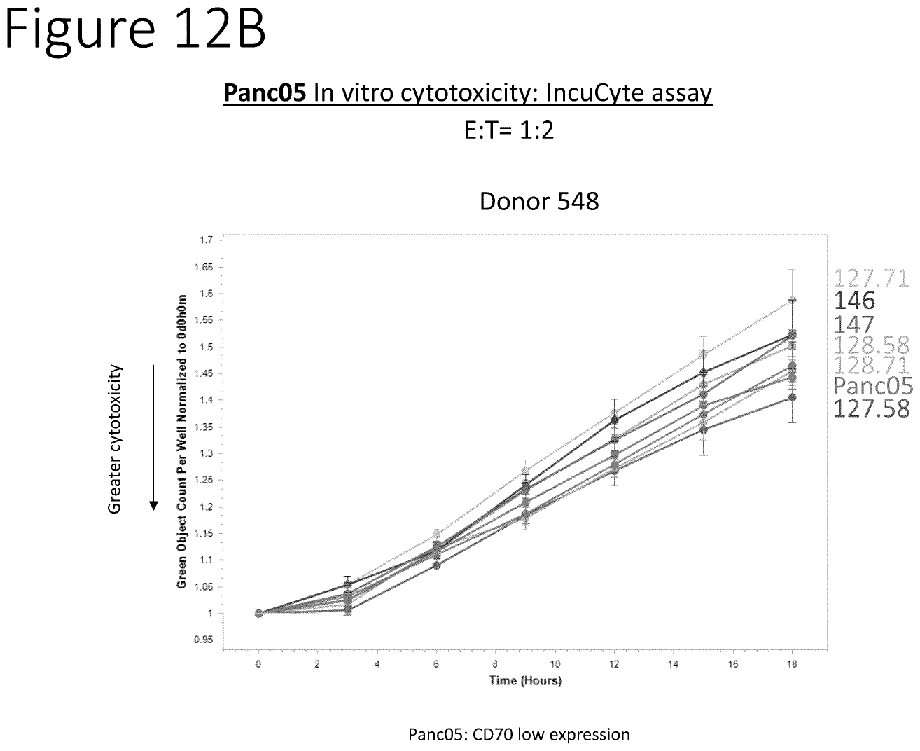

FIGS. 12A-12B show cytotoxicity data collected from two different donors with a 1-day IncuCyte® assay, when NK cells and target cells (Panc05) were present at a ratio of 1:2. As shown, each experimental group reduced tumor cell number (Panc05) as compared to the controls, with the NK127.58 and NK127.71 constructs appearing to be most effective.

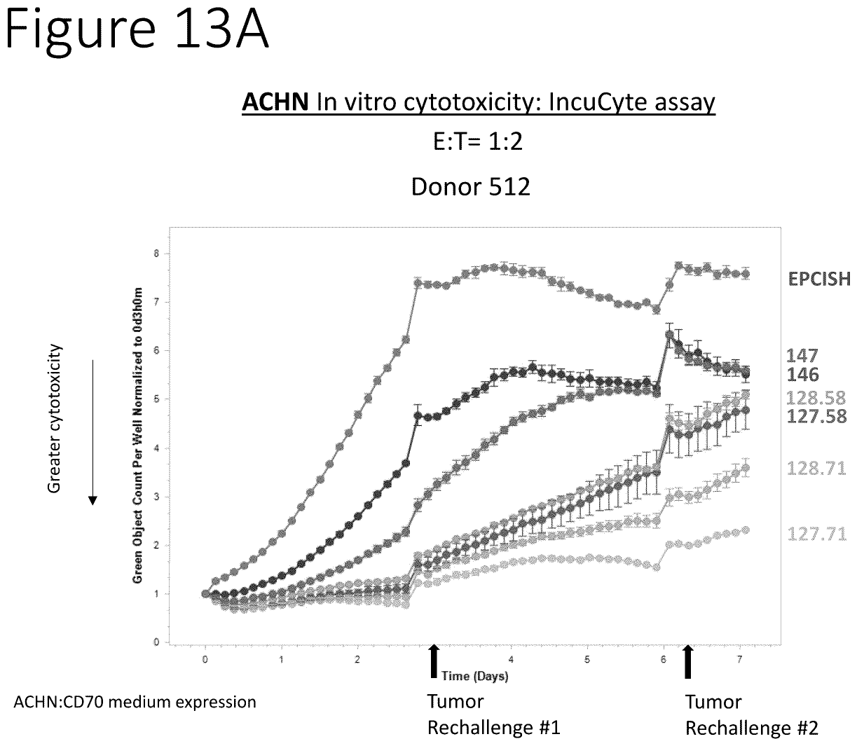

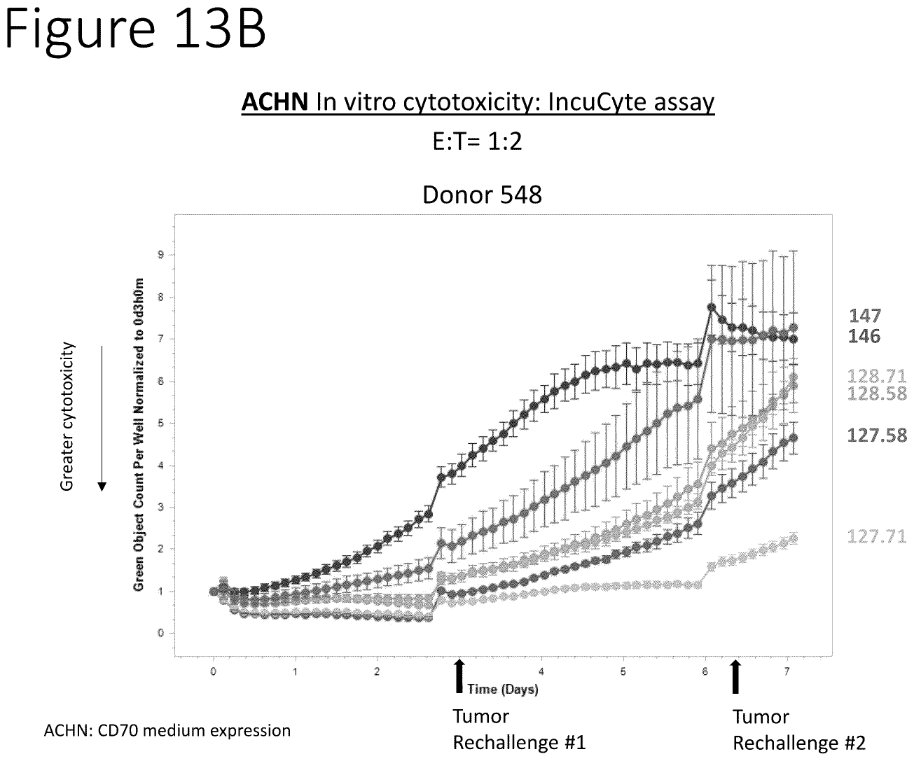

Additional experiments were conducted to evaluate the efficacy of the CD70 CAR-expressing gene edited NK cells against ACHN tumor cells, which express moderate amounts of CD70. As shown in FIGS. 13A and 13B, where data were collected from two different donors, respectively, there were two rechallenges after the initial co-culture. The E:T ratio here was 1:2 and similar to the Bright-Glo™ assay with Panc05 cells, each of the CD70 CAR-expressing groups reduced ACHN tumor cell numbers to a greater degree than control, with the 127.71 construct appearing to be the most potent.

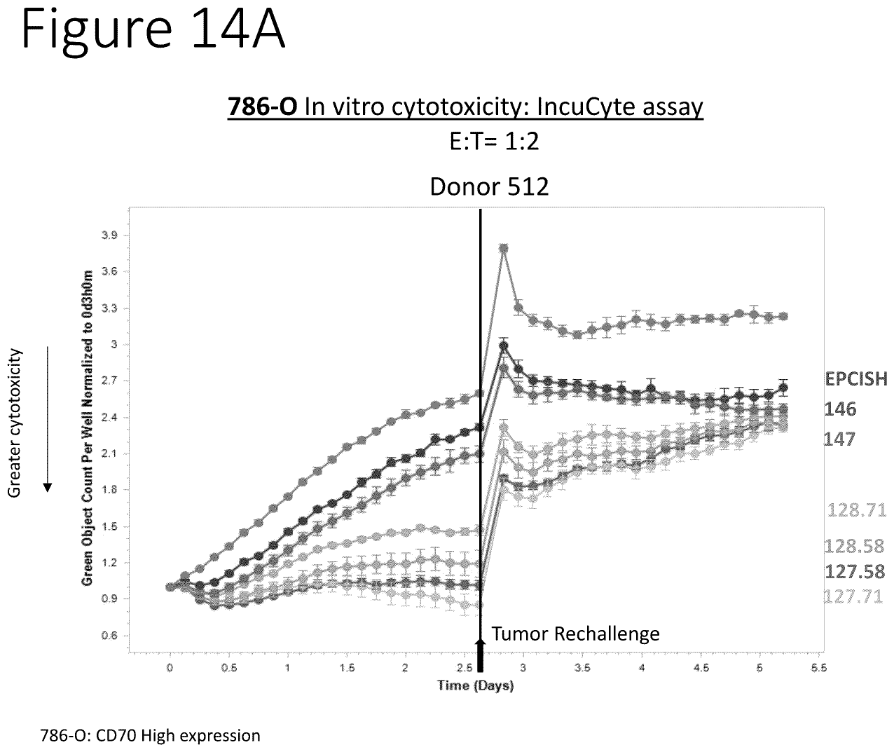

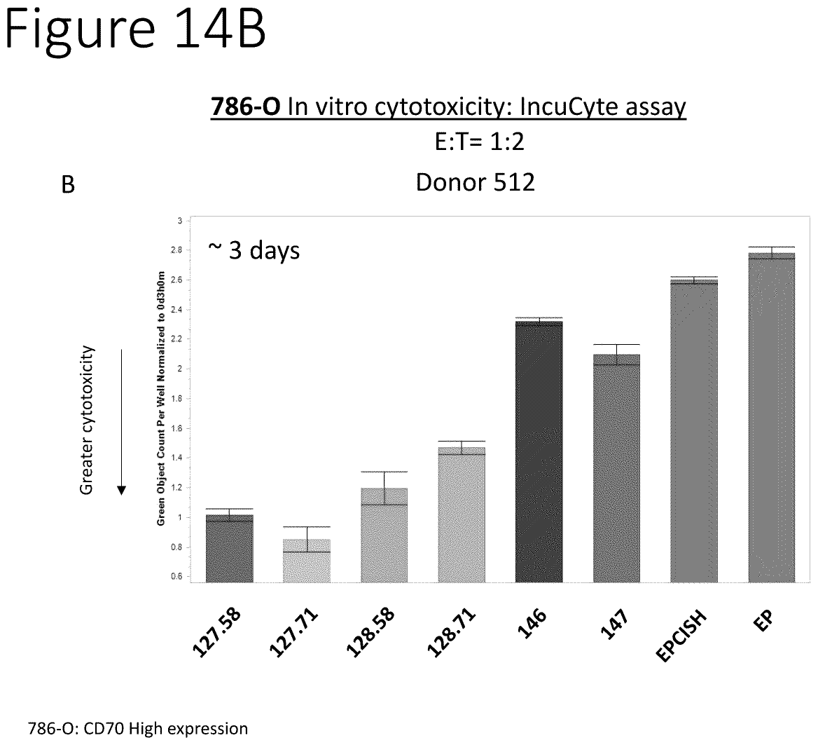

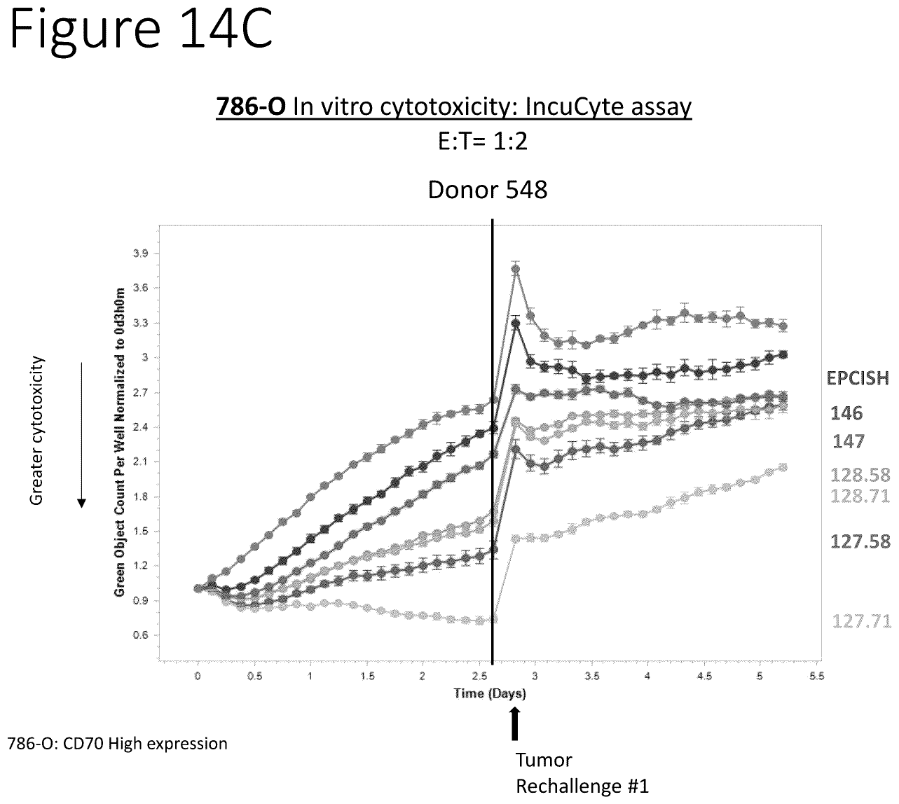

Another assay was performed using 786-O tumor cells (high levels of CD70) with an initial co-culture and a single rechallenge. At the close of the experiment, each of the constructs appeared to exhibit similar cytotoxicity (see FIGS. 14A and 14C from two different donors, respectively). At an intermediate time point (vertical line of 14A, ˜3 days) an assessment was made, with the resultant tumor cell populations depicted in the histogram shown in FIG. 14B for the first donor. At this intermediate time point, each of the CD70 CARs exhibited cytotoxicity greater than CISH-edited NK cells not expressing a CAR (CISH EP) and mock electroporated NK cells (EP). The NK127 and 128 series constructs appeared more potent at this intermediate time point.

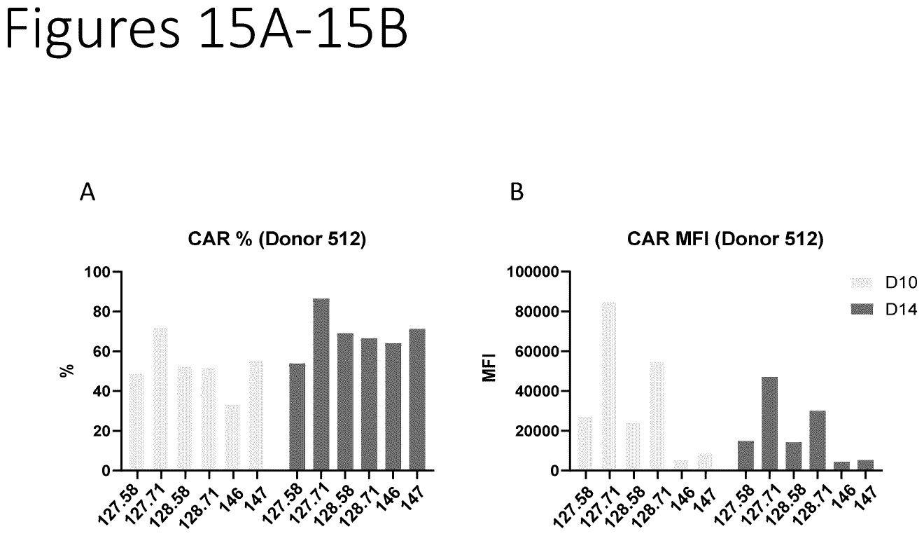

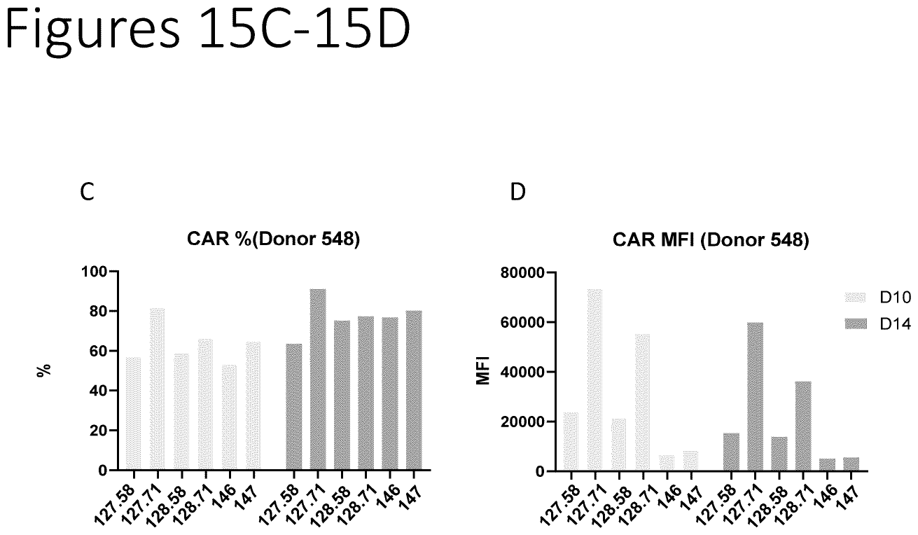

FIGS. 15A-15D show data from two different donors related to the percentage of NK cells expressing the CAR (15A and 15C, respectively) and the density of expression by those positive cells (15B and 15D, respectively) by mean fluorescence intensity (MFI). These data indicate that each of the CAR constructs was expressed relatively consistently within a given donor cell group. The 127 series (127.71) appeared to be slightly more highly expressed across both donors. Notably, the NK127.71 construct appeared to be expressed at higher densities than the other constructs, which may account for the apparent enhanced cytotoxicity of NK cells expressing this construct, against high, mid, or low CD70-expressing tumor lines. Together, these in vitro data demonstrate that expression of a CD70 CAR, in conjunction with gene editing of at least two targets (e.g., CISH and CD70) confers upon NK cells enhanced cytotoxicity and persistence to the therapeutic cells. In several embodiments, this approach is furthered by editing of at least on additional gene, such as CBLB, TGFBR2, and/or an adenosine receptor, in addition to CISH and CD70, imparts further enhanced potency and/or persistence to the NK cells.

### Example 3—In Vivo Characterization of Selected CD70 CARs

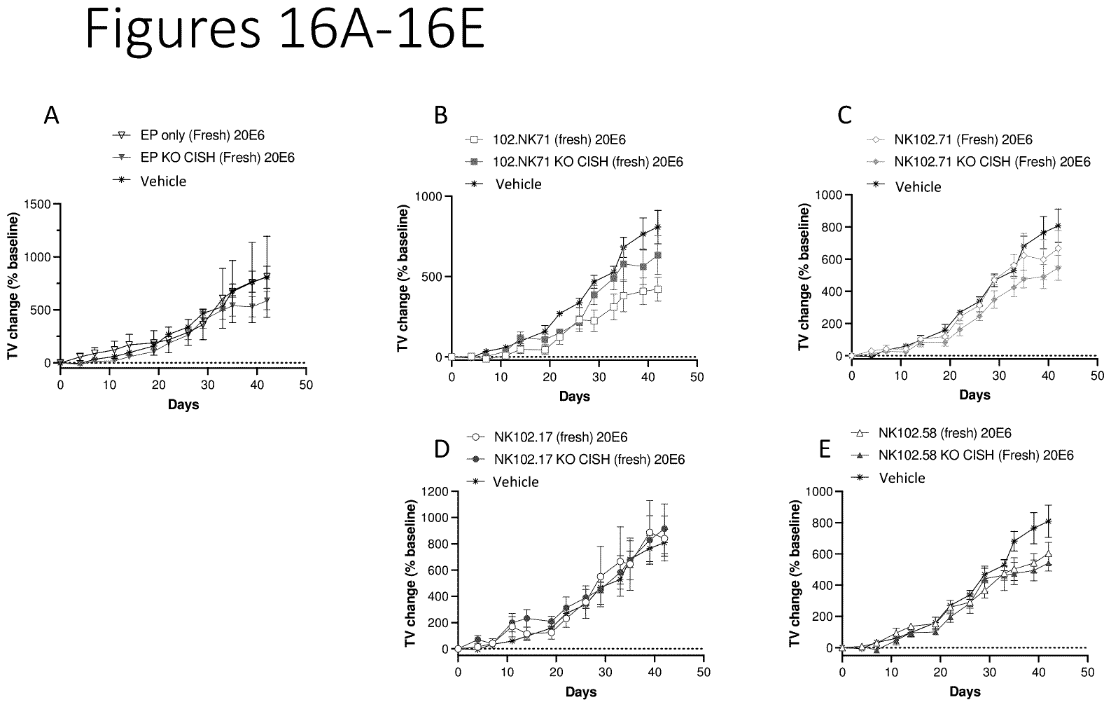

Expanding on the in vitro data discussed above, several in vivo experiments were conducted to evaluate the efficacy of gene edited CD70 CAR-expressing cells. A renal cell carcinoma xenograft model was used in which, 5 days prior to NK cell administration, 786-O renal cell carcinoma cells were injected into mice. A single dose of 20 million NK cells (gene edited at CD70 and CISH and expressing CD70 CARs) was injected at Day 0. FIGS. 16A-16E show representative in vivo data assessing the ability of the indicated CD70 CAR-expressing, gene edited NK cells at controlling tumor growth. The CD70-targeting CARs used in in this experiment share the same VH, VL, and linker sequences as others provided for herein, based on the indicated construct suffix (e.g., “0.71”, “0.58”, or “0.17”, though these specific constructs were manufactured with an alternative production plasmid (denoted by the “102” construct prefix). FIGS. 16A and 16B show control data, and FIGS. 16C and 16E show the CD70 CAR constructs evaluated in the prior experiments. As is notable from these figures, the presence of the CD70 CAR enhances cytotoxicity and editing CISH further enhances the cytotoxic effects. A similar trend was detected in corresponding experiments using an A498 xenograft model (data not shown).

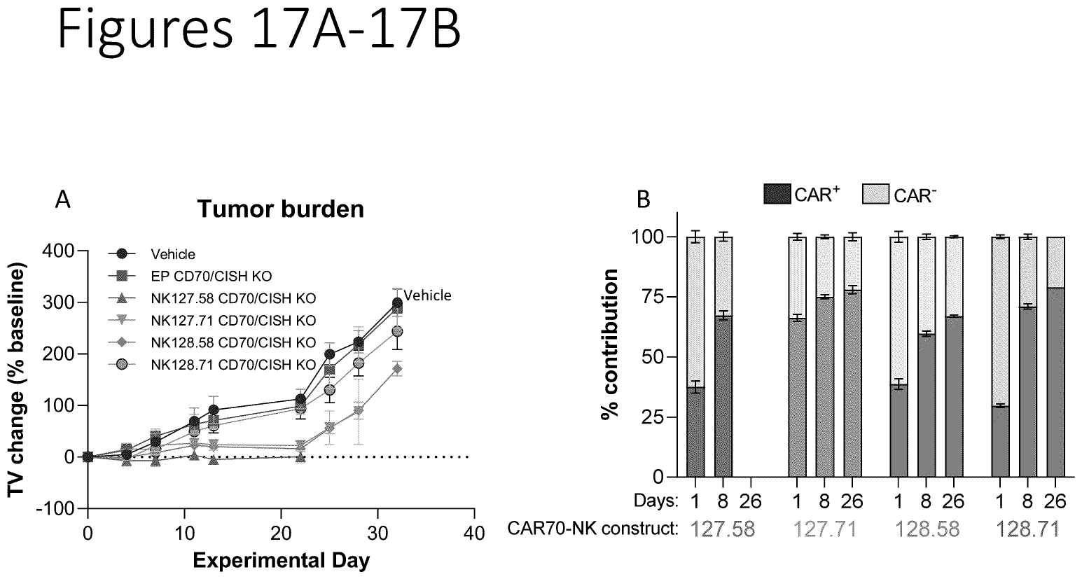

A multi-dose in vivo study was also performed using the 786-O renal cell carcinoma xenograft model. 10 million 786-O cells were injected 3 days before administration of NK cells. Two doses of gene edited CD70 CAR-expressing NK cells were administered: 30 million NK cells at Day 0 and 30 million at Day 7. FIGS. 17A-17B show data related to tumor burden (17A) and the percentage of CAR-expressing NK cells (17B). As shown in FIG. 17A, each of the experimental groups show a reduction in the increase in tumor volume overtime, with the 127.58, 127.71, and 128.58 constructs showing the most control of tumor burden. FIG. 17B shows the persistence of the CAR-positive NK cells. But for the 127.58 group (which was not measured at day 26) each of the groups approached nearly 75% of the total population of NK cells. Each group did show an increase in percentage over the time measured, indicating that the edits to the NK cells enhanced the life span of the edited cell (with the control of tumor burden showing the functionality of the cells over time). Taken together, these data indicate that expression of a CD70 CAR confers upon NK cells enhanced cytotoxicity and editing of the genome of these cells, for example CISH-editing, enhances cytotoxicity and reduces intra-therapeutic cell population fratricide, for example by knocking down CD70. As provided for herein, additional edits to other genes, such as CBLB, TGFBR2, and/or an adenosine receptor, in addition to CISH and CD70 imparts further enhanced potency and/or persistence to the NK cells.

### Example 4—Evaluation of Multiplex Gene Editing in CD70 CAR Expressing NK Cells

As discussed above, in several embodiments, immune cells (e.g., NK cells) are edited, for example to knock down or knock out expression of a plurality of target genes (“multiplex gene editing”). In several embodiments, immune cells, such as NK cells are edited to reduce, substantially reduce, and/or eliminate CD70 expression and engineered to express a CAR that targets CD70. In several embodiments, the immune cells are optionally also edited to reduce, substantially reduce, and/or eliminate CISH expression. In several embodiments, the immune cells are optionally also edited to reduce, substantially reduce, and/or eliminate Casitas B-lineage lymphoma-b (Cbl-b) expression.

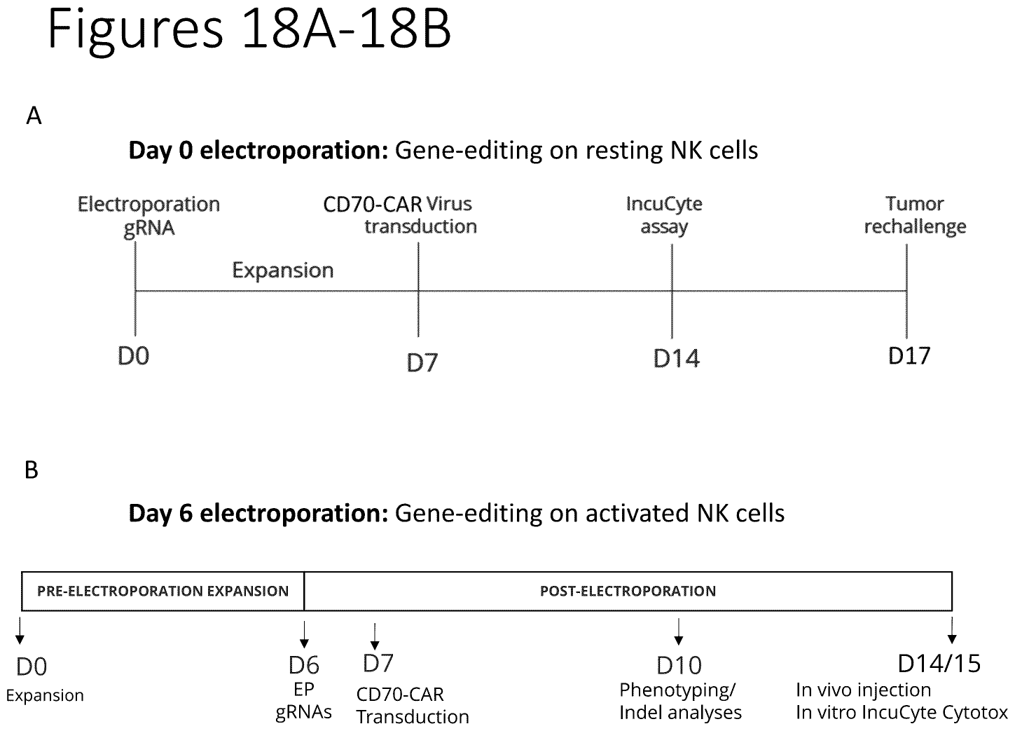

Gene editing can be done at different time points, depending on the embodiment. FIGS. 18A-18B show non-limiting examples of cellular production processes. FIG. 18A shows a Day 0 EP approach, in which the gene editing is performed at Day 0 on resting NK cells. Viral transduction is then performed about 7 days later (after expansion of the edited cells), with in vitro and in vivo evaluation scheduled as shown. In contrast, FIG. 18B shows a Day 6 EP approach, where the NK cells are first expanded (and thus activated) and gene editing is performed one day prior to viral transduction with the CD70 CAR.

The experimental groups for testing the Day 0 and Day 6 multiplex gene editing approaches are shown in Table E2.

TABLE E2
Experimental Groups for Day 0 and Day 6 Multiplex Editing

| | Group (gRNA sequence) |
|---|---|
| 1 | EP only |
| 2 | CD70 KO (e.g., SEQ ID NO: 180) |
| 3 | CD70 KO (e.g., SEQ ID NO: 180) + CISH KO (e.g., |
| | SEQ ID NO: 191) |
| 4 | CD70 KO (e.g., SEQ ID NO: 180) + CBLB KO (e.g., |
| | SEQ ID NO: 195) |
| 5 | CD70 KO (e.g., SEQ ID NO: 180) + CISH KO (e.g., |
| | SEQ ID NO: 191) + CBLB KO (e.g., SEQ ID NO: 195) |

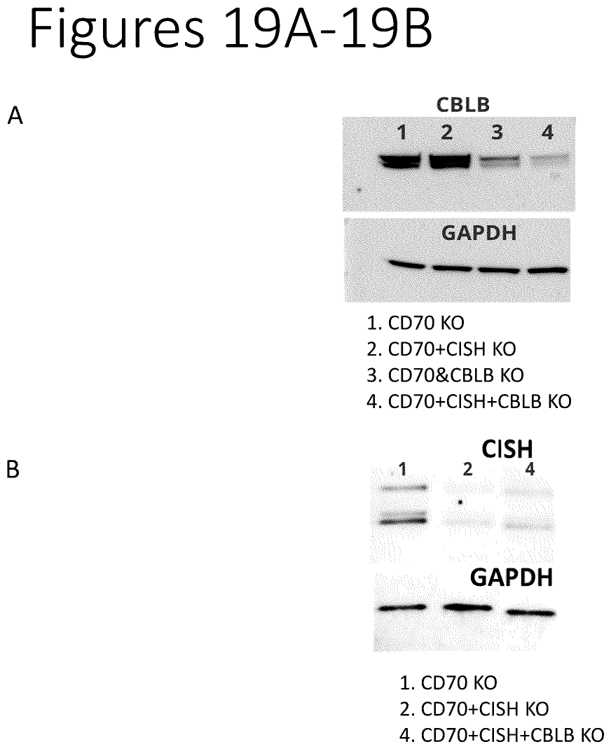

A subset of the gene edited cells were phenotypically characterized at Day 6. As shown in Table E3 and FIGS. 19A-19B, the edited genes were successfully disrupted at both the protein and genomic level.

TABLE E3
Summary of Expression and Indels Using Day 0 Electroporation

| | | Surface FACS | | | |
|---|---|---|---|---|---|
| | | Phenotype | | | |
| | | (Day 11) | TIDE/NGS Indels (Day 6) | | |
| | Group | CD70% | CD70 | CISH | CBLB |
| 1 | EP only | 66.2% | — | — | — |
| 2 | CD70 KO | 16.9% | 81.1% | — | — |
| 3 | CD70 + CISH KO | 17.2% | 76.5% | 68.9% | — |
| 4 | CD70 + CBLB KO | 29.4% | — | — | 87.8% |
| 5 | CD70 + CISH + | 30.1% | 75.1% | 67.0% | 81.0% |
| | CBLB KO | | | | |

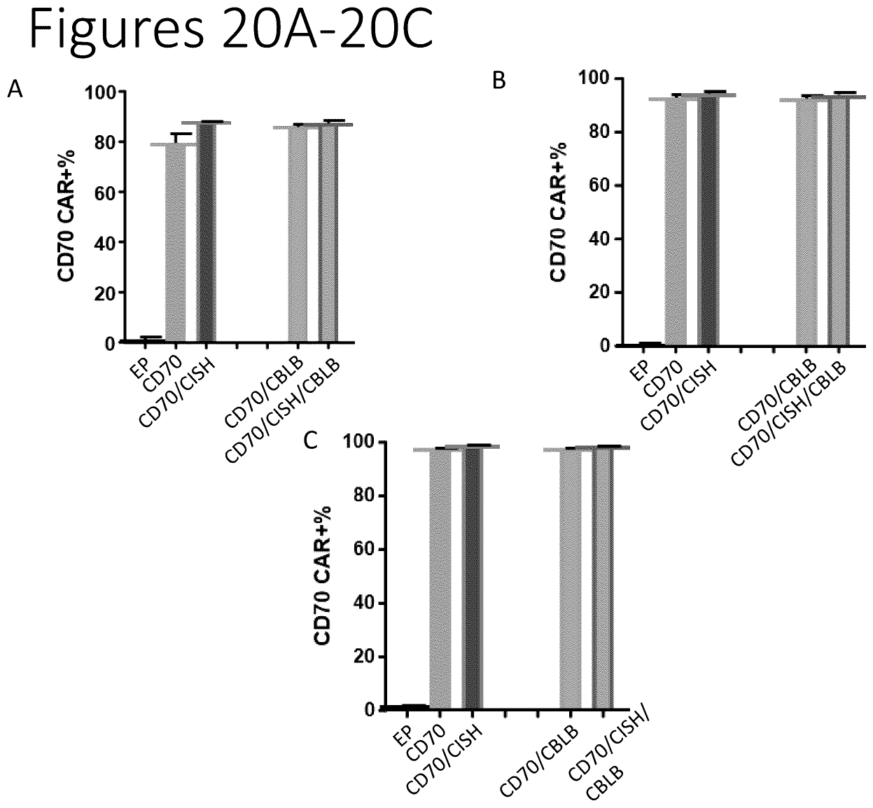

As shown in Table E3, the surface expression of CD70 in the edited groups was reduced by ˜70-85%, depending on the group (measured at 11 days post-EP). Six days after EP, TIDE indel analysis showed an indel frequency of between about 75-82% for CD70, about 67-68% for CISH, and about 80-88% for CBLB. As shown in lanes 1-4 of the western blot of FIG. 19A, CBLB protein expression was reduced in analysis groups 4 (CD70/CBLB KO) and 5 (CD70/CISH/CBLB KO), as compared to CBLB protein expression in groups 1 (CD70 KO) and 2 (CD70/CISH KO). Similarly, as shown in lanes 1, 2 and 4 in FIG. 19B, CIS protein expression was reduced in groups 2 (CD70/CISH KO) and 4 (CD70/CISH/CBLB KO), as compared to CIS protein expression in group 1 (CD70 KO). Expression of the CD70 CAR caused the NK cells expressing the CAR to be enriched in culture overtime. Shown in FIGS. 20A-20C, the percentage of the culture that was CD70 CAR-positive increased from Day 11 (20A) to Day 21 (20B) and even to Day 28 (20C). The resultant culture at Day 28 was nearly 100% positive for the CD70 CAR.

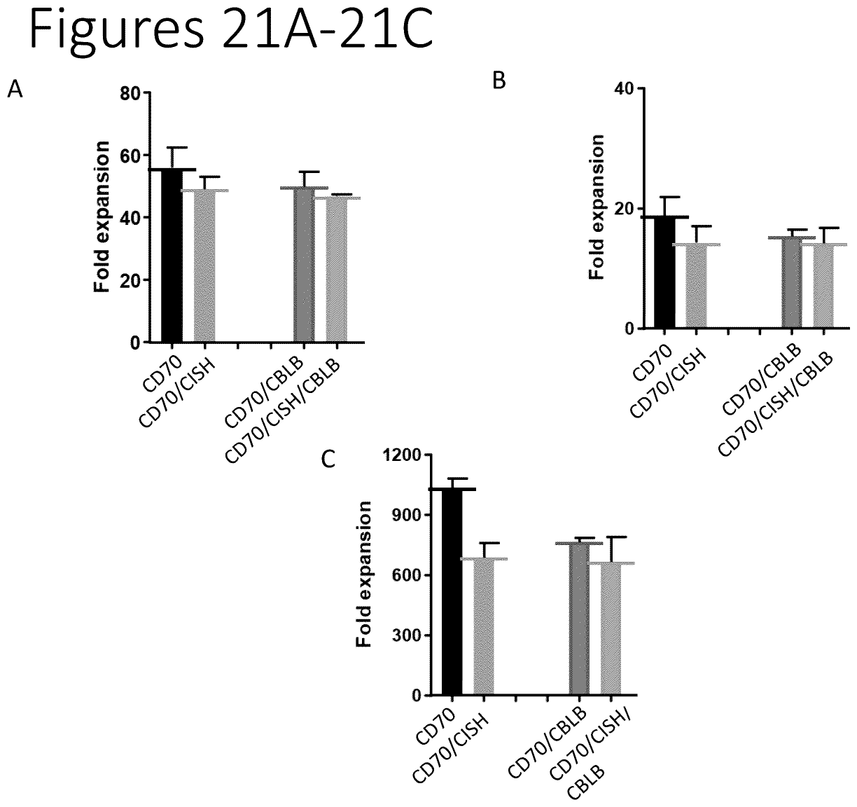

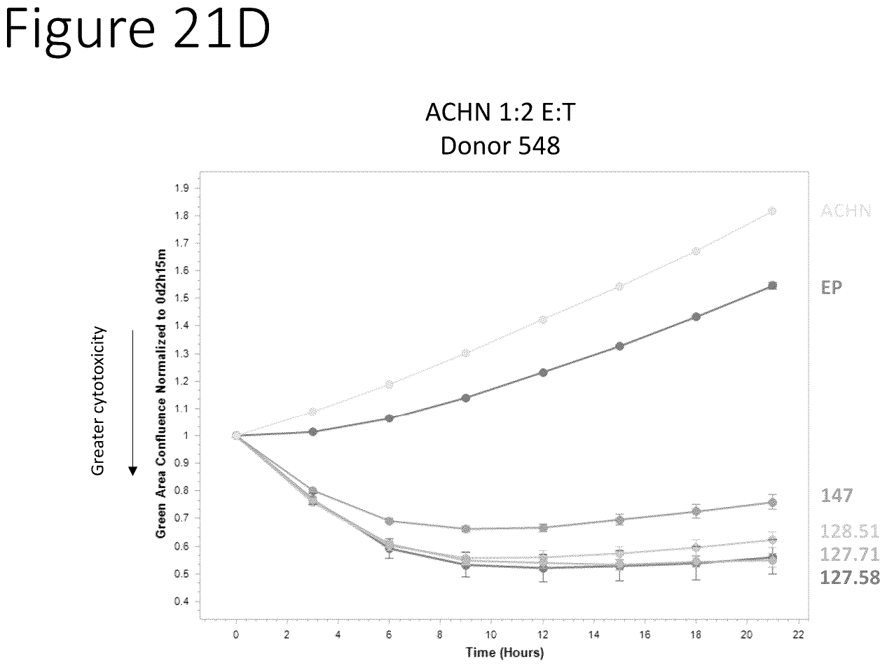

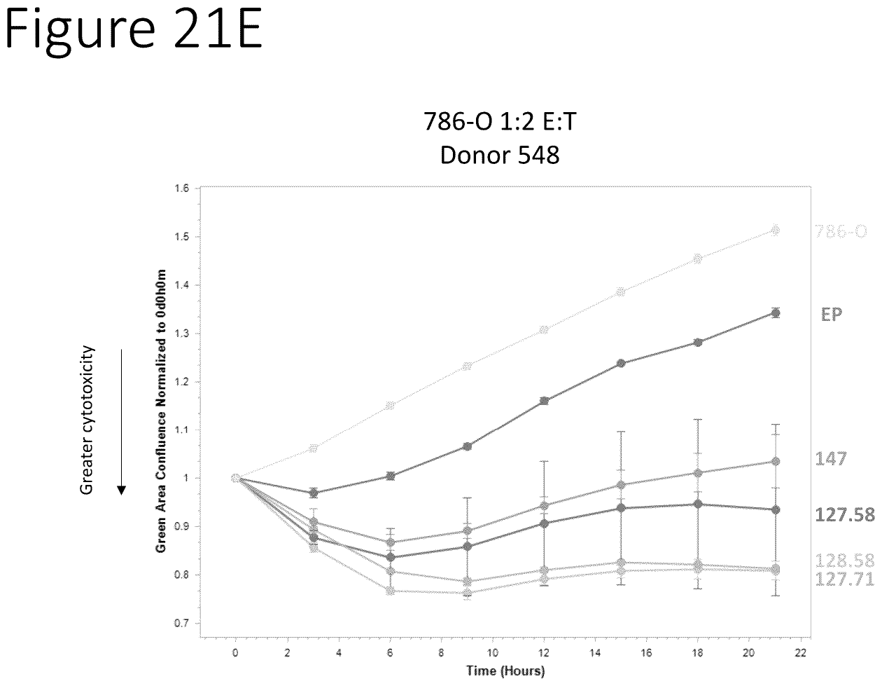

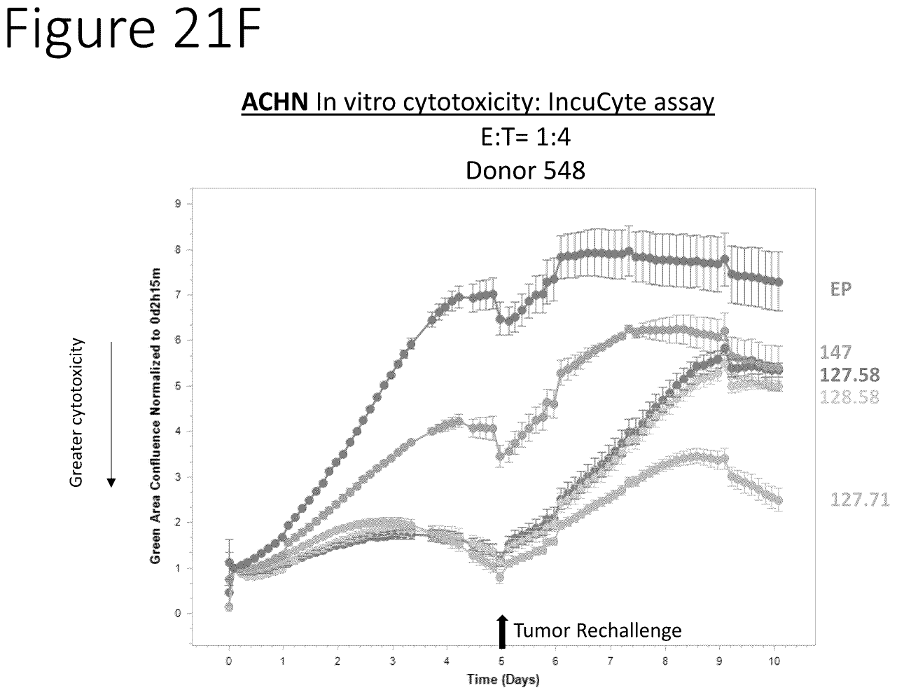

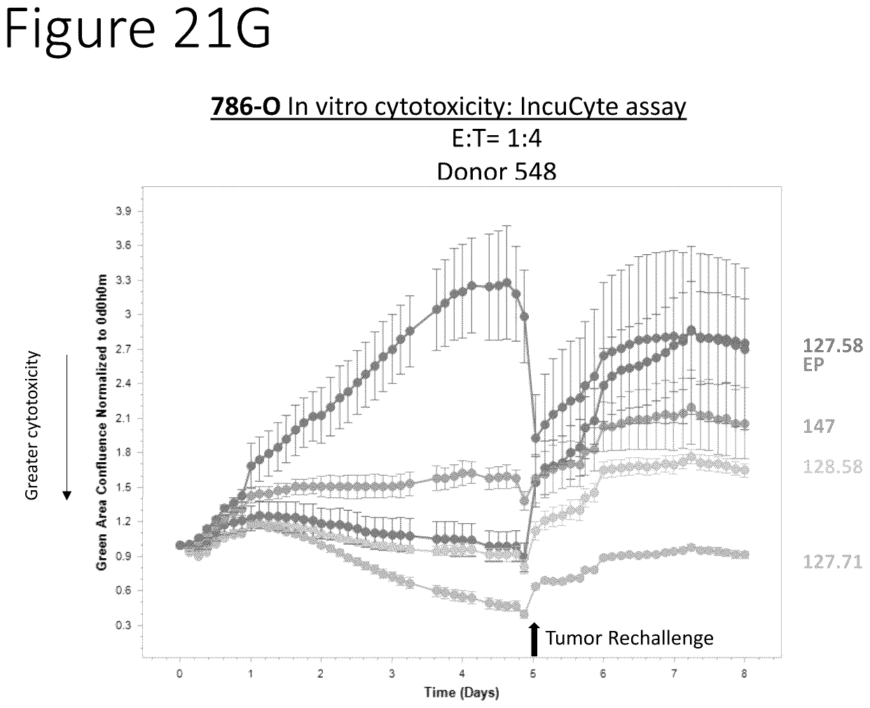

An evaluation of the various editing combinations was performed with respect to the ability of the edited NK cells to expand in culture. FIG. 21A shows data for the indicated edit combinations with respect to fold expansion pre-transduction. While there was some variance, each of the treatment groups showed generally similar expansion. FIG. 21B shows the degree of expansion of each treatment group post-transduction. It is noted that the reduction in expansion could be a refractory response to the transduction protocol. However, by day 14 the overall fold expansion (21C) recovered and was approximately 1000-fold in the single edit to CD70 group, with the dual and triple edit groups being approximately 650-fold. It is notable that there does not appear to be a substantial negative impact on expansion potential when editing three genes (as opposed to two).

The functionality of triple edited NK cells expressing different non-limiting CD70 CARs was performed by in vitro IncuCyte® cytotoxicity assay using ACHN and 786-O cells (mid-level and high CD70 expression, respectively) at a 1:2 E:T ratio. As shown in FIGS. 21D-E, each experimental group reduced ACHN and 786-O cells, respectively, compared to control (EP). A similar assay was performed with an E:T ratio of 1:4 and using one tumor rechallenge provided on day 5 after the initial co-culture. As shown in FIGS. 21F-G, each of the CD70 CAR-expressing groups reduced ACHN and 786-O tumor cell numbers, respectively, to a greater degree than control (EP), with the 127.71 CAR appearing most potent.

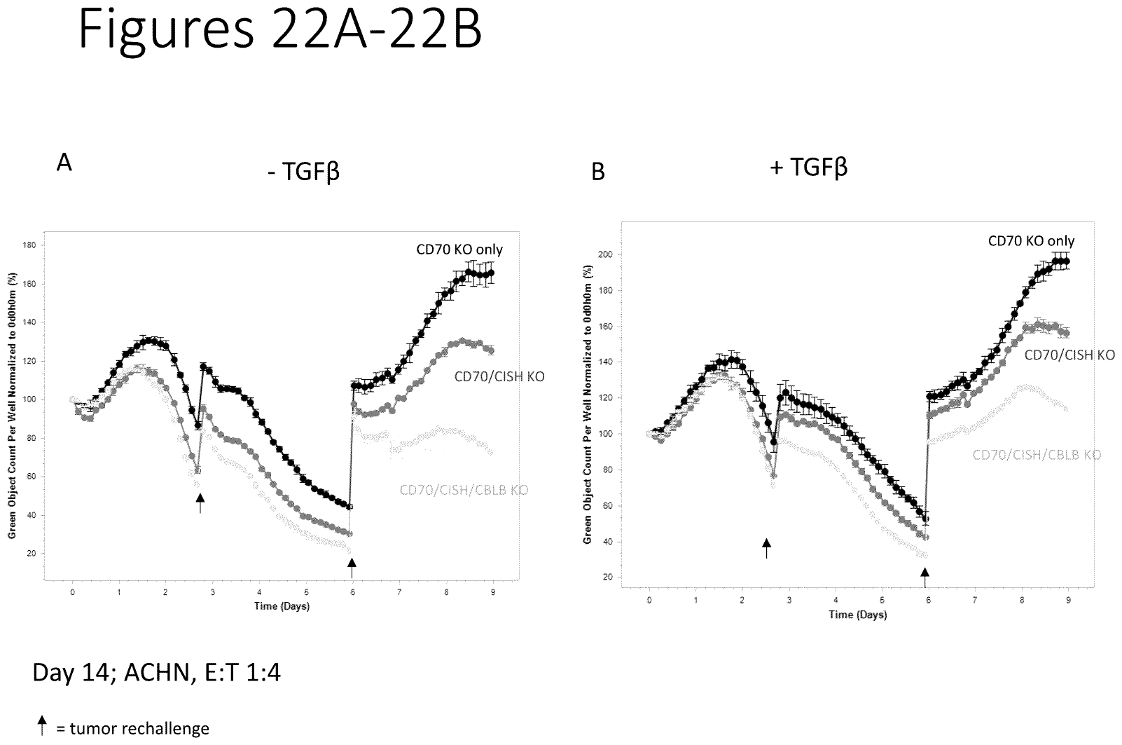

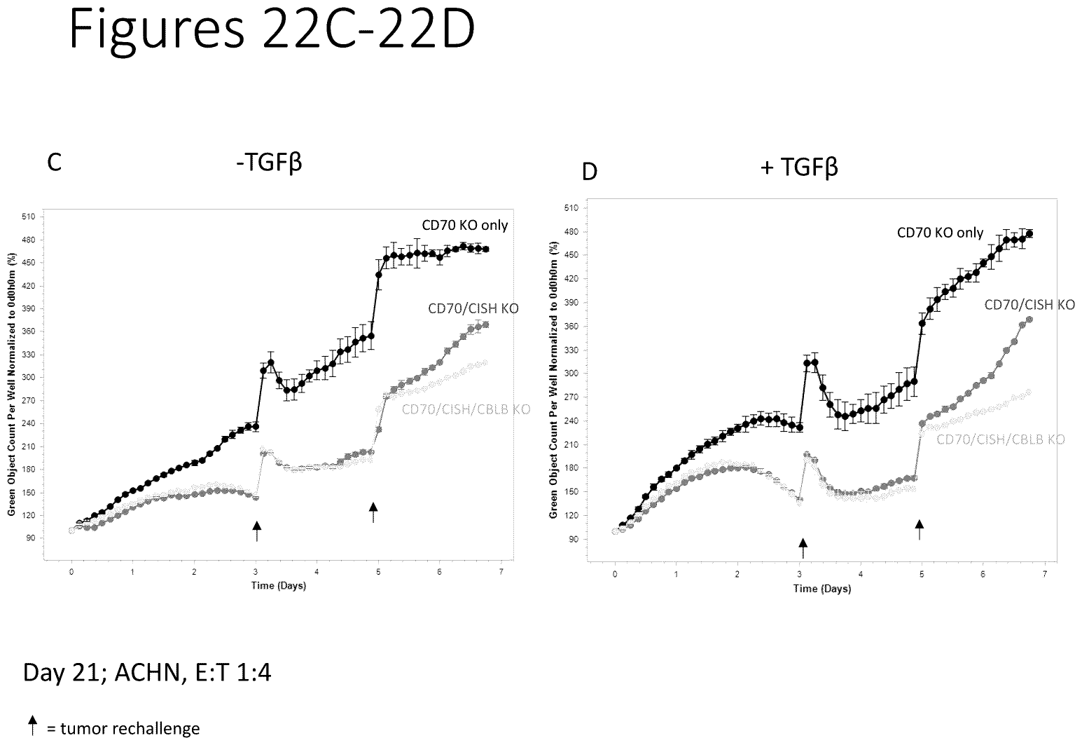

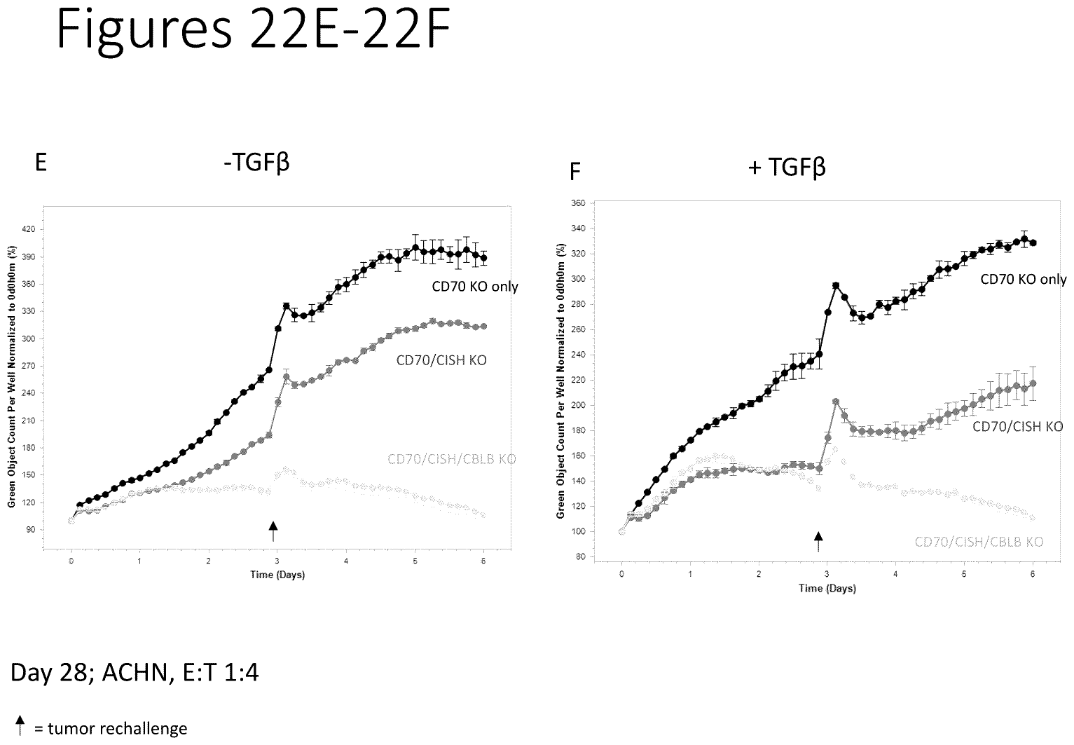

Assessing the functionality of the engineered and edited cells was further performed in an in vitro cytotoxicity assay using ACHN cells (mid-level CD70 expression) at a 1:4 E:T ratio, and beginning on day 14 post-editing, with two rechallenges. As shown in FIG. 22B, the CD70/CISH edited group showed substantially enhanced cytotoxicity as compared to the CD70 only edited group. Moreover, despite two rechallenges with fresh tumor cells, the triple edit to CD70/CISH/CBLB imparted significantly greater cytotoxicity as compared to the other groups. While the conditions in FIG. 22A were in the absence of TGF beta, even in the presence of cytotoxicity-inhibiting TGF beta, the triple edit to CD70/CISH/CBLB still exhibited the most substantial cytotoxicity of the experimental groups. Taking the assessment out to begin 21 days post-editing, a similar pattern is shown in FIG. 22C-22D. Even beginning the assessment at 28 days post-editing, the triple edited NK cells (CD70/CISH/CBLB) still show robust control of tumor growth, despite a rechallenge at three days (22E) and also in the presence of TGF beta (22F). These data show that the triple edit causes substantial enhancement of potency and persistence.

Using the same experimental groups as laid out in Table E2, experiments were conducted using the Day 6 EP approach (see FIG. 18B). Evaluated at Day 10 (from expansion, day 4 post-edit, day 3 post transduction), gene editing successfully disrupted expression at the protein and genomic level.

TABLE E4
Summary of Expression and Indels Using Day 6 Electroporation

| | | Surface | | | |
|---|---|---|---|---|---|
| | | FACS | | | |
| | Group | Phenotype | TIDE/NGS Indels | | |
| | (gRNA sequence) | CD70% | CD70 | CISH | CBLB |
| 1 | EP only | 63.0% | — | — | — |
| 2 | CD70 KO (e.g., SEQ ID | 2.45% | 86.5% | — | — |
| | NO: 180) | | | | |
| 3 | CD70 KO (e.g., SEQ ID | 2.94% | 85.3% | 94.8% | — |
| | NO: 180) + CISH KO | | | | |
| | (e.g., SEQ ID NO: 191) | | | | |
| 4 | CD70 (e.g., SEQ ID | 2.84% | — | — | 79.4% |
| | NO: 180) + CBLB KO | | | | |
| | (e.g., SEQ ID NO: 195) | | | | |
| 5 | CD70 KO (e.g., SEQ ID | 3.10% | 80.7% | 89.3% | 72.3% |
| | NO: 180) + CISH (e.g., | | | | |
| | SEQ ID NO: 191) + | | | | |
| | CBLB KO (e.g., SEQ ID | | | | |
| | NO: 195) | | | | |

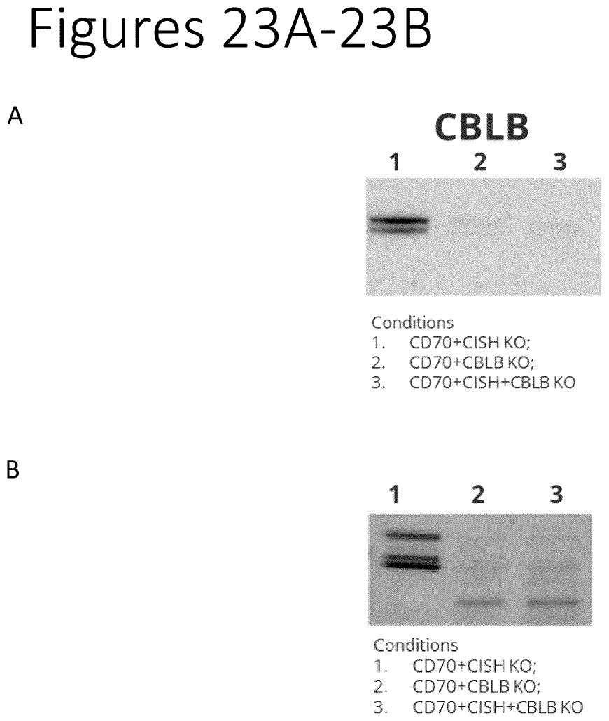

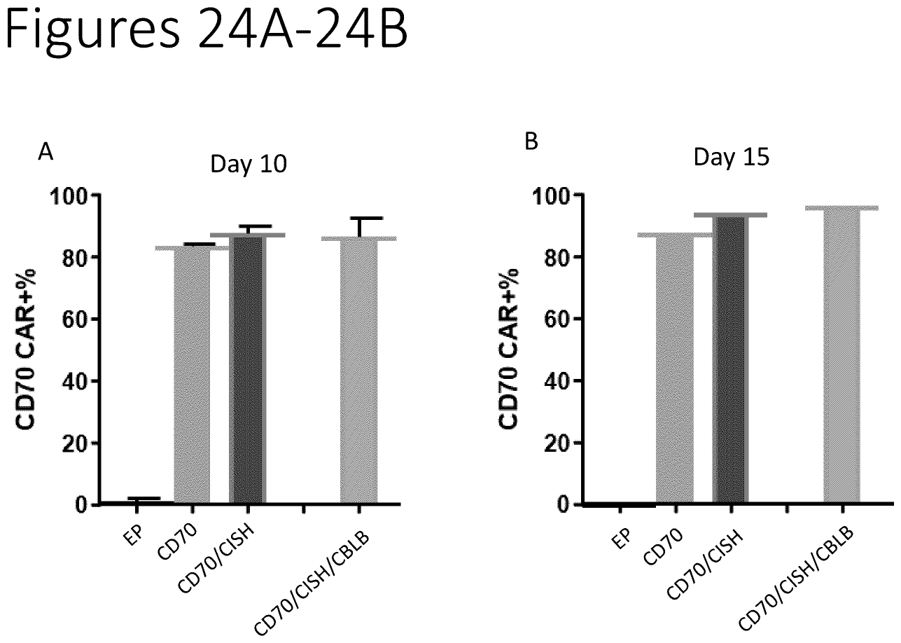

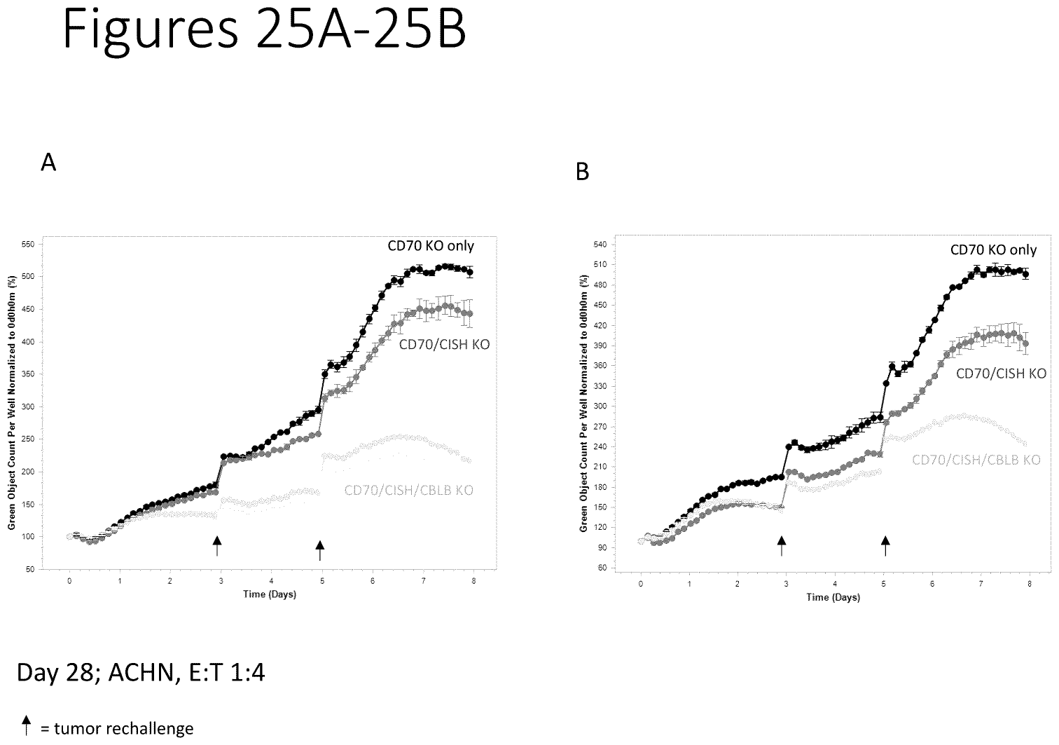

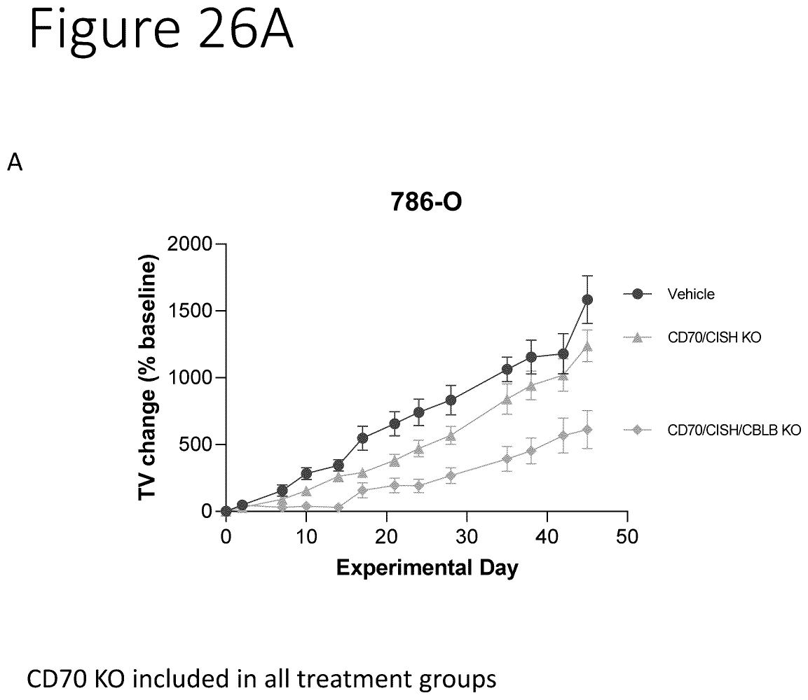

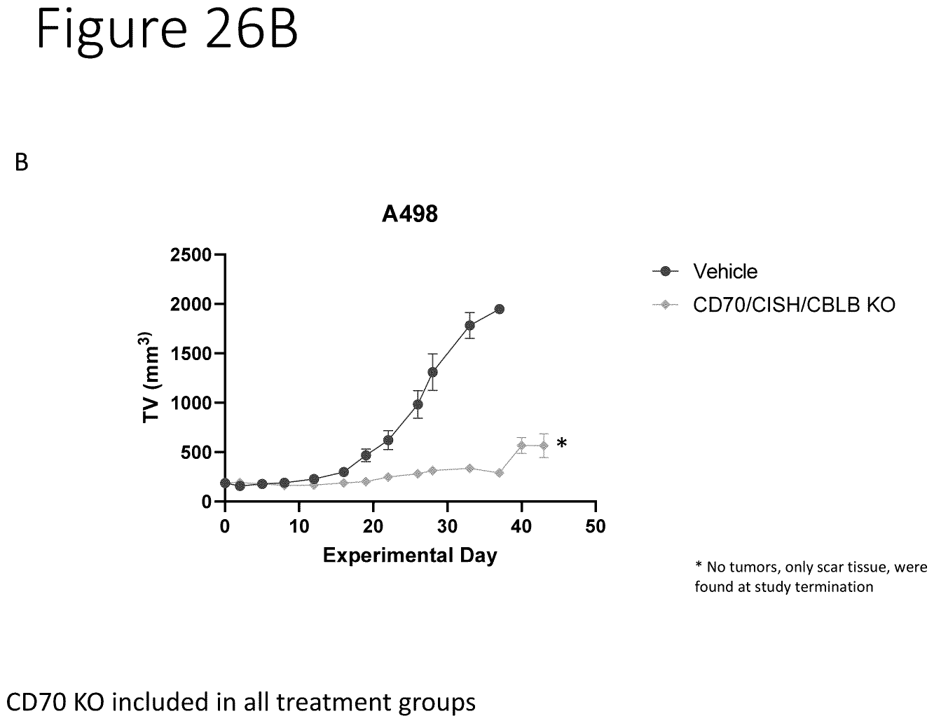

As shown in Table E4, the surface expression of CD70 in the edited groups was reduced by ˜over 95%. TIDE indel analysis showed an indel frequency of between about 80-87% for CD70, about 90-95% for CISH, and about 70-80% for CBLB. As shown in lanes 1-3 in the western blot of FIG. 23A, CBLB protein expression was reduced in analysis groups 2 (CD70/CBLB KO) and 3 (CD70/CISH/CBLB KO), as compared to CBLB protein expression in group 1 (CD70/CISH KO). Similarly, as shown in lanes 1-3 in FIG. 23B, CIS protein expression was reduced (see groups 2 (CD70/CBLB KO) and 4 (CD70/CISH/CBLB KO)). Expression of the CD70 CAR caused the NK cells expressing the CAR to be enriched in culture over time. Shown in FIGS. 24A-24B, the percentage of cells in the culture that were CD70 CAR-positive was approximately 80% on day 10 (FIG. 24A), which increased to over 90% on Day 15 (FIG. 24B). Similar to the data shown using the Day 0 EP approach, the Day 6 EP cell groups, in particular the triple edit (CD70/CISH/CBLB) showed significant cytotoxicity against ACHN cells (FIG. 25A), even in the presence of TGF beta (FIG. 25B). This enhanced cytotoxicity was not limited to in vitro settings, as two xenograft models were used to assess the effectiveness of the various treatment groups. As shown in a 786-O (high CD70 expression) xenograft model, the triple edit (CD70/CISH/CBLB) CD70 CAR-expressing NK cells had the lowest increase in tumor volume over approximately 45 days (FIG. 26A). Similarly, in an A-498 xenograft (also high CD70 expression), the triple edit (CD70/CISH/CBLB) CD70 CAR-expressing NK cells prevented substantial increases in tumor volume, notably resulting in no tumors, but only scar tissue, at the termination of the experiment (FIG. 26B). Taken together, these in vitro and in vivo data demonstrate that multiplex editing does not adversely affect the ability of NK cells to expand in culture and that triple edits, such as CD70/CISH/CBLB are particularly effective in enhancing the cytotoxicity and persistence of NK cells engineered to express a CD70 CAR. Thus, in several embodiments, provided for herein are CD70 CAR-expressing NK cells edited to reduce or eliminate endogenous CD70, CISH and CBLB for enhanced cancer therapy.

### Example 5—Off-Target Gene Editing Analysis

While the triple edits discussed above were shown to be unexpectedly effective in enhancing the cytotoxicity and persistence of the NK cells, additional studies were undertaken to evaluate the off target editing that may occur using exemplary gRNAs.

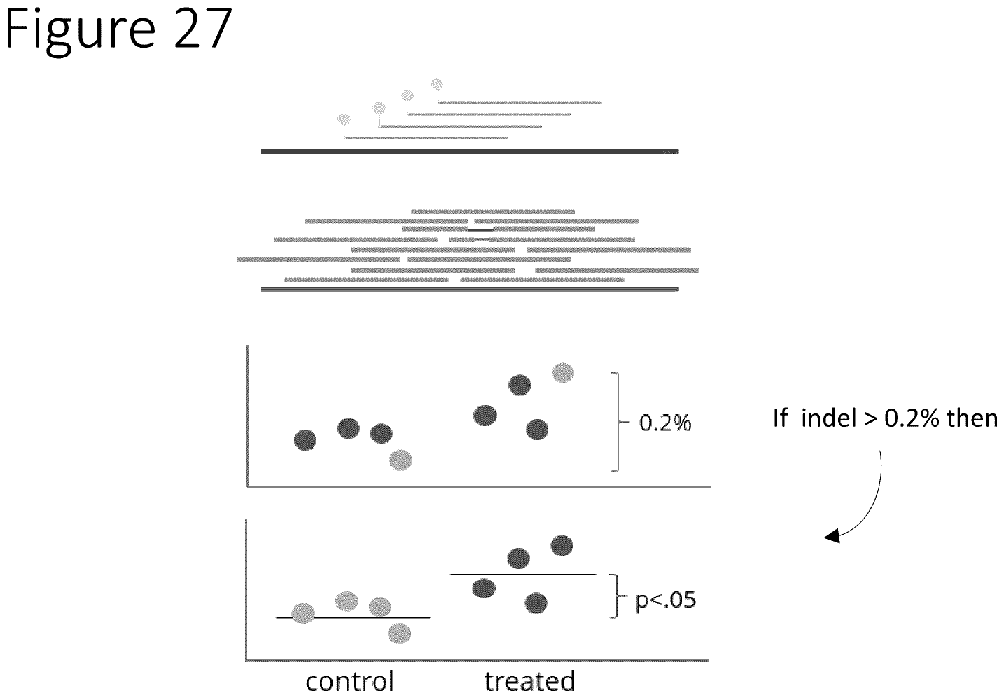

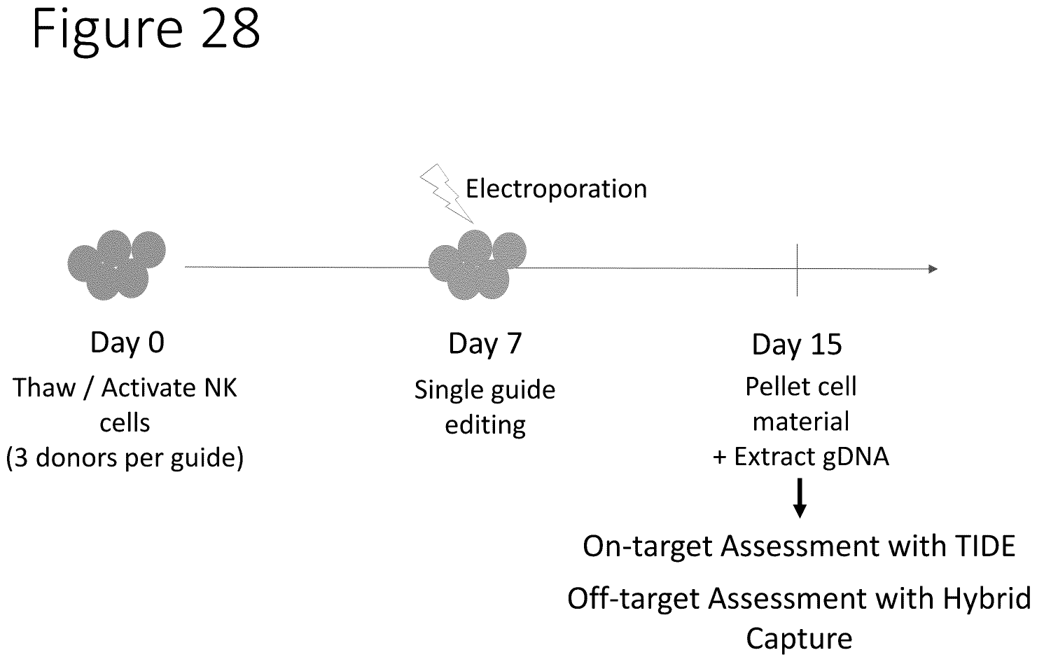

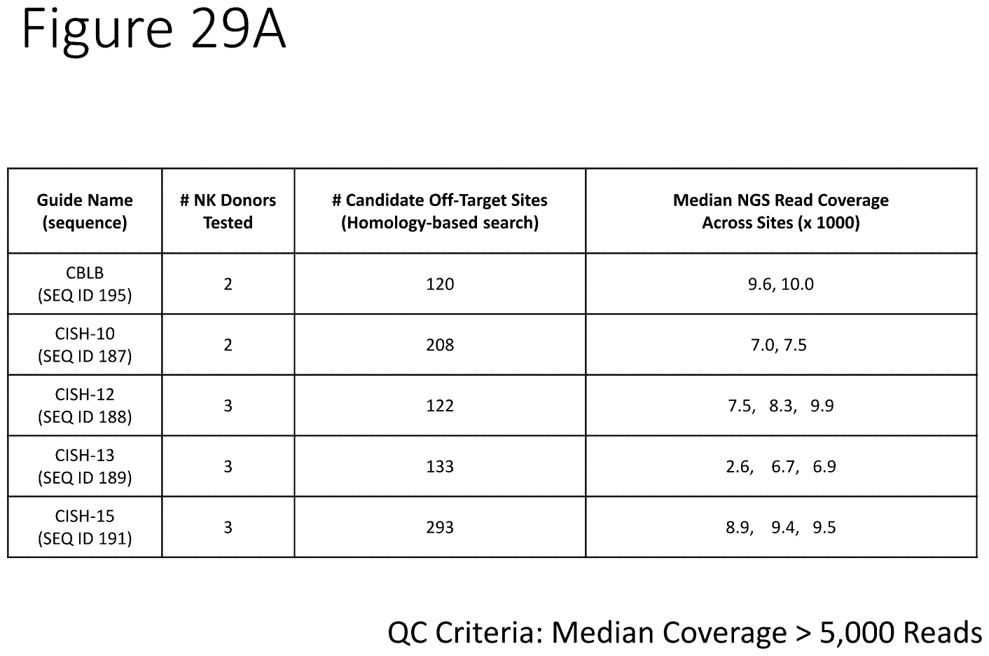

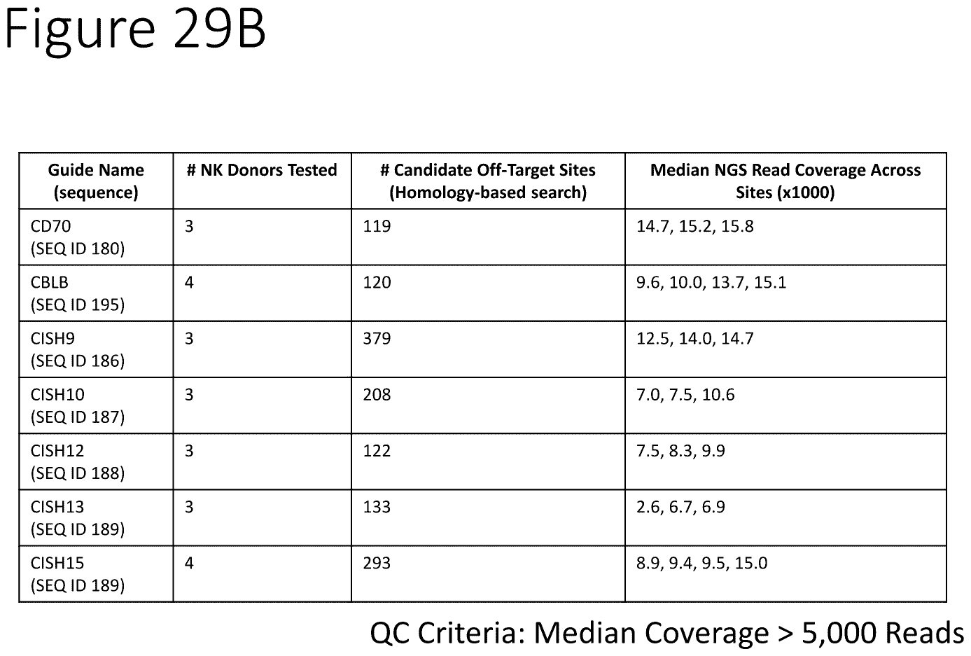

FIG. 27 shows a schematic for analysis of off-target editing by hybrid capture. A series of probes is generated and tiled across each potential off-target site. Based on the probe signal, the targeted regions are enriched and sequenced. The total number of sequencing reads with indels is calculated and divided by the number of total reads at each potential off-target site. If the frequency of indels (to total reads in donor matched control) in an edited sample is greater than 0.2%, additional statistical analysis is performed. For example, a paired, one-sided T test can be performed to compare the control and treated samples, and sites with P<0.05 are confirmed to be off-target edits. FIG. 28 shows a non-limiting off-target analysis process flow. FIG. 29A sets forth information regarding the possible off target sites and estimated NGS read coverage for selected gRNAs provided for herein. FIG. 29B shows a summary of the previous data provided in FIG. 29A, along with additional data for more donors for selected gRNAs. As indicated, a QCcriteria for NGS analysis is median coverage of more than 5000 reads, for which all samples surpassed other than one iteration of CISH-13 gRNA.

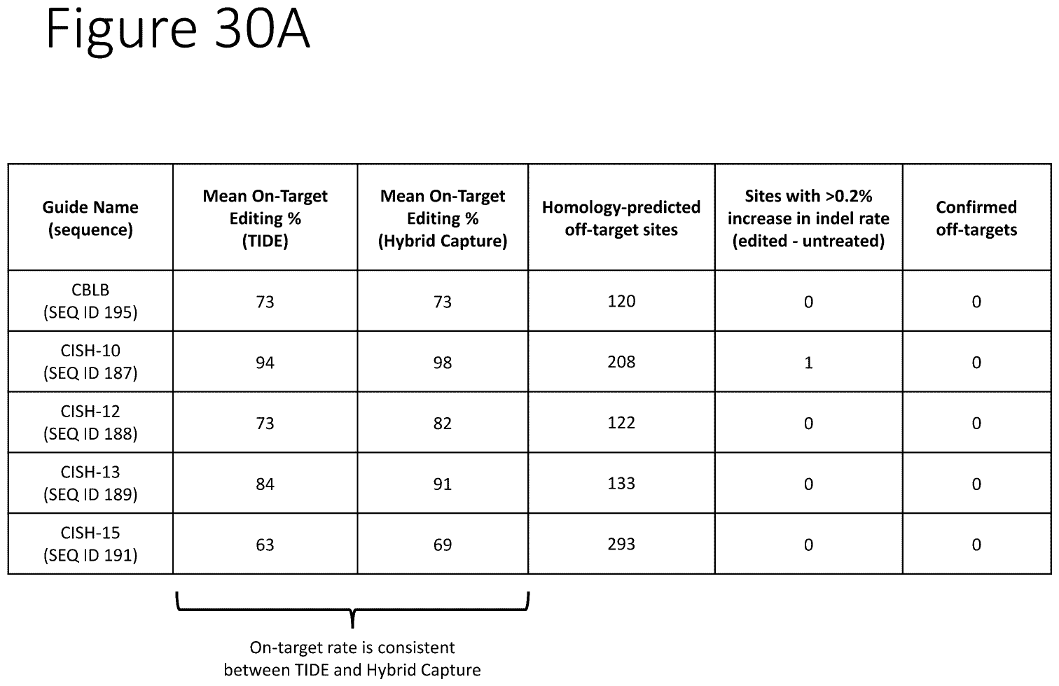

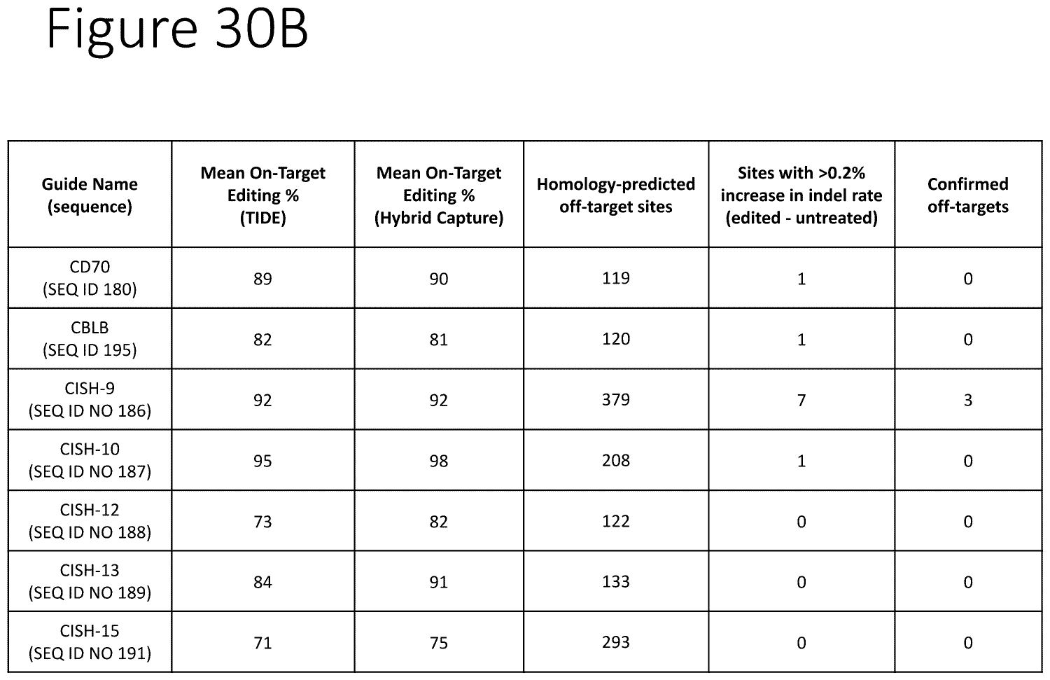

FIGS. 30A and 30B show the results of off-target analysis for the selected gRNAs shown in FIGS. 29A and 29B, respectively. Whether calculated by TIDE analysis or hybrid captures, the calculated on-target editing rate was consistent for each gRNA. As shown, only a single sample (CISH-10 gRNA) required the more detailed statistical comparison due to exceeding the 0.2% indel ceiling. However, that analysis still confirmed no off-target edits. Therefore, these data confirm the accuracy and specificity of these non-limiting embodiments of gRNAs for gene editing.

### Example 6—Analysis of Multiplex Gene Edited NK Cells

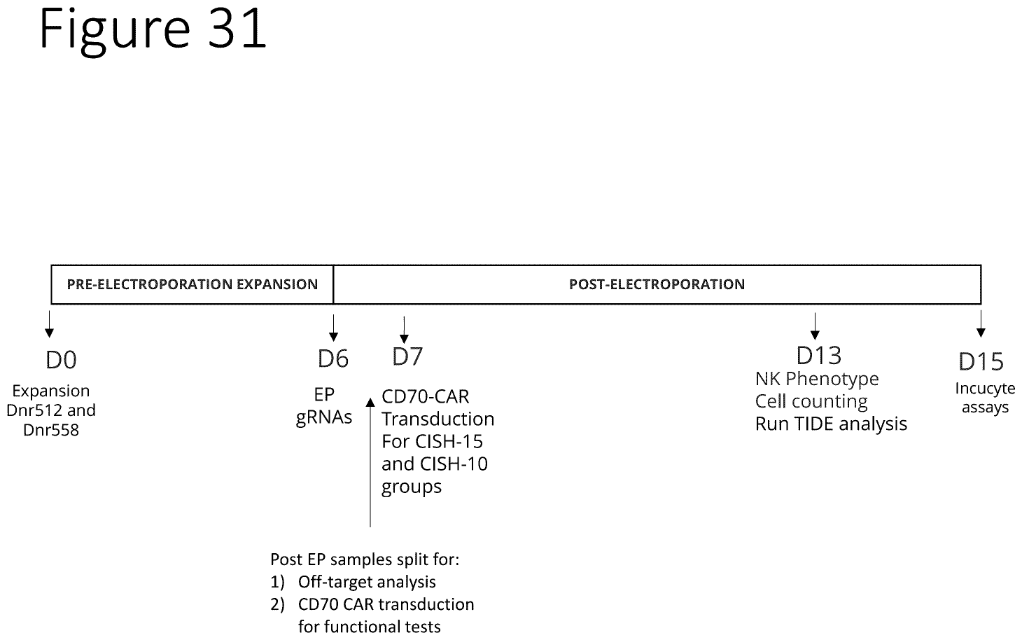

FIG. 31 shows a non-limiting process flow for producing experimental samples for assessing the impact of multiplex gene editing. Edited cells will be generated using the Day 6 EP approach and will therefore be edited after 6 days of expansion. Samples will be split after EP and a subset will be used for off target analysis and a subset will be transduced with CD70 CAR candidates and subject to functional testing.

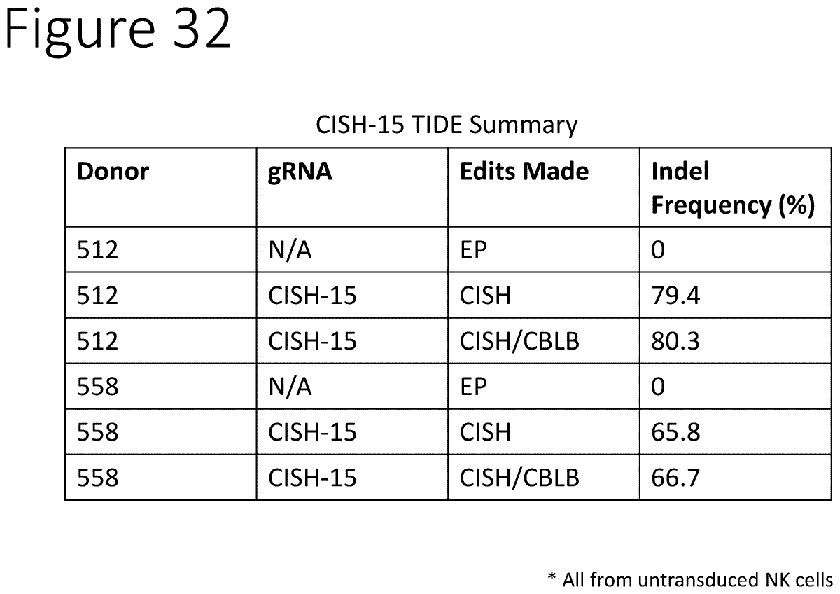

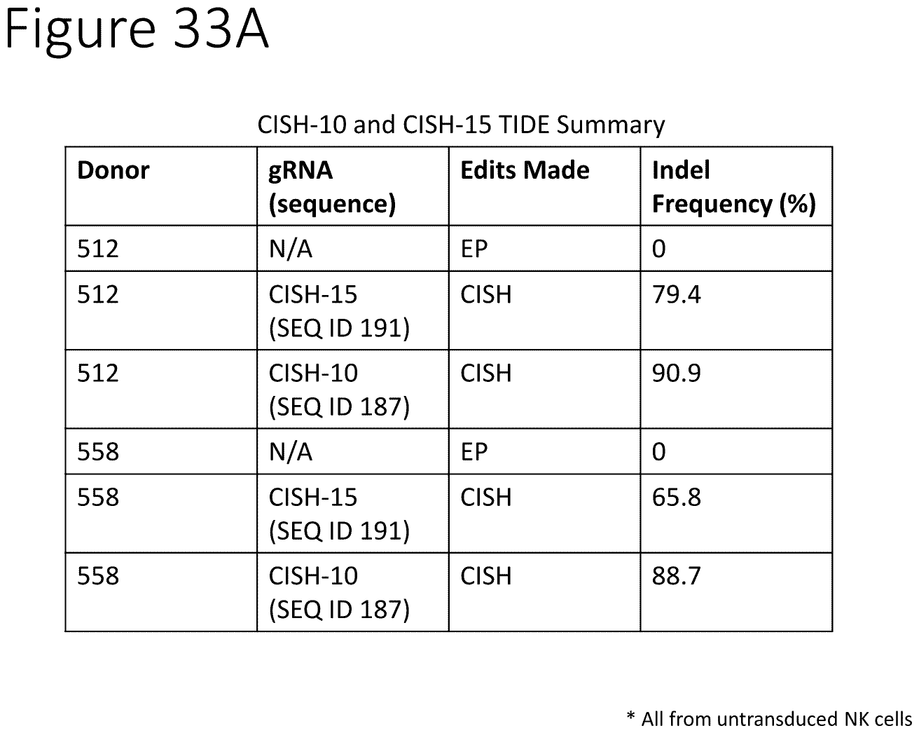

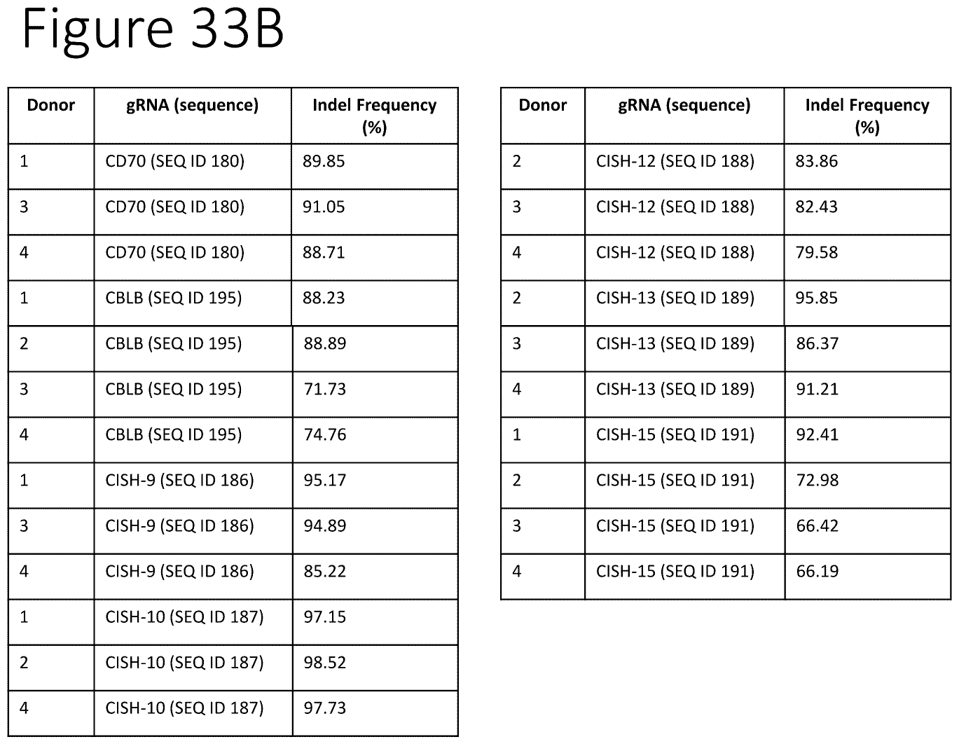

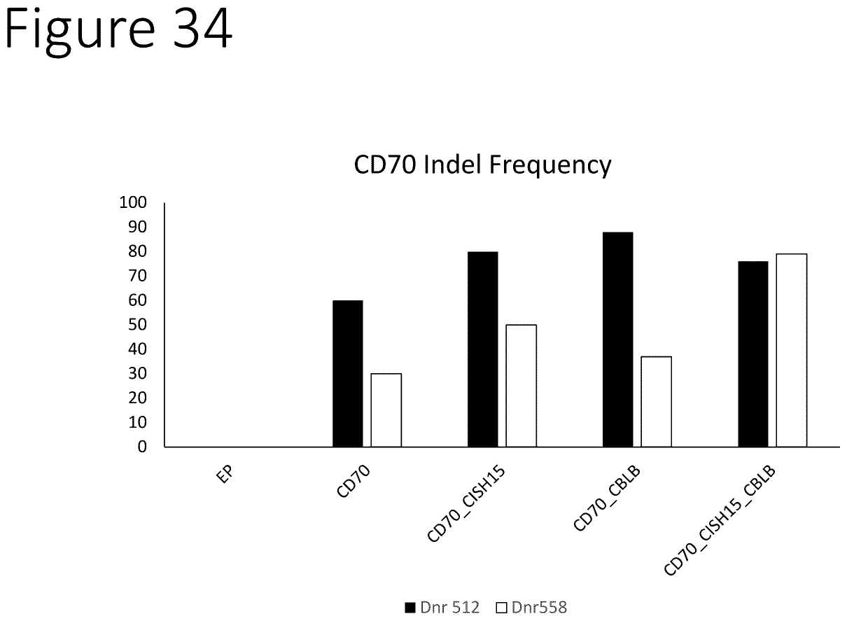

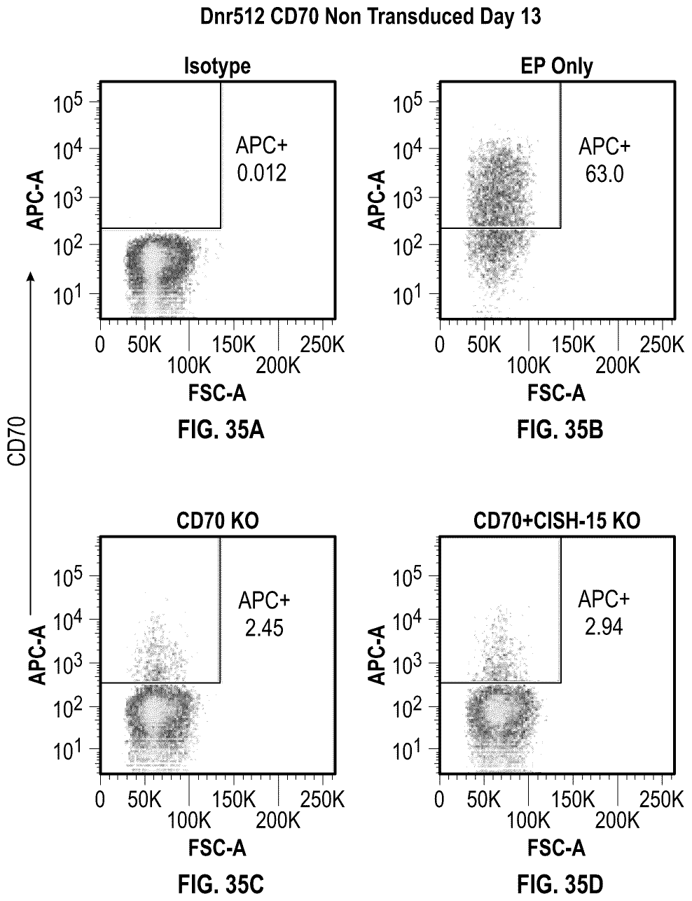

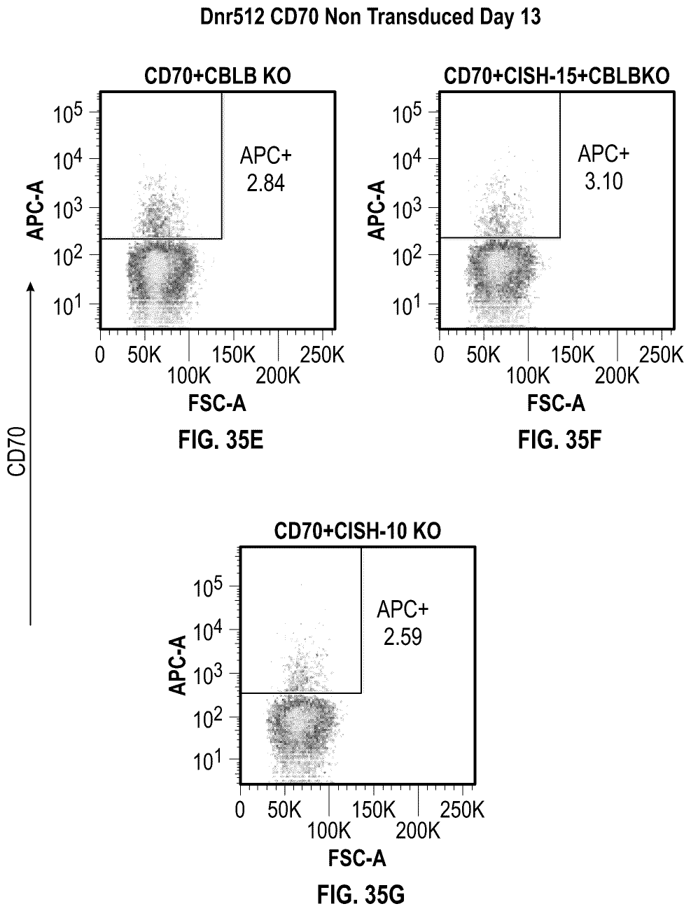

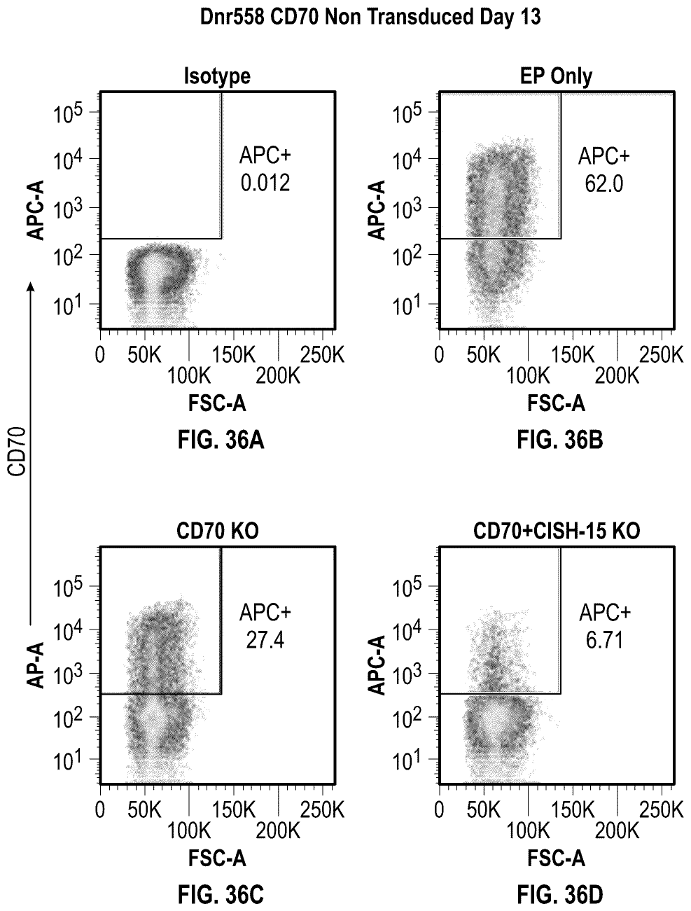

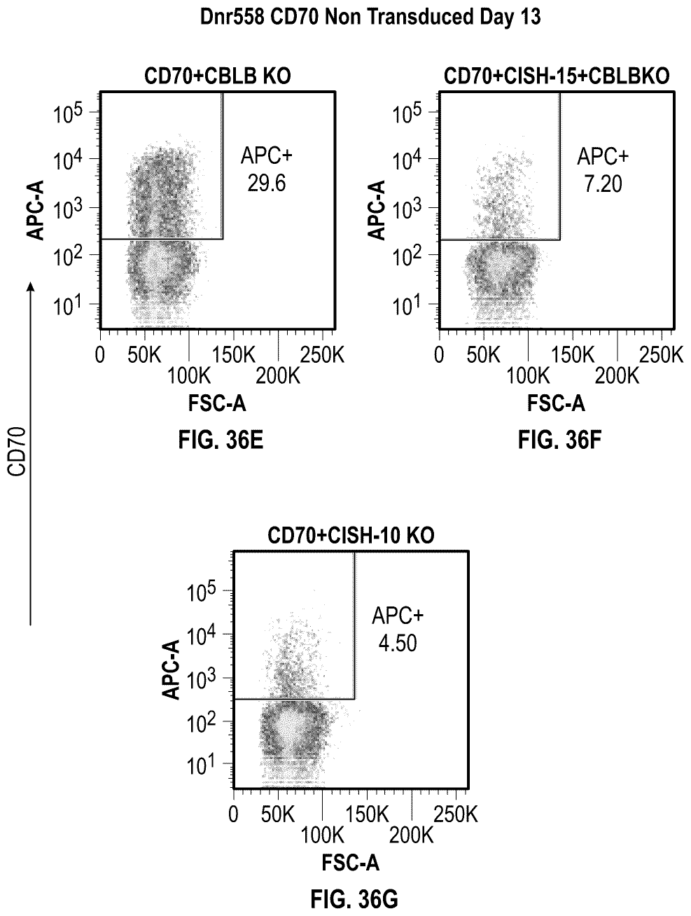

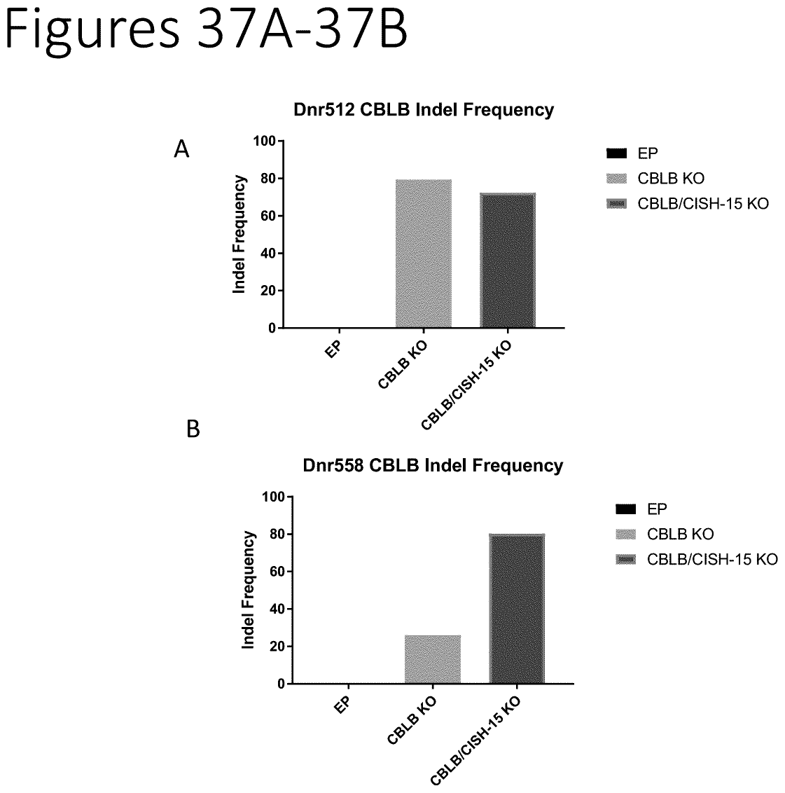

FIG. 32 summarizes the TIDE analysis of the CISH-15 gRNA in two sets of donor NK cells. As seen from the data the indel frequency was not negatively impacted by including a second edit (as was seen with the second and third edits discussed above). FIG. 33A shows a comparison of the indel frequency of CISH-10 versus the CISH-15 gRNA. The indel frequency of additional gRNAs from four different donors is shown in FIG. 33B. FIG. 34 depicts a representation of the CD70 indel frequency for two donors. FIGS. 35A-35G show the degree of CD70 expression in the indicated edit contexts (non-transduced cells). As can be seen from these data, whether making a single edit (e.g., just to CD70) or CD70 in combination with CISH (35D), CBLB (35E), or both (35F), the presence of a multiplex editing does not reduce the effectiveness of the individual edits as measured by flow cytometry. Corresponding data is shown in FIGS. 36A-36G for another donor. These trends are confirmed by indel frequency analysis, which is summarized in FIGS. 37A and 37B for two donors. These data show that a second edit either does not diminish (37A) or appears to enhance (37B) the primary edit. Overall the reduced expression of each gene is relatively consistent, confirming that multiplex gene editing is feasible and effective.

### Example 7—Analysis of Chromosomal Translocation in Multiplex Edited NK Cells and Optimization

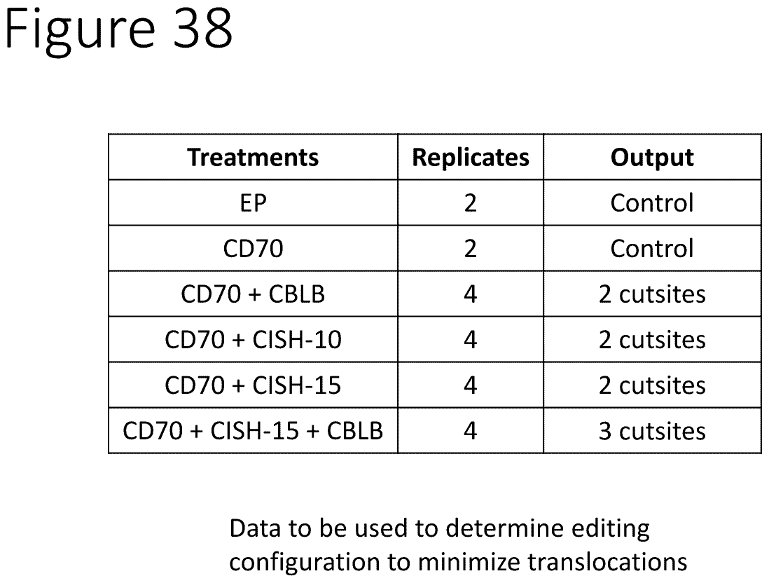

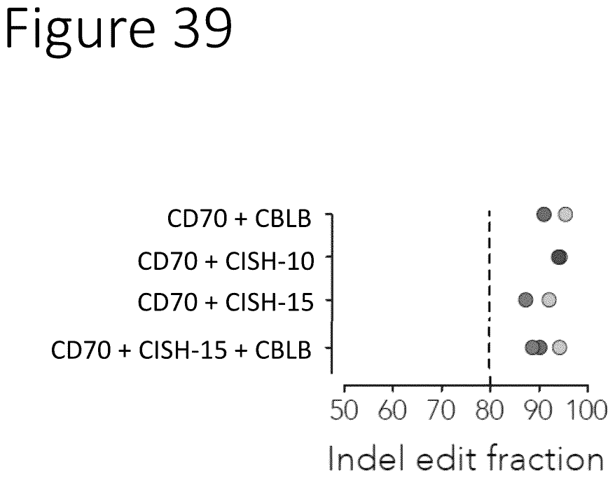

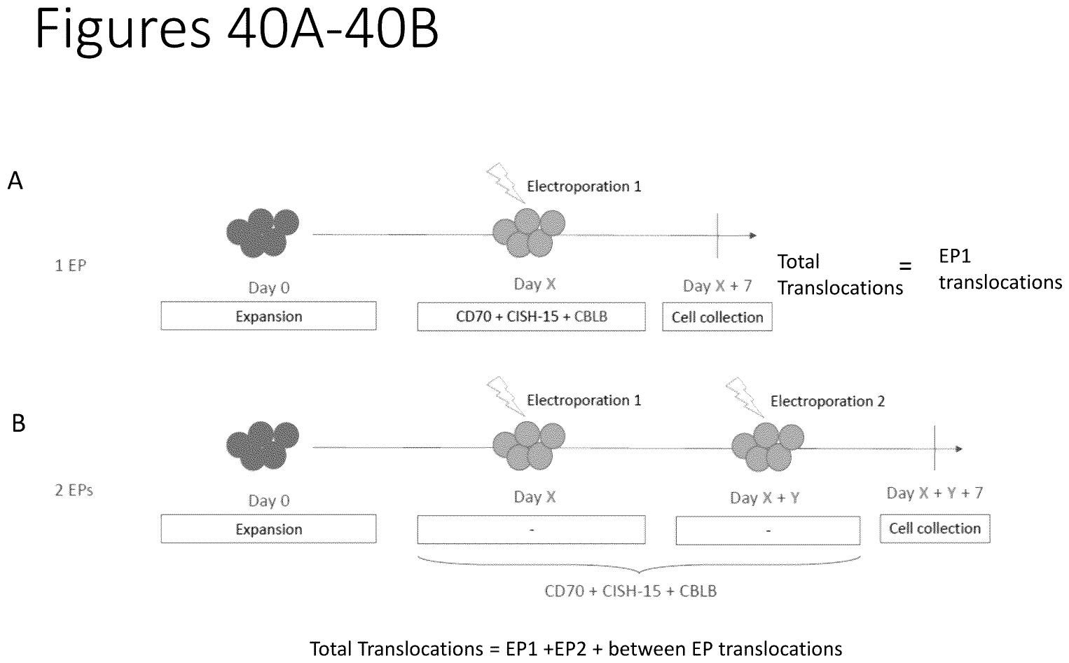

When multiple gene edits are made, as in the prior examples, there is a risk that the double strand breaks in multiple locations allow for translocation of chromosomal material. Following the non-limiting process flow of FIG. 31, edited cells will be generated using the Day 6 EP approach and will therefore be edited after 6 days of expansion. FIG. 38 outlines a series of experimental groups to assess possibility of chromosomal translocation. FIG. 39 depicts the percentage of on target editing for each contemplated edit combination, each of which is well above a desired threshold of 80%. The importance of on-target editing lies in the ability to more accurately assess the risk for translocations (e.g., reduced off-target cutting should decrease the number of “free” chromosomal matter). Moreover, potential for translocation events impacts how many editing cycles can be used. Use of two gRNAs in a single editing cycle could result in 4 resultant species. For example, if a gRNA targets an endonuclease to cut chromosome 9 and a second gRNA guides an endonuclease to cut chromosome 11, there are four resultant chromosomal fragments that result—9A, 9B, 11A, and 11B. If translocation occurs, there could be a 9A-11B combination, a 9B-11B combination, a 9A-11A combination, and a 9A-11B combination. When using three gRNAs in a single editing cycle, there are 6 resultant chromosomal fragments, that can combine (translocate) into 12 different species. For clinical product and safety, a goal is to minimize translocations. To accomplish a triple edit, like the CD70/CISH/CBLB combination discussed above, two general approaches could be undertaken, which are schematically shown in FIGS. 40A-40B (not shown is an additional alternative of three distinct editing events). FIG. 40A shows a single electroporation event to accomplish a triple edit. The total number of translocations for this approach is the number that results from that single editing event. FIG. 40B shows and alternative approach wherein two editing events (EP1 and EP2) are used to accomplish the totality of the contemplated triple edit (here CD70/CISH/CBLB). The total possible translocations in this context is the total of those occurring at the first and the second editing event. Thus, in several embodiments, the combination of edits is selected to reduce the probability of translocations based, for example on the gRNAs used in combination in a given editing event.

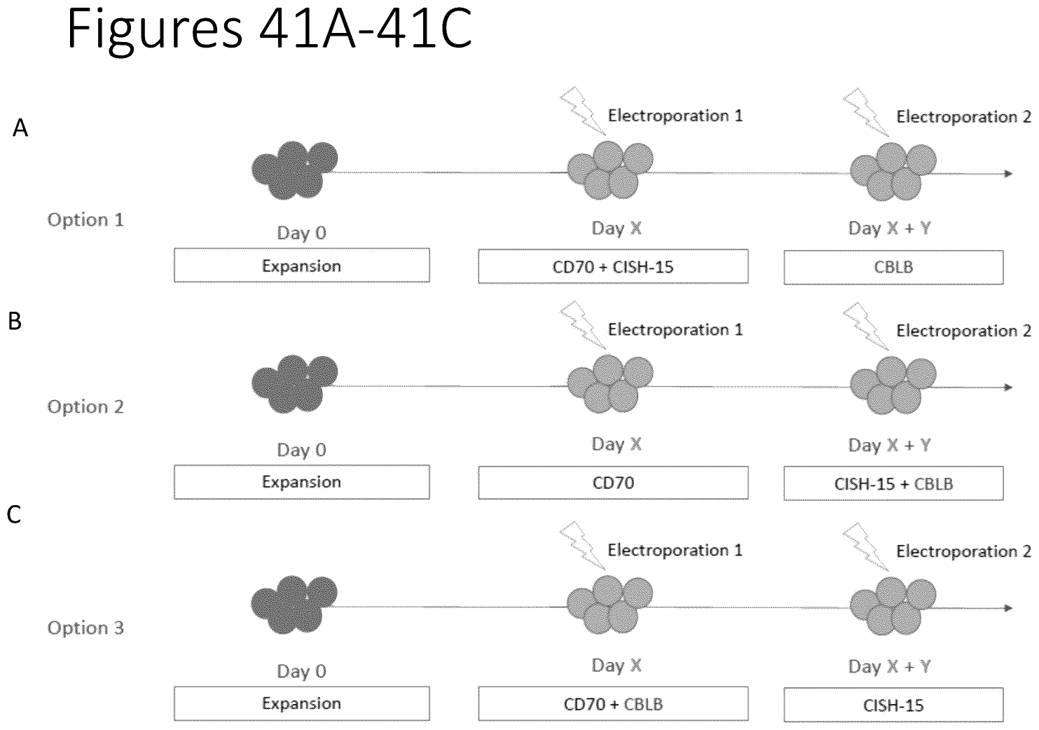

FIGS. 41A-41C lay out non-limiting combinations of possible editing schema to accomplish a triple edit, here CD70/CISH/CBLB. FIG. 41A employs a first (dual) edit to CD70 and CISH (using for example the CISH-15 gRNA sequence given by SEQ ID NO:191) and a second edit to CBLB (using for example the CBLB gRNA sequence given by SEQ ID NO:195). FIG. 41B shows a first (single) edit to CD70 (using for example the CD70 gRNA sequence given by SEQ ID NO:180) and a second (dual) edit to CISH and CBLB. FIG. 41C shows a first (dual) edit to CD70 and CBLB and a second edit to CISH. There are also provided, for example, editing structures where the first and the second edits listed above are performed in an inverse order.

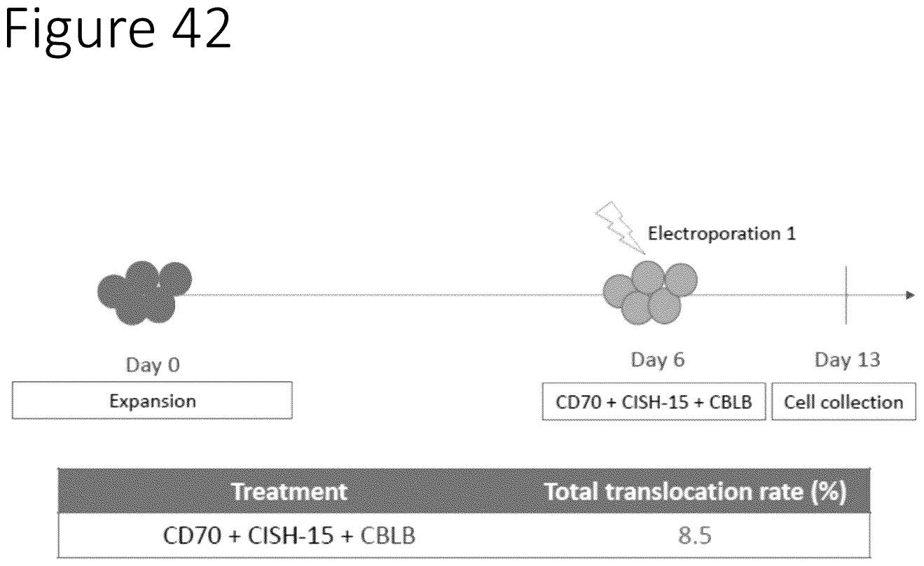

Following the schema of FIG. 40A, a single electroporation was performed to accomplish a triple edit, here CD70/CISH/CBLB. The resultant detected translocation rate was 8.5% (see FIG. 42), which is above the desired acceptable range of <4-6%. However, in some embodiments wherein different genes are edited, an acceptable translocation rate is achieved. The results here may be in part because both CISH and CBLB are on the same chromosome, which could increase the probability of translocation events (e.g., due to relative localization of double strand breaks and “free” chromosomal fragments).

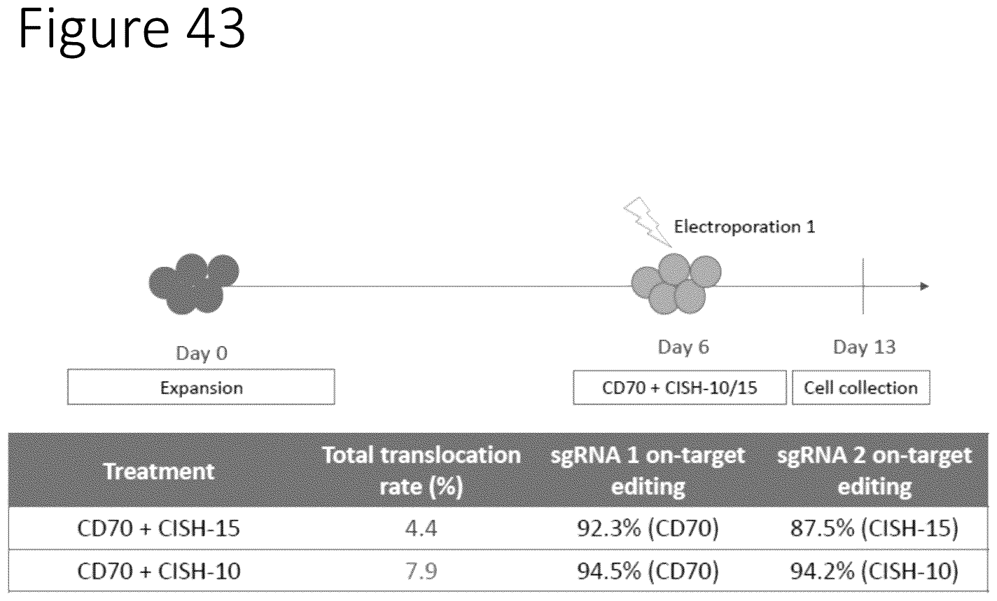

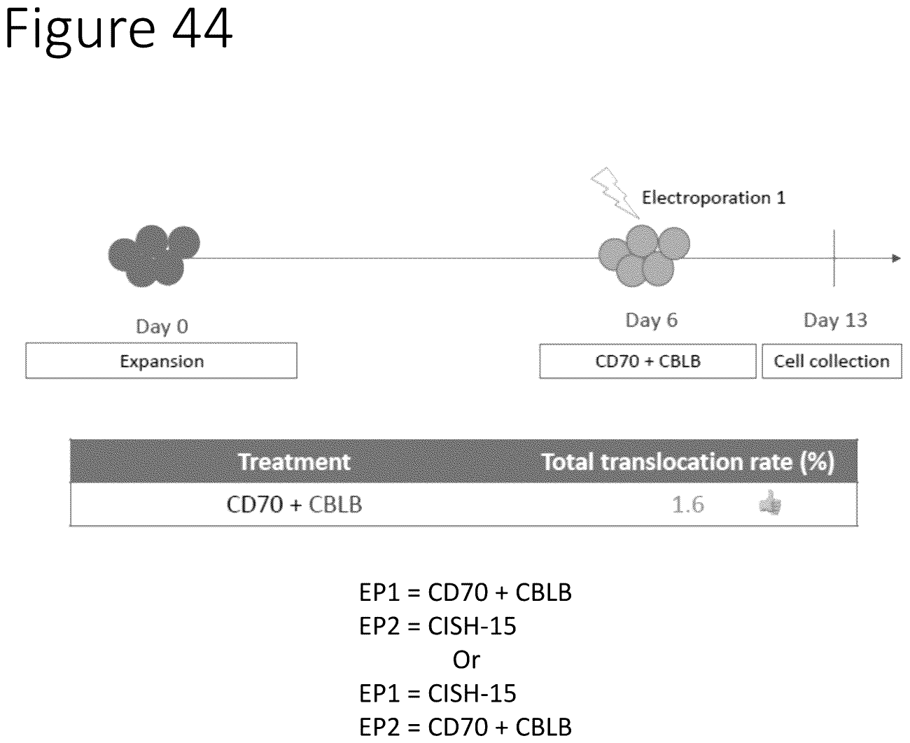

A dual edit schema was set up (only the first edit was tested) with a combination of CD70 and CISH edits being made (using either the CISH-10 or the CISH-15 gRNA). As shown in FIG. 43, the translocation rate using the CISH-10 gRNA (having the sequence given by SEQ ID NO:187) was above a desired threshold, but the CISH-15 gRNA (in combination with a gRNA for CD70) yielded an acceptable low translocation rate of ˜4.4%. Further desiring to reduce the translocation event, a combination of CD70 and CBLB was tested in a single editing event. These data, shown in FIG. 44, show a very desirable translocation frequency of less than 2 percent (1.6% as tested). With this relatively low percentage event when editing CD70 and CBLB, the second edit (which would be to CISH) alone, would be expected to result in fewer, if any, any translocation events due to the single cut. Thus, as shown in FIG. 44, the dual edit could either be performed first, or second, depending on the embodiment. While the single edits would be expected to generate few, if any, translocation events, in several embodiments, the total number of translocation events can be further reduced by, for example, optimizing (e.g., increasing) the time between editing events. In several embodiments, the EP1 and EP2 are separated by about 12 hours, about 24 hours, about 36 hours, about 48 hours, about 3 days, about 4 days, about 5 days, about 6 days, or about 7 days (or any time in between those times listed). In several embodiments, more than 7 days elapses between editing events. When considered as a whole, these data show that multiplex gene editing to accomplish a triple edit, for example CD70/CISH/CBLB, can be accomplished with a sufficiently low rate of translocation and an effective amount of gene expression reduction, as well as expression of a cytotoxic CAR, to result in a highly cytotoxic cell population. According to several embodiments, a CD70 CAR-expressing population is edited at CD70, CISH, and CBLB to generate a highly active and persistent engineered and edited cell population. In several embodiments, the cells comprise NK cells.

### Example 8—In Vivo Analysis of Multiplex Gene Edited NK Cells

CD70 CAR-expressing NK cells knocked out for CD70, CISH, and CBLB (CD70/CISH/CBLB KO) were analyzed for knockout efficiency, in vitro cytokine secretion and persistence, and in vivo efficacy and persistence.

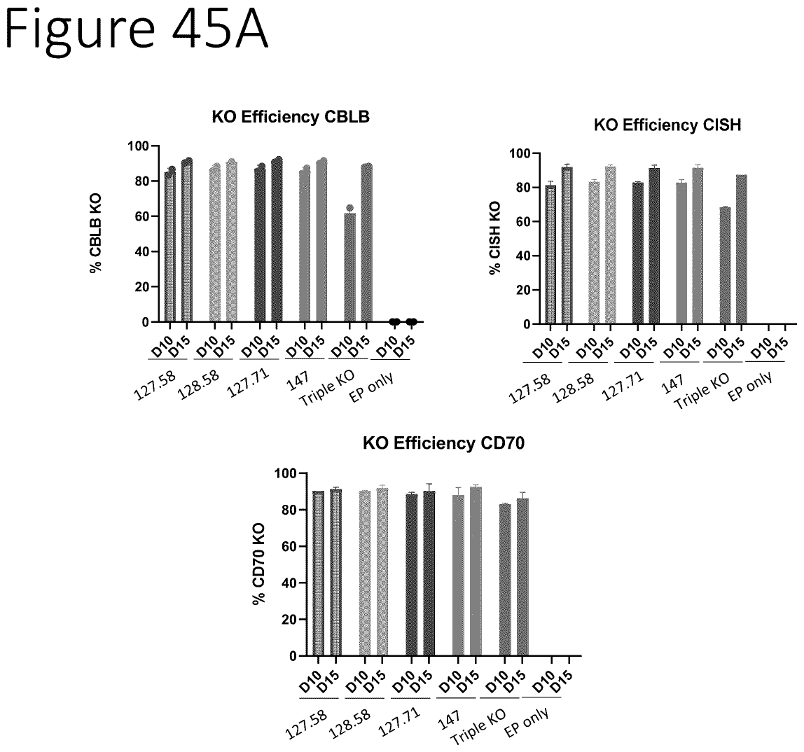

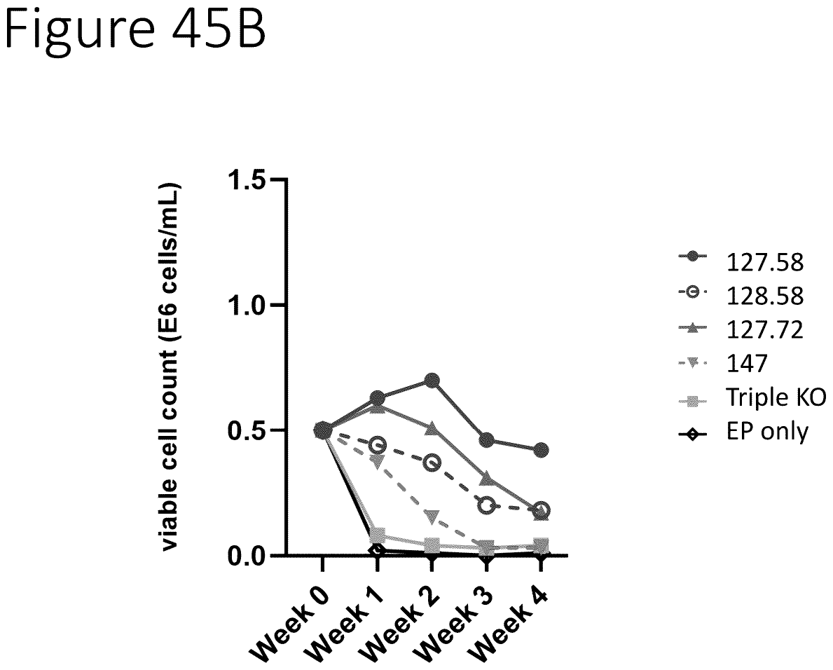

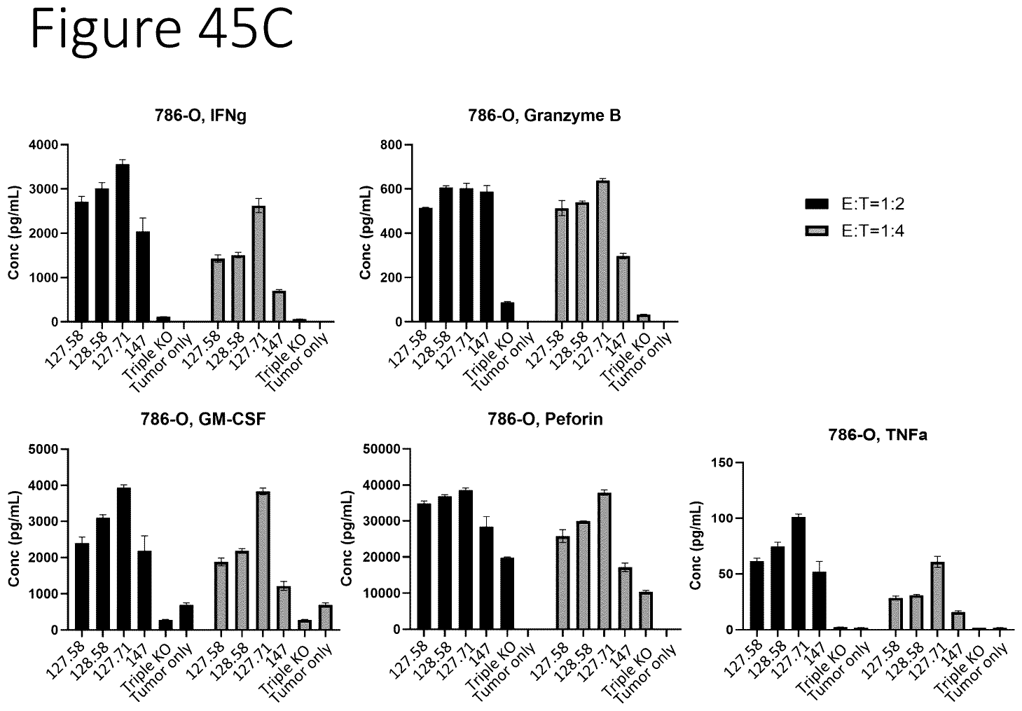

Briefly, NK cells were knocked out for CD70, CISH, and CBLB using the exemplary CD70, CISH-15, and CBLB gRNA sequences described herein (e.g., SEQ ID NOS: 180, 191, and 195, respectively), and subsequently engineered to express the 127.58, 128.58, 127.71, or 147 CD70-targeting CAR. Knockout efficiency of each gene was assessed at 10 and 15 days post-electroporation in NK cells expressing the different CD70 CARs. For each of CD70, CISH, and CBLB, knockout efficiency was similar among the different CAR constructs (FIG. 45A). Further, by 15 days post-electroporation, the knockout efficiency of each of CD70, CISH, and CBLB was comparable between triple knocked out cells not expressing a CAR (Triple KO) and triple knocked out cells expressing a CAR (FIG. 45A). Triple KO NK cells engineered to express exemplary CD70 CARs were assessed for their persistence in vitro in the absence of IL-2. Triple KO NK cells not expressing a CAR (Triple KO) or cells mock electroporated (EP only) served as controls. As shown in FIG. 45B, cells expressing the 127 and 128 series CARs tended to exhibit the greatest persistence. CD70/CISH/CBLB KO NK cells expressing the exemplary CD70 CARs were co-cultured with 786-O target cells at an E:T ratio of 1:2 (dark bars) or 1:4 (light bars) and secretion of molecules indicative of NK cell activation was analyzed (FIG. 45C). Cells expressing the 127 and 128 series CARs, and particularly the 127.71 CAR, tended to secrete more molecules associated with activation.

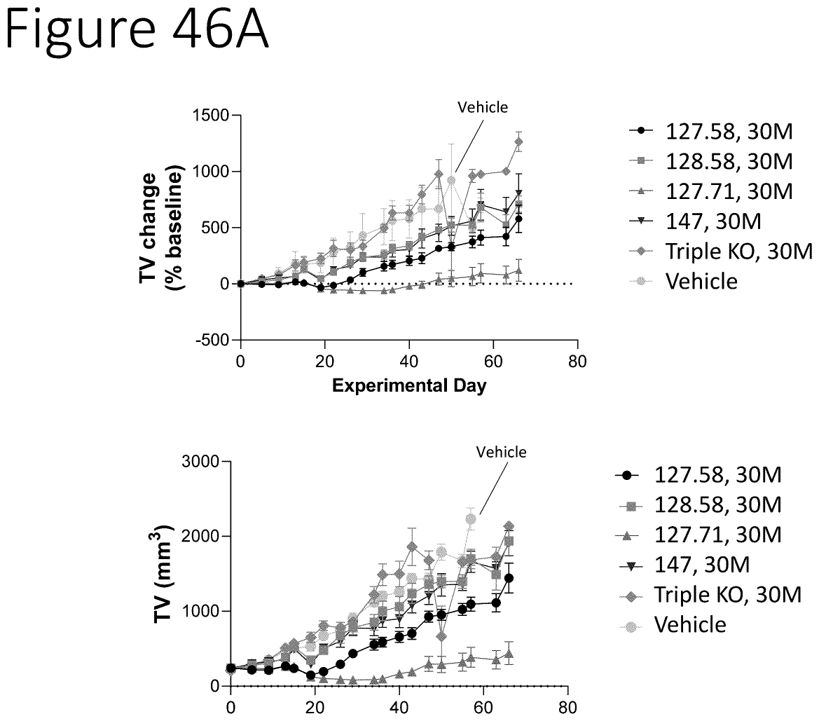

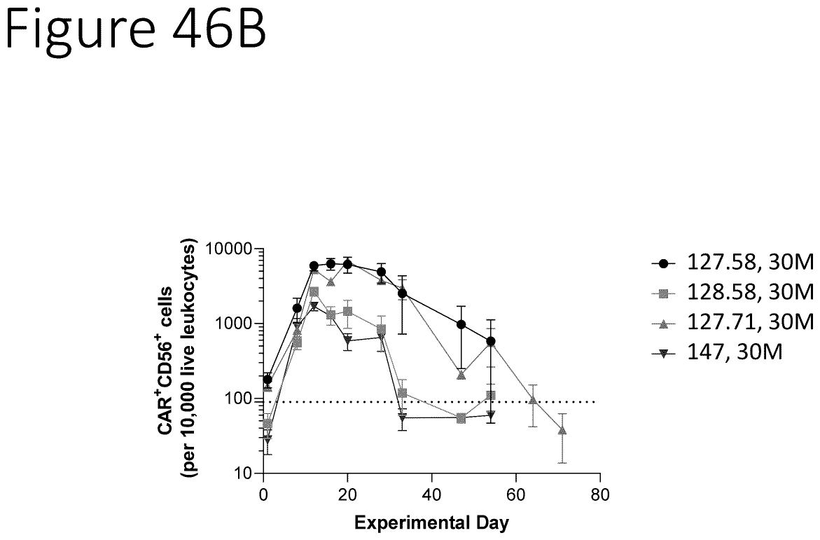

Ten million 786-O tumor cells were injected into NOD scid gamma (NSG) mice on Day −5 and allowed to engraft. On Day 0, mice were injected with a single dose of 30×10⁶ CD70 CAR NK cells (CD70/CISH/CBLB KO, e.g., at SEQ ID NOS: 180, 191, and 195, respectively)). Tumor volume and NK cell persistence were assessed until approximately Day 70. As controls, mice were injected with an equal number of NK cells knocked out for CD70/CISH/CBLB but not expressing a CAR (triple KO) or vehicle only. As shown in FIGS. 46A-B, NK cells expressing the 127.71 CAR exhibited the greater tumor volume (TV) control and in vivo persistence, respectively.

It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. In addition, where features or aspects of the disclosure are described in terms of Markush groups, those skilled in the art will recognize that the disclosure is also thereby described in terms of any individual member or subgroup of members of the Markush group.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 90%” includes “90%.” In some embodiments, at least 95% sequence identity or homology includes 96%, 97%, 98%, 99%, and 100% sequence identity or homology to the reference sequence. In addition, when a sequence is disclosed as “comprising” a nucleotide or amino acid sequence, such a reference shall also include, unless otherwise indicated, that the sequence “comprises”, “consists of” or “consists essentially of” the recited sequence. Any titles or subheadings used herein are for organization purposes and should not be used to limit the scope of embodiments disclosed herein.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

### Sequences

In several embodiments, there are provided amino acid sequences that correspond to any of the nucleic acids disclosed herein (and/or included in the accompanying sequence listing), while accounting for degeneracy of the nucleic acid code. Furthermore, those sequences (whether nucleic acid or amino acid) that vary from those expressly disclosed herein (and/or included in the accompanying sequence listing), but have functional similarity or equivalency are also contemplated within the scope of the present disclosure. The foregoing includes mutants, truncations, substitutions, codon optimization, or other types of modifications.

In accordance with some embodiments described herein, any of the sequences may be used, or a truncated or mutated form of any of the sequences disclosed herein (and/or included in the accompanying sequence listing) may be used and in any combination. Sequences provided for herein that include an identifier, such as a tag or other detectable sequence (e.g., a Flag tag) are also provided for herein with the absence of such a tag or other detectable sequence (e.g., excluding the Flag tag from the listed sequence). A Sequence Listing in electronic format is submitted herewith. Some of the sequences provided in the Sequence Listing may be designated as Artificial Sequences by virtue of being non-naturally occurring fragments or portions of other sequences, including naturally occurring sequences. Some of the sequences provided in the Sequence Listing may be designated as Artificial Sequences by virtue of being combinations of sequences from different origins, such as humanized antibody sequences.

| SEQ | | |
|---|---|---|
| ID NO | Sequence | Description |
| 1 | accacgacgccagcgccgcgaccaccaacaccggcgcccaccatcgcgtcgcagcccct | CD8alpha |
| | gtccctgcgcccagaggcgtgccggccagcggggggggcgcagtgcacacgaggggg | hinge |
| | ctggacttcgcctgtgat | |
| 2 | TTTPAPRPPTPAPTIASQPLSLRPEACRPAAGGAVHTRGLDFACD | CD8a hinge |
| 3 | atctacatctgggcgcccttggccgggacttgtggggtccttctcctgtcactggttatcacc | CD8 TM |
| 4 | IYIWAPLAGTCGVLLLSLVIT | CD8 TM |
| 5 | cggagggaccagaggctgccccccgatgcccacaagccccctgggggaggcagtttccg | OX40 |
| | gacccccatccaagaggagcaggccgacgcccactccaccctggccaagatc | |
| 6 | RRDQRLPPDAHKPPGGGSFRTPIQEEQADAHSTLAKI | OX40 |
| 7 | agagtgaagttcagcaggagcgcagacgcccccgcgtaccagcagggccagaaccagct | CD3zeta |
| | ctataacgagctcaatctaggacgaagagaggagtacgatgttttggacaagagacgtggc | |
| | cgggaccctgagatggggggaaagccgagaaggaagaaccctcaggaaggcctgtaca | |
| | atgaactgcagaaagataagatggcggaggcctacagtgagattgggatgaaaggcgagc | |
| | gccggaggggcaaggggcacgatggcctttaccagggtctcagtacagccaccaaggac | |
| | acctacgacgcccttcacatgcaggccctgccccctcgc | |
| 8 | RVKFSRSADAPAYQQGQNQLYNELNLGRREEYDVLDKRRGRDPE | CD3zeta |
| | MGGKPRRKNPQEGLYNELQKDKMAEAYSEIGMKGERRRGKGHD | |
| | GLYQGLSTATKDTYDALHMQALPPR | |
| 9 | ggctctggcgagggaaggggttccctgcttacttgcggcgacgtcgaagagaatcccggtcc | T2A |
| | g | |
| 10 | GSGEGRGSLLTCGDVEENPGP | T2A |
| 11 | aactgggtcaacgtgattagcgatttgaagaaaatcgaggaccttatacagtctatgcatattg | Native IL15 |
| | acgctacactgtatactgagagtgatgtacacccgtcctgtaaggtaacggccatgaaatgctt | |
| | tcttctggagctccaggtcatcagcttggagtctggggacgcaagcatccacgatacggttga | |
| | aaacctcatcatccttgcgaacaactctctctcatctaatggaaacgttacagagagtgggtgt | |
| | aaggagtgcgaagagttggaagaaaaaaacatcaaagaatttcttcaatccttcgttcacata | |
| | gtgcaaatgttcattaacacgtcc | |
| 12 | NWVNVISDLKKIEDLIQSMHIDATLYTESDVHPSCKVTAMKCFLLEL | Native IL15 |
| | QVISLESGDASIHDTVENLIILANNSLSSNGNVTESGCKECEELEEK | |
| | NIKEFLQSFVHIVQMFINTS | |
| 13 | actaccacacccgccccgaggccacctacgccggcaccgactatcgccagtcaacccctct | CD8hinge/TM |
| | ctctgcgccccgaggcttgccggcctgcggctggtggggcggtccacacccggggcctggat | |
| | tttgcgtgcgatatatacatctgggcacctcttgccggcacctgcggagtgctgcttctctcactc | |
| | gttattacgctgtactgc | |
| 14 | TTTPAPRPPTPAPTIASQPLSLRPEACRPAAGGAVHTRGLDFACDI | CD8hinge/TM |
| | YIWAPLAGTCGVLLLSLVIT | |
| 15 | ggcggtggtggctctggtggtggcggcagc | Linker |
| 16 | GGGGSGGGGS | Linker |
| 17 | ggccaggccggctccggaggaggaggatcc | Linker |
| 18 | GQAGSGGGGS | Linker |
| 19 | ggccaggccggc | Linker |
| 20 | GQAG | Linker |
| 21 | ggcggcggcggtagcggtggtggcggctccgga | Linker |
| 22 | GGGGSGGGGSG | Linker |
| 23 | caggtcaccttgaaggagtctggtcctgtgctggtgaaacccacagagaccctcacgctgac | anti-CD70 scFv |
| | ctgcaccgtctctgggttctcactcagtaatgctagaatgggtgtgacctggatccgtcagcccc | |
| | cagggaaggccctggagtggcttgcacacattttttcgaatgacgaaaaatcctacagtacat | |
| | ctctgaagagcaggctcaccatctccaaggacacttccaaaacccaggtggtccttaccatg | |
| | accaacatggaccctgtggacacagccacatattactgtgcacggatacgagattactatga | |
| | cattagtagttattatgactactggggccagggaaccctggtcagcgtctcctcaggcggtggt | |
| | ggctctggtggtggcggcagcggcggtggtggctctgacatccagatgacccagtctccatct | |
| | gccatgtctgcatctgtaggagacagagtcaccatcacttgtcgggcgagtcaggacattagc | |
| | aattatttagcctggtttcagcagaaaccagggaaagtccctaagcgcctgatctatgctgcat | |
| | ccagtttgcaaagtggggtcccatcaaggttcagcggcagtggatcggggacagagttcact | |
| | ctcacaatcagcagcctgctgcctgaagattttgcaacttattactgtctacagcttaatagtttcc | |
| | cgttcacttttggcggagggaccaaggtggagatcaac | |
| 24 | gaagtgcagcttgtccagagcggagccgaagtgaagaagcctggcgagagcctgaagatc | anti CD70 scFv |
| | agctgcaagggctcaggttacggattcacaagttattggataggttgggtgcgccagatgcct | |
| | ggtaagggactggaatggatgggtatcattcatcccgatgatagcgacaccaaatacagccc | |
| | aagctttcagggccaggtcacaatcagcgctgacaagagcatcagcaccgcctaccttcagt | |
| | ggtcgtctctgaaggccagcgacaccgcaatgtactactgtgcctctagctatttgcgtggcttgt | |
| | ggggaggctattttgactattggggccaaggtacactggtcaccgtcagcagcggcggtggtg | |
| | gctctggtggtggcggcagcggcggtggtggctctgagctccagagcgtgctgacccagcct | |
| | cctagcgcaagcggcacccctggacagcgtgtgacaattagctgtagcggatcaagctcaa | |
| | acattggctcaaattatgtgaattggtatcagcaattgcccggtacagcacccaaactgctcatt | |
| | tatggagattatcaacgacctagcggagtgcctgatcgttttagcggtagcaaaagcggcacc | |
| | agcgcatcactggcaatttcaggcctgcgtagcgaagatgaggcggattattactgtgctacc | |
| | cgcgacgattcgttatctgggtctgtcgtttttggcaccggtacaaaactgaccgtgctg | |
| 25 | QVTLKESGPVLVKPTETLTLTCTVSGFSLSNARMGVTWIRQPPGK | anti CD70 scFv |
| | ALEWLAHIFSNDEKSYSTSLKSRLTISKDTSKTQVVLTMTNMDPVD | |
| | TATYYCARIRDYYDISSYYDYWGQGTLVSVSSGGGGGGGGSGG | |
| | GGSDIQMTQSPSAMSASVGDRVTITCRASQDISNYLAWFQQKPG | |
| | KVPKRLIYAASSLQSGVPSRFSGSGSGTEFTLTISSLLPEDFATYYC | |
| | LQLNSFPFTFGGGTKVEIN | |
| 26 | EVQLVQSGAEVKKPGESLKISCKGSGYGFTSYWIGWVRQMPGKG | anti CD70 scFv |
| | LEWMGIIHPDDSDTKYSPSFQGQVTISADKSISTAYLQWSSLKASD | |
| | TAMYYCASSYLRGLWGGYFDYWGQGTLVTVSSGGGGSGGGGS | |
| | GGGGSELQSVLTQPPSASGTPGQRVTISCSGSSSNIGSNYVNWY | |
| | QQLPGTAPKLLIYGDYQRPSGVPDRFSGSKSGTSASLAISGLRSE | |
| | DEADYYCATRDDSLSGSVVFGTGTKLTVL | |
| 27 | atggccctcccagtaactgccctccttttgcccctcgcactccttcttcatgccgctcgccccaac | Membrane- |
| | tgggtcaacgtgattagcgatttgaagaaaatcgaggaccttatacagtctatgcatattgacg | bound human |
| | ctacactgtatactgagagtgatgtacacccgtcctgtaaggtaacggccatgaaatgctttctt | IL15 (mbIL15) |
| | ctggagctccaggtcatcagcttggagtctggggacgcaagcatccacgatacggttgaaaa | |
| | cctcatcatccttgcgaacaactctctctcatctaatggaaacgttacagagagtgggtgtaag | |
| | gagtgcgaagagttggaagaaaaaaacatcaaagaatttcttcaatccttcgttcacatagtg | |
| | caaatgttcattaacacgtccactaccacacccgccccgaggccacctacgccggcaccga | |
| | ctatcgccagtcaacccctctctctgcgccccgaggcttgccggcctgcggctggtggggcgg | |
| | tccacacccggggcctggattttgcgtgcgatatatacatctgggcacctcttgccggcacctg | |
| | cggagtgctgcttctctcactcgttattacgctgtactgc | |
| 28 | MALPVTALLLPLALLLHAARPNWVNVISDLKKIEDLIQSMHIDATLYT | Membrane- |
| | ESDVHPSCKVTAMKCFLLELQVISLESGDASIHDTVENLIILANNSL | bound human |
| | SSNGNVTESGCKECEELEEKNIKEFLQSFVHIVQMFINTSTTTPAP | IL15 (mbIL15) |
| | RPPTPAPTIASQPLSLRPEACRPAAGGAVHTRGLDFACDIYIWAPL | |
| | AGTCGVLLLSLVITLYC | |
| 29 | DYKDDDDK | Flag tag |
| 30 | caggtgcagctggtgcagtctggggctgaggtgaagaagcctggggcctcagtgaaggtttc | anti CD70 scFv |
| | ctgcaaggcatctggatacaccttcaccgattactatatgcactgggtgcgacaggcccctgg | |
| | acaagggcttgagtggatgggaataatcaaccctaatagtggtggaacaaattacgcacag | |
| | aagttccagggcagagtcaccatgaccagggacacgtccacgagcacagtctacatggag | |
| | ctgagcagcctgagatctgaggacacggccgtgtattactgtgctagagattttagtgtgtatgc | |
| | tgctgcagatggcatggatgtctggggccaagggacattggtcaccgtctcttcaggtggagg | |
| | cggttcaggcggaggtggctctggcggtggcggatcggacatccagatgacccagtctccat | |
| | cctccctgtctgcatctgtaggagacagagtcaccatcacttgccgggcaagtcagagcatta | |
| | gcagctatttaaattggtatcagcagaaaccagggaaagcccctaagctcctgatctatgacg | |
| | catccaatttggccaccggggtcccatcaaggttcagtggcagtggatctgggacagatttca | |
| | ctctcaccatcagcagtctgcaacctgaagattttgcaacttactactgtcaacagagtgatagt | |
| | tcacctctcactttcggccaagggaccaaggggagatcaaa | |
| 31 | caggtgcagctggtgcagtctggggctgaggtgaagaagcctggggcctcagtgaaggtttc | anti CD70 scFv |
| | ctgcaaggcatctggatacaccttcaccagctacgacattaattgggtgcgacaggcccctgg | |
| | acaagggcttgagtggatgggagtgatcaaccctagtggtggaggtacaagctacgcacag | |
| | aagttccagggcagagtcaccatgaccagggacacgtccacgagcacagtctacatggag | |
| | ctgagcagcctgagatctgaggacacggccgtgtattactgtgctagatattctggctatgattg | |
| | ggtgtttgactactggggccaagggaccctggtcaccgtctcctcaggtggaggcggttcagg | |
| | cggaggtggctctggcggtggcggatcggacatccagatgacccagtctccatcctccctgtc | |
| | tgcatctgtaggagacagagtcaccatcacttgccgggcaagtcagggcattggcagctggtt | |
| | agcttggtatcagcagaaaccagggaaagcccctaagctcctgatctatgctgcatccacatt | |
| | gcaaagtggggtcccatcaaggttcagtggcagtggatctgggacagatttcactctcaccat | |
| | cagcagtctgcaacctgaagattttgcaacttactactgtcaacagagttacagtaccccttgg | |
| | actttcggccctgggaccaaagtggatatcaaa | |
| 32 | caggtgcagctggtgcagtctggggctgaggtgaagaagcctggggcctcagtgaaggtttc | anti CD70 scFv |
| | ctgcaaggcatctggatacaccttcaccagctacgacatcaattgggtgcgacaggcccctg | |
| | gacaagggcttgagtggatgggagtcatcagccctagtggtggtggaacaagctacgcaca | |
| | gaagttccagggcagagtcaccatgaccagggacacgtccacgagcacagtctacatgga | |
| | gctgagcagcctgagatctgaggacacggccgtgtattactgtgctagatactacggcggca | |
| | accccgaagggtggtacttcgatctctggggccgtggcaccctggtcactgtctcctcaggtgg | |
| | aggcggttcaggcggaggtggctctggcggtggcggatcggacatccagatgacccagtct | |
| | ccatcctccctgtctgcatctgtaggagacagagtcaccatcacttgccgggcaagtcagagc | |
| | attggaaattggttagcctggtatcagcagaaaccagggaaagcccctaagctcctgatctat | |
| | gacgcatccgatttggaaaccggggtcccatcaaggttcagtggcagtggatctgggacaga | |
| | tttcactctcaccatcagcagtctgcaacctgaagattttgcaacttactactgtcaacagagtta | |
| | cactaccccttggacgttcggccaagggaccaaggtggaaatcaaa | |
| 33 | ggcagcaccagcggcagcggcaagcccggctccggagagggcagcaccaagggc | Linker |
| 34 | gacatccagatgacccagtctccatcctccctgtctgcatctgtaggagacagagtcaccatc | anti CD70 scFv |
| | acttgccgggcaagtcagggcattggcagctggttagcttggtatcagcagaaaccagggaa | |
| | agcccctaagctcctgatctatgctgcatccacattgcaaagtggggtcccatcaaggttcagt | |
| | ggcagtggatctgggacagatttcactctcaccatcagcagtctgcaacctgaagattttgcaa | |
| | cttactactgtcaacagagttacagtaccccttggactttcggccctgggaccaaagtggatatc | |
| | aaaggcagcaccagcggcagcggcaagcccggctccggagagggcagcaccaagggc | |
| | caggtgcagctggtgcagtctggggctgaggtgaagaagcctggggcctcagtgaaggtttc | |
| | ctgcaaggcatctggatacaccttcaccagctacgacattaattgggtgcgacaggcccctgg | |
| | acaagggcttgagtggatgggagtgatcaaccctagtggtggaggtacaagctacgcacag | |
| | aagttccagggcagagtcaccatgaccagggacacgtccacgagcacagtctacatggag | |
| | ctgagcagcctgagatctgaggacacggccgtgtattactgtgctagatattctggctatgattg | |
| | ggtgtttgactactggggccaagggaccctggtcaccgtctcctca | |
| 35 | gacatccagatgacccagtctccatcctccctgtctgcatctgtaggagacagagtcaccatc | anti CD70 scFv |
| | acttgccgggcaagtcagagcattggaaattggttagcctggtatcagcagaaaccaggga | |
| | aagcccctaagctcctgatctatgacgcatccgatttggaaaccggggtcccatcaaggttca | |
| | gtggcagtggatctgggacagatttcactctcaccatcagcagtctgcaacctgaagattttgc | |
| | aacttactactgtcaacagagttacactaccccttggacgttcggccaagggaccaaggtgg | |
| | aaatcaaaggcagcaccagcggcagcggcaagcccggctccggagagggcagcacca | |
| | agggccaggtgcagctggtgcagtctggggctgaggtgaagaagcctggggcctcagtga | |
| | aggtttcctgcaaggcatctggatacaccttcaccagctacgacatcaattgggtgcgacagg | |
| | cccctggacaagggcttgagtggatgggagtcatcagccctagtggtggtggaacaagctac | |
| | gcacagaagttccagggcagagtcaccatgaccagggacacgtccacgagcacagtctac | |
| | atggagctgagcagcctgagatctgaggacacggccgtgtattactgtgctagatactacggc | |
| | ggcaaccccgaagggtggtacttcgatctctggggccgtggcaccctggtcactgtctcctca | |
| 36 | gacatagttatgacccaatccccagatagtttggcggtttctctgggcgagagggcaacgatta | anti CD70 scFv |
| | attgtcgcgcatcaaagagcgtttcaacgagcggatattcttttatgcattggtaccagcaaaaa | |
| | cccggacaaccgccgaagctgctgatctacttggcttcaaatcttgagtctggggtgccggac | |
| | cgattttctggtagtggaagcggaactgactttacgctcacgatcagttcactgcaggctgagg | |
| | atgtagcggtctattattgccagcacagtagagaagtcccctggaccttcggtcaaggcacga | |
| | aagtagaaattaaaggcagcaccagcggcagcggcaagcccggctccggagagggcag | |
| | caccaagggccaggtccagttggtgcaaagcggggcggaggtgaaaaaacccggcgctt | |
| | ccgtgaaggtgtcctgtaaggcgtccggttatacgttcacgaactacgggatgaattgggttcg | |
| | ccaagcgccggggcagggactgaaatggatggggtggataaatacctacaccggcgaac | |
| | ctacatacgccgacgcttttaaagggcgagtcactatgacgcgcgataccagcatatccacc | |
| | gcatacatggagctgtcccgactccggtcagacgacacggctgtctactattgtgctcgggact | |
| | atggcgattatggcatggactactggggtcagggtacgactgtaacagttagtagt | |
| 37 | caggtccagttggtgcaaagcggggcggaggtgaaaaaacccggcgcttccgtgaaggtgt | anti CD70 scFv |
| | cctgtaaggcgtccggttatacgttcacgaactacgggatgaattgggttcgccaagcgccgg | |
| | ggcagggactgaaatggatggggtggataaatacctacaccggcgaacctacatacgccg | |
| | acgcttttaaagggcgagtcactatgacgcgcgataccagcatatccaccgcatacatggag | |
| | ctgtcccgactccggtcagacgacacggctgtctactattgtgctcgggactatggcgattatgg | |
| | catggactactggggtcagggtacgactgtaacagttagtagtggtggaggcggttcaggcg | |
| | gaggtggctctggcggtggcggatcggacatagttatgacccaatccccagatagtttggcgg | |
| | tttctctgggcgagagggcaacgattaattgtcgcgcatcaaagagcgtttcaacgagcggat | |
| | attcttttatgcattggtaccagcaaaaacccggacaaccgccgaagctgctgatctacttggct | |
| | tcaaatcttgagtctggggtgccggaccgattttctggtagtggaagcggaactgactttacgct | |
| | cacgatcagttcactgcaggctgaggatgtagcggtctattattgccagcacagtagagaagt | |
| | cccctggaccttcggtcaaggcacgaaagtagaaattaaa | |
| 38 | atggccttaccagtgaccgccttgctcctgccgctggccttgctgctccacgccgccaggccg | Anti-CD70 |
| | gactacaaagacgatgacgataaaggcggtggtggctctggtggtggcggatcccaggtgc | CAR |
| | agctggtgcagtctggggctgaggtgaagaagcctggggcctcagtgaaggtttcctgcaag | |
| | gcatctggatacaccttcaccgattactatatgcactgggtgcgacaggcccctggacaaggg | |
| | cttgagtggatgggaataatcaaccctaatagtggtggaacaaattacgcacagaagttcca | |
| | gggcagagtcaccatgaccagggacacgtccacgagcacagtctacatggagctgagca | |
| | gcctgagatctgaggacacggccgtgtattactgtgctagagattttagtgtgtatgctgctgca | |
| | gatggcatggatgtctggggccaagggacattggtcaccgtctcttcaggtggaggcggttca | |
| | ggcggaggtggctctggcggtggcggatcggacatccagatgacccagtctccatcctccct | |
| | gtctgcatctgtaggagacagagtcaccatcacttgccgggcaagtcagagcattagcagct | |
| | atttaaattggtatcagcagaaaccagggaaagcccctaagctcctgatctatgacgcatcca | |
| | atttggccaccggggtcccatcaaggttcagtggcagtggatctgggacagatttcactctcac | |
| | catcagcagtctgcaacctgaagattttgcaacttactactgtcaacagagtgatagttcacctc | |
| | tcactttcggccaagggaccaaggtggagatcaaaaccacgacgccagcgccgcggcca | |
| | ccaacaccggcgcccaccatcgcgtcgcagcccctgtccctgcgcccagaggcgtgccgg | |
| | ccagcggcggggggcgcagtgcacacgagggggctggacttcgcctgtgatatctacatct | |
| | gggcgcccttggccgggacttgtggggtccttctcctgtcactggttatcaccctttactgccgga | |
| | gggaccagaggctgccccccgatgcccacaagccccctgggggaggcagtttccggaccc | |
| | ccatccaagaggagcaggccgacgcccactccaccctggccaagatcagagtgaagttca | |
| | gcaggagcgcagacgcccccgcgtaccagcagggccagaaccagctctataacgagctc | |
| | aatctaggacgaagagaggagtacgatgttttggacaagagacgtggccgggaccctgag | |
| | atggggggaaagccgagaaggaagaaccctcaggaaggcctgtacaatgaactgcaga | |
| | aagataagatggcggaggcctacagtgagattgggatgaaaggcgagcgccggaggggc | |
| | aaggggcacgatggcctttaccagggtctcagtacagccaccaaggacacctacgacgcc | |
| | cttcacatgcaggccctgccccctcgcggctctggcgagggaaggggttccctgcttacttgcg | |
| | gcgacgtcgaagagaatcccggtccgatggccctcccagtaactgccctccttttgcccctcg | |
| | cactccttcttcatgccgctcgccccaactgggtcaacgtgattagcgatttgaagaaaatcga | |
| | ggaccttatacagtctatgcatattgacgctacactgtatactgagagtgatgtacacccgtcct | |
| | gtaaggtaacggccatgaaatgctttcttctggagctccaggtcatcagcttggagtctgggga | |
| | cgcaagcatccacgatacggttgaaaacctcatcatccttgcgaacaactctctctcatctaat | |
| | ggaaacgttacagagagtgggtgtaaggagtgcgaagagttggaagaaaaaaacatcaa | |
| | agaatttcttcaatccttcgttcacatagtgcaaatgttcattaacacgtccactaccacacccgc | |
| | cccgaggccacctacgccggcaccgactatcgccagtcaacccctctctctgcgccccgag | |
| | gcttgccggcctgcggctggtggggcggtccacacccggggcctggattttgcgtgcgatatat | |
| | acatctgggcacctcttgccggcacctgcggagtgctgcttctctcactcgttattacgctgtact | |
| | gctaa | |
| 39 | atggccttaccagtgaccgccttgctcctgccgctggccttgctgctccacgccgccaggccg | Anti-CD70 |
| | gactacaaagacgatgacgataaaggcggtggtggctctggtggtggcggatcccaggtgc | CAR |
| | agctggtgcagtctggggctgaggtgaagaagcctggggcctcagtgaaggtttcctgcaag | |
| | gcatctggatacaccttcaccagctacgacattaattgggtgcgacaggcccctggacaagg | |
| | gcttgagtggatgggagtgatcaaccctagtggtggaggtacaagctacgcacagaagttcc | |
| | agggcagagtcaccatgaccagggacacgtccacgagcacagtctacatggagctgagc | |
| | agcctgagatctgaggacacggccgtgtattactgtgctagatattctggctatgattgggtgttt | |
| | gactactggggccaagggaccctggtcaccgtctcctcaggtggaggcggttcaggcggag | |
| | gtggctctggcggtggcggatcggacatccagatgacccagtctccatcctccctgtctgcatc | |
| | tgtaggagacagagtcaccatcacttgccgggcaagtcagggcattggcagctggttagcttg | |
| | gtatcagcagaaaccagggaaagcccctaagctcctgatctatgctgcatccacattgcaaa | |
| | gtggggtcccatcaaggttcagtggcagtggatctgggacagatttcactctcaccatcagca | |
| | gtctgcaacctgaagattttgcaacttactactgtcaacagagttacagtaccccttggactttcg | |
| | gccctgggaccaaagtggatatcaaaaccacgacgccagcgccgcggccaccaacaccg | |
| | gcgcccaccatcgcgtcgcagcccctgtccctgcgcccagaggcgtgccggccagcggcg | |
| | gggggcgcagtgcacacgagggggctggacttcgcctgtgatatctacatctgggcgccctt | |
| | ggccgggacttgtggggtccttctcctgtcactggttatcaccctttactgccggagggaccaga | |
| | ggctgccccccgatgcccacaagccccctgggggaggcagtttccggacccccatccaag | |
| | aggagcaggccgacgcccactccaccctggccaagatcagagtgaagttcagcaggagc | |
| | gcagacgcccccgcgtaccagcagggccagaaccagctctataacgagctcaatctagga | |
| | cgaagagaggagtacgatgttttggacaagagacgtggccgggaccctgagatgggggga | |
| | aagccgagaaggaagaaccctcaggaaggcctgtacaatgaactgcagaaagataagat | |
| | ggcggaggcctacagtgagattgggatgaaaggcgagcgccggaggggcaaggggcac | |
| | gatggcctttaccagggtctcagtacagccaccaaggacacctacgacgcccttcacatgca | |
| | ggccctgccccctcgcggctctggcgagggaaggggttccctgcttacttgcggcgacgtcg | |
| | aagagaatcccggtccgatggccctcccagtaactgccctccttttgcccctcgcactccttcttc | |
| | atgccgctcgccccaactgggtcaacgtgattagcgatttgaagaaaatcgaggaccttatac | |
| | agtctatgcatattgacgctacactgtatactgagagtgatgtacacccgtcctgtaaggtaacg | |
| | gccatgaaatgctttcttctggagctccaggtcatcagcttggagtctggggacgcaagcatcc | |
| | acgatacggttgaaaacctcatcatccttgcgaacaactctctctcatctaatggaaacgttaca | |
| | gagagtgggtgtaaggagtgcgaagagttggaagaaaaaaacatcaaagaatttcttcaat | |
| | ccttcgttcacatagtgcaaatgttcattaacacgtccactaccacacccgccccgaggccac | |
| | ctacgccggcaccgactatcgccagtcaacccctctctctgcgccccgaggcttgccggcctg | |
| | cggctggtggggcggtccacacccggggcctggattttgcgtgcgatatatacatctgggcac | |
| | ctcttgccggcacctgcggagtgctgcttctctcactcgttattacgctgtactgctaa | |
| 40 | atggccttaccagtgaccgccttgctcctgccgctggccttgctgctccacgccgccaggccg | Anti-CD70 |
| | gactacaaagacgatgacgataaaggcggtggtggctctggtggtggcggatcccaggtgc | CAR |
| | agctggtgcagtctggggctgaggtgaagaagcctggggcctcagtgaaggtttcctgcaag | |
| | gcatctggatacaccttcaccagctacgacatcaattgggtgcgacaggcccctggacaagg | |
| | gcttgagtggatgggagtcatcagccctagtggtggtggaacaagctacgcacagaagttcc | |
| | agggcagagtcaccatgaccagggacacgtccacgagcacagtctacatggagctgagc | |
| | agcctgagatctgaggacacggccgtgtattactgtgctagatactacggcggcaaccccga | |
| | agggtggtacttcgatctctggggccgtggcaccctggtcactgtctcctcaggtggaggcggt | |
| | tcaggcggaggtggctctggcggtggcggatcggacatccagatgacccagtctccatcctc | |
| | cctgtctgcatctgtaggagacagagtcaccatcacttgccgggcaagtcagagcattggaa | |
| | attggttagcctggtatcagcagaaaccagggaaagcccctaagctcctgatctatgacgcat | |
| | ccgatttggaaaccggggtcccatcaaggttcagtggcagtggatctgggacagatttcactct | |
| | caccatcagcagtctgcaacctgaagattttgcaacttactactgtcaacagagttacactacc | |
| | ccttggacgttcggccaagggaccaaggtggaaatcaaaaccacgacgccagcgccgcg | |
| | gccaccaacaccggcgcccaccatcgcgtcgcagcccctgtccctgcgcccagaggcgtg | |
| | ccggccagcggcggggggcgcagtgcacacgagggggctggacttcgcctgtgatatctac | |
| | atctgggcgcccttggccgggacttgtggggtccttctcctgtcactggttatcaccctttactgcc | |
| | ggagggaccagaggctgccccccgatgcccacaagccccctgggggaggcagtttccgg | |
| | acccccatccaagaggagcaggccgacgcccactccaccctggccaagatcagagtgaa | |
| | gttcagcaggagcgcagacgcccccgcgtaccagcagggccagaaccagctctataacg | |
| | agctcaatctaggacgaagagaggagtacgatgttttggacaagagacgtggccgggacc | |
| | ctgagatggggggaaagccgagaaggaagaaccctcaggaaggcctgtacaatgaactg | |
| | cagaaagataagatggcggaggcctacagtgagattgggatgaaaggcgagcgccggag | |
| | gggcaaggggcacgatggcctttaccagggtctcagtacagccaccaaggacacctacga | |
| | cgcccttcacatgcaggccctgccccctcgcggctctggcgagggaaggggttccctgcttac | |
| | ttgcggcgacgtcgaagagaatcccggtccgatggccctcccagtaactgccctccttttgccc | |
| | ctcgcactccttcttcatgccgctcgccccaactgggtcaacgtgattagcgatttgaagaaaat | |
| | cgaggaccttatacagtctatgcatattgacgctacactgtatactgagagtgatgtacacccgt | |
| | cctgtaaggtaacggccatgaaatgctttcttctggagctccaggtcatcagcttggagtctggg | |
| | gacgcaagcatccacgatacggttgaaaacctcatcatccttgcgaacaactctctctcatcta | |
| | atggaaacgttacagagagtgggtgtaaggagtgcgaagagttggaagaaaaaaacatca | |
| | aagaatttcttcaatccttcgttcacatagtgcaaatgttcattaacacgtccactaccacacccg | |
| | ccccgaggccacctacgccggcaccgactatcgccagtcaacccctctctctgcgccccga | |
| | ggcttgccggcctgcggctggtggggcggtccacacccggggcctggattttgcgtgcgatat | |
| | atacatctgggcacctcttgccggcacctgcggagtgctgcttctctcactcgttattacgctgta | |
| | ctgctaa | |
| 41 | atggccttaccagtgaccgccttgctcctgccgctggccttgctgctccacgccgccaggccg | Anti-CD70 |
| | gacatccagatgacccagtctccatcctccctgtctgcatctgtaggagacagagtcaccatc | CAR |
| | acttgccgggcaagtcagggcattggcagctggttagcttggtatcagcagaaaccagggaa | |
| | agcccctaagctcctgatctatgctgcatccacattgcaaagtggggtcccatcaaggttcagt | |
| | ggcagtggatctgggacagatttcactctcaccatcagcagtctgcaacctgaagattttgcaa | |
| | cttactactgtcaacagagttacagtaccccttggactttcggccctgggaccaaagtggatatc | |
| | aaaggcagcaccagcggcagcggcaagcccggctccggagagggcagcaccaagggc | |
| | caggtgcagctggtgcagtctggggctgaggtgaagaagcctggggcctcagtgaaggtttc | |
| | ctgcaaggcatctggatacaccttcaccagctacgacattaattgggtgcgacaggcccctgg | |
| | acaagggcttgagtggatgggagtgatcaaccctagtggtggaggtacaagctacgcacag | |
| | aagttccagggcagagtcaccatgaccagggacacgtccacgagcacagtctacatggag | |
| | ctgagcagcctgagatctgaggacacggccgtgtattactgtgctagatattctggctatgattg | |
| | ggtgtttgactactggggccaagggaccctggtcaccgtctcctcagcggccgcaaccacga | |
| | cgccagcgccgcggccaccaacaccggcgcccaccatcgcgtcgcagcccctgtccctgc | |
| | gcccagaggcgtgccggccagcggcggggggcgcagtgcacacgagggggctggacttc | |
| | gcctgtgatatctacatctgggcgcccttggccgggacttgtggggtccttctcctgtcactggtta | |
| | tcaccctttactgccggagggaccagaggctgccccccgatgcccacaagccccctggggg | |
| | aggcagtttccggacccccatccaagaggagcaggccgacgcccactccaccctggccaa | |
| | gatcagagtgaagttcagcaggagcgcagacgcccccgcgtaccagcagggccagaacc | |
| | agctctataacgagctcaatctaggacgaagagaggagtacgatgttttggacaagagacgt | |
| | ggccgggaccctgagatggggggaaagccgagaaggaagaaccctcaggaaggcctgt | |
| | acaatgaactgcagaaagataagatggcggaggcctacagtgagattgggatgaaaggcg | |
| | agcgccggaggggcaaggggcacgatggcctttaccagggtctcagtacagccaccaag | |
| | gacacctacgacgcccttcacatgcaggccctgccccctcgcggctctggcgagggaaggg | |
| | gttccctgcttacttgcggcgacgtcgaagagaatcccggtccgatggccctcccagtaactg | |
| | ccctccttttgcccctcgcactccttcttcatgccgctcgccccaactgggtcaacgtgattagcg | |
| | atttgaagaaaatcgaggaccttatacagtctatgcatattgacgctacactgtatactgagagt | |
| | gatgtacacccgtcctgtaaggtaacggccatgaaatgctttcttctggagctccaggtcatca | |
| | gcttggagtctggggacgcaagcatccacgatacggttgaaaacctcatcatccttgcgaac | |
| | aactctctctcatctaatggaaacgttacagagagtgggtgtaaggagtgcgaagagttggaa | |
| | gaaaaaaacatcaaagaatttcttcaatccttcgttcacatagtgcaaatgttcattaacacgtc | |
| | cactaccacacccgccccgaggccacctacgccggcaccgactatcgccagtcaacccct | |
| | ctctctgcgccccgaggcttgccggcctgcggctggtggggcggtccacacccggggcctgg | |
| | attttgcgtgcgatatatacatctgggcacctcttgccggcacctgcggagtgctgcttctctcact | |
| | cgttattacgctgtactgc | |
| 42 | atggccttaccagtgaccgccttgctcctgccgctggccttgctgctccacgccgccaggccg | Anti-CD70 |
| | gacatccagatgacccagtctccatcctccctgtctgcatctgtaggagacagagtcaccatc | CAR |
| | acttgccgggcaagtcagagcattggaaattggttagcctggtatcagcagaaaccaggga | |
| | aagcccctaagctcctgatctatgacgcatccgatttggaaaccggggtcccatcaaggttca | |
| | gtggcagtggatctgggacagatttcactctcaccatcagcagtctgcaacctgaagattttgc | |
| | aacttactactgtcaacagagttacactaccccttggacgttcggccaagggaccaaggtgg | |
| | aaatcaaaggcagcaccagcggcagcggcaagcccggctccggagagggcagcacca | |
| | agggccaggtgcagctggtgcagtctggggctgaggtgaagaagcctggggcctcagtga | |
| | aggtttcctgcaaggcatctggatacaccttcaccagctacgacatcaattgggtgcgacagg | |
| | cccctggacaagggcttgagtggatgggagtcatcagccctagtggtggtggaacaagctac | |
| | gcacagaagttccagggcagagtcaccatgaccagggacacgtccacgagcacagtctac | |
| | atggagctgagcagcctgagatctgaggacacggccgtgtattactgtgctagatactacggc | |
| | ggcaaccccgaagggtggtacttcgatctctggggccgtggcaccctggtcactgtctcctca | |
| | gcggccgcaaccacgacgccagcgccgcggccaccaacaccggcgcccaccatcgcgt | |
| | cgcagcccctgtccctgcgcccagaggcgtgccggccagcggcggggggcgcagtgcac | |
| | acgagggggctggacttcgcctgtgatatctacatctgggcgcccttggccgggacttgtggg | |
| | gtccttctcctgtcactggttatcaccctttactgccggagggaccagaggctgccccccgatgc | |
| | ccacaagccccctgggggaggcagtttccggacccccatccaagaggagcaggccgacg | |
| | cccactccaccctggccaagatcagagtgaagttcagcaggagcgcagacgcccccgcgt | |
| | accagcagggccagaaccagctctataacgagctcaatctaggacgaagagaggagtac | |
| | gatgttttggacaagagacgtggccgggaccctgagatggggggaaagccgagaaggaa | |
| | gaaccctcaggaaggcctgtacaatgaactgcagaaagataagatggcggaggcctacag | |
| | tgagattgggatgaaaggcgagcgccggaggggcaaggggcacgatggcctttaccagg | |
| | gtctcagtacagccaccaaggacacctacgacgcccttcacatgcaggccctgccccctcgc | |
| | ggctctggcgagggaaggggttccctgcttacttgcggcgacgtcgaagagaatcccggtcc | |
| | gatggccctcccagtaactgccctccttttgcccctcgcactccttcttcatgccgctcgccccaa | |
| | ctgggtcaacgtgattagcgatttgaagaaaatcgaggaccttatacagtctatgcatattgac | |
| | gctacactgtatactgagagtgatgtacacccgtcctgtaaggtaacggccatgaaatgctttct | |
| | tctggagctccaggtcatcagcttggagtctggggacgcaagcatccacgatacggttgaaa | |
| | acctcatcatccttgcgaacaactctctctcatctaatggaaacgttacagagagtgggtgtaa | |
| | ggagtgcgaagagttggaagaaaaaaacatcaaagaatttcttcaatccttcgttcacatagt | |
| | gcaaatgttcattaacacgtccactaccacacccgccccgaggccacctacgccggcaccg | |
| | actatcgccagtcaacccctctctctgcgccccgaggcttgccggcctgcggctggtggggcg | |
| | gtccacacccggggcctggattttgcgtgcgatatatacatctgggcacctcttgccggcacct | |
| | gcggagtgctgcttctctcactcgttattacgctgtactgc | |
| 43 | atggccttaccagtgaccgccttgctcctgccgctggccttgctgctccacgccgccaggccg | Anti-CD70 |
| | gacatagttatgacccaatccccagatagtttggcggtttctctgggcgagagggcaacgatta | CAR |
| | attgtcgcgcatcaaagagcgtttcaacgagcggatattcttttatgcattggtaccagcaaaaa | |
| | cccggacaaccgccgaagctgctgatctacttggcttcaaatcttgagtctggggtgccggac | |
| | cgattttctggtagtggaagcggaactgactttacgctcacgatcagttcactgcaggctgagg | |
| | atgtagcggtctattattgccagcacagtagagaagtcccctggaccttcggtcaaggcacga | |
| | aagtagaaattaaaggcagcaccagcggcagcggcaagcccggctccggagagggcag | |
| | caccaagggccaggtccagttggtgcaaagcggggcggaggtgaaaaaacccggcgctt | |
| | ccgtgaaggtgtcctgtaaggcgtccggttatacgttcacgaactacgggatgaattgggttcg | |
| | ccaagcgccggggcagggactgaaatggatggggtggataaatacctacaccggcgaac | |
| | ctacatacgccgacgcttttaaagggcgagtcactatgacgcgcgataccagcatatccacc | |
| | gcatacatggagctgtcccgactccggtcagacgacacggctgtctactattgtgctcgggact | |
| | atggcgattatggcatggactactggggtcagggtacgactgtaacagttagtagtgcggccg | |
| | caaccacgacgccagcgccgcggccaccaacaccggcgcccaccatcgcgtcgcagcc | |
| | cctgtccctgcgcccagaggcgtgccggccagcggcggggggcgcagtgcacacgaggg | |
| | ggctggacttcgcctgtgatatctacatctgggcgcccttggccgggacttgtggggtccttctcc | |
| | tgtcactggttatcaccctttactgccggagggaccagaggctgccccccgatgcccacaagc | |
| | cccctgggggaggcagtttccggacccccatccaagaggagcaggccgacgcccactcca | |
| | ccctggccaagatcagagtgaagttcagcaggagcgcagacgcccccgcgtaccagcag | |
| | ggccagaaccagctctataacgagctcaatctaggacgaagagaggagtacgatgttttgg | |
| | acaagagacgtggccgggaccctgagatggggggaaagccgagaaggaagaaccctca | |
| | ggaaggcctgtacaatgaactgcagaaagataagatggcggaggcctacagtgagattgg | |
| | gatgaaaggcgagcgccggaggggcaaggggcacgatggcctttaccagggtctcagtac | |
| | agccaccaaggacacctacgacgcccttcacatgcaggccctgccccctcgcggctctggc | |
| | gagggaaggggttccctgcttacttgcggcgacgtcgaagagaatcccggtccgatggccct | |
| | cccagtaactgccctccttttgcccctcgcactccttcttcatgccgctcgccccaactgggtcaa | |
| | cgtgattagcgatttgaagaaaatcgaggaccttatacagtctatgcatattgacgctacactgt | |
| | atactgagagtgatgtacacccgtcctgtaaggtaacggccatgaaatgctttcttctggagctc | |
| | caggtcatcagcttggagtctggggacgcaagcatccacgatacggttgaaaacctcatcat | |
| | ccttgcgaacaactctctctcatctaatggaaacgttacagagagtgggtgtaaggagtgcga | |
| | agagttggaagaaaaaaacatcaaagaatttcttcaatccttcgttcacatagtgcaaatgttc | |
| | attaacacgtccactaccacacccgccccgaggccacctacgccggcaccgactatcgcca | |
| | gtcaacccctctctctgcgccccgaggcttgccggcctgcggctggtggggcggtccacacc | |
| | cggggcctggattttgcgtgcgatatatacatctgggcacctcttgccggcacctgcggagtgct | |
| | gcttctctcactcgttattacgctgtactgc | |
| 44 | atggccttaccagtgaccgccttgctcctgccgctggccttgctgctccacgccgccaggccgc | Anti-CD70 |
| | aggtgcagctggtgcagtctggggctgaggtgaagaagcctggggcctcagtgaaggtttcc | CAR |
| | tgcaaggcatctggatacaccttcaccagctacgacattaattgggtgcgacaggcccctgga | |
| | caagggcttgagtggatgggagtgatcaaccctagtggtggaggtacaagctacgcacaga | |
| | agttccagggcagagtcaccatgaccagggacacgtccacgagcacagtctacatggagct | |
| | gagcagcctgagatctgaggacacggccgtgtattactgtgctagatattctggctatgattgg | |
| | gtgtttgactactggggccaagggaccctggtcaccgtctcctcaggtggaggcggttcaggc | |
| | ggaggtggctctggcggtggcggatcggacatccagatgacccagtctccatcctccctgtct | |
| | gcatctgtaggagacagagtcaccatcacttgccgggcaagtcagggcattggcagctggtt | |
| | agcttggtatcagcagaaaccagggaaagcccctaagctcctgatctatgctgcatccacatt | |
| | gcaaagtggggtcccatcaaggttcagtggcagtggatctgggacagatttcactctcaccat | |
| | cagcagtctgcaacctgaagattttgcaacttactactgtcaacagagttacagtaccccttgg | |
| | actttcggccctgggaccaaagtggatatcaaaaccacgacgccagcgccgcgaccacca | |
| | acaccggcgcccaccatcgcgtcgcagcccctgtccctgcgcccagaggcgtgccggcca | |
| | gcggcggggggcgcagtgcacacgagggggctggacttcgcctgtgatatctacatctggg | |
| | cgcccttggccgggacttgtggggtccttctcctgtcactggttatcaccctttactgccggaggg | |
| | accagaggctgccccccgatgcccacaagccccctgggggaggcagtttccggaccccca | |
| | tccaagaggagcaggccgacgcccactccaccctggccaagatcagagtgaagttcagca | |
| | ggagcgcagacgcccccgcgtaccagcagggccagaaccagctctataacgagctcaat | |
| | ctaggacgaagagaggagtacgatgttttggacaagagacgtggccgggaccctgagatg | |
| | gggggaaagccgagaaggaagaaccctcaggaaggcctgtacaatgaactgcagaaag | |
| | ataagatggcggaggcctacagtgagattgggatgaaaggcgagcgccggaggggcaag | |
| | gggcacgatggcctttaccagggtctcagtacagccaccaaggacacctacgacgcccttc | |
| | acatgcaggccctgccccctcgcggctctggcgagggaaggggttccctgcttacttgcggc | |
| | gacgtcgaagagaatcccggtccgatggccctcccagtaactgccctccttttgcccctcgca | |
| | ctccttcttcatgccgctcgccccaactgggtcaacgtgattagcgatttgaagaaaatcgagg | |
| | accttatacagtctatgcatattgacgctacactgtatactgagagtgatgtacacccgtcctgta | |
| | aggtaacggccatgaaatgctttcttctggagctccaggtcatcagcttggagtctggggacgc | |
| | aagcatccacgatacggttgaaaacctcatcatccttgcgaacaactctctctcatctaatgga | |
| | aacgttacagagagtgggtgtaaggagtgcgaagagttggaagaaaaaaacatcaaaga | |
| | atttcttcaatccttcgttcacatagtgcaaatgttcattaacacgtccactaccacacccgcccc | |
| | gaggccacctacgccggcaccgactatcgccagtcaacccctctctctgcgccccgaggctt | |
| | gccggcctgcggctggtggggcggtccacacccggggcctggattttgcgtgcgatatataca | |
| | tctgggcacctcttgccggcacctgcggagtgctgcttctctcactcgttattacgctgtactgc | |
| 45 | atggccttaccagtgaccgccttgctcctgccgctggccttgctgctccacgccgccaggccgc | Anti-CD70 |
| | aggtgcagctggtgcagtctggggctgaggtgaagaagcctggggcctcagtgaaggtttcc | CAR |
| | tgcaaggcatctggatacaccttcaccagctacgacatcaattgggtgcgacaggcccctgg | |
| | acaagggcttgagtggatgggagtcatcagccctagtggtggtggaacaagctacgcacag | |
| | aagttccagggcagagtcaccatgaccagggacacgtccacgagcacagtctacatggag | |
| | ctgagcagcctgagatctgaggacacggccgtgtattactgtgctagatactacggcggcaa | |
| | ccccgaagggtggtacttcgatctctggggccgtggcaccctggtcactgtctcctcaggtgga | |
| | ggcggttcaggcggaggtggctctggcggtggcggatcggacatccagatgacccagtctc | |
| | catcctccctgtctgcatctgtaggagacagagtcaccatcacttgccgggcaagtcagagca | |
| | ttggaaattggttagcctggtatcagcagaaaccagggaaagcccctaagctcctgatctatg | |
| | acgcatccgatttggaaaccggggtcccatcaaggttcagtggcagtggatctgggacagatt | |
| | tcactctcaccatcagcagtctgcaacctgaagattttgcaacttactactgtcaacagagttac | |
| | actaccccttggacgttcggccaagggaccaaggtggaaatcaaaaccacgacgccagcg | |
| | ccgcgaccaccaacaccggcgcccaccatcgcgtcgcagcccctgtccctgcgcccagag | |
| | gcgtgccggccagcggcggggggcgcagtgcacacgagggggctggacttcgcctgtgat | |
| | atctacatctgggcgcccttggccgggacttgtggggtccttctcctgtcactggttatcacccttt | |
| | actgccggagggaccagaggctgccccccgatgcccacaagccccctgggggaggcagt | |
| | ttccggacccccatccaagaggagcaggccgacgcccactccaccctggccaagatcaga | |
| | gtgaagttcagcaggagcgcagacgcccccgcgtaccagcagggccagaaccagctctat | |
| | aacgagctcaatctaggacgaagagaggagtacgatgttttggacaagagacgtggccgg | |
| | gaccctgagatggggggaaagccgagaaggaagaaccctcaggaaggcctgtacaatg | |
| | aactgcagaaagataagatggcggaggcctacagtgagattgggatgaaaggcgagcgc | |
| | cggaggggcaaggggcacgatggcctttaccagggtctcagtacagccaccaaggacacc | |
| | tacgacgcccttcacatgcaggccctgccccctcgcggctctggcgagggaaggggttccct | |
| | gcttacttgcggcgacgtcgaagagaatcccggtccgatggccctcccagtaactgccctcctt | |
| | ttgcccctcgcactccttcttcatgccgctcgccccaactgggtcaacgtgattagcgatttgaag | |
| | aaaatcgaggaccttatacagtctatgcatattgacgctacactgtatactgagagtgatgtac | |
| | acccgtcctgtaaggtaacggccatgaaatgctttcttctggagctccaggtcatcagcttgga | |
| | gtctggggacgcaagcatccacgatacggttgaaaacctcatcatccttgcgaacaactctct | |
| | ctcatctaatggaaacgttacagagagtgggtgtaaggagtgcgaagagttggaagaaaaa | |
| | aacatcaaagaatttcttcaatccttcgttcacatagtgcaaatgttcattaacacgtccactacc | |
| | acacccgccccgaggccacctacgccggcaccgactatcgccagtcaacccctctctctgc | |
| | gccccgaggcttgccggcctgcggctggtggggcggtccacacccggggcctggattttgcg | |
| | tgcgatatatacatctgggcacctcttgccggcacctgcggagtgctgcttctctcactcgttatta | |
| | cgctgtactgc | |
| 46 | atggccttaccagtgaccgccttgctcctgccgctggccttgctgctccacgccgccaggccgc | Anti-CD70 |
| | aggtccagttggtgcaaagcggggcggaggtgaaaaaacccggcgcttccgtgaaggtgtc | CAR |
| | ctgtaaggcgtccggttatacgttcacgaactacgggatgaattgggttcgccaagcgccggg | |
| | gcagggactgaaatggatggggtggataaatacctacaccggcgaacctacatacgccga | |
| | cgcttttaaagggcgagtcactatgacgcgcgataccagcatatccaccgcatacatggagct | |
| | gtcccgactccggtcagacgacacggctgtctactattgtgctcgggactatggcgattatggc | |
| | atggactactggggtcagggtacgactgtaacagttagtagtggtggaggcggttcaggcgg | |
| | aggtggctctggcggtggcggatcggacatagttatgacccaatccccagatagtttggcggtt | |
| | tctctgggcgagagggcaacgattaattgtcgcgcatcaaagagcgtttcaacgagcggatat | |
| | tcttttatgcattggtaccagcaaaaacccggacaaccgccgaagctgctgatctacttggcttc | |
| | aaatcttgagtctggggtgccggaccgattttctggtagtggaagcggaactgactttacgctca | |
| | cgatcagttcactgcaggctgaggatgtagcggtctattattgccagcacagtagagaagtcc | |
| | cctggaccttcggtcaaggcacgaaagtagaaattaaaaccacgacgccagcgccgcgac | |
| | caccaacaccggcgcccaccatcgcgtcgcagcccctgtccctgcgcccagaggcgtgcc | |
| | ggccagcggcggggggcgcagtgcacacgagggggctggacttcgcctgtgatatctacat | |
| | ctgggcgcccttggccgggacttgtggggtccttctcctgtcactggttatcaccctttactgccg | |
| | gagggaccagaggctgccccccgatgcccacaagccccctgggggaggcagtttccgga | |
| | cccccatccaagaggagcaggccgacgcccactccaccctggccaagatcagagtgaagt | |
| | tcagcaggagcgcagacgcccccgcgtaccagcagggccagaaccagctctataacgag | |
| | ctcaatctaggacgaagagaggagtacgatgttttggacaagagacgtggccgggaccctg | |
| | agatggggggaaagccgagaaggaagaaccctcaggaaggcctgtacaatgaactgca | |
| | gaaagataagatggcggaggcctacagtgagattgggatgaaaggcgagcgccggaggg | |
| | gcaaggggcacgatggcctttaccagggtctcagtacagccaccaaggacacctacgacg | |
| | cccttcacatgcaggccctgccccctcgcggctctggcgagggaaggggttccctgcttacttg | |
| | cggcgacgtcgaagagaatcccggtccgatggccctcccagtaactgccctccttttgcccct | |
| | cgcactccttcttcatgccgctcgccccaactgggtcaacgtgattagcgatttgaagaaaatc | |
| | gaggaccttatacagtctatgcatattgacgctacactgtatactgagagtgatgtacacccgtc | |
| | ctgtaaggtaacggccatgaaatgctttcttctggagctccaggtcatcagcttggagtctgggg | |
| | acgcaagcatccacgatacggttgaaaacctcatcatccttgcgaacaactctctctcatctaa | |
| | tggaaacgttacagagagtgggtgtaaggagtgcgaagagttggaagaaaaaaacatcaa | |
| | agaatttcttcaatccttcgttcacatagtgcaaatgttcattaacacgtccactaccacacccgc | |
| | cccgaggccacctacgccggcaccgactatcgccagtcaacccctctctctgcgccccgag | |
| | gcttgccggcctgcggctggtggggcggtccacacccggggcctggattttgcgtgcgatatat | |
| | acatctgggcacctcttgccggcacctgcggagtgctgcttctctcactcgttattacgctgtact | |
| | gc | |
| 47 | QVQLVQSGAEVKKPGASVKVSCKASGYTFTDYYMHWVRQAPGQ | Anti-CD70 |
| | GLEWMGIINPNSGGTNYAQKFQGRVTMTRDTSTSTVYMELSSLR | scFv |
| | SEDTAVYYCARDFSVYAAADGMDVWGQGTLVTVSSGGGGSGGG | |
| | GSGGGGSDIQMTQSPSSLSASVGDRVTITCRASQSISSYLNWYQ | |
| | QKPGKAPKLLIYDASNLATGVPSRFSGSGSGTDFTLTISSLQPEDF | |
| | ATYYCQQSDSSPLTFGQGTKVEIK | |
| 48 | QVQLVQSGAEVKKPGASVKVSCKASGYTFTSYDINWVRQAPGQG | Anti-CD70 |
| | LEWMGVINPSGGGTSYAQKFQGRVTMTRDTSTSTVYMELSSLRS | scFv |
| | EDTAVYYCARYSGYDWVFDYWGQGTLVTVSSGGGGSGGGGSG | |
| | GGGSDIQMTQSPSSLSASVGDRVTITCRASQGIGSWLAWYQQKP | |
| | GKAPKLLIYAASTLQSGVPSRFSGSGSGTDFTLTISSLQPEDFATY | |
| | YCQQSYSTPWTFGPGTKVDIK | |
| 49 | QVQLVQSGAEVKKPGASVKVSCKASGYTFTSYDINWVRQAPGQG | Anti-CD70 |
| | LEWMGVISPSGGGTSYAQKFQGRVTMTRDTSTSTVYMELSSLRS | scFv |
| | EDTAVYYCARYYGGNPEGWYFDLWGRGTLVTVSSGGGGSGGG | |
| | GSGGGGSDIQMTQSPSSLSASVGDRVTITCRASQSIGNWLAWYQ | |
| | QKPGKAPKLLIYDASDLETGVPSRFSGSGSGTDFTLTISSLQPEDF | |
| | ATYYCQQSYTTPWTFGQGTKVEIK | |
| 50 | GSTSGSGKPGSGEGSTKG | Linker |
| 51 | DIQMTQSPSSLSASVGDRVTITCRASQGIGSWLAWYQQKPGKAP | Anti-CD70 |
| | KLLIYAASTLQSGVPSRFSGSGSGTDFTLTISSLQPEDFATYYCQQ | scFv |
| | SYSTPWTFGPGTKVDIKGSTSGSGKPGSGEGSTKGQVQLVQSGA | |
| | EVKKPGASVKVSCKASGYTFTSYDINWVRQAPGQGLEWMGVINP | |
| | SGGGTSYAQKFQGRVTMTRDTSTSTVYMELSSLRSEDTAVYYCA | |
| | RYSGYDWVFDYWGQGTLVTVSS | |
| 52 | DIQMTQSPSSLSASVGDRVTITCRASQSIGNWLAWYQQKPGKAPK | Anti-CD70 |
| | LLIYDASDLETGVPSRFSGSGSGTDFTLTISSLQPEDFATYYCQQS | scFv |
| | YTTPWTFGQGTKVEIKGSTSGSGKPGSGEGSTKGQVQLVQSGAE | |
| | VKKPGASVKVSCKASGYTFTSYDINWVRQAPGQGLEWMGVISPS | |
| | GGGTSYAQKFQGRVTMTRDTSTSTVYMELSSLRSEDTAVYYCAR | |
| | YYGGNPEGWYFDLWGRGTLVTVSS | |
| 53 | DIVMTQSPDSLAVSLGERATINCRASKSVSTSGYSFMHWYQQKP | Anti-CD70 |
| | GQPPKLLIYLASNLESGVPDRFSGSGSGTDFTLTISSLQAEDVAVY | scFv |
| | YCQHSREVPWTFGQGTKVEIKGSTSGSGKPGSGEGSTKGQVQL | |
| | VQSGAEVKKPGASVKVSCKASGYTFTNYGMNWVRQAPGQGLKW | |
| | MGWINTYTGEPTYADAFKGRVTMTRDTSISTAYMELSRLRSDDTA | |
| | VYYCARDYGDYGMDYWGQGTTVTVSS | |
| 54 | QVQLVQSGAEVKKPGASVKVSCKASGYTFTNYGMNWVRQAPGQ | Anti-CD70 |
| | GLKWMGWINTYTGEPTYADAFKGRVTMTRDTSISTAYMELSRLRS | scFv |
| | DDTAVYYCARDYGDYGMDYWGQGTTVTVSSGGGGSGGGGSGG | |
| | GGSDIVMTQSPDSLAVSLGERATINCRASKSVSTSGYSFMHWYQ | |
| | QKPGQPPKLLIYLASNLESGVPDRFSGSGSGTDFTLTISSLQAEDV | |
| | AVYYCQHSREVPWTFGQGTKVEIK | |
| 55 | MALPVTALLLPLALLLHAARPDYKDDDDKGGGGSGGGGSQVQLV | Anti-CD70 |
| | QSGAEVKKPGASVKVSCKASGYTFTDYYMHWVRQAPGQGLEWM | CAR |
| | GIINPNSGGTNYAQKFQGRVTMTRDTSTSTVYMELSSLRSEDTAV | |
| | YYCARDFSVYAAADGMDVWGQGTLVTVSSGGGGSGGGGSGGG | |
| | GSDIQMTQSPSSLSASVGDRVTITCRASQSISSYLNWYQQKPGKA | |
| | PKLLIYDASNLATGVPSRFSGSGSGTDFTLTISSLQPEDFATYYCQ | |
| | QSDSSPLTFGQGTKVEIKTTTPAPRPPTPAPTIASQPLSLRPEACR | |
| | PAAGGAVHTRGLDFACDIYIWAPLAGTCGVLLLSLVITLYCRRDQR | |
| | LPPDAHKPPGGGSFRTPIQEEQADAHSTLAKIRVKFSRSADAPAY | |
| | QQGQNQLYNELNLGRREEYDVLDKRRGRDPEMGGKPRRKNPQE | |
| | GLYNELQKDKMAEAYSEIGMKGERRRGKGHDGLYQGLSTATKDT | |
| | YDALHMQALPPRGSGEGRGSLLTCGDVEENPGPMALPVTALLLPL | |
| | ALLLHAARPNWVNVISDLKKIEDLIQSMHIDATLYTESDVHPSCKVT | |
| | AMKCFLLELQVISLESGDASIHDTVENLIILANNSLSSNGNVTESGC | |
| | KECEELEEKNIKEFLQSFVHIVQMFINTSTTTPAPRPPTPAPTIASQ | |
| | PLSLRPEACRPAAGGAVHTRGLDFACDIYIWAPLAGTCGVLLLSLV | |
| | ITLYC | |
| 56 | MALPVTALLLPLALLLHAARPDYKDDDDKGGGGSGGGGSQVQLV | Anti-CD70 |
| | QSGAEVKKPGASVKVSCKASGYTFTSYDINWVRQAPGQGLEWM | CAR |
| | GVINPSGGGTSYAQKFQGRVTMTRDTSTSTVYMELSSLRSEDTAV | |
| | YYCARYSGYDWVFDYWGQGTLVTVSSGGGGGGGGSGGGGSD | |
| | IQMTQSPSSLSASVGDRVTITCRASQGIGSWLAWYQQKPGKAPKL | |
| | LIYAASTLQSGVPSRFSGSGSGTDFTLTISSLQPEDFATYYCQQSY | |
| | STPWTFGPGTKVDIKTTTPAPRPPTPAPTIASQPLSLRPEACRPAA | |
| | GGAVHTRGLDFACDIYIWAPLAGTCGVLLLSLVITLYCRRDQRLPP | |
| | DAHKPPGGGSFRTPIQEEQADAHSTLAKIRVKFSRSADAPAYQQG | |
| | QNQLYNELNLGRREEYDVLDKRRGRDPEMGGKPRRKNPQEGLY | |
| | NELQKDKMAEAYSEIGMKGERRRGKGHDGLYQGLSTATKDTYDA | |
| | LHMQALPPRGSGEGRGSLLTCGDVEENPGPMALPVTALLLPLALL | |
| | LHAARPNWVNVISDLKKIEDLIQSMHIDATLYTESDVHPSCKVTAM | |
| | KCFLLELQVISLESGDASIHDTVENLIILANNSLSSNGNVTESGCKE | |
| | CEELEEKNIKEFLQSFVHIVQMFINTSTTTPAPRPPTPAPTIASQPLS | |
| | LRPEACRPAAGGAVHTRGLDFACDIYIWAPLAGTCGVLLLSLVITLY | |
| | C | |
| 57 | MALPVTALLLPLALLLHAARPDYKDDDDKGGGGSGGGGSQVQLV | Anti-CD70 |
| | QSGAEVKKPGASVKVSCKASGYTFTSYDINWVRQAPGQGLEWM | CAR |
| | GVISPSGGGTSYAQKFQGRVTMTRDTSTSTVYMELSSLRSEDTAV | |
| | YYCARYYGGNPEGWYFDLWGRGTLVTVSSGGGGSGGGGSGGG | |
| | GSDIQMTQSPSSLSASVGDRVTITCRASQSIGNWLAWYQQKPGK | |
| | APKLLIYDASDLETGVPSRFSGSGSGTDFTLTISSLQPEDFATYYC | |
| | QQSYTTPWTFGQGTKVEIKTTTPAPRPPTPAPTIASQPLSLRPEAC | |
| | RPAAGGAVHTRGLDFACDIYIWAPLAGTCGVLLLSLVITLYCRRDQ | |
| | RLPPDAHKPPGGGSFRTPIQEEQADAHSTLAKIRVKFSRSADAPA | |
| | YQQGQNQLYNELNLGRREEYDVLDKRRGRDPEMGGKPRRKNPQ | |
| | EGLYNELQKDKMAEAYSEIGMKGERRRGKGHDGLYQGLSTATKD | |
| | TYDALHMQALPPRGSGEGRGSLLTCGDVEENPGPMALPVTALLL | |
| | PLALLLHAARPNWVNVISDLKKIEDLIQSMHIDATLYTESDVHPSCK | |
| | VTAMKCFLLELQVISLESGDASIHDTVENLIILANNSLSSNGNVTES | |
| | GCKECEELEEKNIKEFLQSFVHIVQMFINTSTTTPAPRPPTPAPTIA | |
| | SQPLSLRPEACRPAAGGAVHTRGLDFACDIYIWAPLAGTCGVLLLS | |
| | LVITLYC | |
| 58 | MALPVTALLLPLALLLHAARPDIQMTQSPSSLSASVGDRVTITCRAS | Anti-CD70 |
| | QGIGSWLAWYQQKPGKAPKLLIYAASTLQSGVPSRFSGSGSGTD | CAR |
| | FTLTISSLQPEDFATYYCQQSYSTPWTFGPGTKVDIKGSTSGSGK | |
| | PGSGEGSTKGQVQLVQSGAEVKKPGASVKVSCKASGYTFTSYDI | |
| | NWVRQAPGQGLEWMGVINPSGGGTSYAQKFQGRVTMTRDTSTS | |
| | TVYMELSSLRSEDTAVYYCARYSGYDWVFDYWGQGTLVTVSSAA | |
| | ATTTPAPRPPTPAPTIASQPLSLRPEACRPAAGGAVHTRGLDFAC | |
| | DIYIWAPLAGTCGVLLLSLVITLYCRRDQRLPPDAHKPPGGGSFRT | |
| | PIQEEQADAHSTLAKIRVKFSRSADAPAYQQGQNQLYNELNLGRR | |
| | EEYDVLDKRRGRDPEMGGKPRRKNPQEGLYNELQKDKMAEAYS | |
| | EIGMKGERRRGKGHDGLYQGLSTATKDTYDALHMQALPPRGSGE | |
| | GRGSLLTCGDVEENPGPMALPVTALLLPLALLLHAARPNWVNVIS | |
| | DLKKIEDLIQSMHIDATLYTESDVHPSCKVTAMKCFLLELQVISLES | |
| | GDASIHDTVENLIILANNSLSSNGNVTESGCKECEELEEKNIKEFLQ | |
| | SFVHIVQMFINTSTTTPAPRPPTPAPTIASQPLSLRPEACRPAAGG | |
| | AVHTRGLDFACDIYIWAPLAGTCGVLLLSLVITLYC | |
| 59 | MALPVTALLLPLALLLHAARPDIQMTQSPSSLSASVGDRVTITCRAS | Anti-CD70 |
| | QSIGNWLAWYQQKPGKAPKLLIYDASDLETGVPSRFSGSGSGTDF | CAR |
| | TLTISSLQPEDFATYYCQQSYTTPWTFGQGTKVEIKGSTSGSGKP | |
| | GSGEGSTKGQVQLVQSGAEVKKPGASVKVSCKASGYTFTSYDIN | |
| | WVRQAPGQGLEWMGVISPSGGGTSYAQKFQGRVTMTRDTSTST | |
| | VYMELSSLRSEDTAVYYCARYYGGNPEGWYFDLWGRGTLVTVSS | |
| | AAATTTPAPRPPTPAPTIASQPLSLRPEACRPAAGGAVHTRGLDFA | |
| | CDIYIWAPLAGTCGVLLLSLVITLYCRRDQRLPPDAHKPPGGGSFR | |
| | TPIQEEQADAHSTLAKIRVKFSRSADAPAYQQGQNQLYNELNLGR | |
| | REEYDVLDKRRGRDPEMGGKPRRKNPQEGLYNELQKDKMAEAY | |
| | SEIGMKGERRRGKGHDGLYQGLSTATKDTYDALHMQALPPRGSG | |
| | EGRGSLLTCGDVEENPGPMALPVTALLLPLALLLHAARPNWVNVIS | |
| | DLKKIEDLIQSMHIDATLYTESDVHPSCKVTAMKCFLLELQVISLES | |
| | GDASIHDTVENLIILANNSLSSNGNVTESGCKECEELEEKNIKEFLQ | |
| | SFVHIVQMFINTSTTTPAPRPPTPAPTIASQPLSLRPEACRPAAGG | |
| | AVHTRGLDFACDIYIWAPLAGTCGVLLLSLVITLYC | |
| 60 | MALPVTALLLPLALLLHAARPDIVMTQSPDSLAVSLGERATINCRAS | Anti-CD70 |
| | KSVSTSGYSFMHWYQQKPGQPPKLLIYLASNLESGVPDRFSGSG | CAR |
| | SGTDFTLTISSLQAEDVAVYYCQHSREVPWTFGQGTKVEIKGSTS | |
| | GSGKPGSGEGSTKGQVQLVQSGAEVKKPGASVKVSCKASGYTFT | |
| | NYGMNWVRQAPGQGLKWMGWINTYTGEPTYADAFKGRVTMTR | |
| | DTSISTAYMELSRLRSDDTAVYYCARDYGDYGMDYWGQGTTVTV | |
| | SSAAATTTPAPRPPTPAPTIASQPLSLRPEACRPAAGGAVHTRGL | |
| | DFACDIYIWAPLAGTCGVLLLSLVITLYCRRDQRLPPDAHKPPGGG | |
| | SFRTPIQEEQADAHSTLAKIRVKFSRSADAPAYQQGQNQLYNELN | |
| | LGRREEYDVLDKRRGRDPEMGGKPRRKNPQEGLYNELQKDKMA | |
| | EAYSEIGMKGERRRGKGHDGLYQGLSTATKDTYDALHMQALPPR | |
| | GSGEGRGSLLTCGDVEENPGPMALPVTALLLPLALLLHAARPNWV | |
| | NVISDLKKIEDLIQSMHIDATLYTESDVHPSCKVTAMKCFLLELQVIS | |
| | LESGDASIHDTVENLIILANNSLSSNGNVTESGCKECEELEEKNIKE | |
| | FLQSFVHIVQMFINTSTTTPAPRPPTPAPTIASQPLSLRPEACRPAA | |
| | GGAVHTRGLDFACDIYIWAPLAGTCGVLLLSLVITLYC | |
| 61 | MALPVTALLLPLALLLHAARPQVQLVQSGAEVKKPGASVKVSCKA | Anti-CD70 |
| | SGYTFTSYDINWVRQAPGQGLEWMGVINPSGGGTSYAQKFQGR | CAR |
| | VTMTRDTSTSTVYMELSSLRSEDTAVYYCARYSGYDWVFDYWGQ | |
| | GTLVTVSSGGGGSGGGGSGGGGSDIQMTQSPSSLSASVGDRVTI | |
| | TCRASQGIGSWLAWYQQKPGKAPKLLIYAASTLQSGVPSRFSGS | |
| | GSGTDFTLTISSLQPEDFATYYCQQSYSTPWTFGPGTKVDIKTTTP | |
| | APRPPTPAPTIASQPLSLRPEACRPAAGGAVHTRGLDFACDIYIWA | |
| | PLAGTCGVLLLSLVITLYCRRDQRLPPDAHKPPGGGSFRTPIQEEQ | |
| | ADAHSTLAKIRVKFSRSADAPAYQQGQNQLYNELNLGRREEYDVL | |
| | DKRRGRDPEMGGKPRRKNPQEGLYNELQKDKMAEAYSEIGMKG | |
| | ERRRGKGHDGLYQGLSTATKDTYDALHMQALPPRGSGEGRGSLL | |
| | TCGDVEENPGPMALPVTALLLPLALLLHAARPNWVNVISDLKKIED | |
| | LIQSMHIDATLYTESDVHPSCKVTAMKCFLLELQVISLESGDASIHD | |
| | TVENLIILANNSLSSNGNVTESGCKECEELEEKNIKEFLQSFVHIVQ | |
| | MFINTSTTTPAPRPPTPAPTIASQPLSLRPEACRPAAGGAVHTRGL | |
| | DFACDIYIWAPLAGTCGVLLLSLVITLYC | |
| 62 | MALPVTALLLPLALLLHAARPQVQLVQSGAEVKKPGASVKVSCKA | Anti-CD70 |
| | SGYTFTSYDINWVRQAPGQGLEWMGVISPSGGGTSYAQKFQGR | CAR |
| | VTMTRDTSTSTVYMELSSLRSEDTAVYYCARYYGGNPEGWYFDL | |
| | WGRGTLVTVSSGGGGSGGGGSGGGGSDIQMTQSPSSLSASVGD | |
| | RVTITCRASQSIGNWLAWYQQKPGKAPKLLIYDASDLETGVPSRF | |
| | SGSGSGTDFTLTISSLQPEDFATYYCQQSYTTPWTFGQGTKVEIK | |
| | TTTPAPRPPTPAPTIASQPLSLRPEACRPAAGGAVHTRGLDFACDI | |
| | YIWAPLAGTCGVLLLSLVITLYCRRDQRLPPDAHKPPGGGSFRTPI | |
| | QEEQADAHSTLAKIRVKFSRSADAPAYQQGQNQLYNELNLGRRE | |
| | EYDVLDKRRGRDPEMGGKPRRKNPQEGLYNELQKDKMAEAYSEI | |
| | GMKGERRRGKGHDGLYQGLSTATKDTYDALHMQALPPRGSGEG | |
| | RGSLLTCGDVEENPGPMALPVTALLLPLALLLHAARPNWVNVISDL | |
| | KKIEDLIQSMHIDATLYTESDVHPSCKVTAMKCFLLELQVISLESGD | |
| | ASIHDTVENLIILANNSLSSNGNVTESGCKECEELEEKNIKEFLQSF | |
| | VHIVQMFINTSTTTPAPRPPTPAPTIASQPLSLRPEACRPAAGGAV | |
| | HTRGLDFACDIYIWAPLAGTCGVLLLSLVITLYC | |
| 63 | MALPVTALLLPLALLLHAARPQVQLVQSGAEVKKPGASVKVSCKA | Anti-CD70 |
| | SGYTFTNYGMNWVRQAPGQGLKWMGWINTYTGEPTYADAFKGR | CAR |
| | VTMTRDTSISTAYMELSRLRSDDTAVYYCARDYGDYGMDYWGQG | |
| | TTVTVSSGGGGGGGGSGGGGSDIVMTQSPDSLAVSLGERATIN | |
| | CRASKSVSTSGYSFMHWYQQKPGQPPKLLIYLASNLESGVPDRF | |
| | SGSGSGTDFTLTISSLQAEDVAVYYCQHSREVPWTFGQGTKVEIK | |
| | TTTPAPRPPTPAPTIASQPLSLRPEACRPAAGGAVHTRGLDFACDI | |
| | YIWAPLAGTCGVLLLSLVITLYCRRDQRLPPDAHKPPGGGSFRTPI | |
| | QEEQADAHSTLAKIRVKFSRSADAPAYQQGQNQLYNELNLGRRE | |
| | EYDVLDKRRGRDPEMGGKPRRKNPQEGLYNELQKDKMAEAYSEI | |
| | GMKGERRRGKGHDGLYQGLSTATKDTYDALHMQALPPRGSGEG | |
| | RGSLLTCGDVEENPGPMALPVTALLLPLALLLHAARPNWVNVISDL | |
| | KKIEDLIQSMHIDATLYTESDVHPSCKVTAMKCFLLELQVISLESGD | |
| | ASIHDTVENLIILANNSLSSNGNVTESGCKECEELEEKNIKEFLQSF | |
| | VHIVQMFINTSTTTPAPRPPTPAPTIASQPLSLRPEACRPAAGGAV | |
| | HTRGLDFACDIYIWAPLAGTCGVLLLSLVITLYC | |
| 64 | MALPVTALLLPLALLLHAARPDYKDDDDKGGGGSGGGGSQVQLV | Anti-CD70 |
| | QSGAEVKKPGASVKVSCKASGYTFTDYYMHWVRQAPGQGLEWM | CAR |
| | GIINPNSGGTNYAQKFQGRVTMTRDTSTSTVYMELSSLRSEDTAV | |
| | YYCARDFSVYAAADGMDVWGQGTLVTVSSGGGGSGGGGSGGG | |
| | GSDIQMTQSPSSLSASVGDRVTITCRASQSISSYLNWYQQKPGKA | |
| | PKLLIYDASNLATGVPSRFSGSGSGTDFTLTISSLQPEDFATYYCQ | |
| | QSDSSPLTFGQGTKVEIKTTTPAPRPPTPAPTIASQPLSLRPEACR | |
| | PAAGGAVHTRGLDFACDIYIWAPLAGTCGVLLLSLVITLYCRRDQR | |
| | LPPDAHKPPGGGSFRTPIQEEQADAHSTLAKIRVKFSRSADAPAY | |
| | QQGQNQLYNELNLGRREEYDVLDKRRGRDPEMGGKPRRKNPQE | |
| | GLYNELQKDKMAEAYSEIGMKGERRRGKGHDGLYQGLSTATKDT | |
| | YDALHMQALPPR | |
| 65 | MALPVTALLLPLALLLHAARPDYKDDDDKGGGGGGGGSQVQLV | Anti-CD70 |
| | QSGAEVKKPGASVKVSCKASGYTFTSYDINWVRQAPGQGLEWM | CAR |
| | GVINPSGGGTSYAQKFQGRVTMTRDTSTSTVYMELSSLRSEDTAV | |
| | YYCARYSGYDWVFDYWGQGTLVTVSSGGGGSGGGGSGGGGSD | |
| | IQMTQSPSSLSASVGDRVTITCRASQGIGSWLAWYQQKPGKAPKL | |
| | LIYAASTLQSGVPSRFSGSGSGTDFTLTISSLQPEDFATYYCQQSY | |
| | STPWTFGPGTKVDIKTTTPAPRPPTPAPTIASQPLSLRPEACRPAA | |
| | GGAVHTRGLDFACDIYIWAPLAGTCGVLLLSLVITLYCRRDQRLPP | |
| | DAHKPPGGGSFRTPIQEEQADAHSTLAKIRVKFSRSADAPAYQQG | |
| | QNQLYNELNLGRREEYDVLDKRRGRDPEMGGKPRRKNPQEGLY | |
| | NELQKDKMAEAYSEIGMKGERRRGKGHDGLYQGLSTATKDTYDA | |
| | LHMQALPPR | |
| 66 | MALPVTALLLPLALLLHAARPDYKDDDDKGGGGSGGGGSQVQLV | Anti-CD70 |
| | QSGAEVKKPGASVKVSCKASGYTFTSYDINWVRQAPGQGLEWM | CAR |
| | GVISPSGGGTSYAQKFQGRVTMTRDTSTSTVYMELSSLRSEDTAV | |
| | YYCARYYGGNPEGWYFDLWGRGTLVTVSSGGGGSGGGGSGGG | |
| | GSDIQMTQSPSSLSASVGDRVTITCRASQSIGNWLAWYQQKPGK | |
| | APKLLIYDASDLETGVPSRFSGSGSGTDFTLTISSLQPEDFATYYC | |
| | QQSYTTPWTFGQGTKVEIKTTTPAPRPPTPAPTIASQPLSLRPEAC | |
| | RPAAGGAVHTRGLDFACDIYIWAPLAGTCGVLLLSLVITLYCRRDQ | |
| | RLPPDAHKPPGGGSFRTPIQEEQADAHSTLAKIRVKFSRSADAPA | |
| | YQQGQNQLYNELNLGRREEYDVLDKRRGRDPEMGGKPRRKNPQ | |
| | EGLYNELQKDKMAEAYSEIGMKGERRRGKGHDGLYQGLSTATKD | |
| | TYDALHMQALPPR | |
| 67 | MALPVTALLLPLALLLHAARPDIQMTQSPSSLSASVGDRVTITCRAS | Anti-CD70 |
| | QGIGSWLAWYQQKPGKAPKLLIYAASTLQSGVPSRFSGSGSGTD | CAR |
| | FTLTISSLQPEDFATYYCQQSYSTPWTFGPGTKVDIKGSTSGSGK | |
| | PGSGEGSTKGQVQLVQSGAEVKKPGASVKVSCKASGYTFTSYDI | |
| | NWVRQAPGQGLEWMGVINPSGGGTSYAQKFQGRVTMTRDTSTS | |
| | TVYMELSSLRSEDTAVYYCARYSGYDWVFDYWGQGTLVTVSSAA | |
| | ATTTPAPRPPTPAPTIASQPLSLRPEACRPAAGGAVHTRGLDFAC | |
| | DIYIWAPLAGTCGVLLLSLVITLYCRRDQRLPPDAHKPPGGGSFRT | |
| | PIQEEQADAHSTLAKIRVKFSRSADAPAYQQGQNQLYNELNLGRR | |
| | EEYDVLDKRRGRDPEMGGKPRRKNPQEGLYNELQKDKMAEAYS | |
| | EIGMKGERRRGKGHDGLYQGLSTATKDTYDALHMQALPPR | |
| 68 | MALPVTALLLPLALLLHAARPDIQMTQSPSSLSASVGDRVTITCRAS | Anti-CD70 |
| | QSIGNWLAWYQQKPGKAPKLLIYDASDLETGVPSRFSGSGSGTDF | CAR |
| | TLTISSLQPEDFATYYCQQSYTTPWTFGQGTKVEIKGSTSGSGKP | |
| | GSGEGSTKGQVQLVQSGAEVKKPGASVKVSCKASGYTFTSYDIN | |
| | WVRQAPGQGLEWMGVISPSGGGTSYAQKFQGRVTMTRDTSTST | |
| | VYMELSSLRSEDTAVYYCARYYGGNPEGWYFDLWGRGTLVTVSS | |
| | AAATTTPAPRPPTPAPTIASQPLSLRPEACRPAAGGAVHTRGLDFA | |
| | CDIYIWAPLAGTCGVLLLSLVITLYCRRDQRLPPDAHKPPGGGSFR | |
| | TPIQEEQADAHSTLAKIRVKFSRSADAPAYQQGQNQLYNELNLGR | |
| | REEYDVLDKRRGRDPEMGGKPRRKNPQEGLYNELQKDKMAEAY | |
| | SEIGMKGERRRGKGHDGLYQGLSTATKDTYDALHMQALPPR | |
| 69 | MALPVTALLLPLALLLHAARPDIVMTQSPDSLAVSLGERATINCRAS | Anti-CD70 |
| | KSVSTSGYSFMHWYQQKPGQPPKLLIYLASNLESGVPDRFSGSG | CAR |
| | SGTDFTLTISSLQAEDVAVYYCQHSREVPWTFGQGTKVEIKGSTS | |
| | GSGKPGSGEGSTKGQVQLVQSGAEVKKPGASVKVSCKASGYTFT | |
| | NYGMNWVRQAPGQGLKWMGWINTYTGEPTYADAFKGRVTMTR | |
| | DTSISTAYMELSRLRSDDTAVYYCARDYGDYGMDYWGQGTTVTV | |
| | SSAAATTTPAPRPPTPAPTIASQPLSLRPEACRPAAGGAVHTRGL | |
| | DFACDIYIWAPLAGTCGVLLLSLVITLYCRRDQRLPPDAHKPPGGG | |
| | SFRTPIQEEQADAHSTLAKIRVKFSRSADAPAYQQGQNQLYNELN | |
| | LGRREEYDVLDKRRGRDPEMGGKPRRKNPQEGLYNELQKDKMA | |
| | EAYSEIGMKGERRRGKGHDGLYQGLSTATKDTYDALHMQALPPR | |
| 70 | MALPVTALLLPLALLLHAARPQVQLVQSGAEVKKPGASVKVSCKA | Anti-CD70 |
| | SGYTFTSYDINWVRQAPGQGLEWMGVINPSGGGTSYAQKFQGR | CAR |
| | VTMTRDTSTSTVYMELSSLRSEDTAVYYCARYSGYDWVFDYWGQ | |
| | GTLVTVSSGGGGSGGGGSGGGGSDIQMTQSPSSLSASVGDRVTI | |
| | TCRASQGIGSWLAWYQQKPGKAPKLLIYAASTLQSGVPSRFSGS | |
| | GSGTDFTLTISSLQPEDFATYYCQQSYSTPWTFGPGTKVDIKTTTP | |
| | APRPPTPAPTIASQPLSLRPEACRPAAGGAVHTRGLDFACDIYIWA | |
| | PLAGTCGVLLLSLVITLYCRRDQRLPPDAHKPPGGGSFRTPIQEEQ | |
| | ADAHSTLAKIRVKFSRSADAPAYQQGQNQLYNELNLGRREEYDVL | |
| | DKRRGRDPEMGGKPRRKNPQEGLYNELQKDKMAEAYSEIGMKG | |
| | ERRRGKGHDGLYQGLSTATKDTYDALHMQALPPR | |
| 71 | MALPVTALLLPLALLLHAARPQVQLVQSGAEVKKPGASVKVSCKA | Anti-CD70 |
| | SGYTFTSYDINWVRQAPGQGLEWMGVISPSGGGTSYAQKFQGR | CAR |
| | VTMTRDTSTSTVYMELSSLRSEDTAVYYCARYYGGNPEGWYFDL | |
| | WGRGTLVTVSSGGGGSGGGGSGGGGSDIQMTQSPSSLSASVGD | |
| | RVTITCRASQSIGNWLAWYQQKPGKAPKLLIYDASDLETGVPSRF | |
| | SGSGSGTDFTLTISSLQPEDFATYYCQQSYTTPWTFGQGTKVEIK | |
| | TTTPAPRPPTPAPTIASQPLSLRPEACRPAAGGAVHTRGLDFACDI | |
| | YIWAPLAGTCGVLLLSLVITLYCRRDQRLPPDAHKPPGGGSFRTPI | |
| | QEEQADAHSTLAKIRVKFSRSADAPAYQQGQNQLYNELNLGRRE | |
| | EYDVLDKRRGRDPEMGGKPRRKNPQEGLYNELQKDKMAEAYSEI | |
| | GMKGERRRGKGHDGLYQGLSTATKDTYDALHMQALPPR | |
| 72 | MALPVTALLLPLALLLHAARPQVQLVQSGAEVKKPGASVKVSCKA | Anti-CD70 |
| | SGYTFTNYGMNWVRQAPGQGLKWMGWINTYTGEPTYADAFKGR | CAR |
| | VTMTRDTSISTAYMELSRLRSDDTAVYYCARDYGDYGMDYWGQG | |
| | TTVTVSSGGGGSGGGGSGGGGSDIVMTQSPDSLAVSLGERATIN | |
| | CRASKSVSTSGYSFMHWYQQKPGQPPKLLIYLASNLESGVPDRF | |
| | SGSGSGTDFTLTISSLQAEDVAVYYCQHSREVPWTFGQGTKVEIK | |
| | TTTPAPRPPTPAPTIASQPLSLRPEACRPAAGGAVHTRGLDFACDI | |
| | YIWAPLAGTCGVLLLSLVITLYCRRDQRLPPDAHKPPGGGSFRTPI | |
| | QEEQADAHSTLAKIRVKFSRSADAPAYQQGQNQLYNELNLGRRE | |
| | EYDVLDKRRGRDPEMGGKPRRKNPQEGLYNELQKDKMAEAYSEI | |
| | GMKGERRRGKGHDGLYQGLSTATKDTYDALHMQALPPR | |
| 73 | QVQLVQSGAEVKKPGASVKVSCKASG | FW-H1_1 |
| 74 | QVQLVQSGAEVKKPGSSVKVSCKASG | FW-H1_2 |
| 75 | EVQLLESGGGLVQPGGSLRLSCAASG | FW-H1_3 |
| 76 | EVQLVESGGGLVKPGGSLRLSCAASG | FW-H1_4 |
| 77 | WVRQAPGQGLEWM | FW-H2_1 |
| 78 | WVRQAPGQGLEWV | FW-H2_2 |
| 79 | WVRQAPGKGLEWV | FW-H2_3 |
| 80 | WRQAPGQGLEWI | FW-H2_4 |
| 81 | QKFQGRVTMTRDTSTSTVYMELSSLRSEDTAVYY | FW-H3_1 |
| 82 | QRFQGRVTMTRDTSTSTVYMELSSLRSEDTAVYY | FW-H3_2 |
| 83 | HKFQGRVTMTRDTSTSTVYMELSSLRSEDTAVYY | FW-H3_3 |
| 84 | QKFQGRVTITADESTSTAYMELSSLRSEDTAVYY | FW-H3_4 |
| 85 | LKFQGRVTMTRDTSTSTVYMELSSLRSEDTAVYY | FW-H3_5 |
| 86 | QNFQGRVTMTRDTSTSTVYMELSSLRSEDTAVYY | FW-H3_6 |
| 87 | QMFQGRVTMTRDTSTSTVYMELSSLRSEDTAVYY | FW-H3_7 |
| 88 | QEFQGRVTMTRDTSTSTVYMELSSLRSEDTAVYY | FW-H3_8 |
| 89 | EEFQGRVTITADESTSTAYMELSSLRSEDTAVYY | FW-H3_9 |
| 90 | QTFQGRVTMTRDTSTSTVYMELSSLRSEDTAVYY | FW-H3_10 |
| 91 | QSFQGRVTMTRDTSTSTVYMELSSLRSEDTAVYY | FW-H3_11 |
| 92 | DSVKGRFTISRDNSKNTLYLQMNSLRAEDTAVYY | FW-H3_12 |
| 93 | QNLQGRVTMTRDTSTSTVYMELSSLRSEDTAVYY | FW-H3_13 |
| 94 | PKFQGRVTITADESTSTAYMELSSLRSEDTAVYY | FW-H3_14 |
| 95 | DSVKGRFTISRDDSKNTLYLQMNSLKTEDTAVYY | FW-H3_15 |
| 96 | DKFQGRVTMTRDTSTSTVYMELSSLRSEDTAVYY | FW-H3_16 |
| 97 | GQGTLVTVSS | FW-H4_1 |
| 98 | GRGTLVTVSS | FW-H4_2 |
| 99 | GQGTTVTVSS | FW-H4_3 |
| 100 | GQGTMVTVSS | FW-H4_4 |
| 101 | GKGTTVTVSS | FW-H4_5 |
| 102 | YTFTDYYMH | CDR-H1 |
| 103 | YTFTSYDIN | CDR-H1 |
| 104 | GIINPNSGGTNYA | CDR-H2 |
| 105 | GVINPSGGGTSYA | CDR-H2 |
| 106 | GVISPSGGGTSYA | CDR-H2 |
| 107 | CARDFSVYAAADGMDVW | CDR-H3 |
| 108 | CARYSGYDWVFDYW | CDR-H3 |
| 109 | CARYYGGNPEGWYFDLW | CDR-H3 |
| 110 | NYGMN | CDR-H1 |
| 111 | WINTYTGEPTYADAFKG | CDR-H2 |
| 112 | GDYGMDY | CDR-H3 |
| 113 | DIQMTQSPSSLSASVGDRVTITC | FW-L1_1 |
| 114 | DIVMTQSPLSLPVTPGEPASISC | FW-L1_2 |
| 115 | EIVMTQSPATLSVSPGERATLSC | FW-L1_3 |
| 116 | DIVMTQSPDSLAVSLGERATINC | FW-L1_4 |
| 117 | WYQQKPGKAPKLLIY | FW-L2_1 |
| 118 | WYLQKPGQSPQLLIY | FW-L2_2 |
| 119 | WYQQKPGQAPRLLIY | FW-L2_3 |
| 120 | WYQQKPGQPPKLLIY | FW-L2_4 |
| 121 | GVPSRFSGSGSGTDFTLTISSLQPEDFATYY | FW-L3_1 |
| 122 | GVPDRFSGSGSGTDFTLKISRVEAEDVGVYY | FW-L3_2 |
| 123 | GIPARFSGSGSGTEFTLTISSLQSEDFAVYY | FW-L3_3 |
| 124 | GVPDRFSGSGSGTDFTLTISSLQAEDVAVYY | FW-L3_4 |
| 125 | GQGTRLEIK | FW-L4_1 |
| 126 | GQGTKLEIK | FW-L4_2 |
| 127 | GQGTKVEIK | FW-L4_3 |
| 128 | GGGTKVEIK | FW-L4_4 |
| 129 | GPGTKVDIK | FW-L4_5 |
| 130 | GGGTKLEIK | FW-L4_6 |
| 131 | RASQSISSYLN | CDR-L1 |
| 132 | RASQGIGSWLA | CDR-L1 |
| 133 | RASQSIGNWLA | CDR-L1 |
| 134 | DASNLAT | CDR-L2 |
| 135 | AASTLQS | CDR-L2 |
| 136 | DASDLET | CDR-L2 |
| 137 | CQQSDSSPLTF | CDR-L3 |
| 138 | CQQSYSTPWTF | CDR-L3 |
| 139 | CQQSYTTPWTF | CDR-L3 |
| 140 | RASKSVSTSGYSFMH | CDR-L1 |
| 141 | LASNLES | CDR-L2 |
| 142 | QHSREVPWT | CDR-L3 |
| 143 | caggtgcagctggtgcagtctggggctgaggtgaagaagcctggggcctcagtgaaggtttc | Anti-CD70 VH |
| | ctgcaaggcatctggatacaccttcaccgattactatatgcactgggtgcgacaggcccctgg | |
| | acaagggcttgagtggatgggaataatcaaccctaatagtggtggaacaaattacgcacag | |
| | aagttccagggcagagtcaccatgaccagggacacgtccacgagcacagtctacatggag | |
| | ctgagcagcctgagatctgaggacacggccgtgtattactgtgctagagattttagtgtgtatgc | |
| | tgctgcagatggcatggatgtctggggccaagggacattggtcaccgtctcttca | |
| 144 | caggtgcagctggtgcagtctggggctgaggtgaagaagcctggggcctcagtgaaggtttc | Anti-CD70 VH |
| | ctgcaaggcatctggatacaccttcaccagctacgacattaattgggtgcgacaggcccctgg | |
| | acaagggcttgagtggatgggagtgatcaaccctagtggtggaggtacaagctacgcacag | |
| | aagttccagggcagagtcaccatgaccagggacacgtccacgagcacagtctacatggag | |
| | atgtagcggtctattattgccagcacagtagagaagtcccctggaccttcggtcaaggcacga | |
| | aagtagaaattaaa | |
| 151 | QVQLVQSGAEVKKPGASVKVSCKASGYTFTDYYMHWVRQAPGQ | Anti-CD70 VH |
| | GLEWMGIINPNSGGTNYAQKFQGRVTMTRDTSTSTVYMELSSLR | |
| | SEDTAVYYCARDFSVYAAADGMDVWGQGTLVTVSS | |
| 152 | QVQLVQSGAEVKKPGASVKVSCKASGYTFTSYDINWVRQAPGQG | Anti-CD70 VH |
| | LEWMGVINPSGGGTSYAQKFQGRVTMTRDTSTSTVYMELSSLRS | |
| | EDTAVYYCARYSGYDWVFDYWGQGTLVTVSS | |
| 153 | QVQLVQSGAEVKKPGASVKVSCKASGYTFTSYDINWVRQAPGQG | Anti-CD70 VH |
| | LEWMGVISPSGGGTSYAQKFQGRVTMTRDTSTSTVYMELSSLRS | |
| | EDTAVYYCARYYGGNPEGWYFDLWGRGTLVTVSS | |
| 154 | DIQMTQSPSSLSASVGDRVTITCRASQSISSYLNWYQQKPGKAPK | Anti-CD70 VL |
| | LLIYDASNLATGVPSRFSGSGSGTDFTLTISSLQPEDFATYYCQQS | |
| | DSSPLTFGQGTKVEIK | |
| 155 | DIQMTQSPSSLSASVGDRVTITCRASQGIGSWLAWYQQKPGKAP | Anti-CD70 VL |
| | KLLIYAASTLQSGVPSRFSGSGSGTDFTLTISSLQPEDFATYYCQQ | |
| | SYSTPWTFGPGTKVDIK | |
| 156 | DIQMTQSPSSLSASVGDRVTITCRASQSIGNWLAWYQQKPGKAPK | Anti-CD70 VL |
| | LLIYDASDLETGVPSRFSGSGSGTDFTLTISSLQPEDFATYYCQQS | |
| | YTTPWTFGQGTKVEIK | |
| 157 | QVQLVQSGAEVKKPGASVKVSCKASGYTFTNYGMNWVRQAPGQ | Anti-CD70 VH |
| | GLKWMGWINTYTGEPTYADAFKGRVTMTRDTSISTAYMELSRLRS | |
| | DDTAVYYCARDYGDYGMDYWGQGTTVTVSS | |
| 158 | DIVMTQSPDSLAVSLGERATINCRASKSVSTSGYSFMHWYQQKP | Anti-CD70 VL |
| | GQPPKLLIYLASNLESGVPDRFSGSGSGTDFTLTISSLQAEDVAVY | |
| | YCQHSREVPWTFGQGTKVEIK | |
| 159 | ccgatcgtgaagcgcctgct | SMAD3 gRNA |
| 160 | cgagaaggcggtcaagagcc | SMAD3 gRNA |
| 161 | cttggtgttgacgttctgcg | SMAD3 gRNA |
| 162 | ctctgctgtttcaccatcca | MAPKAPK |
| | | gRNA |
| 163 | cccggcttgggcggtgctcc | MAPKAPK |
| | | gRNA |
| 164 | cgactaccagttgtccaagc | MAPKAPK |
| | | gRNA |
| 165 | gactgagttattggcgtggc | CEACAM1 |
| | | gRNA |
| 166 | gaatgttccattgataagcc | CEACAM1 |
| | | gRNA |
| 167 | gagaggctgaggtttgcccc | CEACAM1 |
| | | gRNA |
| 168 | cctcaccatgcctagccttt | DDIT4 gRNA |
| 169 | cgatctgggggggagttcg | DDIT4 gRNA |
| 170 | gtttgaccgctccacgagcc | DDIT4 gRNA |
| 171 | cccctaccatgactttattc | TGFBR2 gRNA |
| 172 | attgcactcatcagagctac | TGFBR2 gRNA |
| 173 | agtcatggtaggggagcttg | TGFBR2 gRNA |
| 174 | tgctggcgatacgcgtccac | TGFBR2 gRNA |
| 175 | gtgagcaatcccccgggcga | TGFBR2 gRNA |
| 176 | aacgtgcggtgggatcgtgc | TGFBR2 gRNA |
| 177 | tcaccaagcccgcgaccaatggg | CD70 gRNA |
| 178 | gctttggtcccattggtcgcggg | CD70 gRNA |
| 179 | accctcctccggcatcgccgcgg | CD70 gRNA |
| 180 | gctttggtcccattggtcgc | CD70 gRNA |
| 181 | ctcaccagattcccgaaggt | CISH gRNA |
| 182 | ccgccttgtcatcaaccgtc | CISH gRNA |
| 183 | tctgcgttcaggggtaagcg | CISH gRNA |
| 184 | gcgcttacccctgaacgcag | CISH gRNA |
| 185 | cgcagaggaccatgtccccg | CISH gRNA |
| 186 | agattcccgaaggtaggaga | CISH gRNA |
| 187 | ctggtattggggttccatta | CISH gRNA |
| 188 | tactcaatgcgtacattggt | CISH gRNA |
| 189 | atactcaatgcgtacattgg | CISH gRNA |
| 190 | tccggaaaggccaggatgcg | CISH gRNA |
| 191 | cttgtccaggccacgcatcc | CISH gRNA |
| 192 | taatctggtggacctcatgaagg | CBLB gRNA |
| 193 | tcggttggcaaacgtccgaaagg | CBLB gRNA |
| 194 | agcaagctgccgcagatcgcagg | CBLB gRNA |
| 195 | agcaagctgccgcagatcgc | CBLB gRNA |
| 196 | ggagctgatggtaaatctgc | NKG2A gRNA |
| 197 | ttgaaggtttaattccgcat | NKG2A gRNA |
| 198 | aacaactatcgttaccacag | NKG2A gRNA |
| 199 | cgaggagcccatgatgggca | A2AR gRNA |
| 200 | gggcaatgtgctggtgtgct | A2AR gRNA |
| 201 | cagtgacaccacaaagtagt | A2AR gRNA |
| 202 | tcaccaagcccgcgaccaatggg | mock gRNA |
| 203 | ucaccaagcccgcgaccaauggg | mock gRNA |
| 204 | QGIGSW | CDR-L3 |
| 205 | GYTFTSYD | CDR-H1 |
| 206 | ISPSGGGT | CDR-H2 |
| 207 | ARYYGGNPEGWYFDL | CDR-H3 |
| 208 | GGGGSGGGGSGGGGS | Linker |
| 209 | VINPSGGGTSYAQKFQGQSIGNW | CDR-L1 |
| 210 | YSGYDWVFDYDA | CDR-L2 |
| 211 | QQSYTTPWT | CDR-L3 |
| 212 | agtggttcgggcgctggttaccc | Target |
| | | sequence |
| 213 | NWVNVISDLKKIEDLIQSMHIDATLYTESDVHPSCKVTAMKCFLLEL | Membrane- |
| | QVISLESGDASIHDTVENLIILANNSLSSNGNVTESGCKECEELEEK | bound human |
| | NIKEFLQSFVHIVQMFINTSTTTPAPRPPTPAPTIASQPLSLRPEAC | IL15 (mbIL15) |
| | RPAAGGAVHTRGLDFACDIYIWAPLAGTCGVLLLSLVITLYC | |
| 214 | QVQLVQSGAEVKKPGASVKVSCKASGYTFTDYYMHWVRQAPGQ | Anti-CD70 |
| | GLEWMGIINPNSGGTNYAQKFQGRVTMTRDTSTSTVYMELSSLR | CAR |
| | SEDTAVYYCARDFSVYAAADGMDVWGQGTLVTVSSGGGGSGGG | |
| | GSGGGGSDIQMTQSPSSLSASVGDRVTITCRASQSISSYLNWYQ | |
| | QKPGKAPKLLIYDASNLATGVPSRFSGSGSGTDFTLTISSLQPEDF | |
| | ATYYCQQSDSSPLTFGQGTKVEIKTTTPAPRPPTPAPTIASQPLSL | |
| | RPEACRPAAGGAVHTRGLDFACDIYIWAPLAGTCGVLLLSLVITLY | |
| | CRRDQRLPPDAHKPPGGGSFRTPIQEEQADAHSTLAKIRVKFSRS | |
| | ADAPAYQQGQNQLYNELNLGRREEYDVLDKRRGRDPEMGGKPR | |
| | RKNPQEGLYNELQKDKMAEAYSEIGMKGERRRGKGHDGLYQGL | |
| | STATKDTYDALHMQALPPR | |
| 215 | QVQLVQSGAEVKKPGASVKVSCKASGYTFTSYDINWVRQAPGQG | Anti-CD70 |
| | LEWMGVINPSGGGTSYAQKFQGRVTMTRDTSTSTVYMELSSLRS | CAR |
| | EDTAVYYCARYSGYDWVFDYWGQGTLVTVSSGGGGSGGGGSG | |
| | GGGSDIQMTQSPSSLSASVGDRVTITCRASQGIGSWLAWYQQKP | |
| | GKAPKLLIYAASTLQSGVPSRFSGSGSGTDFTLTISSLQPEDFATY | |
| | YCQQSYSTPWTFGPGTKVDIKTTTPAPRPPTPAPTIASQPLSLRPE | |
| | ACRPAAGGAVHTRGLDFACDIYIWAPLAGTCGVLLLSLVITLYCRR | |
| | DQRLPPDAHKPPGGGSFRTPIQEEQADAHSTLAKIRVKFSRSADA | |
| | PAYQQGQNQLYNELNLGRREEYDVLDKRRGRDPEMGGKPRRKN | |
| | PQEGLYNELQKDKMAEAYSEIGMKGERRRGKGHDGLYQGLSTAT | |
| | KDTYDALHMQALPPR | |
| 216 | QVQLVQSGAEVKKPGASVKVSCKASGYTFTSYDINWVRQAPGQG | Anti-CD70 |
| | LEWMGVISPSGGGTSYAQKFQGRVTMTRDTSTSTVYMELSSLRS | CAR |
| | EDTAVYYCARYYGGNPEGWYFDLWGRGTLVTVSSGGGGSGGG | |
| | GSGGGGSDIQMTQSPSSLSASVGDRVTITCRASQSIGNWLAWYQ | |
| | QKPGKAPKLLIYDASDLETGVPSRFSGSGSGTDFTLTISSLQPEDF | |
| | ATYYCQQSYTTPWTFGQGTKVEIKTTTPAPRPPTPAPTIASQPLSL | |
| | RPEACRPAAGGAVHTRGLDFACDIYIWAPLAGTCGVLLLSLVITLY | |
| | CRRDQRLPPDAHKPPGGGSFRTPIQEEQADAHSTLAKIRVKFSRS | |
| | ADAPAYQQGQNQLYNELNLGRREEYDVLDKRRGRDPEMGGKPR | |
| | RKNPQEGLYNELQKDKMAEAYSEIGMKGERRRGKGHDGLYQGL | |
| | STATKDTYDALHMQALPPR | |
| 217 | DIQMTQSPSSLSASVGDRVTITCRASQGIGSWLAWYQQKPGKAP | Anti-CD70 |
| | KLLIYAASTLQSGVPSRFSGSGSGTDFTLTISSLQPEDFATYYCQQ | CAR |
| | SYSTPWTFGPGTKVDIKGSTSGSGKPGSGEGSTKGQVQLVQSGA | |
| | EVKKPGASVKVSCKASGYTFTSYDINWVRQAPGQGLEWMGVINP | |
| | SGGGTSYAQKFQGRVTMTRDTSTSTVYMELSSLRSEDTAVYYCA | |
| | RYSGYDWVFDYWGQGTLVTVSSAAATTTPAPRPPTPAPTIASQPL | |
| | SLRPEACRPAAGGAVHTRGLDFACDIYIWAPLAGTCGVLLLSLVITL | |
| | YCRRDQRLPPDAHKPPGGGSFRTPIQEEQADAHSTLAKIRVKFSR | |
| | SADAPAYQQGQNQLYNELNLGRREEYDVLDKRRGRDPEMGGKP | |
| | RRKNPQEGLYNELQKDKMAEAYSEIGMKGERRRGKGHDGLYQG | |
| | LSTATKDTYDALHMQALPPR | |
| 218 | DIQMTQSPSSLSASVGDRVTITCRASQSIGNWLAWYQQKPGKAPK | Anti-CD70 |
| | LLIYDASDLETGVPSRFSGSGSGTDFTLTISSLQPEDFATYYCQQS | CAR |
| | YTTPWTFGQGTKVEIKGSTSGSGKPGSGEGSTKGQVQLVQSGAE | |
| | VKKPGASVKVSCKASGYTFTSYDINWVRQAPGQGLEWMGVISPS | |
| | GGGTSYAQKFQGRVTMTRDTSTSTVYMELSSLRSEDTAVYYCAR | |
| | YYGGNPEGWYFDLWGRGTLVTVSSAAATTTPAPRPPTPAPTIASQ | |
| | PLSLRPEACRPAAGGAVHTRGLDFACDIYIWAPLAGTCGVLLLSLV | |
| | ITLYCRRDQRLPPDAHKPPGGGSFRTPIQEEQADAHSTLAKIRVKF | |
| | SRSADAPAYQQGQNQLYNELNLGRREEYDVLDKRRGRDPEMGG | |
| | KPRRKNPQEGLYNELQKDKMAEAYSEIGMKGERRRGKGHDGLY | |
| | QGLSTATKDTYDALHMQALPPR | |
| 219 | DIVMTQSPDSLAVSLGERATINCRASKSVSTSGYSFMHWYQQKP | Anti-CD70 |
| | GQPPKLLIYLASNLESGVPDRFSGSGSGTDFTLTISSLQAEDVAVY | CAR |
| | YCQHSREVPWTFGQGTKVEIKGSTSGSGKPGSGEGSTKGQVQL | |
| | VQSGAEVKKPGASVKVSCKASGYTFTNYGMNWVRQAPGQGLKW | |
| | MGWINTYTGEPTYADAFKGRVTMTRDTSISTAYMELSRLRSDDTA | |
| | VYYCARDYGDYGMDYWGQGTTVTVSSAAATTTPAPRPPTPAPTI | |
| | ASQPLSLRPEACRPAAGGAVHTRGLDFACDIYIWAPLAGTCGVLLL | |
| | SLVITLYCRRDQRLPPDAHKPPGGGSFRTPIQEEQADAHSTLAKIR | |
| | VKFSRSADAPAYQQGQNQLYNELNLGRREEYDVLDKRRGRDPE | |
| | MGGKPRRKNPQEGLYNELQKDKMAEAYSEIGMKGERRRGKGHD | |
| | GLYQGLSTATKDTYDALHMQALPPR | |
| 220 | QVQLVQSGAEVKKPGASVKVSCKASGYTFTSYDINWVRQAPGQG | Anti-CD70 |
| | LEWMGVINPSGGGTSYAQKFQGRVTMTRDTSTSTVYMELSSLRS | CAR |
| | EDTAVYYCARYSGYDWVFDYWGQGTLVTVSSGGGGSGGGGSG | |
| | GGGSDIQMTQSPSSLSASVGDRVTITCRASQGIGSWLAWYQQKP | |
| | GKAPKLLIYAASTLQSGVPSRFSGSGSGTDFTLTISSLQPEDFATY | |
| | YCQQSYSTPWTFGPGTKVDIKTTTPAPRPPTPAPTIASQPLSLRPE | |
| | ACRPAAGGAVHTRGLDFACDIYIWAPLAGTCGVLLLSLVITLYCRR | |
| | DQRLPPDAHKPPGGGSFRTPIQEEQADAHSTLAKIRVKFSRSADA | |
| | PAYQQGQNQLYNELNLGRREEYDVLDKRRGRDPEMGGKPRRKN | |
| | PQEGLYNELQKDKMAEAYSEIGMKGERRRGKGHDGLYQGLSTAT | |
| | KDTYDALHMQALPPR | |
| 221 | QVQLVQSGAEVKKPGASVKVSCKASGYTFTSYDINWVRQAPGQG | Anti-CD70 |
| | LEWMGVISPSGGGTSYAQKFQGRVTMTRDTSTSTVYMELSSLRS | CAR |
| | EDTAVYYCARYYGGNPEGWYFDLWGRGTLVTVSSGGGGSGGG | |
| | GSGGGGSDIQMTQSPSSLSASVGDRVTITCRASQSIGNWLAWYQ | |
| | QKPGKAPKLLIYDASDLETGVPSRFSGSGSGTDFTLTISSLQPEDF | |
| | ATYYCQQSYTTPWTFGQGTKVEIKTTTPAPRPPTPAPTIASQPLSL | |
| | RPEACRPAAGGAVHTRGLDFACDIYIWAPLAGTCGVLLLSLVITLY | |
| | CRRDQRLPPDAHKPPGGGSFRTPIQEEQADAHSTLAKIRVKFSRS | |
| | ADAPAYQQGQNQLYNELNLGRREEYDVLDKRRGRDPEMGGKPR | |
| | RKNPQEGLYNELQKDKMAEAYSEIGMKGERRRGKGHDGLYQGL | |
| | STATKDTYDALHMQALPPR | |
| 222 | QVQLVQSGAEVKKPGASVKVSCKASGYTFTNYGMNWVRQAPGQ | Anti-CD70 |
| | GLKWMGWINTYTGEPTYADAFKGRVTMTRDTSISTAYMELSRLRS | CAR |
| | DDTAVYYCARDYGDYGMDYWGQGTTVTVSSGGGGSGGGGSGG | |
| | GGSDIVMTQSPDSLAVSLGERATINCRASKSVSTSGYSFMHWYQ | |
| | QKPGQPPKLLIYLASNLESGVPDRFSGSGSGTDFTLTISSLQAEDV | |
| | AVYYCQHSREVPWTFGQGTKVEIKTTTPAPRPPTPAPTIASQPLSL | |
| | RPEACRPAAGGAVHTRGLDFACDIYIWAPLAGTCGVLLLSLVITLY | |
| | CRRDQRLPPDAHKPPGGGSFRTPIQEEQADAHSTLAKIRVKFSRS | |
| | ADAPAYQQGQNQLYNELNLGRREEYDVLDKRRGRDPEMGGKPR | |
| | RKNPQEGLYNELQKDKMAEAYSEIGMKGERRRGKGHDGLYQGL | |
| | STATKDTYDALHMQALPPR | |
| 223 | AA | CDR-L2 |
| 224 | QQSYSTPWT | CDR-L3 |
| 225 | INPSGGGT | CDR-H2 |
| 226 | ARYSGYDWVFDY | CDR-H3 |

1-98. (canceled)

99. An anti-CD70 binding domain that is a single-chain variable fragment (scFv) comprising a variable heavy chain region (VH) and a variable light chain region (VL) coupled by a linker comprising the amino acid sequence set forth in SEQ ID NO: 50, wherein:
(i) the VH comprises a complementarity-determining region (CDR) 1, a CDR2, and a CDR3 comprising the amino acid sequences set forth in SEQ ID NOS: 205, 206, and 207, respectively; and the VL comprises a CDR1, a CDR2, and a CDR3 comprising the amino acid sequences set forth in SEQ ID NOS: 209, 210, and 211, respectively;
(ii) the VH comprises a CDR1, a CDR2, and a CDR3 comprising the sequences of SEQ ID NOS: 205, 225, and 226, respectively; and the VL comprises a CDR1, a CDR2, and a CDR3 comprising the sequences of SEQ ID NOS: 204, 223, and 224, respectively; or
(iii) the VH comprises a CDR1, a CDR2, and a CDR3 comprising the sequences of SEQ ID NOS: 110, 111, and 112, respectively; and the VL comprises a CDR1, a CDR2, and a CDR3 comprising the sequences of SEQ ID NOS: 140, 141, and 142, respectively.

100. The anti-CD70 binding domain of claim 99, wherein:
(i) the VH comprises an amino acid sequence having at least 90% sequence identity to SEQ ID NO: 153, and the VL comprises an amino acid sequence having at least 90% sequence identity to SEQ ID NO: 156;
(ii) the VH comprises an amino acid sequence having at least 90% sequence identity to SEQ ID NO: 152, and the VL comprises an amino acid sequence having at least 90% sequence identity to SEQ ID NO: 155; or
(iii) the VH comprises an amino acid sequence having at least 90% sequence identity to SEQ ID NO: 157, and the VL comprises an amino acid sequence having at least 90% sequence identity to SEQ ID NO: 158.

101. The anti-CD70 binding domain of claim 99, wherein:
(i) the VH comprises the amino acid sequence set forth in SEQ ID NO: 153, and the VL comprises the amino acid sequence set forth in SEQ ID NO: 156;
(ii) the VH comprises the amino acid sequence set forth in SEQ ID NO: 152, and the VL comprises the amino acid sequence set forth in SEQ ID NO: 155; or
(iii) the VH comprises the amino acid sequence set forth in SEQ ID NO: 157, and the VL comprises the amino acid sequence set forth in SEQ ID NO: 158.

102. The anti-CD70 binding domain of claim 99, wherein the scFv comprises the amino acid sequence set forth in SEQ ID NO: 52, SEQ ID NO: 51, or SEQ ID NO: 53.

103. An anti-CD70 chimeric antigen receptor (CAR) comprising the anti-CD70 binding domain of claim 99.

104. The anti-CD70 CAR of claim 103, comprising (a) a transmembrane domain and (b) a cytotoxic signaling complex comprising a CD3zeta subdomain.

105. The anti-CD70 CAR of claim 104, wherein the cytotoxic signaling complex comprises an OX40 signaling domain, a CD28 signaling domain, or a 4-1BB signaling domain.

106. An immune cell comprising the anti-CD70 CAR of claim 103.

107. The immune cell of claim 106, wherein the immune cell is genetically edited to reduce expression of CD70, CISH, and/or CBLB.

108. The immune cell of claim 106, wherein the immune cell is a natural killer (NK) cell.

109. A composition comprising a plurality of the immune cells of claim 106.

110. A method of treating a cancer in a subject comprising administering the composition of claim 109 to the subject.

111. A population of genetically engineered natural killer (NK) cells comprising a plurality of NK cells engineered to express a chimeric antigen receptor (CAR) comprising a tumor binding domain that targets CD70, a transmembrane domain, and a cytotoxic signaling complex, wherein the genetically engineered NK cells comprise:
a genomic disruption within a target sequence of a CD70-encoding gene, the target sequence comprising any one of SEQ ID NOS: 180 or 177-179; and
a genomic disruption within a target sequence of a cytokine-inducible SH2-containing (CIS)-encoding gene, the target sequence comprising any one of SEQ ID NOS: 191 or 186-190.

112. The population of genetically engineered NK cells of claim 111, wherein the genetically engineered NK cells comprise a genomic disruption with a target sequence of a Casitas B-lineage lymphoma-b (CBLB)-encoding gene.

113. The population of genetically engineered NK cells of claim 112, wherein the target sequence of the CBLB-encoding gene comprises any one of SEQ ID NOS: 195 and 192-194.

114. The population of genetically engineered NK cells of claim 112, wherein the genomic disruption within the target sequence of the CD70-encoding gene, the target sequence of the CIS-encoding gene, and/or the target sequence of the CBLB-encoding gene comprises an endonuclease-mediated indel.

115. The population of genetically engineered NK cells of claim 111, wherein the tumor binding domain comprises a single-chain variable fragment (scFv) comprising a heavy chain variable region (VH) and a light chain variable region (VL), wherein:
(i) the VH comprises a complementarity-determining region (CDR) 1, a CDR2, and a CDR3 comprising the sequences of SEQ ID NOS: 205, 206, and 207, respectively; and the VL comprises a CDR1, a CDR2, and a CDR3 comprising the sequences of SEQ ID NOS: 209, 210, and 211, respectively;
(ii) the VH comprises a CDR1, a CDR2, and a CDR3 comprising the sequences of SEQ ID NOS: 205, 225, and 226, respectively; and the VL comprises a CDR1, a CDR2, and a CDR3 comprising the sequences of SEQ ID NOS: 204, 223, and 224, respectively; or
(iii) the VH comprises a CDR1, a CDR2, and a CDR3 comprising the sequences of SEQ ID NOS: 110, 111, and 112, respectively; and the VL comprises a CDR1, a CDR2, and a CDR3 comprising the sequences of SEQ ID NOS: 140, 141, and 142, respectively.

116. The population of genetically engineered NK cells of claim 115, wherein:
(i) the VH comprises an amino acid sequence having at least 90% sequence identity to SEQ ID NO: 153, and the VL comprises an amino acid sequence having at least 90% sequence identity to SEQ ID NO: 156;
(ii) the VH comprises an amino acid sequence having at least 90% sequence identity to SEQ ID NO: 152, and the VL comprises an amino acid sequence having at least 90% sequence identity to SEQ ID NO: 155; or
(iii) the VH comprises an amino acid sequence having at least 90% sequence identity to SEQ ID NO: 157, and the VL comprises an amino acid sequence having at least 90% sequence identity to SEQ ID NO: 158.

117. The population of genetically engineered NK cells of claim 115, wherein:
(i) the VH comprises the amino acid sequence set forth in SEQ ID NO: 153, and the VL comprises the amino acid sequence set forth in SEQ ID NO: 156;
(ii) the VH comprises the amino acid sequence set forth in SEQ ID NO: 152, and the VL comprises the amino acid sequence set forth in SEQ ID NO: 155; or
(iii) the VH comprises the amino acid sequence set forth in SEQ ID NO: 157, and the VL comprises the amino acid sequence set forth in SEQ ID NO: 158.

118. A composition comprising the population of genetically engineered NK cells of claim 111.

119. A method of treating a cancer in a subject comprising administering the population of genetically engineered NK cells of claim 111 to the subject.

120. A method for generating a population of genetically engineered immune cells, comprising:
expanding the immune cells in culture for a period of time;
introducing an endonuclease and no more than two unique guide RNAs (gRNAs) into the immune cells to induce a genomic disruption within two distinct gene target sequences;
culturing the immune cells for an additional period of time;
introducing an additional endonuclease and no more than two additional unique gRNAs into the immune cells to induce additional genomic disruptions within no more than two additional gene target sequences;
and transducing the immune cells with a viral vector encoding a CD70-targeting CAR,
wherein the gRNAs target CD70-, CIS-, and/or CBLB-encoding genes.
